(19) United States
(12) Patent Application Publication
(10) Pub. No.: US 2026/0250404 A1
(43) Pub. Date: Aug. 27, 2026

(54) BINDING AGENTS CAPABLE OF BINDING TO CD27 IN COMBINATION THERAPY

(71) Applicants: GENMAB A/S, Valby (DK); BIONTECH SE, Mainz (DE)

(72) Inventors: Esther C. W. BREIJ, Utrecht (NL); Ugur SAHIN, Mainz (DE); Isil ALTINTAS, Utrecht (NL); Patricia GARRIDO CASTRO, Utrecht (NL); Jordan BLUM, Utrecht (NL); Anna WOJTUSZKIEWICZ, Utrecht (NL); Lars GUELEN, Utrecht (NL); Joost J. NEIJSSEN, Utrecht (NL); Andreea IOAN, Utrecht (NL); Frank BEURSKENS, Utrecht (NL); Rob N. DE JONG, Utrecht (NL); Janine SCHUURMAN, Utrecht (NL); Pauline Linda DE GOEJE, Utrecht (NL); David SATIJN, Utrecht (NL); Peter BOROSS, Utrecht (NL); Bart-Jan DE KREUK, Utrecht (NL); Richard HIBBERT, Utrecht (NL); Aran F. LABRIJN, Utrecht (NL); Kristina NÜRMBERGER, Mainz (DE); Sina FELLERMEIER-KOPF, Mainz (DE); Friederike GIESEKE, Mainz (DE); Alexander MUIK, Mainz (DE); Karsten BECKMANN, Mainz (DE); Claudia PAULMANN, Mainz (DE); Ivan KUZMANOV, Mainz (DE)

(21) Appl. No.: 18/861,665

(22) PCT Filed: May 12, 2023
(86) PCT No.: PCT/EP2023/062793

§ 371 Date: Oct. 30, 2024

Related U.S. Application Data
(60) Provisional application No. 63/341,397, filed on May 12, 2022.

(51) Int. Cl. C07K 16/28

(52) U.S. Cl. CPC C07K 16/2878; C07K 16/2818; C07K 16/2827; C07K 2317/31; C07K 2317/565; C07K 2317/75; C07K 2317/76; C07K 2317/92

(57) ABSTRACT

The present invention provides combination therapy using a binding agent comprises at least one binding region binding to CD27 in combination with a PD1/PD-L 1 inhibitor to reduce progression or prevent progression of a tumor or treating cancer.

### RELATED APPLICATIONS

This application is a 35 U.S.C. § 371 filing of International Patent Application No. PCT/EP2023/062793, filed May 12, 2023, which claims priority to U.S. Provisional Patent Application Ser. No. 63/341,397, filed May 12, 2022, the entire disclosures of which are hereby incorporated herein by reference.

### SEQUENCE LISTING

The instant application contains a Sequence Listing which has been submitted electronically in XML format and is hereby incorporated by reference in its entirety. Said XML file, created on Oct. 28, 2024, is named 758985_GMB9-013US_ST26.xml and is 147,880 bytes in size.

### TECHNICAL FIELD

The present invention relates to combination therapy using a binding agent comprises at least one binding region binding to CD27 in combination with a PD1/PD-L1 inhibitor to reduce progression or prevent progression of a tumor or treating cancer.

### BACKGROUND

Cluster of differentiation (CD)27 (TNFRSF7) is a 55 kDa type I transmembrane protein member of the tumor necrosis factor (TNF) receptor superfamily (TNFRSF) which co-stimulates T-cell activation after binding to its ligand CD70. It is expressed in humans on the cell membrane of T, B, natural killer (NK) cells, and their immediate precursors, all of them part of the lymphoid lineage. On human T cells, CD27 is expressed on resting ap CD4+(Treg and conventional T cells), CD8⁺ T cells, stem-cell memory cells, and central-memory-like cells. On human B cells, CD27 is a memory B cell marker and CD27 signaling promotes differentiation of B cells into plasma cells.

The only known ligand for CD27 is the type II transmembrane protein CD70 (tumor necrosis factor superfamily member 7, TNFSF7; CD27 ligand, CD27L), which is quite restrictively and only transiently expressed on activated immune cells, including T, B, NK, and dendritic cells (DCs).

CD27 plays a role in early generation of a primary immune response and is required for generation and long-term maintenance of T-cell immunity. CD27-CD70 binding leads to activation of nuclear factor kappa-light-chain-enhancer of activated B cells (NF-κB) and mitogen-activated protein kinase (MAPK)8/Jun N-terminal kinase (JNK) pathways. Adaptor proteins TNF receptor-associated protein (TRAF)2 and TRAF5 have been shown to mediate the signaling resulting from CD27 engagement.

To unlock their effector functions, T cells require T-cell antigen receptor-mediated recognition of their cognate antigen in the context of major histocompatibility complex (MHC) molecules on the surface of antigen presenting cells (APCs), and activation of costimulatory receptors. CD27 and CD28 are considered the most important costimulatory receptors expressed on T cells.

In mice, CD27 stimulation during the priming phase of T-cell activation, has been found to promote clonal expansion of antigen-specific CD4⁺ and CD8⁺ T cells by interleukin (IL)-2-independent survival signaling (Carr J M et al, Proc Natl Acad Sci USA 2006 Dec. 19; 130(51):19454-9). CD27 also counteracts apoptosis of activated T cells throughout successive divisions and was also shown to play an important role in memory differentiation of mouse CD8⁺ T cells. (Van de Ven K, Borst J. Immunotherapy 2015; 7(6):655-67). As a result, CD27 stimulation promotes the generation of effector T cells in lymphoid organs and broadens the responder T-cell repertoire. In human naïve T cells, CD27 stimulation promotes T helper-1 (Th1) differentiation of CD4⁺ T cells and supports effector differentiation of cytotoxic T-lymphocytes (Oosterwijk et al, Int Immunol. 2007 June; 19(6):713-8).

Contrarily to its presence on tumor cells in some hematological malignancies, CD27 expression has not been detected on tumor cells in solid malignancies. However, CD27-expressing lymphoid cells have been described in the tumor microenvironment (TME) of both hematological malignancies and solid cancers.

In the treatment of cancer, engagement and stimulation of the immune response has been shown to induce and/or enhance anti-tumor immunity resulting in clinical responses, as exemplified by the clinical success of immune checkpoint inhibitors (CPIs). An active immune response and/or existing anti-tumor immunity can be increased by providing costimulatory signaling, for example CD27 costimulatory signaling.

In mouse tumor models, T-cell functions and therefore antitumor immunity can be enhanced by agonistic CD27 antibodies. In human CD27 (hCD27)-transgenic lymphoma mouse models, CD27 activation using agonistic antibodies showed potent antitumor activity and induction of protective immunity, which is dependent on CD4⁺ and CD8⁺ T cells (He L Z et al., J Immunol. 2013 Oct. 15; 191(8):4174-83). Furthermore, CD27 activation using monoclonal antibodies prevented tumor growth in mouse xenografts, including models derived from leukemia (Vitale et al, Keler T. Clin Cancer Res. 2012 Jul. 15; 18(14):3812-21), melanoma (Roberts D J, et al., J Immunother. 2010 October; 33(8):769-79), colon carcinoma, and thymoma (He L Z, et al., J Immunol. 2013 Oct. 15; 191(8):4174-83), among others.

Monoclonal immunoglobulin G (IgG)1 agonistic antibodies against human CD27 have been disclosed in the prior art.

In WO2012/004367 a humanized anti-human CD27 agonistic antibody (designated hCD27.15) is described. It is reported that hCD27.15 does not require crosslinking by fragment crystallizable (Fc) gamma receptor (FcyR)-expressing cells to activate CD27-mediated costimulation of the immune response. However, this antibody does not bind to a frequently occurring single nucleotide polymorphism (SNP) in hCD27 (A59T) and does not bind to cynomolgus monkey CD27.

WO2011/130434 discloses a human agonistic anti-human CD27 antibody designated 1F5, which activates CD27 upon crosslinking by FcyR-expressing cells and further blocks the binding of soluble CD70 (sCD70) ligand binding. 1F5 is reported to have Fc-mediated effector function activity, including complement-dependent cytotoxicity (CDC) and antibody-dependent cellular cytotoxicity (ADCC) on target cells and to enhance the immune response and to have anti-tumor activity in mouse models.

WO2018/058022 discloses the agonistic murine anti-human CD27 antibody 131A and humanized versions thereof. It is disclosed that 131A binds the frequently occurring hCD27 SNP A59T and to cynomolgus monkey CD27. WO2018/058022 further discloses that in a mouse tumor model, antibody 131A had greater antitumor response compared with the antibody 1F5.

WO2019/195452 discloses the non-ligand blocking agonistic anti-human CD27 antibody designated BMS-986215, which is reported to have a higher affinity for human and cynomolgus monkey CD27 than the CD27 antibody 1F5 mentioned above. It is disclosed that in the presence of BMS-986215, CD27 costimulation of T cells occurs by binding to its ligand CD70. It is further disclosed that BMS-986215 reduces the suppression of CD4⁺ responder T cells by regulatory T cells (Tregs) and that BMS-986215 binds C1q and induces CDC, modest ADCC and low levels of antibody-dependent cellular phagocytosis (ADCP). It is further disclosed that BMS-986215 only has weak agonist activity in the absence of FcyR and in the absence of sCD70.

Cancer cells can avoid and suppress immune responses through upregulation of inhibitory immune checkpoint proteins, such as programmed cell death protein 1 (PD-1), and cytotoxic T-lymphocyte-associated protein 4 (CTLA-4) on T cells or programmed cell death 1 ligand 1 (PD-L1) and/or programmed cell death 1 ligand 2 (PD-L2) on tumor cells, tumor stroma or other cells within the TME. CTLA-4 and PD-1 are known to transmit signals that suppresses T-cell activation. Blocking the activities of these proteins with monoclonal antibodies, and thus restoring T-cell function, has delivered breakthrough therapies against cancer.

PD-1 (also known as CD279) is an immunoregulatory receptor expressed on the surface of activated T cells, B cells, and monocytes. The protein PD-1 has two naturally occurring ligands, which are known as PD-L1 (also referred to as CD274) and PD-L2 (also known as CD273). A wide variety of cancers express PD-L1, including melanoma, lung, renal, bladder, esophageal, gastric and other cancers. Thus, upon the interaction of PD-L1 with PD-1 in cancer, the PD-1/PD-L1 system can inhibit the proliferation of T lymphocytes, release of cytokines, and cytotoxicity, thereby providing cancer cells the opportunity to avoid a T-cell-mediated immune response.

Monoclonal antibodies suitable for regulating the activity of the PD-1/PD-L1 axis are known. The PD-1/PD-L1 interaction can be inhibited by PD-1-targeting antibodies, such as pembrolizumab (also named MK-3475, lambrolizumab or Keytruda) and nivolumab (also named ONO-4538, BMS-936558 or Opdivo), or monoclonal antibodies developed to bind PD-L1, such as e.g., atezolizumab (also named MPDL3280A, RG7446 or Tecentriq).

Anti-CD27 antibodies must induce clustering of CD27 on the plasma membrane to induce CD27 agonism. In the case of wild type IgG1 antibodies, clustering of CD27 may be achieved through interaction of membrane-bound CD27 antibodies with FcyR-bearing cells, such as monocytes, macrophages, B cells and other immune cells. As a consequence, anti-CD27 IgG1 molecules may be less efficient when the number of FcyR-expressing cells is limited. Optimization of the effector functions by modifications of the Fc region of the antibody may improve the effectivity of therapeutic antibodies for treating cancer or other diseases, e.g., to improve the ability of an antibody to elicit an immune response to antigen-expressing cells. Such efforts are described in, e.g., WO 2013/004842 A2; WO 2014/108198 A1; WO2018/146317; WO2018/083126; WO 2018/031258 A1; Dall'Acqua, Cook et al. J Immunol 2006, 177(2): 1129-1138; Moore, Chen et al. MAbs 2010 2(2): 181-189; Desjarlais and Lazar, Exp Cell Res 2011, 317(9): 1278-1285; Kaneko and Niwa, BioDrugs 2011, 25(1): 1-11; Song, Myojo et al., Antiviral Res 2014, 111: 60-68; Brezski and Georgiou, Curr Opin Immunol 2016, 40: 62-69; Sondermann and Szymkowski, Curr Opin Immunol 2016, 40: 78-87; Zhang, Armstrong et al. MAbs 2017, 9(7): 1129-1142.; Wang, Mathieu et al. Protein & Cell 2018, 9(1): 63-73; Diebolder F J et al., Science. 2014 Mar. 14; 343(6176):1260-3).

By activating the immune system, immune CPIs may also cause autoimmune side effects in some patients. In addition, engagement of the Fc domain with Fc receptors or components of the complement system may also result in undesired effector functions, such as activation of ADCC, ADCP, and CDC, which may cause unwanted depletion of CD27-positive T cells. Therefore, the activation of Fc-mediated effector function may be undesired in the context of monoclonal antibodies blocking the PD-1/PD-L1 interaction. A wide range of IgG antibody formats containing an Fc domain that does not engage Fc receptors and/or the complement system have been developed in which amino acid substitutions, and combinations thereof (i.e., non-activating mutations), have been introduced in the constant heavy chain region of an IgG1 isotype antibody to eliminate Fc-mediated effector functions (e.g., Chiu et al., Antibodies 2019 December; 8(4): 55; Liu et al., Antibodies, 2020 Nov. 17; 9(4):64; 29(10):457-66). Examples of such substitutions include the introduction of L234A-L235A-P329G non-activating mutations (Schlothauer et al., Protein Eng. Design and Selection 2016; 29(10):457-66), or L234F-L235E-D265A non-activating mutations (Also referred to as FEA or FEA format herein, Engelberts et al., EBioMedicine 2020; 52:102625; U.S. Ser. No. 10/590,206B2). Other non-activating formats were developed using human IgG4, one of the human IgG subclasses with reduced effector functions, in combination with amino acid substitutions in the constant heavy chain region of the antibody to further eliminate Fc-mediated effector functions (e.g., introduction of E233P-F234V-L235A-G236del non-activating mutations described in WO2015/143079, or introduction of F234A-L235A non-activating mutations described by Vafa et al. Methods 2014; 65: 114-126).

Amongst others, Garber et al discussed opportunities for combination therapies consisting of agonistic antibodies targeting costimulatory receptors on T cells, e.g., 4-1BB (CD137), OX40, glucocorticoid-induced tumor necrosis factor receptor family-related receptor (GITR) and independent co-stimulation (ICOS), and monoclonal antibodies blocking the PD-1/PD-L1 axis (Garber et al. Nat Rev Drug Discov. 2020 January; 19(1):3-5). Azpilikueta et al. (J Thorac Oncol 2016; 11:524-36) have published preclinical data from a combination therapy comprising a PD-1-blocking antibody and a 4-1BB-targeting antibody in a mouse lung carcinoma model, showing that the combination therapy outperformed single-agent treatment.

WO2008/051424A2 provides methods comprising the administration of a CD27-targeting agonistic antibody alone, or combined with other immunomodulatory agents, such as antibodies targeting CD40, OX40, 4-1BB or CTLA-4.

U.S. Ser. No. 10/668,152B2 provides methods for treating cancer using combination therapies comprising administering an anti-PD-1 antibody and an anti-CD27 antibody.

CDX-527 is a PD-L1×CD27 bispecific IgG1 antibody (Vitale et al., Cancer Immunol Immunother 2020).

WO2018/127916 provides PD1-CD70 dual signal fusion proteins based on the MIRP technology (Multifunctional Immune Recruitment Protein) (DSP-106).

WO2015/016718A1 provides treatments of any condition known or expected to be ameliorated by stimulation of CD27⁺ immune cells or by inhibition of one or more immune checkpoint proteins, for example by administering an anti-CD27 antibody combined with an antibody blocking PD1/PD-L1 interactions.

Despite these and other efforts in the art, however, there is a need for improved antibody-based immunotherapies with increased agonism and/or increased potency to engage CD27, provided together as a combination therapy with other immunomodulatory antibodies or antibodies blocking immune checkpoints.

### SUMMARY OF THE INVENTION

The present invention concerns binding agent capable of binding to CD27 in combination therapy.

In a first aspect, the present disclosure provides a method for reducing progression or preventing progression of a tumor or treating cancer in a subject, said method comprising administering to said subject i) a binding agent comprises at least one binding region binding to CD27; and ii) a PD1/PD-L1 inhibitor.

In a second aspect, the present disclosure provides a kit comprising i) a binding agent comprising at least one binding region binding to CD27 and ii) a PD1/PD-L1 inhibitor.

In a third aspect, the present disclosure provides a kit for use in a method for reducing progression or preventing progression of a tumor or treating cancer in a subject, said kit comprising i) a binding agent comprising at least one binding region binding to CD27 and ii) a PD1/PD-L1 inhibitor.

In a fourth aspect, the present disclosure provides a pharmaceutical composition comprising i) a binding agent comprising at least one binding region binding to CD27; ii) a PD1/PD-L1 inhibitor; and iii) optionally a pharmaceutical acceptable carrier.

In a fifth aspect, the present disclosure provides a pharmaceutical composition for use in a method for reducing progression or preventing progression of a tumor or treating cancer in a subject, said pharmaceutical composition comprising i) a binding agent comprising at least one binding region binding to CD27 and ii) a PD1/PD-L1 inhibitor.

In a sixth aspect, the present disclosure provides a binding agent for use in a method for reducing progression or preventing progression of a tumor or treating cancer in a subject, said method comprising administering to said subject i) the binding agent comprising at least one binding region binding to CD27; and ii) a PD1/PD-L1 inhibitor.

In a seventh aspect, the present disclosure provides a PD1/PD-L1 inhibitor for use in a method for reducing progression or preventing progression of a tumor or treating cancer in a subject, said method comprising administering to said subject i) a binding agent comprising at least one binding region binding to CD27; and ii) the PD1/PD-L1 inhibitor.

### DETAILED DESCRIPTION OF THE INVENTION

### Definitions

The term “antibody” (Ab) in the context of the present invention refers to an immunoglobulin molecule, a fragment of an immunoglobulin molecule, or a derivative of either thereof, which has the ability to specifically bind to an antigen. The antibody of the present invention comprises an Fc-domain of an immunoglobulin and an antigen-binding region. An antibody generally contains two CH2-CH3 regions and a connecting region, e.g., a hinge region, e.g. at least an Fc-domain. Thus, the antibody of the present invention may comprise an Fc region and an antigen-binding region. The variable regions of the heavy and light chains of the immunoglobulin molecule contain a binding domain that interacts with an antigen. The constant or “Fc” regions of the antibodies may mediate the binding of the immunoglobulin to host tissues or factors, including various cells of the immune system (such as effector cells) and components of the complement system such as C1q, the first component in the classical pathway of complement activation. As used herein, unless contradicted by context, the Fc region of an immunoglobulin typically contains at least a CH2 domain and a CH3 domain of an immunoglobulin CH, and may comprise a connecting region, e.g., a hinge region. An Fc-region is typically in dimerized form via, e.g., disulfide bridges connecting the two hinge regions and/or non-covalent interactions between the two CH3 regions. The dimer may be a homodimer (where the two Fc region monomer amino acid sequences are identical) or a heterodimer (where the two Fc region monomer amino acid sequences differ in one or more amino acids). An Fc region-fragment of a full-length antibody can, for example, be generated by digestion of the full-length antibody with papain, as is well-known in the art. An antibody as defined herein may, in addition to an Fc region and an antigen-binding region, further comprise one or both of an immunoglobulin CH1 region and a CL region. An antibody may also be a multi-specific antibody, such as a bispecific antibody or similar molecule. The term “bispecific antibody” refers to an antibody having specificities for at least two different, typically non-overlapping, epitopes. Such epitopes may be on the same or different targets. If the epitopes are on different targets, such targets may be on the same cell or different cells or cell types. As indicated above, unless otherwise stated or clearly contradicted by the context, the term antibody herein includes fragments of an antibody which comprise at least a portion of an Fc-region and which retain the ability to specifically bind to the antigen. Such fragments may be provided by any known technique, such as enzymatic cleavage, peptide synthesis and recombinant expression techniques. It has been shown that the antigen-binding function of an antibody may be performed by fragments of a full-length antibody. Examples of binding fragments encompassed within the term “Ab” or “antibody” include, without limitation, monovalent antibodies (described in WO2007059782 by Genmab); heavy-chain antibodies, consisting only of two heavy chains and naturally occurring in e.g. camelids (e.g., Hamers-Casterman (1993) Nature 363:446); ThioMabs, Roche, WO2011069104); strand-exchange engineered domain (SEED or Seed-body) which are asymmetric and bispecific antibody-like molecules (Merck, WO2007110205); Triomab (Pharma/Fresenius Biotech, Lindhofer et al. 1995 J Immunol 155:219; WO2002020039); FcAAdp (Regeneron, WO2010151792); Azymetric Scaffold (Zymeworks/Merck, WO2012/058768); mAb-Fv (Xencor, WO2011/028952); Xmab (Xencor); Dual variable domain immunoglobulin (Abbott, DVD-Ig, U.S. Pat. No. 7,612,181); Dual domain double head antibodies (Unilever; Sanofi Aventis, WO20100226923); Di-diabody (ImClone/Eli Lilly); Knobs-into-holes antibody formats (Genentech, WO9850431); DuoBody (Genmab, WO 2011/131746); Bispecific IgG1 and IgG2 (Pfizer/Rinat, WO11143545); DuetMab (MedImmune, US2014/0348839); Electrostatic steering antibody formats (Amgen, EP1870459 and WO 2009089004; Chugai, US201000155133; Oncomed, WO2010129304A2); bispecific IgG1 and IgG2 (Rinat neurosciences Corporation, WO11143545); CrossMAbs (Roche, WO2011117329); LUZ-Y (Genentech); Biclonic (Merus, WO2013157953); Dual Targeting domain antibodies (GSK/Domantis); Two-in-one Antibodies or Dual action Fabs recognizing two targets (Genentech, NovImmune, Adimab); Cross-linked Mabs (Karmanos Cancer Center); covalently fused mAbs (AIMM); CovX-body (CovX/Pfizer); FynomAbs (Covagen/Janssen ilag); DutaMab (Dutalys/Roche); iMab (MedImmune); IgG-like Bispecific (ImClone/Eli Lilly, Shen, J., et al. J Immunol Methods, 2007. 318(1-2): p. 65-74); TIG-body, DIG-body and PIG-body (Pharmabcine); Dual-affinity retargeting molecules (Fc-DART or Ig-DART, Macrogenics, WO/2008/157379, WO/2010/080538); BEAT (Glenmark); Zybodies (Zyngenia); approaches with common light chain (Crucell/Merus, U.S. Pat. No. 7,262,028) or common heavy chains (κλBodies by NovImmune, WO2012023053), as well as fusion proteins comprising a polypeptide sequence fused to an antibody fragment containing an Fc-region like scFv-fusions, like BsAb by ZymoGenetics/BMS, HERCULES by Biogen Idec (U.S. Pat. No. 7,951,918); SCORPIONS (Emergent BioSolutions/Trubion and Zymogenetics/BMS); Ts2Ab (MedImmune/AZ (Dimasi, N., et al. J Mol Biol, 2009. 393(3): p. 672-92); scFv fusion (Genentech/Roche); scFv fusion (Novartis); scFv fusion (Immunomedics); scFv fusion (Changzhou Adam Biotech Inc, CN 102250246); TvAb (Roche, WO 2012025525, WO 2012025530); mAb2 (f-Star, WO2008/003116); and dual scFv-fusion. It should be understood that the term antibody, unless otherwise specified, includes monoclonal antibodies (such as human monoclonal antibodies), polyclonal antibodies, chimeric antibodies, humanized antibodies, monospecific antibodies (such as bivalent monospecific antibodies), bispecific antibodies, antibodies of any isotype and/or allotype; antibody mixtures (recombinant polyclonals) for instance generated by technologies exploited by Symphogen and Merus (Oligoclonics), multimeric Fc proteins as described in WO2015/158867, and fusion proteins as described in WO2014/031646. While these different antibody fragments and formats are generally included within the meaning of antibody, they collectively and each independently are unique features of the present invention, exhibiting different biological properties and utility.

An “agonistic antibody” for a natural receptor is a compound which binds the receptor to form a receptor-antibody complex and which activates said receptor, thereby initiating a pathway signaling and further biological process.

The term “agonism” and “agonistic” are used interchangeably herein and refer to or describe an antibody which is capable of, directly or indirectly, substantially inducing, promoting, or enhancing CD27 biological activity or activation. Optionally, an “agonistic CD27 antibody” is an antibody which is capable of activating CD27 receptor by a similar mechanism as the ligand for CD27, known as CD70 (Tumor Necrosis Factor Superfamily member 7, TNFSF7; CD27 ligand, CD27L), which results in an activation of one or more intracellular signaling pathway which may include activation of NF-κB and MAPK8/JNK pathways. “Agonism” as defined herein may be determined according to Example 2 herein.

A “CD27 antibody” or “anti-CD27 antibody” as described herein is an antibody which binds specifically to the protein CD27, in particular to human CD27.

A “variant” as used herein refers to a protein or polypeptide sequence which differs in one or more amino acid residues from a parent or reference sequence. A variant may, for example, have a sequence identity of at least 80%, such as 90%, or 95%, or 97%, or 98%, or 99%, to a parent or reference sequence. Also, or alternatively, a variant may differ from the parent or reference sequence by 12 or less, such as 11, 10, 9, 8, 7, 6, 5, 4, 3, 2 or 1 mutation(s) such as substitutions, insertions, or deletions of amino acid residues. Accordingly, a “variant antibody” or an “antibody variant”, used interchangeably herein, refers to an antibody that differs in one or more amino acid residues as compared to a parent or reference antibody, e.g., in the antigen-binding region, Fc-region or both. Likewise, a “variant Fc region” or “Fc region variant” refers to an Fc region that differs in one or more amino acid residues as compared to a parent or reference Fc region, optionally differing from the parent or reference Fc region amino acid sequence by 12 or less, such as 11, 10, 9, 8, 7, 6, 5, 4, 3, 2 or 1 mutation(s) such as substitutions, insertions, or deletions of amino acid residues. The parent or reference Fc region is typically the Fc region of a human wild-type antibody which, depending on the context, may be a particular isotype. A variant Fc region may, in dimerized form, be a homodimer or heterodimer, e.g., where one of the amino acid sequences of the dimerized Fc region comprises a mutation while the other is identical to a parent or reference wild-type amino acid sequence. Examples of wild-type (typically a parent or reference sequence) IgG CH and variant IgG constant region amino acid sequences, which comprise Fc region amino acid sequences, are set out in Table 3.

The term “immunoglobulin heavy chain” or“heavy chain of an immunoglobulin” as used herein is intended to refer to one of the heavy chains of an immunoglobulin. A heavy chain is typically comprised of a heavy chain variable region (abbreviated herein as VH) and a heavy chain constant region (abbreviated herein as CH) which defines the isotype of the immunoglobulin. The heavy chain constant region typically is comprised of three domains, CH1, CH2, and CH3. The term “immunoglobulin” as used herein is intended to refer to a class of structurally related glycoproteins consisting of two pairs of polypeptide chains, one pair of light (L) low molecular weight chains and one pair of heavy (H) chains, all four potentially inter-connected by disulfide bonds. The structure of immunoglobulins has been well characterized (see for instance Fundamental Immunology Ch. 7 Paul, W., 2nd ed. Raven Press, N.Y. 1989). Within the structure of the immunoglobulin, the two heavy chains are inter-connected via disulfide bonds in the so-called “hinge region”. Equally to the heavy chains, each light chain is typically comprised of several regions; a light chain variable region (abbreviated herein as VL) and a light chain constant region. The light chain constant region typically is comprised of one domain, CL. Furthermore, the VH and VL regions may be further subdivided into regions of hypervariability (or hypervariable regions which may be hypervariable in sequence and/or form of structurally defined loops), also termed complementarity determining regions (CDRs), interspersed with regions that are more conserved, termed framework regions (FRs). Each VH and VL is typically composed of three CDRs and four FRs, arranged from amino-terminus to carboxy-terminus in the following order: FR1, CDR1, FR2, CDR2, FR3, CDR3, FR4. CDR sequences herein are defined according to IMGT (see Lefranc M P. et al., Nucleic Acids Research, 27, 209-212, 1999] and Brochet X. Nucl. Acids Res. 36, W503-508 (2008)), unless otherwise stated or contradicted by context.

When used herein, the terms “half molecule”, “Fab-arm” and “arm” refer to one heavy chain-light chain pair. When a bispecific antibody is described to comprise a half-molecule antibody “derived from” a first antibody, and a half-molecule antibody “derived from” a second antibody, the term “derived from” indicates that the bispecific antibody was generated by recombining, by any known method, said half-molecules from each of said first and second antibodies into the resulting bispecific antibody. In this context, “recombining” is not intended to be limited by any particular method of recombining and thus includes all of the methods for producing bispecific antibodies described herein below, including for example recombining by “half-molecule exchange” also described in the art as “Fab-arm exchange” and the DuoBody® method, as well as recombining at nucleic acid level and/or through co-expression of two half-molecules in the same cells.

The term “antigen-binding region” or “binding region” or antigen-binding domain as used herein, refers to the region of an antibody which is capable of binding to the antigen. This binding region is typically defined by the VH and VL domains of the antibody which may be further subdivided into regions of hypervariability (or hypervariable regions which may be hypervariable in sequence and/or form of structurally defined loops), also termed complementarity determining regions (CDRs), interspersed with regions that are more conserved, termed framework regions (FRs). The antigen can be any molecule, such as a polypeptide, e.g., present on a cell, bacterium, or virion. The terms “antigen-binding region” and “antigen-binding site” and “antigen-binding domain” may, unless contradicted by the context, be used interchangeably in the context of the present invention.

The terms “antigen” and “target” may, unless contradicted by the context, be used interchangeably in the context of the present invention.

The term “binding” as used herein refers to the binding of an antibody to a predetermined antigen or target, typically with a binding affinity corresponding to a K_{D} of 1E⁶ M or less, e.g. 5E⁷ M or less, 1E⁷ M or less, such as 5E⁸ M or less, such as 1E⁸ M or less, such as 5E⁹ M or less, or such as 1E⁹ M or less, when determined by biolayer interferometry using the antibody as the ligand and the antigen as the analyte and binds to the predetermined antigen with an affinity corresponding to a K_{D} that is at least ten-fold lower, such as at least 100-fold lower, for instance at least 1,000-fold lower, such as at least 10,000-fold lower, for instance at least 100,000-fold lower than its affinity for binding to a non-specific antigen (e.g., BSA, casein) other than the predetermined antigen or a closely-related antigen.

The term “K_{D}” (M), as used herein, refers to the dissociation equilibrium constant of a particular antibody-antigen interaction, and is obtained by dividing k_{d} by kₐ.

The term “k_{d}” (sec⁻¹), as used herein, refers to the dissociation rate constant of a particular antibody-antigen interaction. Said value is also referred to as the k_{off} value or off-rate.

The term “kₐ” (M⁻¹×sec⁻¹), as used herein, refers to the association rate constant of a particular antibody-antigen interaction. Said value is also referred to as the kₒₙ value or on-rate.

The term “CD27” as used herein, refers to the human protein entitled CD27, also known as tumor necrosis factor receptor superfamily member 7 (TNFRSF7). In the amino acid sequence shown in SEQ ID NO: 1 (Uniprot ID P26842), amino acid residues 1-19 are a signal peptide, and amino acid residues 20-240 are the mature polypeptide. Unless contradicted by context, CD27 may also refer to variants of CD27, isoforms and orthologs thereof. A naturally occurring variant of human CD27 comprising a A59T mutation is shown in SEQ ID NO: 2.

In cynomolgus monkey (Macaca fascicularis), the CD27 protein has the amino acid sequence shown in SEQ ID NO: 3 (Genbank XP_005569963). In the 240 amino acid sequence shown in SEQ ID NO: 3, the signal peptide is not defined.

The term “antibody binding region” refers to a region of the antigen, which comprises the epitope to which the antibody binds. An antibody binding region may be determined by epitope binding using biolayer interferometry, by alanine scan, or by shuffle assays (using antigen constructs in which regions of the antigen are exchanged with that of another species and determining whether the antibody still binds to the antigen or not). The amino acids within the antibody binding region that are involved in the interaction with the antibody may be determined by hydrogen/deuterium exchange mass spectrometry and by crystallography of the antibody bound to its antigen.

The term “epitope” means an antigenic determinant which is specifically bound by an antibody. Epitopes usually consist of surface groupings of molecules such as amino acids, sugar side chains or a combination thereof and usually have specific three-dimensional structural characteristics, as well as specific charge characteristics. Conformational and non-conformational epitopes are distinguished in that the binding to the former but not the latter is lost in the presence of denaturing solvents. The epitope may comprise amino acid residues which are directly involved in the binding, and other amino acid residues, which are not directly involved in the binding, such as amino acid residues which are effectively blocked or covered by the antibody when it is bound to the antigen (in other words, the amino acid residue is within or closely adjacent to the footprint of the specific antibody).

The terms “monoclonal antibody”, “monoclonal Ab”, “monoclonal antibody composition”, “mAb”, or the like, as used herein refer to a preparation of antibody molecules of single molecular composition. A monoclonal antibody composition displays a single binding specificity and affinity for a particular epitope. Accordingly, the term “human monoclonal antibody” refers to antibodies displaying a single binding specificity which have variable and constant regions derived from human germline immunoglobulin sequences. The human monoclonal antibodies may be produced by a hybridoma which includes a B cell obtained from a transgenic or trans-chromosomal non-human animal, such as a transgenic mouse or rat, having a genome comprising a human heavy chain transgene and a light chain transgene, fused to an immortalized cell. Monoclonal antibodies may also be produced from recombinantly modified host cells, or systems that use cellular extracts supporting in vitro transcription and/or translation of nucleic acid sequences encoding the antibody.

The term “isotype” as used herein refers to the immunoglobulin class (for instance IgG, IgG1, IgG2, IgG3, IgG4, IgD, IgA, IgE, or IgM) or any allotypes thereof, such as IgG1m(za) and IgG1m(f)) that is encoded by heavy chain constant region genes. Further, each heavy chain isotype can be combined with either a kappa (κ) or lambda (λ) light chain.

The term “full-length antibody” when used herein, indicates that the antibody is not a fragment, but contains all of the domains of the particular isotype normally found for that isotype in nature, e.g., the VH, CH1, CH2, CH3, hinge, VL and CL domains for an IgG1 antibody. In a full-length variant antibody, the heavy and light chain constant and variable domains may in particular contain amino acid substitutions that improve the functional properties of the antibody when compared to the full-length parent or wild type antibody. A full-length antibody according to the present invention may be produced by a method comprising the steps of (i) cloning the CDR sequences into a suitable vector comprising complete heavy chain sequences and complete light chain sequence, and (ii) expressing the complete heavy and light chain sequences in suitable expression systems. It is within the knowledge of the skilled person to produce a full-length antibody when starting out from either CDR sequences or full variable region sequences. Thus, the skilled person would know how to generate a full-length antibody according to the present invention.

The term “human antibody”, as used herein, is intended to include antibodies comprising variable and framework regions derived from human germline immunoglobulin sequences and a human immunoglobulin constant domain. The human antibodies of the invention may include amino acid residues not encoded by human germline immunoglobulin sequences (e.g., mutations, insertions or deletions introduced by random or site-specific mutagenesis in vitro or by somatic mutation in vivo). However, the term “human antibody”, as used herein, is not intended to include antibodies in which CDR sequences derived from the germline of another non-human species, such as a mouse, have been grafted onto human framework sequences. The term “humanized antibody” as used herein, refers to a genetically engineered non-human antibody, which contains human antibody constant domains and non-human variable domains modified to contain a high level of sequence homology to human variable domains. This can be achieved by grafting of the six non-human antibody complementarity-determining regions (CDRs), which together form the antigen binding site, onto a homologous human acceptor framework region (FR) (see WO92/22653 and EP0629240). In order to fully reconstitute the binding affinity and specificity of the parental antibody, the substitution of framework residues from the parental antibody (i.e., the non-human antibody) into the human framework regions (back-mutations) may be required. Structural homology modeling may help to identify the amino acid residues in the framework regions that are important for the binding properties of the antibody. Thus, a humanized antibody may comprise non-human CDR sequences, primarily human framework regions optionally comprising one or more amino acid back-mutations to the non-human amino acid sequence, and fully human constant regions. Optionally, additional amino acid modifications, which are not necessarily back-mutations, may be applied to obtain a humanized antibody with preferred characteristics, such as affinity and biochemical properties.

The term “Fc region” or “Fc domain” as used herein may be used interchangeably and refers to a region of the heavy chain constant region comprising, in the direction from the N- to C-terminal end of the antibody, at least a hinge region, a CH2 region and a CH3 region. An Fc region of the antibody may mediate the binding of the immunoglobulin to host tissues or factors, including various cells of the immune system (such as effector cells) and components of the complement system.

The term “parent polypeptide” or “parent antibody”, is to be understood as a polypeptide or antibody, which is identical to a polypeptide or antibody according to the invention, but where the parent polypeptide or parent antibody is without mutations, unless otherwise stated or clearly contradicted by the context. For example, the antibody IgG1-CD27-A of the invention is the parent antibody of IgG1-CD27-A-P329R-E345R.

The term “hinge region” as used herein refers to the hinge region of an immunoglobulin heavy chain. Thus, for example the hinge region of a human IgG1 antibody corresponds to amino acids 216-230 according to the Eu numbering (Eu-index) as set forth in Kabat, E. A. et al., Sequences of proteins of immunological interest. 5th Edition—US Department of Health and Human Services, NIH publication No. 91-3242, pp 662,680,689 (1991). However, the hinge region may also be any of the other subtypes as described herein.

The term “CH1 region” or “CH1 domain” as used herein refers to the CH1 region of an immunoglobulin heavy chain. Thus, for example the CH1 region of a human IgG1 antibody corresponds to amino acids 118-215 according to the Eu numbering as set forth in Kabat (ibid). However, the CH1 region may also be any of the other subtypes as described herein.

The term “CH2 region” or “CH2 domain” as used herein refers to the CH2 region of an immunoglobulin heavy chain. Thus, for example the CH2 region of a human IgG1 antibody corresponds to amino acids 231-340 according to the Eu numbering as set forth in Kabat (ibid). However, the CH2 region may also be any of the other subtypes as described herein.

The term “CH3 region” or “CH3 domain” as used herein refers to the CH3 region of an immunoglobulin heavy chain. Thus, for example the CH3 region of a human IgG1 antibody corresponds to amino acids 341-447 according to the Eu numbering as set forth in Kabat (ibid). However, the CH3 region may also be any of the other subtypes as described herein.

The term “Fc-mediated effector functions” or “Fc effector functions” as used herein are used interchangeably and is intended to refer to functions that are a consequence of binding a polypeptide or antibody to its target or antigen on a cell membrane wherein the Fc-mediated effector function is attributable to the Fc region of the polypeptide or antibody. Examples of Fc-mediated effector functions include (i) C1q binding, (ii) complement activation, (iii) complement-dependent cytotoxicity (CDC), (iv) antibody-dependent cell-mediated cytotoxity (ADCC), (v) Fc-gamma receptor (FcγR)-binding, (vi) antibody-dependent, FcγR-mediated antigen crosslinking, (vii) antibody-dependent cellular phagocytosis (ADCP), (viii) complement-dependent cellular cytotoxicity (CDCC), (ix) complement-enhanced cytotoxicity, (x) binding to complement receptor of an opsonized antibody mediated by the antibody, (xi) opsonisation, and (xii) a combination of any of (i) to (xi).

The term “decreased Fc effector function(s)” or “Decreased Fc-mediated effector functions”, as used herein are used interchangeably and is intended to refer to an Fc effector function that is decreased for an antibody when directly compared to the Fc effector function of the parent polypeptide or antibody in the same assay.

The term “inertness”, “inert” or “non-activating” as used herein, refers to an Fc region which is at least not able to bind any FcγR, induce Fc-mediated cross-linking of FcγRs, or induce FcγR-mediated cross-linking of target antigens via two Fc regions of individual antibodies, or is not able to bind C1q. Thus, in certain embodiments of the invention the Fc region is inert. Therefore, in certain embodiments some or all of the Fc-mediated effector functions are attenuated or completely absent.

The term “oligomerization”, as used herein, is intended to refer to a process that converts monomers to a finite degree of polymerization. Antibodies according to the invention can form oligomers, such as hexamers, via non-covalent association of Fc-regions after target binding, e.g., at a cell surface. Oligomerization of anti-CD27 antibodies upon cell surface binding through Fc:Fc interactions may increase CD27 clustering resulting in activation of CD27 intracellular signaling. The capacity of antibodies comprising the E345R or E430G mutation to form oligomers, such as hexamers, upon cell surface binding can be evaluated as described in: de Jong R N et al, PLoS Biol. 2016 Jan. 6; 14(1):e1002344. Fc-Fc-mediated oligomerization of antibodies occurs after target binding on a (cell) surface through the intermolecular association of Fc-regions between neighboring antibodies and is increased by introduction of a E345R or a E430G mutation (numbering according to Eu-index).

The term “clustering”, as used herein, refers to oligomerization of antibodies through non-covalent interactions.

The term “Fc-Fc enhancing”, as used herein, is intended to refer to increasing the binding strength between, or stabilizing the interaction between, the Fc regions of two Fc-region containing antibodies so that the antibodies form oligomers such as hexamers on the cell surface. This enhancement can be obtained by certain amino acid mutations in the Fc regions of the antibodies, such as E345R or E430G. The term “monovalent antibody”, in the context of the present invention, refers to an antibody molecule that can interact with a specific epitope on an antigen, with only one antigen binding domain (e.g. one Fab arm). In the context of a bispecific antibody, “monovalent antibody binding” refers to the binding of the bispecific antibody to one specific epitope on an antigen with only one antigen binding domain (e.g. one Fab arm).

The term “monospecific antibody” in the context of the present invention, refers to an antibody that has binding specificity to one epitope only. The antibody may be a monospecific, monovalent antibody (i.e. carrying only one antigen binding region) or a monospecifc, bivalent antibody (i.e. an antibody with two identical antigen binding regions).

The term “bispecific antibody” refers to an antibody comprising two non-identical antigen binding domains, e.g. two non-identical Fab-arms or two Fab-arms with non-identical CDR regions. In the context of this invention, bispecific antibodies have specificity for at least two different epitopes. Such epitopes may be on the same or different antigens or targets. If the epitopes are on different antigens, such antigens may be on the same cell or different cells, cell types or structures, such as extracellular matrix or vesicles and soluble protein. A bispecific antibody may thus be capable of crosslinking multiple antigens, e.g. two different cells. A particular bispecific antibody of the present invention is capable of binding to CD27 and a second target.

The term “bivalent antibody” refers to an antibody that has two antigen binding regions, which bind to epitopes on one or two targets or antigens or binds to one or two epitopes on the same antigen. Hence, a bivalent antibody may be a monospecific, bivalent antibody or a bispecific, bivalent antibody.

The term “amino acid” and “amino acid residue” may herein be used interchangeably and are not to be understood limiting. Amino acids are organic compounds containing amine (—NH₂) and carboxyl (—COOH) functional groups, along with a side chain (R group) specific to each amino acid. In the context of the present invention, amino acids may be classified based on structure and chemical characteristics. Thus, classes of amino acids may be reflected in one or both of the following tables:

TABLE 20
Main classification based on structure and general
chemical characterization of R group

| Class | Amino acid |
|---|---|
| Acidic Residues | D and E |
| Basic Residues | K, R, and H |
| Hydrophilic Uncharged Residues | S, T, N, and Q |
| Aliphatic Uncharged Residues | G, A, V, L, and I |
| Non-polar Uncharged Residues | C, M, and P |
| Aromatic Residues | F, Y, and W |

TABLE 21
Alternative Physical and Functional Classifications
of Amino Acid Residues

| Class | Amino acid |
|---|---|
| Hydroxyl group containing | S and T |
| residues | |
| Aliphatic residues | I, L, V, and M |
| Cycloalkenyl-associated | F, H, W, and Y |
| residues | |
| Hydrophobic residues | A, C, F, G, H, I, L, M, R, T, V, W, |
| | and Y |
| Negatively charged residues | D and E |
| Polar residues | C, D, E, H, K, N, Q, R, S, and T |
| Positively charged residues | H, K, and R |
| Small residues | A, C, D, G, N, P, S, T, and V |
| Very small residues | A, G, and S |
| Residues involved in turn | A, C, D, E, G, H, K, N, Q, R, S, P, |
| formation | and T |
| Flexible residues | Q, T, K, S, G, P, D, E, and R |

Substitution of one amino acid for another may be classified as a conservative or non-conservative substitution. In the context of the invention, a “conservative substitution” is a substitution of one amino acid with another amino acid having similar structural and/or chemical characteristics, such substitution of one amino acid residue for another amino acid residue of the same class as defined in any of the two tables above: for example, leucine may be substituted with isoleucine as they are both aliphatic, branched hydrophobes. Similarly, aspartic acid may be substituted with glutamic acid since they are both small, negatively charged residues.

In the context of the present invention, a substitution in an antibody is indicated as:
Original amino acid-position-substituted amino acid;

Referring to the well-recognized nomenclature for amino acids, the three-letter code, or one letter code, is used, including the codes “Xaa” or “X” to indicate any amino acid residue. Thus, Xaa or X may typically represent any of the 20 naturally occurring amino acids. The term “naturally occurring” as used herein refers to any one of the following amino acid residues; glycine, alanine, valine, leucine, isoleucine, serine, threonine, lysine, arginine, histidine, aspartic acid, asparagine, glutamic acid, glutamine, proline, tryptophan, phenylalanine, tyrosine, methionine, and cysteine. Accordingly, the notation “K409R” or “Lys409Arg” means, that the antibody comprises a substitution of Lysine with Arginine in amino acid position 409. Substitution of an amino acid at a given position to any other amino acid is referred to as: Original amino acid-position; or e.g. “K409”

For a modification where the original amino acid(s) and/or substituted amino acid(s) may comprise more than one, but not all amino acid(s), the more than one amino acid may be separated by “,” or “/”. E.g. the substitution of Lysine with Arginine, Alanine, or Phenylalanine in position 409 is:
“Lys409Arg,Ala,Phe” or “Lys409Arg/Ala/Phe” or “K409R,A,F” or “K409R/A/F” or “K409 to R, A, or F”.

Such designation may be used interchangeably in the context of the invention but have the same meaning and purpose.

Furthermore, the term “a substitution” embraces a substitution into any one or the other nineteen natural amino acids, or into other amino acids, such as non-natural amino acids. For example, a substitution of amino acid K in position 409 includes each of the following substitutions: 409A, 409C, 409D, 409E, 409F, 409G, 409H, 409I, 409L, 409M, 409N, 409Q, 409R, 409S, 409T, 409V, 409W, 409P, and 409Y. This is, by the way, equivalent to the designation 409X, wherein the X designates any amino acid other than the original amino acid. These substitutions may also be designated K409A, K409C, etc. or K409A,C, etc. or K409A/C/etc. The same applies by analogy to each and every position mentioned herein, to specifically include herein any one of such substitutions.

The antibody according to the invention may also comprise a deletion of an amino acid residue. Such deletion may be denoted “del”, and includes, e.g., writing as K409del. Thus, in such embodiments, the Lysine in position 409 has been deleted from the amino acid sequence. The term “host cell”, as used herein, is intended to refer to a cell into which an expression vector has been introduced. It should be understood that such terms are intended to refer not only to the particular subject cell, but also to the progeny of such a cell. Because certain modifications may occur in succeeding generations due to either mutation or environmental influences, such progeny may not, in fact, be identical to the parent cell, but are still included within the scope of the term “host cell” as used herein. Recombinant host cells include, for example, transfectomas, such as CHO cells, HEK-293 cells, Expi293F cells, PER.C6 cells, NS0 cells, and lymphocytic cells, and prokaryotic cells such as E. coli and other eukaryotic hosts such as plant cells and fungi.

The term “transfectoma”, as used herein, includes recombinant eukaryotic host cells expressing the antibody or a target antigen, such as CHO cells, PER.C6 cells, NS0 cells, HEK-293 cells, Expi293F cells, plant cells, or fungi, including yeast cells.

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows: (Identical Residues×100)/(Length of Alignment−Total Number of Gaps in Alignment).

The retention of similar residues may also or alternatively be measured by a similarity score, as determined by use of a BLAST program (e.g., BLAST 2.2.8 available through the NCBI using standard settings BLOSUM62, Open Gap=11 and Extended Gap=1). Suitable variants typically exhibit at least about 45%, such as at least about 55%, at least about 65%, at least about 75%, at least about 85%, at least about 90%, at least about 95%, or more (e.g., about 99%) similarity to the parent sequence.

The term “internalized” or “internalization” as used herein, refers to a biological process in which molecules such as the antibody according to the present invention, are engulfed by the cell membrane and drawn into the interior of the cell. Internalization may also be referred to as “endocytosis”.

As used herein, the term “effector cell” refers to an immune cell which is involved in the effector phase of an immune response. Exemplary immune cells include a cell of a myeloid or lymphoid origin, for instance lymphocytes (such as B cells and T cells including cytolytic T cells (CTLs)), killer cells, natural killer cells, macrophages, monocytes, eosinophils, polymorphonuclear cells, such as neutrophils, granulocytes, mast cells, and basophils. Some effector cells express Fc receptors (FcgRs) or complement receptors and carry out specific immune functions. In some embodiments, an effector cell such as, e.g., a natural killer cell, is capable of inducing ADCC. For example, monocytes, macrophages, neutrophils, dendritic cells and Kupffer cells which express FcgRs, are involved in specific killing of target cells and/or presenting antigens to other components of the immune system, or binding to cells that present antigens. In some embodiments the ADCC can be further enhanced by antibody driven classical complement activation resulting in the deposition of activated C3 fragments on the target cell. C3 cleavage products are ligands for complement receptors (CRs), such as CR3, expressed on myeloid cells. The recognition of complement fragments by CRs on effector cells may promote enhanced Fc receptor-mediated ADCC. In some embodiments antibody driven classical complement activation leads to C3 fragments on the target cell. These C3 cleavage products may promote direct complement-dependent cellular cytotoxicity (CDCC). In some embodiments, an effector cell may phagocytose a target antigen, target particle or target cell which may depend on antibody binding and mediated by FcγRs expressed by the effector cells. The expression of a particular FcR or complement receptor on an effector cell may be regulated by humoral factors such as cytokines. For example, expression of FcγRI has been found to be up-regulated by interferon γ (IFN γ) and/or G-CSF. This enhanced expression increases the cytotoxic activity of FcγRI-bearing cells against targets. An effector cell can phagocytose a target antigen or phagocytose or lyse a target cell. In some embodiments antibody driven classical complement activation leads to C3 fragments on the target cell. These C3 cleavage products may promote direct phagocytosis by effector cells or indirectly by enhancing antibody mediated phagocytosis. In certain embodiments herein where the antibody has an inert Fc region the antibody does not induce an Fc-mediated effector function. “Effector T cells” or “Teffs” or “Teff” as used herein refers to T lymphocytes that carry out a function of an immune response, such as killing tumor cells and/or activating an antitumor immune-response which can result in clearance of the tumor cells from the body. Examples of Teff phenotypes include CD3⁺CD4⁺ and CD3⁺CD8⁺. Teffs may secrete, contain, or express markers such as IFNγ, granzyme B and ICOS. It is appreciated that Teffs may not be fully restricted to these phenotypes.

“Memory T cells” as used herein refers to T lymphocytes that remain in the body for a long period of time after an infection is removed. Examples of memory T cells include central memory T cells (CD45RA−CCR7+) and effector memory T cells (CD45RA−CCR7−). It is appreciated that memory T cells may not be fully restricted to these phenotypes.

“Regulatory T cells” or “Tregs” or “Treg” as used herein refers to T lymphocytes that regulate the activity of other T cell(s) and/or other immune cells, usually by suppressing their activity. An example of a Treg phenotype is CD3⁺CD4⁺CD25⁺CD127dim. Tregs may further express Foxp3. It is appreciated that Tregs may not be fully restricted to this phenotype.

As used herein, the term “complement activation” refers to the activation of the classical complement pathway, which is initiated by a large macromolecular complex called C1 binding to antibody-antigen complexes on a surface. C1 is a complex, which consists of 6 recognition proteins C1q and a hetero-tetramer of serine proteases, C1r2C1s2. C1 is the first protein complex in the early events of the classical complement cascade that involves a series of cleavage reactions that starts with the cleavage of C4 into C4a and C4b and C2 into C2a and C2b. C4b is deposited and forms together with C2a an enzymatic active convertase called C3 convertase, which cleaves complement component C3 into C3b and C3a, which forms a C5 convertase This C5 convertase splits C5 in C5a and C5b and the last component is deposited on the membrane and that in turn triggers the late events of complement activation in which terminal complement components C5b, C6, C7, C8 and C9 assemble into the membrane attack complex (MAC). The complement cascade results in the creation of pores in the cell membrane which causes lysis of the cell, also known as complement-dependent cytotoxicity (CDC). In certain embodiments herein where the antibody has an inert Fc region the antibody does not induce complement activation.

Complement activation can be evaluated by using C1q binding efficacy, CDC kinetics CDC assays (as described in WO2013/004842, WO2014/108198) or by the method Cellular deposition of C3b and C4b described in Beurskens et al., J Immunol Apr. 1, 2012 vol. 188 no. 7, 3532-3541.

The term “C1q binding” as used herein, is intended to refer to the binding of C1q in the context of the binding of C1q to an antibody bound to its antigen. The antibody bound to its antigen is to be understood as happening both in vivo and in vitro in the context described herein. C1q binding can be evaluated for example by using antibody immobilized on artificial surfaces or by using antibody bound to a predetermined antigen on a cellular or virion surface, as described in Example 8 herein. The binding of C1q to an antibody oligomer is to be understood herein as a multivalent interaction resulting in high avidity binding. A decrease in C1q binding, for example resulting from the introduction of a mutation in the antibody of the invention, may be measured by comparing the C1q binding of the mutated antibody to the C1q binding of its parent antibody (the antibody of the invention without the mutation within the same assay).

The term “treatment” refers to the administration of an effective amount of a therapeutically active antibody of the present invention with the purpose of easing, ameliorating, arresting, or eradicating (curing) symptoms or disease states.

The term “effective amount” or “therapeutically effective amount” refers to an amount effective, at dosages and for periods of time necessary, to achieve a desired therapeutic result. A therapeutically effective amount of an antibody may vary according to factors such as the disease state, age, sex, and weight of the individual, and the ability of the antibody to elicit a desired response in the individual. A therapeutically effective amount is also one in which any toxic or detrimental effects of the antibody variant are outweighed by the therapeutically beneficial effects.

The term “pharmacokinetic profile” as used herein can be determined as the plasma IgG levels over time as described in Example 12 herein.

The term “CD137” as used herein, refers to CD137 (4-1BB), also referred to as tumor necrosis factor receptor superfamily member 9 (TNFRSF9), which is the receptor for the ligand TNFSF9/4-1BBL. CD137 (4-1BB) is believed to be involved in T-cell activation. Other synonyms for CD137 include, but are not limited to, 4-1BB ligand receptor, CD137, T-cell antigen 4-1BB homolog and T-cell antigen ILA. In one embodiment, CD137 (4-1BB) is human CD137 (4-1BB), having UniProt accession number Q07011. The sequence of human CD137 is also shown in SEQ ID NO: 130. Amino acids 1-23 of SEQ ID NO: 130 correspond to the signal peptide of human CD137; while amino acids 24-186 of SEQ ID NO: 130 correspond to the extracellular domain of human CD137; and the remainder of the protein, i.e. from amino acids 187-213 and 214-255 of SEQ ID NO: 130 are transmembrane and cytoplasmic domain, respectively.

The “Programmed Death-1 (PD-1)” receptor refers to an immuno-inhibitory receptor belonging to the CD28 family.

The term “PD-L1” as used herein includes human PD-L1 (hPD-L1), variants, isoforms, and species homologs of hPD-L1, such as macaque (cynomolgus monkey), African elephant, wild boar and mouse PD-L1 (cf., e.g., Genbank accession no. NP_054862.1, XP_005581836, XP_003413533, XP_005665023 and NP_068693, respectively), and analogs having at least one common epitope with hPD-L1. The sequence of human PD-L1 is also shown in SEQ ID NO: 98 (mature sequence), and in SEQ ID NO: 129, wherein amino acids 1-18 are predicted to be a signal peptide. The term “PD-L2” as used herein includes human PD-L2 (hPD-L2), variants, isoforms, and species homologs of hPD-L2, and analogs having at least one common epitope with hPD-L2. The ligands of PD-1 (PD-L1 and PD-L2) are expressed on the surface of antigen-presenting cells, such as dendritic cells or macrophages, and other immune cells. Binding of PD-1 to PD-L1 or PD-L2 results in downregulation of T cell activation. Cancer cells expressing PD-L1 and/or PD-L2 are able to switch off T cells expressing PD-1 what results in suppression of the anticancer immune response. The interaction between PD-1 and its ligands results in a decrease in tumor infiltrating lymphocytes, a decrease in T cell receptor mediated proliferation, and immune evasion by the cancerous cells. Immune suppression can be reversed by inhibiting the local interaction of PD-1 with PD-L1, and the effect is additive when the interaction of PD-1 with PD-L2 is blocked as well.

The term “PD-1” relates to programmed cell death-1 and includes any variants, conformations, isoforms and species homologs of PD-1 which are naturally expressed by cells or are expressed by cells transfected with the PD-1 gene. Preferably, “PD-1” relates to human PD-1, in particular to a protein having the amino acid sequence (NCBI Reference Sequence: NP_005009.2) as set forth in SEQ ID NO: 58 of the sequence listing, or a protein being preferably encoded by a nucleic acid sequence (NCBI Reference Sequence: NM_005018.2) as set forth in SEQ ID NO: 60 of the sequence listing. Alternative names for “PD-1” include CD279 and SLEB2.

The term “PD-1” includes posttranslationally modified variants, isoforms and species homologs of human PD-1 which are naturally expressed by cells or are expressed in/on cells transfected with the PD-1 gene.

The term “PD-1 variant” shall encompass (i) PD-1 splice variants, (ii) PD-1-posttranslationally modified variants, particularly including variants with different N-glycosylation status, (iii) PD-1 conformation variants. Such variants may include soluble forms of PD-1.

PD-1 is a type I membrane protein that belongs to the immunoglobulin superfamily (The EMBO Journal (1992), vol. 11, issue 11, p. 3887-3895). The human PD-1 protein comprises an extracellular domain composed of the amino acids at positions 24 to 170 of the sequence as set forth in SEQ ID NO: 58 of the sequence listing, a transmembrane domain (amino acids at positions 171 to 191 of the sequence as set forth in SEQ ID NO: 58) and a cytoplasmatic domain (amino acids at positions 192 to 288 of the sequence as set forth in SEQ ID NO: 58). The term “PD-1 fragment” as used herein shall encompass any fragment of a PD-1 protein, preferably an immunogenic fragment. The term also encompasses, for example, the above-mentioned domains of the full length protein or any fragment of these domains, in particular immunogenic fragments. The amino acid sequence of a preferred extracellular domain of the human PD-1 protein is set forth in SEQ ID NO: 59 of the sequence listing.

Fc regions may have at their C-terminus a lysine. The origin of this lysine is a naturally occurring sequence found in humans from which these Fc regions are derived. During cell culture production of recombinant antibodies, this terminal lysine can be cleaved off by proteolysis by endogenous carboxypeptidase(s), resulting in a constant region having the same sequence but lacking the C-terminal lysine. For manufacturing purposes of antibodies, the DNA encoding this terminal lysine can be omitted from the sequence such that antibodies are produced without the lysine. Antibodies produced from nucleic acid sequences that either do, or do not encode a terminal lysine are substantially identical in sequence and in function since the degree of processing of the terminal lysine is typically high when e.g. using antibodies produced in CHO-based production systems (Dick, L. W. et al. Biotechnol. Bioeng. 2008; 100: 1132-1143). Hence, it is understood that proteins in accordance with the invention, such as antibodies, can be generated with or without encoding or having a terminal lysine. It is also understood in accordance with the invention that, sequences with a terminal lysine, such as a constant region sequence having a terminal lysine, can be understood as the corresponding sequences without a terminal lysine, and that sequences without a terminal lysine can also be understood as the corresponding sequences with a terminal lysine.

### ASPECTS AND EMBODIMENTS OF THE PRESENT DISCLOSURE

In a first aspect, the present disclosure provides a method for reducing progression or preventing progression of a tumor or treating cancer in a subject, said method comprising administering to said subject i) a binding agent comprises at least one binding region binding to CD27; and ii) a PD1/PD-L1 inhibitor.

### Binding Agent Binding to CD27

In one embodiment of the invention, the binding agent comprises at least one antigen-binding region capable of binding to human CD27 wherein said binding agent comprises a heavy chain variable (VH) region CDR1, CDR2, and CDR3 comprising the sequences as set forth in SEQ ID NOs: 5, 6, and 7, respectively, and a light chain variable (VL) region CDR1, CDR2, and CDR3 comprising the sequences as set forth in SEQ ID NO: 9, 10 and 11, respectively.

In a further embodiment of the invention, the binding agent comprises two of said antigen-binding regions comprising the VH region CDR1, CDR2, and CDR3 comprising the sequences as set forth in SEQ ID NOs: 5, 6, and 7, respectively, and the VL region CDR1, CDR2, and CDR3 comprising the sequences as set forth in SEQ ID NO: 9, 10 and 11 respectively. Hereby anti-CD27 antibodies are provided which are able to bind to human CD27 and further to bind to a variant of human CD27 comprising a mutation of A59T.

In an embodiment of the invention the binding agent binds CD27 e.g. on T cells and is agonistic upon binding to its target. Hereby a binding agent is provided which stimulates the activation and proliferation of T-cells. The binding agent may further stimulate memory formation and survival of T-cells. Such a binding agent is useful e.g. in the treatment of cancer. The binding agent is further capable of binding to cynomolgus CD27 which is useful for toxicological studies of the binding agent.

In one embodiment the binding agent is an isolated antibody.

In one embodiment the binding agent is an antibody. In another embodiment the binding agent is a human antibody. In another embodiment the binding agent is a humanized antibody. In another embodiment the binding agent is a chimeric antibody.

The binding agent is in a preferred embodiment a full-length antibody. Accordingly, the binding agent of the invention may further comprise a light chain constant region (CL) and a heavy chain constant region (CH). The CH preferably comprises a CH1 region, a hinge region, a CH2 region and a CH3 region.

It is well known in the art that mutations in the VH and VL of an antibody can be made to, for example, increase the affinity of an antibody to its target antigen, reduce its potential immunogenicity and/or to increase the yield of antibodies expressed by a host cell. Accordingly, in some embodiments, binding agents comprising variants of the CDR, VH and/or VL sequences of a binding agent according to the invention are also contemplated, particularly functional variants of the VH and/or VL region as set forth in SEQ ID NO: 4 and SEQ ID NO: 8, respectively. Functional variants may differ in one or more amino acids as compared to the parent VH and/or VL sequence, e.g., in one or more CDRs, but still allows the antigen-binding region to retain at least a substantial proportion (at least about 50 percent, 60 percent, 70 percent, 80 percent, 90 percent, 95 percent or more) or even retain all of the affinity and/or specificity of the parent antibody. Typically, such functional variants retain significant sequence identity to the parent sequence. Exemplary variants include those which differ from the respective parent VH or VL region by 12 or less, such as 11, 10, 9, 8, 7, 6, 5, 4, 3, 2 or 1 mutation(s) such as substitutions, insertions or deletions of amino acid residues. Exemplary variants include those which differ from the VH and/or VL and/or CDR regions of the parent sequences mainly by conservative amino acid substitutions; for instance, 12, such as 11, 10, 9, 8, 7, 6, 5, 4, 3, 2 or 1 of the amino acid substitutions in the variant can be conservative. In a further embodiment of the invention the binding agent may comprise at most 1, 2 or 3 mutations in the VH CDR region and/or in the VL CDR region, respectively. Such mutations may be substitutions. It is preferred that such substitutions do not significantly change the binding affinity and/or binding specificity of the binding agent of the invention. Accordingly, the present invention encompasses variants of the binding agent of the invention which variants have the same functional features as the binding agent comprising the VH region CDR sequences as set forth in SEQ ID NOs: 5, 6, and 7, and the VL region CDR sequences as set forth in SEQ ID NO: 9, 10 and 11.

In another embodiment of the invention the binding agent comprises a VH region comprising a sequence which is at least 80% identical to the VH region as set forth in SEQ ID NO: 4. In another embodiment of the invention the binding agent comprises a VH region comprising a sequence which is at least 85% identical to the VH region as set forth in SEQ ID NO: 4. In another embodiment of the invention the binding agent comprises a VH region comprising a sequence which is at least 90% identical to the VH region as set forth in SEQ ID NO: 4. In another embodiment of the invention the binding agent comprises a VH region comprising a sequence which is at least 95% identical to the VH region as set forth in SEQ ID NO: 4. In another embodiment of the invention the binding agent comprises a VH region comprising a sequence which is at least 96% identical to the VH region as set forth in SEQ ID NO: 4. In another embodiment of the invention the binding agent comprises a VH region comprising a sequence which is at least 97% identical to the VH region as set forth in SEQ ID NO: 4. In another embodiment of the invention the binding agent comprises a VH region comprising a sequence which is at least 98% identical to the VH region as set forth in SEQ ID NO: 4. In another embodiment of the invention the binding agent comprises a VH region comprising a sequence which is at least 99% identical to the VH region as set forth in SEQ ID NO: 4. In another embodiment of the invention the binding agent comprises a VH region comprising a sequence as set forth in SEQ ID NO: 4.

In another embodiment of the invention the binding agent comprises a VH region comprising a sequence which is at least 80% identical to the VH region as set forth in SEQ ID NO: 8. In another embodiment of the invention the binding agent comprises a VH region comprising a sequence which is at least 85% identical to the VH region as set forth in SEQ ID NO: 8. In another embodiment of the invention the binding agent comprises a VH region comprising a sequence which is at least 90% identical to the VH region as set forth in SEQ ID NO: 8. In another embodiment of the invention the binding agent comprises a VH region comprising a sequence which is at least 95% identical to the VH region as set forth in SEQ ID NO: 8. In another embodiment of the invention the binding agent comprises a VH region comprising a sequence which is at least 96% identical to the VH region as set forth in SEQ ID NO: 8. In another embodiment of the invention the binding agent comprises a VH region comprising a sequence which is at least 97% identical to the VH region as set forth in SEQ ID NO: 8. In another embodiment of the invention the binding agent comprises a VH region comprising a sequence which is at least 98% identical to the VH region as set forth in SEQ ID NO: 8. In another embodiment of the invention the binding agent comprises a VH region comprising a sequence which is at least 99% identical to the VH region as set forth in SEQ ID NO: 8. In another embodiment of the invention the binding agent comprises a VH region comprising a sequence as set forth in SEQ ID NO: 8.

In another embodiment of the invention the binding agent comprises the VH and VL regions comprising the sequences as set forth in SEQ ID NO: 4 and SEQ ID NO: 8, respectively. The binding agent used in the method according to the invention may comprise a light chain constant region which is a human kappa light chain. In another embodiment it may comprise a human lambda light chain constant region.

The binding agent may preferably further comprise a heavy chain constant region, which is of a human IgG isotype. It may optionally comprise a modified human IgG constant region. Such human IgG comprise the Fc region which comprise the CH2 and CH3 region. By modifying the IgG constant region in the Fc region, it is for example possible to regulate the Fc effector functions of the antibody or to increase the Fc-Fc interactions and thereby the antibodies tendency to form clusters such as hexamers. In one embodiment of the invention the human IgG or modified human IgG is selected from IgG1, IgG2, IgG3 or IgG4. In one embodiment it is IgG1. In another embodiment it is IgG2. In yet another embodiment it is IgG3. In a further embodiment it is IgG4. In one particular embodiment the IgG is a modified human IgG comprising one or more amino acid substitutions in the Fc region. In one embodiment it may be a human IgG1 comprising one or more amino acid substitutions in the Fc region. In a further embodiment of the invention the IgG1 comprises two or more amino acid substitutions in the Fc region. In one embodiment the IgG1 Fc region has two amino acid substitutions.

In a further embodiment of the invention, the modified human IgG heavy chain constant region comprises in the Fc region at most 10 amino acid substitutions. In another embodiment it comprises at most 9 amino acid substitutions. In another embodiment it comprises at most 8 amino acid substitutions. In another embodiment it comprises at most 7 amino acid substitutions. In another embodiment it comprises at most 6 amino acid substitutions. In another embodiment it comprises at most 5 amino acid substitutions. In another embodiment it comprises at most 4 amino acid substitutions. In another embodiment it comprises at most 3 amino acid substitutions. In another embodiment it comprises at most 2 amino acid substitutions in the Fc region.

Mutations in amino acid residues at positions corresponding to E430, E345 and S440 in a human IgG1 heavy chain, wherein the amino acid residues are numbered according to the EU index, can improve the ability of an antibody to induce CDC. Without being bound by theory, it is believed that by substituting one or more amino acid(s) in these positions, oligomerization of the antibody can be stimulated, thereby modulating Fc-mediated effector functions so as to, e.g., increase C1q binding, complement activation, CDC, ADCP, internalization or other relevant function(s) that may provide in vivo efficacy.

In a further embodiment of the invention, the binding agent is a variant antibody comprising an antigen-binding region and a variant Fc region.

In certain embodiments, an antibody variant binding to human CD27 comprises:
(a) a heavy chain comprising a VH region comprising a VH CDR1 comprising the sequence as set forth in SEQ ID NO:5, a VH CDR2 comprising the sequence as set forth in SEQ ID NO:6, a VH CDR3 comprising the sequence as set forth in SEQ ID NO:7 and a human IgG1 CH region comprising a mutation in one or more of E430, E345 and S440, the amino acid residues being numbered according to the EU index;
(b) a light chain comprising a VL region comprising a VL CDR1 comprising the sequence as set forth in SEQ ID NO:9, a VL CDR2 comprising the sequence as set forth in SEQ ID NO: 10, and a VL CDR3 comprising the sequence as set forth in SEQ ID NO:11.

In other certain embodiments, an antibody variant binding to human CD27 comprises:
(a) a heavy chain comprising a VH region comprising SEQ ID NO:4 and a human IgG1 CH region comprising a mutation in one or more of E430, E345 and S440, the amino acid residues being numbered according to the EU index, and
(b) a light chain comprising a VL region comprising SEQ ID NO:8.

A variant antibody of the present invention, binding to human CD27, comprises a variant Fc region or a variant human IgG1 CH region comprising a mutation in one or more of P329, E430 and E345. In the following, reference to the mutations in the Fc region may similarly apply to the mutation(s) in the human IgG1 CH region and vice versa.

As described herein, the position of an amino acid to be mutated in the Fc region can be given in relation to (i.e., “corresponding to”) its position in a naturally occurring (wildtype) human IgG1 heavy chain, when numbered according to the Eu index. So, if the parent Fc region already contains one or more mutations and/or if the parent Fc region is, for example, an IgG2, IgG3 or IgG4 Fc region, the position of the amino acid corresponding to an amino acid residue such as, e.g., E430 in a human IgG1 heavy chain numbered according to the Eu index can be determined by alignment. Specifically, the parent Fc region is aligned with a wild-type human IgG1 heavy chain sequence so as to identify the residue in the position corresponding to E430 in the human IgG1 heavy chain sequence. Any wildtype human IgG1 constant region amino acid sequence can be useful for this purpose, including any one of the different human IgG1 allotypes set forth in Table 3.

In one embodiment of the invention the modification in the IgG Fc region induces increased CD27 agonism compared to the identical antibody but comprising a wild type IgG Fc region of the same isotype, such as IgG1. This may for example be obtained by introducing an amino acid other than E at the amino acid position corresponding to position E345 and/or E430 in a human IgG1 heavy chain according to Eu numbering. In one embodiment of the invention the amino acid residue at the position corresponding to position E345 in a human IgG1 heavy chain according to Eu numbering is selected from the group comprising: A, C, D, F, G, H, I, K, L, M, N, Q, P, R, S, T, V, W and Y. In another embodiment of the invention the amino acid residue at the position corresponding to position E430 in a human IgG1 heavy chain according to Eu numbering is selected from the group comprising: A, C, D, F, G, H, I, K, L, M, N, Q, P, R, S, T, V, W.

In a preferred embodiment the amino acid residue at the position corresponding to position E345 in a human IgG1 heavy chain according to Eu numbering is R. Accordingly, the binding agent of the invention may comprise an E345R substitution in the Fc region. In another embodiment of the invention the amino acid residue at the position corresponding to position E430 in a human IgG1 heavy chain according to Eu numbering is G. Accordingly, the binding agent of the invention may comprise a E430G substitution in the Fc region. In another embodiment, the binding agent comprises an amino acid substitution selected from the group comprising E430G, E345K, E430S, E430F, E430T, E345Q, E345R, E345Y.

Hereby, a binding agent is provided in the form of an antibody or antibodies, which have enhanced Fc-Fc interaction which may lead to antibody-dependent clustering of CD27 on the cell surface upon antibody binding, thereby increasing the agonism of the binding agent of the invention.

In another embodiment of the binding agent used according to the invention the amino acid residue at the position corresponding to position P329 in a human IgG1 heavy chain according to Eu numbering is substituted with an amino acid selected from the group comprising: A, C, D, E, F, G, H, I, K, L, M, N, Q, R, S, T, V, W and Y. Accordingly, the binding agent used according to the invention may further comprise a mutation in position 329.

In a further embodiment of the invention the binding agent has the amino acid residue R at the position corresponding to position P329 in a human IgG1 heavy chain according to Eu numbering. Accordingly, the binding agent of the invention may have a P329R substitution in the Fc region. Without being bound by theory, it is believed that the binding agent comprising an E345R mutation in the Fc region (as e.g. set out in SEQ ID NO: 13) has increased serum clearance. The inventors found that further introducing a mutation at position 329, such as P329R (as e.g. set out in SEQ ID NO: 15) restored the clearance of the binding agent to the level of the binding agent comprising a wt IgG1 as e.g. set out in SEQ ID NO: 12.

In another preferred embodiment the amino acid residues at the positions corresponding to positions P329 and E345 in a human IgG1 heavy chain according to Eu numbering are both R. Hereby a binding agent is provided which has increased CD27 receptor agonism and comparable pharmacokinetic properties, such as e.g. serum clearance, when compared to a binding agent comprising the same VH and VL region and comprising an identical IgG1 heavy chain constant region with the exception of comprising the wildtype amino acid P at position 329 and the wildtype amino acid E at position 345.

Thus, in an embodiment the binding agent has increased receptor agonism upon binding to CD27 and further has pharmacokinetic properties which are comparable, such as similar or even identical pharmacokinetic properties, when compared to the pharmacokinetic properties of a binding agent comprising the same VH and VL region but comprising a wild type IgG1 heavy chain constant region such as e.g. set out in SEQ ID NO: 12. In other words the binding agent may have pharmacokinetic properties which are not significantly different than the pharmacokinetic properties of an identical binding agent except for comprising a wild type IgG1 heavy chain constant region.

In other embodiments of the invention the binding agent comprises a variant Fc region according to any one of the preceding sections, which variant Fc region is a variant of a human IgG Fc region selected from the group consisting of a human IgG1, IgG2, IgG3 and IgG4 Fc region. That is, the mutation in one or more of the amino acid residues corresponding to E430 and E345 and P329 is/are made in a parent Fc region which is a human IgG Fc region selected from the group consisting of an IgG1, IgG2, IgG3 and IgG4 Fc region. Preferably, the parent Fc region is a naturally occurring (wildtype) human IgG Fc region, such as a human wildtype IgG1, IgG2, IgG3 or IgG4 Fc region, or a mixed isotype thereof. Thus, the variant Fc region may, except for the recited mutation (in one or more of the amino acid residues selected from E430 and E345 and P329), be a human IgG1, IgG2, IgG3 or IgG4 isotype, or a mixed isotype thereof.

In one embodiment, the parent Fc region and/or human IgG1 CH region is a wild-type human IgG1 isotype.

Thus, the variant Fc region may except for the recited mutation (in E430 or E345 or P329), be a human IgG1 Fc region.

In a specific embodiment, the parent Fc region and/or human IgG1 CH region is a human wild-type IgG1m(f) isotype.

In a specific embodiment, the parent Fc region and/or human IgG1 CH region is a human wild-type IgG1m(z) isotype.

In a specific embodiment, the parent Fc region and/or human IgG1 CH region is a human wild-type IgG1m(a) isotype.

In a specific embodiment, the parent Fc region and/or human IgG1 CH region is a human wild-type IgG1m(x) isotype.

In a specific embodiment, the parent Fc region and/or human IgG1 CH region is a human wild-type IgG1 of a mixed allotype, such as IgG1m(za), IgG1m(zax), IgG1m(fa), or the like.

Thus, the variant Fc region and/or human IgG1 CH region may, except for the recited mutation (in E430 or E345 or P329), be a human IgG1m(f), IgG1m(a), IgG1m(x), IgG1m(z) allotype or a mixed allotype of any two or more thereof.

In a specific embodiment, the parent Fc region and/or human IgG1 CH region is a human wild-type IgG1m(za) isotype.

In a specific embodiment, the parent Fc region is a human wild-type IgG2 isotype.

In a specific embodiment, the parent Fc region is a human wild-type IgG3 isotype.

In a specific embodiment, the parent Fc region is a human wild-type IgG4 isotype.

CH region amino acid sequences of specific examples of wild-type human IgG isotypes and IgG1 allotypes are set forth in Table 3.

In another embodiment the binding agent comprises a heavy chain constant region comprising an amino acid sequence selected from the group comprising: SEQ ID Nos 12, 13, 14, 15, 18, 19, 20, 21, 22, 23, 27, 28, 29, 30, 31, 32, 33, 34 and 36. In one embodiment the heavy chain constant region has the amino acid sequence of SEQ ID NO: 12. In one embodiment the heavy chain constant region has the amino acid sequence of SEQ ID NO: 13. In one embodiment the heavy chain constant region has the amino acid sequence of SEQ ID NO: 14. In one embodiment the heavy chain constant region has the amino acid sequence of SEQ ID NO: 15. In one embodiment the heavy chain constant region has the amino acid sequence of SEQ ID NO: 18. In one embodiment the heavy chain constant region has the amino acid sequence of SEQ ID NO: 19. In one embodiment the heavy chain constant region has the amino acid sequence of SEQ ID NO: 20. In one embodiment the heavy chain constant region has the amino acid sequence of SEQ ID NO: 21. In one embodiment the heavy chain constant region has the amino acid sequence of SEQ ID NO: 22. In one embodiment the heavy chain constant region has the amino acid sequence of SEQ ID NO: 23. In one embodiment the heavy chain constant region has the amino acid sequence of SEQ ID NO: 27. In one embodiment the heavy chain constant region has the amino acid sequence of SEQ ID NO: 28. In one embodiment the heavy chain constant region has the amino acid sequence of SEQ ID NO: 29. In one embodiment the heavy chain constant region has the amino acid sequence of SEQ ID NO: 30. In one embodiment the heavy chain constant region has the amino acid sequence of SEQ ID NO: 31. In one embodiment the heavy chain constant region has the amino acid sequence of SEQ ID NO: 32. In one embodiment the heavy chain constant region has the amino acid sequence of SEQ ID NO: 33. In one embodiment the heavy chain constant region has the amino acid sequence of SEQ ID NO: 34. In one embodiment the heavy chain constant region has the amino acid sequence of SEQ ID NO: 36.

In an embodiment the binding agent comprises:
a. The VH region comprising the amino acid sequence set forth in SEQ ID No: 4
b. The VL region comprising the amino acid sequence set forth in SEQ ID No: 8
c. The CH region comprising the amino acid sequence set forth in SEQ ID No: 15 and
d. The CL region comprising the amino acid sequence set forth in SEQ ID No: 16.

In another embodiment the binding agent comprises:
a. The VH region comprising the amino acid sequence set forth in SEQ ID No: 4
b. The VL region comprising the amino acid sequence set forth in SEQ ID No: 8
c. The CH region comprising the amino acid sequence set forth in SEQ ID No: 12 and
d. The CL region comprising the amino acid sequence set forth in SEQ ID No: 16.

In another embodiment the first binding comprises:
a. The VH region comprising the amino acid sequence set forth in SEQ ID No: 4
b. The VL region comprising the amino acid sequence set forth in SEQ ID No: 8
c. The CH region comprising the amino acid sequence set forth in SEQ ID No: 13 and
d. The CL region comprising the amino acid sequence set forth in SEQ ID No: 16.

In another embodiment the binding agent comprises:
a. The VH region comprising the amino acid sequence set forth in SEQ ID No: 4
b. The VL region comprising the amino acid sequence set forth in SEQ ID No: 8
c. The CH region comprising the amino acid sequence set forth in SEQ ID No: 14 and
d. The CL region comprising the amino acid sequence set forth in SEQ ID No: 16.

In another embodiment the binding agent comprises:
a. The VH region comprising the amino acid sequence set forth in SEQ ID No: 4
b. The VL region comprising the amino acid sequence set forth in SEQ ID No: 8
c. The CH region comprising the amino acid sequence set forth in SEQ ID No: 18 and
d. The CL region comprising the amino acid sequence set forth in SEQ ID No: 16.

In another embodiment the binding agent comprises:
a. The VH region comprising the amino acid sequence set forth in SEQ ID No: 4
b. The VL region comprising the amino acid sequence set forth in SEQ ID No: 8
c. The CH region comprising the amino acid sequence set forth in SEQ ID No: 19 and
d. The CL region comprising the amino acid sequence set forth in SEQ ID No: 16.

In another embodiment the binding agent comprises:
a. The VH region comprising the amino acid sequence set forth in SEQ ID No: 4
b. The VL region comprising the amino acid sequence set forth in SEQ ID No: 8
c. The CH region comprising the amino acid sequence set forth in SEQ ID No: 20 and
d. The CL region comprising the amino acid sequence set forth in SEQ ID No: 16.

In another embodiment the binding agent comprises:
a. The VH region comprising the amino acid sequence set forth in SEQ ID No: 4
b. The VL region comprising the amino acid sequence set forth in SEQ ID No: 8
c. The CH region comprising the amino acid sequence set forth in SEQ ID No: 21 and
d. The CL region comprising the amino acid sequence set forth in SEQ ID No: 16.

In another embodiment the binding agent comprises:
a. The VH region comprising the amino acid sequence set forth in SEQ ID No: 4
b. The VL region comprising the amino acid sequence set forth in SEQ ID No: 8
c. The CH region comprising the amino acid sequence set forth in SEQ ID No: 22 and
d. The CL region comprising the amino acid sequence set forth in SEQ ID No: 16.

In another embodiment the binding agent comprises:
a. The VH region comprising the amino acid sequence set forth in SEQ ID No: 4
b. The VL region comprising the amino acid sequence set forth in SEQ ID No: 8
c. The CH region comprising the amino acid sequence set forth in SEQ ID No: 23 and
d. The CL region comprising the amino acid sequence set forth in SEQ ID No: 16.

In another embodiment the binding agent comprises:
a. The VH region comprising the amino acid sequence set forth in SEQ ID No: 4
b. The VL region comprising the amino acid sequence set forth in SEQ ID No: 8
c. The CH region comprising the amino acid sequence set forth in SEQ ID No: 27 and
d. The CL region comprising the amino acid sequence set forth in SEQ ID No: 16.

In another embodiment the binding agent comprises:
a. The VH region comprising the amino acid sequence set forth in SEQ ID No: 4
b. The VL region comprising the amino acid sequence set forth in SEQ ID No: 8
c. The CH region comprising the amino acid sequence set forth in SEQ ID No: 28 and
d. The CL region comprising the amino acid sequence set forth in SEQ ID No: 16.

In another embodiment the binding agent comprises:
a. The VH region comprising the amino acid sequence set forth in SEQ ID No: 4
b. The VL region comprising the amino acid sequence set forth in SEQ ID No: 8
c. The CH region comprising the amino acid sequence set forth in SEQ ID No: 29 and
d. The CL region comprising the amino acid sequence set forth in SEQ ID No: 16.

In another embodiment the binding agent according to the invention comprises:
a. The VH region comprising the amino acid sequence set forth in SEQ ID No: 4
b. The VL region comprising the amino acid sequence set forth in SEQ ID No: 8
c. The CH region comprising the amino acid sequence set forth in SEQ ID No: 30 and
d. The CL region comprising the amino acid sequence set forth in SEQ ID No: 16.

In another embodiment the binding agent comprises:
a. The VH region comprising the amino acid sequence set forth in SEQ ID No: 4
b. The VL region comprising the amino acid sequence set forth in SEQ ID No: 8
c. The CH region comprising the amino acid sequence set forth in SEQ ID No: 31 and
d. The CL region comprising the amino acid sequence set forth in SEQ ID No: 16.

In another embodiment the binding agent comprises:
a. The VH region comprising the amino acid sequence set forth in SEQ ID No: 4
b. The VL region comprising the amino acid sequence set forth in SEQ ID No: 8
c. The CH region comprising the amino acid sequence set forth in SEQ ID No: 32 and
d. The CL region comprising the amino acid sequence set forth in SEQ ID No: 16.

In another embodiment the binding agent comprises:
a. The VH region comprising the amino acid sequence set forth in SEQ ID No: 4
b. The VL region comprising the amino acid sequence set forth in SEQ ID No: 8
c. The CH region comprising the amino acid sequence set forth in SEQ ID No: 33 and
d. The CL region comprising the amino acid sequence set forth in SEQ ID No: 16.

In another embodiment the binding agent comprises:
a. The VH region comprising the amino acid sequence set forth in SEQ ID No: 4
b. The VL region comprising the amino acid sequence set forth in SEQ ID No: 8
c. The CH region comprising the amino acid sequence set forth in SEQ ID No: 34 and
d. The CL region comprising the amino acid sequence set forth in SEQ ID No: 16.

In another embodiment the binding agent comprises:
a. The VH region comprising the amino acid sequence set forth in SEQ ID No: 4
b. The VL region comprising the amino acid sequence set forth in SEQ ID No: 8
c. The CH region comprising the amino acid sequence set forth in SEQ ID No: 36 and
d. The CL region comprising the amino acid sequence set forth in SEQ ID No: 16.

In alternative embodiments the CL region may be the amino acid sequence set forth in SEQ ID No: 17.

In an embodiment the binding agent comprises:
e. The VH region comprising the amino acid sequence set forth in SEQ ID No: 4
f. The VL region comprising the amino acid sequence set forth in SEQ ID No: 8
g. The CH region comprising the amino acid sequence set forth in SEQ ID No: 15 and
h. The CL region comprising the amino acid sequence set forth in SEQ ID No: 17.

In another embodiment the binding agent comprises:
e. The VH region comprising the amino acid sequence set forth in SEQ ID No: 4
f. The VL region comprising the amino acid sequence set forth in SEQ ID No: 8
g. The CH region comprising the amino acid sequence set forth in SEQ ID No: 12 and
h. The CL region comprising the amino acid sequence set forth in SEQ ID No: 17.

In another embodiment the binding agent comprises:
e. The VH region comprising the amino acid sequence set forth in SEQ ID No: 4
f. The VL region comprising the amino acid sequence set forth in SEQ ID No: 8
g. The CH region comprising the amino acid sequence set forth in SEQ ID No: 13 and
h. The CL region comprising the amino acid sequence set forth in SEQ ID No: 17.

In another embodiment the binding agent comprises:
e. The VH region comprising the amino acid sequence set forth in SEQ ID No: 4
f. The VL region comprising the amino acid sequence set forth in SEQ ID No: 8
g. The CH region comprising the amino acid sequence set forth in SEQ ID No: 14 and
h. The CL region comprising the amino acid sequence set forth in SEQ ID No: 17.

In another embodiment the binding agent comprises:
e. The VH region comprising the amino acid sequence set forth in SEQ ID No: 4
f. The VL region comprising the amino acid sequence set forth in SEQ ID No: 8
g. The CH region comprising the amino acid sequence set forth in SEQ ID No: 18 and
h. The CL region comprising the amino acid sequence set forth in SEQ ID No: 17.

In another embodiment the binding agent comprises:
e. The VH region comprising the amino acid sequence set forth in SEQ ID No: 4
f. The VL region comprising the amino acid sequence set forth in SEQ ID No: 8
g. The CH region comprising the amino acid sequence set forth in SEQ ID No: 19 and
h. The CL region comprising the amino acid sequence set forth in SEQ ID No: 17.

In another embodiment the binding agent comprises:
e. The VH region comprising the amino acid sequence set forth in SEQ ID No: 4
f. The VL region comprising the amino acid sequence set forth in SEQ ID No: 8
g. The CH region comprising the amino acid sequence set forth in SEQ ID No: 20 and
h. The CL region comprising the amino acid sequence set forth in SEQ ID No: 17.

In another embodiment the binding agent comprises:
e. The VH region comprising the amino acid sequence set forth in SEQ ID No: 4
f. The VL region comprising the amino acid sequence set forth in SEQ ID No: 8
g. The CH region comprising the amino acid sequence set forth in SEQ ID No: 21 and
h. The CL region comprising the amino acid sequence set forth in SEQ ID No: 17.

In another embodiment the binding agent comprises:
e. The VH region comprising the amino acid sequence set forth in SEQ ID No: 4
f. The VL region comprising the amino acid sequence set forth in SEQ ID No: 8
g. The CH region comprising the amino acid sequence set forth in SEQ ID No: 22 and
h. The CL region comprising the amino acid sequence set forth in SEQ ID No: 17.

In another embodiment the binding agent comprises:
e. The VH region comprising the amino acid sequence set forth in SEQ ID No: 4
f. The VL region comprising the amino acid sequence set forth in SEQ ID No: 8
g. The CH region comprising the amino acid sequence set forth in SEQ ID No: 23 and
h. The CL region comprising the amino acid sequence set forth in SEQ ID No: 17.

In another embodiment the binding agent comprises:
e. The VH region comprising the amino acid sequence set forth in SEQ ID No: 4
f. The VL region comprising the amino acid sequence set forth in SEQ ID No: 8
g. The CH region comprising the amino acid sequence set forth in SEQ ID No: 27 and
h. The CL region comprising the amino acid sequence set forth in SEQ ID No: 17.

In another embodiment the binding agent comprises:
e. The VH region comprising the amino acid sequence set forth in SEQ ID No: 4
f. The VL region comprising the amino acid sequence set forth in SEQ ID No: 8
g. The CH region comprising the amino acid sequence set forth in SEQ ID No: 28 and
h. The CL region comprising the amino acid sequence set forth in SEQ ID No: 17.

In another embodiment the binding agent comprises:
e. The VH region comprising the amino acid sequence set forth in SEQ ID No: 4
f. The VL region comprising the amino acid sequence set forth in SEQ ID No: 8
g. The CH region comprising the amino acid sequence set forth in SEQ ID No: 29 and
h. The CL region comprising the amino acid sequence set forth in SEQ ID No: 17.

In another embodiment the binding agent comprises:
e. The VH region comprising the amino acid sequence set forth in SEQ ID No: 4
f. The VL region comprising the amino acid sequence set forth in SEQ ID No: 8
g. The CH region comprising the amino acid sequence set forth in SEQ ID No: 30 and
h. The CL region comprising the amino acid sequence set forth in SEQ ID No: 17.

In another embodiment the binding agent comprises:
e. The VH region comprising the amino acid sequence set forth in SEQ ID No: 4
f. The VL region comprising the amino acid sequence set forth in SEQ ID No: 8
g. The CH region comprising the amino acid sequence set forth in SEQ ID No: 31 and
h. The CL region comprising the amino acid sequence set forth in SEQ ID No: 17.

In another embodiment the binding agent comprises:
e. The VH region comprising the amino acid sequence set forth in SEQ ID No: 4
f. The VL region comprising the amino acid sequence set forth in SEQ ID No: 8
g. The CH region comprising the amino acid sequence set forth in SEQ ID No: 32 and
h. The CL region comprising the amino acid sequence set forth in SEQ ID No: 17.

In another embodiment the binding agent comprises:
e. The VH region comprising the amino acid sequence set forth in SEQ ID No: 4
f. The VL region comprising the amino acid sequence set forth in SEQ ID No: 8
g. The CH region comprising the amino acid sequence set forth in SEQ ID No: 33 and
h. The CL region comprising the amino acid sequence set forth in SEQ ID No: 17.

In another embodiment the binding agent comprises:
e. The VH region comprising the amino acid sequence set forth in SEQ ID No: 4
f. The VL region comprising the amino acid sequence set forth in SEQ ID No: 8
g. The CH region comprising the amino acid sequence set forth in SEQ ID No: 34 and
h. The CL region comprising the amino acid sequence set forth in SEQ ID No: 17.

In another embodiment the binding agent comprises:
e. The VH region comprising the amino acid sequence set forth in SEQ ID No: 4
f. The VL region comprising the amino acid sequence set forth in SEQ ID No: 8
g. The CH region comprising the amino acid sequence set forth in SEQ ID No: 36 and
h. The CL region comprising the amino acid sequence set forth in SEQ ID No: 17.

In another embodiment the binding agent comprises a heavy chain comprising the amino acid sequence set forth in SEQ ID NO: 24 and a light chain comprising the amino acid sequence set forth in SEQ ID NO: 25.

In another embodiment the binding agent comprises a heavy chain comprising the amino acid sequence set forth in SEQ ID NO: 35 and a light chain comprising the amino acid sequence set forth in SEQ ID NO: 25.

In yet another embodiment the binding agent comprises a heavy chain constant region that is modified so that the binding agent induces an Fc-mediated effector function to a lesser extent relative to an identical binding agent except for the modification. An example hereof is the CD27 binding antibody of the invention comprising a P329R and an E345R substitution. Such antibody induces one or more Fc-mediated effector function(s) to a lesser extent compared to the antibody comprising the same sequence except not comprising the P329R substitution and also compared to the same antibody comprising the same sequence except not comprising the P329R and E345R substitutions, such as a wildtype IgG1 heavy chain. In one embodiment the Fc-mediated effector function is decreased by at least 20%. In another embodiment the Fc-mediated effector function is decreased by at least 30%. In another embodiment the Fc-mediated effector function is decreased by at least 40%. In another embodiment the Fc-mediated effector function is decreased by at least 50%. In another embodiment the Fc-mediated effector function is decreased by at least 60%. In another embodiment the Fc-mediated effector function is decreased by at least 70%. In another embodiment the Fc-mediated effector function is decreased by at least 80%. In another embodiment the Fc-mediated effector function is decreased by at least 90%. In another embodiment the binding agent does not induce one or more Fc-mediated effector functions. The one or more Fc-effector functions that are decreased or not at all induced may be selected from the following group: complement-dependent cytotoxicity (CDC), complement-dependent cell-mediated cytotoxicity (CDCC), complement activation, antibody-dependent cell-mediated cytotoxicity (ADCC), antibody-dependent cell-mediated phagocytosis (ADCP), C1q binding and FcγR binding. Accordingly, in one embodiment the binding agent induces CDC to a degree which is decreased by at least 20%, such as at least 30%, or at least 40%, or at least 50%, or at least 60%, or at least 70%, or at least 80%, or decreased by at least 90% relative to the identical binding agent but a wildtype IgG1 HC constant region. In another embodiment the binding agent does not induce CDC.

In another embodiment, the binding agent induces CDCC to a degree which is decreased by at least 20%, such as at least 30%, or at least 40%, or at least 50%, or at least 60%, or at least 70%, or at least 80%, or decreased by at least 90% relative to the identical binding agent but having a wildtype IgG1 HC constant region. In another embodiment the binding agent does not induce CDCC.

In another embodiment, the binding agent induces ADCC to a degree which is decreased by at least 20%, such as at least 30%, or at least 40%, or at least 50%, or at least 60%, or at least 70%, or at least 80%, or decreased by at least 90% relative to the identical binding agent but having a wildtype IgG1 HC constant region. In another embodiment the binding agent does not induce ADCC.

In another embodiment, the binding agent induces ADCP to a degree which is decreased by at least 20%, such as at least 30%, or at least 40%, or at least 50%, or at least 60%, or at least 70%, or at least 80%, or decreased by at least 90% relative to the identical binding agent but having a wildtype IgG1 HC constant region. In another embodiment the binding agent does not induce ADCP.

In another embodiment, the binding agent induces C1q binding to a degree which is decreased by at least 20%, such as at least 30%, or at least 40%, or at least 50%, or at least 60%, or at least 70%, or at least 80%, or decreased by at least 90% relative to the identical binding agent but having a wildtype IgG1 HC constant region. In another embodiment the binding agent does not induce C1q binding. Preferably the C1q binding is determined as in example 8.

In another embodiment, the binding agent induces FcγR binding to a degree which is decreased by at least 20%, such as at least 30%, or at least 40%, or at least 50%, or at least 60%, or at least 70%, or at least 80%, or decreased by at least 90% relative to the identical binding agent but having a wildtype IgG1 HC constant region. In another embodiment the binding agent does not induce FcγR binding. Preferably the FcγR binding is determined as in example 9.

In one embodiment the binding agent has reduced C1q binding and reduced FcγR binding compared to the binding agent comprising the same amino acid sequences except not comprising the P329R substitution.

In one embodiment, the binding agent used in any aspect or embodiment herein is, except for the recited mutations, a human antibody.

In an embodiment of the invention the binding agent is a monovalent antibody.

In another embodiment the binding agent is a bivalent antibody.

Further, the binding agent of the invention may be a monospecific antibody.

In one embodiment, the binding agent used in any aspect or embodiment herein is a monoclonal antibody, such as a human monoclonal antibody, such as a human bivalent monoclonal antibody, such as a human bivalent full-length monoclonal antibody.

In a preferred embodiment, the binding agent used in any aspect or embodiment herein is, except for the optional recited mutations in the Fc region, an IgG1 antibody, such as a full length IgG1 antibody, such as a human full-length IgG1 antibody, optionally a human monoclonal full-length bivalent IgG1,K antibody, e.g. a human monoclonal full-length bivalent IgG1m(f),κ antibody.

A binding agent used in relation to the present invention is advantageously in a bivalent monospecific format, comprising two antigen-binding regions binding to the same epitope. However, bispecific formats where one of the antigen-binding regions binds to a different epitope are also contemplated. So, the binding agent used according to any aspect or embodiment herein can, unless contradicted by context, be either a monospecific antibody or a bispecific antibody.

Accordingly, in another embodiment, the binding agent is a bispecific antibody comprising a first antigen binding region capable of binding human CD27 as described herein and comprising a second antigen binding region capable of binding to a different epitope on human CD27. In another embodiment, the binding agent is a bispecific antibody comprising a first antigen binding region capable of binding human CD27 as described herein and comprising a second antigen binding region capable of binding a different target. Such target may be on a different cell or on the same cell as CD27.

In an embodiment of the invention the binding agent is capable of binding to human CD27 having the sequence as set forth in SEQ ID NO: 1. However, human CD27 may in some individuals be expressed as a variant hereof. Thus, in another embodiment the binding agent is further capable of binding to a human CD27 variant, such as for example the human CD27 variant as set forth in SEQ ID NO: 2. In another embodiment, the binding agent if further capable of binding to cynomolgus CD27, such as set forth in SEQ ID NO: 3.

In a further embodiment of the invention the binding agent is capable of binding CD27-expressing human T cells.

In another embodiment of the invention the binding agent is capable of binding CD27-expressing cynomolgus T cells.

In one embodiment of the invention the full length IgG1 antibody has had the C-terminal Lysine of the HC cleaved off. Such an antibody is also considered a “full length antibody”.

In another embodiment of the invention the binding agent is capable of inducing proliferation of human T cells such as CD4⁺ and CD8⁺ T-cells, such as T helper cells and cytotoxic T cells.

Such activity may be assayed as described in Example 6 or 7 herein.

In another embodiment of the invention the binding agent is capable of inducing activation of human CD27-expressing Jurkat reporter T cells such as described in Example 2 herein.

In another embodiment of the invention the binding agent is capable of inducing activation of human CD27-expressing Jurkat reporter T cells in the absence of Fcγ receptor IIb cross-linking such as described in Example 11 herein.

In another embodiment of the invention the binding agent is capable of inducing proliferation of CD4⁺ and CD8⁺ T cells with a central memory T cell phenotype.

In another embodiment of the invention the binding agent is capable of inducing IFN gamma production.

In another embodiment of the invention the binding agent is in a composition or formulation comprising acetate, sorbitol, polysorbate 80, and has a pH from 5 to 6, preferably 5.5.

### PD1/PD-L1 Inhibitor

In one embodiment, the PD1/PD-L1 inhibitor prevents inhibitory signals associated with PD-1. In one embodiment, the PD1/PD-L1 inhibitor is an antibody, or fragment thereof that disrupts or inhibits inhibitory signaling associated with PD-1. In one embodiment, the PD1/PD-L1 inhibitor is a small molecule inhibitor that disrupts or inhibits inhibitory signaling. In one embodiment, the PD1/PD-L1 inhibitor is a peptide-based inhibitor that disrupts or inhibits inhibitory signaling. In one embodiment, the PD1/PD-L1 inhibitor is an inhibitory nucleic acid molecule that disrupts or inhibits inhibitory signaling.

Inhibiting or blocking of PD-1 signaling, as described herein, results in preventing or reversing immune-suppression and establishment or enhancement of T cell immunity against cancer cells. In one embodiment, inhibition of PD-1 signaling, as described herein, reduces or inhibits dysfunction of the immune system. In one embodiment, inhibition of PD-1 signaling, as described herein, renders dysfunctional immune cells less dysfunctional. In one embodiment, inhibition of PD-1 signaling, as described herein, renders a dysfunctional T cell less dysfunctional.

In one embodiment, PD-L1 is human PD-L1, in particular human PD-L1 comprising the sequence set forth in SEQ ID NO: 98.

In one embodiment, PD1 is human PD1. Preferably PD1 has or comprises the amino acid sequence as set forth in SEQ ID NO: 58 or SEQ ID NO: 59, or the amino acid sequence of PD1 has at least 70%, at least 75%, at least 80%, at least 85%, at least 90%, at least 95%, at least 97%, at least 99%, or 100% identity to the amino acid sequence as set forth in SEQ ID NO: 58 or SEQ ID NO: 59, or is an immunogenic fragment thereof.

In one embodiment, the PD1/PD-L1 inhibitor prevents the interaction between PD-1 and PD-L1.

The PD1/PD-L1 inhibitor may be an antibody, an antigen-binding fragment thereof, or a construct thereof comprising an antibody portion with an antigen-binding fragment of the required specificity. Antibodies or antigen-binding fragments thereof are as described herein.

Antibodies or antigen-binding fragments thereof that are PD1/PD-L1 inhibitors include in particular antibodies or antigen-binding fragments thereof that bind to PD-1, and antibodies or antigen-binding fragments thereof that bind to PD-L1. Antibodies or antigen-binding fragments may also be conjugated to further moieties, as described herein. In particular, antibodies or antigen-binding fragments thereof are chimerized, humanized or human antibodies.

In one embodiment, an antibody that is a PD1/PD-L1 inhibitor is an isolated antibody.

In one embodiment, the PD1/PD-L1 inhibitor is an antibody, a fragment or construct thereof that prevents the interaction between PD-1 and PD-L1.

The PD1/PD-L1 inhibitor may be an inhibitory nucleic acid molecule, such as an oligonucleotide, siRNA, shRNA, an antisense DNA or RNA molecule, and an aptamer (e.g., DNA or RNA aptamer), in particular an antisense-oligonucleotide. In one embodiment, the PD1/PD-L1 inhibitor being siRNA interferes with mRNA therefore blocking translation, e.g., translation of PD-1 protein.

In one embodiment, the PD1/PD-L1 inhibitor is an antibody, an antigen-binding portion thereof or a construct thereof that disrupts or inhibits the interaction between the PD-1 receptor and one or more of its ligands, PD-L1 and/or PD-L2. Antibodies which bind to PD-1 or PD-L1 and disrupt or inhibit the interaction between PD-1 and one or more of its ligands are known in the art. In certain embodiments, the antibody, antigen-binding portion thereof or a construct thereof binds specifically to PD-1. In certain embodiments, the antibody, antigen-binding portion thereof or a construct thereof binds specifically to PD-L1.

In certain preferred embodiments, the PD1/PD-L1 inhibitor is an antibody that binds to PD-1, such as a PD-1 blocking antibody. In certain preferred embodiments, the PD1/PD-L1 inhibitor is an antibody that binds to PD-L1, such as a PD-L1 blocking antibody.

Exemplary PD1/PD-L1 inhibitors include, without limitation, anti-PD-1 antibodies such as BGB-A317 (BeiGene; see U.S. Pat. No. 8,735,553, WO 2015/35606 and US 2015/0079109), lambrolizumab (e.g., disclosed as hPD109A and its humanized derivatives h409A1, h409A16 and h409A17 in WO2008/156712), AB137132 (Abcam), EH12.2H7 and RMP1-14 (#BE0146; Bioxcell Lifesciences Pvt. LTD.), MIH4 (Affymetrix eBioscience), nivolumab (OPDIVO, BMS-936558; Bristol Myers Squibb; see U.S. Pat. No. 8,008,449; WO 2013/173223; WO 2006/121168), pembrolizumab (KEYTRUDA; MK-3475; Merck; see WO 2008/156712), pidilizumab (CT-011; CureTech; see Hardy et al., 1994, Cancer Res., 54(22):5793-6 and WO 2009/101611), PDR001 (Novartis; see WO 2015/112900), MEDI0680 (AMP-514; AstraZeneca; see WO 2012/145493), TSR-042 (see WO 2014/179664), cemiplimab (REGN-2810; Regeneron; H4H7798N; cf. US 2015/0203579 and WO 2015/112800), JS001 (TAIZHOU JUNSHI PHARMA; see Si-Yang Liu et al., 2007, J. Hematol. Oncol. 70: 136), AMP-224 (GSK-2661380; cf. Li et al., 2016, Int J Mol Sci 17(7):1151 and WO 2010/027827 and WO 2011/066342), PF-06801591 (Pfizer), tislelizumab (BGB-A317; BeiGene; see WO 2015/35606, U.S. Pat. No. 9,834,606, and US 2015/0079109), BI 754091, SHR-1210 (see WO2015/085847), and antibodies 17D8, 2D3, 4H1, 4A11, 7D3, and 5F4 as described in WO 2006/121168, INCSHR1210 (Jiangsu Hengrui Medicine; also known as SHR-1210; see WO 2015/085847), TSR-042 (Tesaro Biopharmaceutical; also known as ANB011; see WO2014/179664), GLS-010 (Wuxi/Harbin Gloria Pharmaceuticals; also known as WBP3055; see Si-Yang et al., 2017, J. Hematol. Oncol. 70: 136), STI-1110 (Sorrento Therapeutics; see WO 2014/194302), AGEN2034 (Agenus; see WO 2017/040790), MGA012 (Macrogenics; see WO 2017/19846), IBI308 (Innovent; see WO 2017/024465, WO 2017/025016, WO 2017/132825, and WO 2017/133540), cetrelimab (JNJ-63723283; JNJ-3283; see Calvo et al., J. Clin. Oncol. 36, no. 5_suppl (2018) 58), genolimzumab (CBT-501; see Patel et al., J. ImmunoTher. Cancer, 2017, 5(Suppl 2):P242), sasanlimab (PF-06801591; see Youssef et al., Proc. Am. Assoc. Cancer Res. Ann. Meeting 2017; Cancer Res 2017; 77(13 Suppl):Abstract), toripalimab (JS-001; see US 2016/0272708), camrelizumab (SHR-1210; INCSHR-1210; see US 2016/376367; Huang et al., Clin. Cancer Res. 2018; 24(6):1296-1304), spartalizumab (PDR001; see WO 2017/106656; Naing et al., J. Clin. Oncol. 34, no. 15_suppl (2016) 3060-3060), BCD-100 (JSC BIOCAD, Russia; see WO 2018/103017), balstilimab (AGEN2034; see WO 2017/040790), sintilimab (IBI-308; see WO 2017/024465 and WO 2017/133540), ezabenlimab (BI-754091; see US 2017/334995; Johnson et al., J. Clin. Oncol. 36, no. 5_suppl (2018) 212-212), zimberelimab (GLS-010; see WO 2017/025051), LZM-009 (see US 2017/210806), AK-103 (see WO 2017/071625, WO 2017/166804, and WO 2018/036472), retifanlimab (MGA-012; see WO 2017/019846), Sym-021 (see WO 2017/055547), CS1003 (see CN107840887), anti-PD-1 antibodies as described, e.g., in U.S. Pat. Nos. 7,488,802, 8,008,449, 8,168,757, WO 03/042402, WO 2010/089411 (further disclosing anti-PD-L1 antibodies), WO 2010/036959, WO 2011/159877 (further disclosing antibodies against TIM-3), WO 2011/082400, WO 2011/161699, WO 2009/014708, WO 03/099196, WO 2009/114335, WO 2012/145493 (further disclosing antibodies against PD-L1), WO 2015/035606, WO 2014/055648 (further disclosing anti-KIR antibodies), US 2018/0185482 (further disclosing anti-PD-L1 and anti-TIGIT antibodies), U.S. Pat. Nos. 8,008,449, 8,779,105, 6,808,710, 8,168,757, US 2016/0272708, and U.S. Pat. No. 8,354,509, small molecule antagonists to the PD-1 signaling pathway as disclosed, e.g., in Shaabani et al., 2018, Expert Op Ther Pat., 28(9):665-678 and Sasikumar and Ramachandra, 2018, BioDrugs, 32(5):481-497, siRNAs directed to PD-1 as disclosed, e.g., in WO 2019/000146 and WO 2018/103501, soluble PD-1 proteins as disclosed in WO 2018/222711 and oncolytic viruses comprising a soluble form of PD-1 as described, e.g., in WO 2018/022831.

In a certain embodiment, the PD1/PD-L1 inhibitor is nivolumab (OPDIVO; BMS-936558) or a biosimilar thereof, pembrolizumab (KEYTRUDA; MK-3475) or a biosimilar thereof, pidilizumab (CT-011), PDR001, MEDI0680 (AMP-514) or a biosimilar thereof, TSR-042, REGN2810, JS001, AMP-224 (GSK-2661380), PF-06801591, BGB-A317, BI 754091, or SHR-1210.

In certain embodiments, the PD1/PD-L1 inhibitor is an anti-PD1 or anti-PD-L1 antibody or antigen-binding fragment thereof comprising the complementary determining regions (CDRs) of one of the anti-PD1 or anti-PD-L1 antibodies or antigen-binding fragments described herein, such as the CDRs of one anti-PD1 or anti-PD-L1 antibody or antigen-binding fragment selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003.

In certain embodiments, the PD1/PD-L1 inhibitor is an anti-PD1 or anti-PD-L1 antibody or antigen-binding fragment thereof comprising the heavy chain variable region and the light chain variable region of one of the anti-PD1 or anti-PD-L1 antibodies or antigen-binding fragments described above, such as the heavy chain variable region and the light chain variable region of one anti-PD1 or anti-PD-L1 antibody or antigen-binding fragment selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003.

In certain embodiments, the PD1/PD-L1 inhibitor is an anti-PD-1 or anti-PD-L1 antibody or antigen-binding fragment thereof selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003.

In certain embodiments, the PD1/PD-L1 inhibitor is an antibody binding to PD1 or PD-L1. In some preferred embodiments, the PD1/PD-L1 inhibitor is an antibody which is an antagonist of PD1/PD-L1 interaction. In some preferred embodiments, the PD1/PD-L1 inhibitor is a PD1 blocking antibody or a PD-L1 blocking antibody.

In certain embodiments, the PD1/PD-L1 inhibitor is an antibody of an isotype selected from the group consisting of IgG1, IgG2, IgG3, and IgG4, such as of the IgG1 isotype. In one embodiment, the PD1/PD-L1 inhibitor is an antibody of IgG1 isotype. In one embodiment, the PD1/PD-L1 inhibitor is an antibody of IgG2 isotype. In one embodiment, the PD1/PD-L1 inhibitor is an antibody of IgG3 isotype. In one embodiment, the PD1/PD-L1 inhibitor is an antibody of IgG4 isotype.

In certain embodiments, the PD1/PD-L1 inhibitor is a full-length antibody or antibody fragment, such as a full-length IgG1 antibody.

In certain embodiments, the PD1/PD-L1 inhibitor is a monospecific antibody.

In one embodiment, the PD1/PD-L1 inhibitor is an antibody binding to PD1 comprising a heavy chain variable region (VH) comprising the CDR1, CDR2 and CDR3 sequences set forth in SEQ ID NO: 99, 100 and 101, respectively, and a light chain variable region (VL) comprising the CDR1, CDR2, and CDR3 sequences set forth in SEQ ID NO: 102, LAS and SEQ ID NO: 103, respectively.

In one embodiment, the PD1/PD-L1 inhibitor is an antibody binding to PD1 comprising a VH region comprising the amino acid sequence of SEQ ID NO: 104 and a VL region comprising the amino acid sequence of SEQ ID NO: 105.

In one embodiment, the PD1/PD-L1 inhibitor is an antibody binding to PD1 comprising a heavy chain comprising the amino acid sequence of SEQ ID NO: 106 and a light chain comprising the amino acid sequence of SEQ ID NO: 107.

In a preferred embodiment, the PD1/PD-L1 inhibitor is Pembrolizumab or a biosimilar thereof.

In a preferred embodiment, the PD1/PD-L1 inhibitor is Nivolumab or a biosimilar thereof.

In a preferred embodiment, the PD1/PD-L1 inhibitor is Atezolizumab or a biosimilar thereof.

In some embodiments, the PD1/PD-L1 inhibitor is a PD1 inhibitor, such as a PD1 blocking antibody. In some embodiments, the PD1/PD-L1 inhibitor is a PD-L1 inhibitor, such as a PD-L1 blocking antibody.

In certain embodiments, the PD1/PD-L1 inhibitor is a PD1 inhibitor selected from Pembrolizumab, Nivolumab, Cemiplimab, Dostarlimab, JTX-4014, Spartalizumab, Camrelizumab, Sintilimab, Tislelizumab, Toripalimab, INCMGA00012 (MGA012), AMP-224, AMP-514, or a respective biosimilar thereof.

In certain embodiments, the PD1 inhibitor is selected from Pembrolizumab, Nivolumab, Cemiplimab, Dostarlimab, JTX-4014, Spartalizumab, Camrelizumab, Sintilimab, Tislelizumab, Toripalimab, INCMGA00012 (MGA012), AMP-514, or a respective biosimilar thereof.

In certain embodiments, the PD1/PD-L1 inhibitor is a PD-L1 inhibitor selected from Atezolizumab, Avelumab, Durvalumab, KN035, CK-301, Acasunlimab, AUNP12, CA-170, BMS-986189, or a respective biosimilar thereof.

In certain embodiments, the PD-L1 inhibitor is selected from Atezolizumab, Avelumab, Durvalumab, KN035, CK-301, Acasunlimab, or a respective biosimilar thereof.

In a further preferred embodiment, the PD1/PD-L1 inhibitor is an antibody binding to PD-1.

The antibody binding to PD-1 may comprise a heavy chain variable region (VH) comprising a HCDR1, HCDR2, and HCDR3 sequence and a light chain variable region (VL) comprising a LCDR1, LCDR2, and LCDR3 sequence, wherein the HCDR1, HCDR2 and HCDR3 sequence comprises or has the sequence as set forth in SEQ ID NO: 49, SEQ ID NO: 46, and SEQ ID NO: 45, respectively, and the LCDR1, LCDR2 and LCDR3 sequence comprises or has the sequence as set forth in SEQ ID NO: 52, QAS, and SEQ ID NO: 50, respectively. A specific, but not limiting example of such an antibody is MAB-19-0202.

The terms “a heavy chain variable region” (also referred to as “VH”) and “a light chain variable region” (also referred to as “VL”) are used here in their most general meaning and comprise any sequences that are able to comprise complementarity determining regions (CDR), interspersed with other regions, which also termed framework regions (FR). The framework reagions inter alia space the CDRs so that they are able to form the antigen-binding site, in particular after folding and pairing of VH and VL. Preferably each VH and VL is composed of three CDRs and four FRs, arranged from amino-terminus to carboxy-terminus in the following order: FR1, CDR1, FR2, CDR2, FR3, CDR3, FR4. That is, the terms “a heavy chain variable region” and “a light chain variable region” are not to be construed to be limited to such sequences as they can be found in a native antibody or in the VH and VL sequences as exemplified herein (SEQ ID NOs: 54 to 57 of the sequence listing). These terms include any sequences capable of comprising and adequately positioning CDRs, for example such sequences as derived from VL and VH regions of native antibodies or as derived from the sequences as set forth in SEQ ID NOs: 54 to 57 of the sequence listing. It will be appreciated by those skilled in the art that in particular the sequences of the framework regions can be modified (includings both variants with regard to amino acid substitutions and variants with regard to the sequence length, i.e., insertion or deletion variants) without losing the charactistics of the VH and VL, respectively. In a preferred embodiment any modification is limited to the framework regions. But, a person skilled in the art is also well aware of the fact that also CDR, hypervariable and variable regions can be modified without losing the ability to bind PD-1. For example, CDR regions will be either identical or highly homologous to the regions specified herein. By “highly homologous” it is contemplated that from 1 to 5, preferably from 1 to 4, such as 1 to 3 or 1 or 2 substitutions may be made in the CDRs. In addition, the hypervariable and variable regions may be modified so that they show substantial homology with the regions specifically disclosed herein.

In the antibody binding to PD-1, the CDRs as specified herein have been identified by using two different CDR identification methods. The first numbering scheme used herein is according to Kabat (Wu and Kabat, 1970; Kabat et al., 1991), the second scheme is the IMGT numbering (Lefranc, 1997; Lefranc et al., 2005). In a third approach, the intersection of both identification schemes has been used.

The antibody binding to PD-1 may comprise one or more CDRs, a set of CDRs or a combination of sets of CDRs as described herein comprises said CDRs together with their intervening framework regions (also referred to as framing region or FR herein) or with portions of said framework regions. Preferably, the portion will include at least about 50% of either or both of the first and fourth framework regions, the 50% being the C-terminal 50% of the first framework region and the N-terminal 50% of the fourth framework region. Construction of antibodies made by recombinant DNA techniques may result in the introduction of residues N- or C-terminal to the variable regions encoded by linkers introduced to facilitate cloning or other manipulation steps, including the introduction of linkers to join variable regions of the disclosure to further protein sequences including immunoglobulin heavy chains, other variable domains (for example in the production of diabodies) or protein labels.

The antibody binding to PD-1 may comprise a heavy chain variable region (VH) comprising a sequence having at least 70%, at least 75%, at least 80%, at least 85%, at least 90%, at least 95%, at least 97%, at least 99%, or 100% identity to the amino acid sequence of the VH sequence as set forth in any one of SEQ ID NO: 56. In one embodiment, the antibody comprises a heavy chain variable region (VH), wherein the VH comprises the sequence as set forth in any one of SEQ ID NO: 56. In one embodiment, the antibody comprises a light chain variable region (VL) comprising a sequence having at least 70%, at least 75%, at least 80%, at least 85%, at least 90%, at least 95%, at least 97%, at least 99%, or 100% identity to the amino acid sequence of the VL sequence as set forth in any one of SEQ ID NO: 57. In one embodiment, the antibody comprises a light chain variable region (VL), wherein the VL comprises the sequence as set forth in any one of SEQ ID NO: 57.

The antibody binding to PD-1 may comprise a heavy chain variable region (VH) and a light chain variable region (VL), wherein the VH comprises or has the sequence as set forth in SEQ ID NO: 56 and the VL comprises or has the sequence as set forth in SEQ ID NO: 57, or respective variants of these sequences. Another example of an antibody binding to PD-1 may comprise a VH comprising or having the sequence as set forth in SEQ ID NO: 56, or a variant thereof, and a VL comprising or having the sequence as set forth in SEQ ID NO: 57, or a variant thereof. A specific, but not limiting example of such an antibody is MAB-19-0618. The antibody MAB-19-0618 has been derived from MAB-19-0202. Also encompassed by the present disclosure are variants of the said heavy chain variable regions (VH) and the said light chain variable regions (VL) and the respective combinations of these variant VHs and VLs.

The antibody binding to PD-1 may comprises a heavy chain and a light chain, which heavy chain comprises a heavy chain constant region comprising or having the sequence as set forth in SEQ ID NO: 38 or 128 and a heavy chain variable region (VH) comprising or having the sequence as set forth in SEQ ID NO: 56, and which light chain comprises a light chain constant region comprising or having the sequence as set forth in SEQ ID NO: 42 and a light chain variable region (VL) comprising or having the sequence as set forth in SEQ ID NO: 57.

The antibody binding to PD-1 may comprises a heavy chain and a light chain, which heavy chain comprises a heavy chain constant region comprising or having the sequence as set forth in SEQ ID NO: 38 or 128 and a heavy chain variable region (VH) comprising the CDR1, CDR2 and CDR3 sequences of the sequence as set forth in SEQ ID NO: 56, and which light chain comprises a light chain constant region comprising or having the sequence as set forth in SEQ ID NO: 42 and a light chain variable region comprising the CDR1, CDR2 and CDR3 sequences of the sequence as set forth in SEQ ID NO: 57. For example, the CDR1, CDR2 and CDR3 sequences are as specified herein.

The antibody binding to PD-1 may be a monoclonal, chimeric or a monoclonal, humanized antibody or a fragment of such an antibody. The antibodies can be whole antibodies or antigen-binding fragments thereof including, for example, bispecific antibodies.

In the antibody binding to PD-lone or more, preferably both heavy chain constant regions may have been modified so that binding of C1q to said antibody is reduced compared to a wild-type antibody, preferably reduced by at least 70%, at least 80%, at least 90%, at least 95%, at least 97%, or 100%. In one embodiment, the C1q binding can be determined by ELISA.

By “wild type” or “WT” or “native” herein is meant an amino acid sequence that is found in nature, including allelic variations. A wild type amino acid sequence, peptide or protein has an amino acid sequence that has not been intentionally modified.

In the antibody binding to PD-1, one or more, preferably both heavy chain constant regions may have been modified so that binding to one or more of the IgG Fc-gamma receptors to the antibody is reduced compared to a wild-type antibody, preferably by at least 70%, at least 80%, at least 90%, at least 95%, at least 97% or 100%. In one embodiment, the one or more IgG Fc-gamma receptors are selected from at least one of Fc-gamma RI, Fc-gamma RII, and Fc-gamma RIII. In one embodiment, the IgG Fc-gamma receptor is Fc-gamma RI.

In one embodiment, the antibody binding to PD-1 is not capable of inducing Fc-gamma RI-mediated effector functions or wherein the induced Fc-gamma RI-mediated effector functions are reduced compared to a wild-type antibody, preferably by at least 70%, at least 80%, at least 90%, at least 95%, at least 97% or 100%.

In one embodiment, the antibody binding to PD-1 is not capable of inducing at least one of complement dependent cytotoxicity (CDC) mediated lysis, antibody dependent cellular cytotoxicity (ADCC) mediated lysis, apoptosis, homotypic adhesion and/or phagocytosis or wherein at least one of complement dependent cytotoxicity (CDC) mediated lysis, antibody dependent cellular cytotoxicity (ADCC) mediated lysis, apoptosis, homotypic adhesion and/or phagocytosis is induced in a reduced extent, preferably reduced by at least 70%, at least 80%, at least 90%, at least 95%, at least 97% or 100%.

Antibody-dependent cell-mediated cytotoxicity is also referred to as “ADCC” herein. ADCC describes the cell-killing ability of effector cells as described herein, in particular lymphocytes, which preferably requires the target cell being marked by an antibody.

ADCC preferably occurs when antibodies bind to antigens on tumor cells and the antibody Fc domains engage Fc receptors (FcR) on the surface of immune effector cells. Several families of Fc receptors have been identified, and specific cell populations characteristically express defined Fc receptors. ADCC can be viewed as a mechanism to directly induce a variable degree of immediate tumor destruction that leads to antigen presentation and the induction of tumor-directed T-cell responses. Preferably, in vivo induction of ADCC will lead to tumor-directed T-cell responses and host-derived antibody responses.

Complement-dependent cytotoxicity is also referred to as “CDC” herein. CDC is another cell-killing method that can be directed by antibodies. IgM is the most effective isotype for complement activation. IgG1 and IgG3 are also both very effective at directing CDC via the classical complement-activation pathway. Preferably, in this cascade, the formation of antigen-antibody complexes results in the uncloaking of multiple C1q binding sites in close proximity on the CH2 domains of participating antibody molecules such as IgG molecules (C1q is one of three subcomponents of complement C1). Preferably these uncloaked C1q binding sites convert the previously low-affinity C1q-IgG interaction to one of high avidity, which triggers a cascade of events involving a series of other complement proteins and leads to the proteolytic release of the effector-cell chemotactic/activating agents C3a and C5a. Preferably, the complement cascade ends in the formation of a membrane attack complex, which creates pores in the cell membrane that facilitate free passage of water and solutes into and out of the cell and may lead to apoptosis.

In one embodiment, the antibody binding to PD-1 has reduced or depleted effector functions. In one embodiment, the antibody does not mediate ADCC or CDC or both.

In one embodiment, one or more, preferably both heavy chain constant regions of the antibody binding to PD-1 have been modified so that binding of neonatal Fc receptor (FcRn) to the antibody is unaffected, as compared to a wild-type antibody.

In one embodiment, the PD-1 to which the antibody is able to bind is human PD-1. In one embodiment, the PD-1 has or comprises the amino acid sequence as set forth in SEQ ID NO: 58 or SEQ ID NO: 59, or the amino acid sequence of PD-1 has at least 70%, at least 75%, at least 80%, at least 85%, at least 90%, at least 95%, at least 97%, at least 99%, or 100% identity to the amino acid sequence as set forth in SEQ ID NO: 58 or SEQ ID NO: 59, or is an immunogenic fragment thereof. In one embodiment, the antibody has the ability to bind to a native epitope of PD-1 present on the surface of living cells.

In one embodiment, the antibody binding to PD-1 comprises a heavy chain constant region, wherein the heavy chain constant region comprises an aromatic or non-polar amino acid at the position corresponding to position 234 in a human IgG1 heavy chain according to EU numbering and an amino acid other than glycine at the position corresponding to position 236 in a human IgG1 heavy chain according to EU numbering.

The term “amino acid corresponding to position . . . ” and similar expressions as used herein refer to an amino acid position number in a human IgG1 heavy chain. Corresponding amino acid positions in other immunoglobulins may be found by alignment with human IgG1. Thus, an amino acid or segment in one sequence that “corresponds to” an amino acid or segment in another sequence is one that aligns with the other amino acid or segment using a standard sequence alignment program such as ALIGN, ClustalW or similar, typically at default settings and has at least 50%, at least 80%, at least 90%, or at least 95% identity to a human IgG1 heavy chain. It is considered well-known in the art how to align a sequence or segment in a sequence and thereby determine the corresponding position in a sequence to an amino acid position according to the present disclosure.

With reference to, e.g., the amino acid sequence according to SEQ ID NO. 38 of the sequence listing of the present disclosure the amino acid positions corresponding to positions 234 to 236 in a human IgG1 heavy chain according to EU numbering are the amino acid positions 117 to 119 of SEQ ID NO. 38, with F being positioned at position 117 (corresponding to positions 234 in a human IgG1 heavy chain according to EU numbering), E being positioned at position 118 (corresponding to positions 235 in a human IgG1 heavy chain according to EU numbering) and R being positioned at position 119 (corresponding to positions 236 in a human IgG1 heavy chain according to EU numbering). In the sequence as shown below, the FER amino acid sequence is underlined and shown in bold letters.

| | |
|---|---|
| ASTKGPSVFPLAPSSKSTSGGTAALGCLVKDYFPEPVTVSWNSGALTSGVHTFPAVLQSS | 60 |
| GLYSLSSVVTVPSSSLGTQTYICNVNHKPSNTKVDKRVEPKSCDKTHTCPPCPAPEFERG | 120 |
| PSVFLFPPKPKDTLMISRTPEVTCVVVDVSHEDPEVKFNWYVDGVEVHNAKTKPREEQYN | 180 |
| STYRVVSVLTVLHQDWLNGKEYKCKVSNKALPAPIEKTISKAKGQPREPQVYTLPPSREE | 240 |
| MTKNQVSLTCLVKGFYPSDIAVEWESNGQPENNYKTTPPVLDSDGSFFLYSKLTVDKSRW | 300 |
| QQGNVFSCSVMHEALHNHYTQKSLSLSPG | 329 |

Unless otherwise indicated herein or otherwise clearly contradicted by the context, all references to amino acid positions in antibody heavy chain constant regions throughout this disclosure refer to the positions corresponding to the respective positions in a human IgG1 heavy chain according to EU numbering as set forth in Kabat (described in Kabat, E. A. et al., Sequences of proteins of immunological interest. 5th Edition—US Department of Health and Human Services, NIH publication No. 91-3242, pp 662,680,689 (1991)).

In one embodiment, the antibody binding to PD-1 comprises a heavy chain constant region which has a reduced or depleted Fc-mediated effector function or which induces Fc-mediated effector function to a lesser extent compared to another antibody comprising the same antigen binding regions and heavy chain constant regions (CHs) comprising human IgG1 hinge, CH2 and CH3 regions.

In one particular embodiment, said heavy chain constant region (CHs) in the antibody binding to PD-1 are modified so that the antibody induces Fc-mediated effector function to a lesser extent compared to an antibody which is identical except for comprising non-modified heavy chain constant regions (CHs).

The term “Fc-mediated effector function” as used herein refers to such functions in particular being selected from the list of IgG Fc receptor (FcgammaR, FcγR) binding, C1q binding, ADCC, CDC and any combinations thereof.

In the context of the present disclosure, the term “has a reduced or depleted Fc-mediated effector function” used in relation to an antibody, including a multispecific antibody, means that the antibody cause an overall decrease of Fc-mediated effector functions, such function in particular being selected from the list of IgG Fc receptor (FcgammaR, FcγR) binding, C1q binding, ADCC or CDC, preferably of 5% or greater, 10% or greater, 20% or greater, more preferably of 50% or greater, and most preferably of 75% or greater, in the level compared to a human IgG1 antibody comprising (i) the same CDR sequences, in particular comprising the same first and second antigen-binding regions, as said antibody and (ii) two heavy chains comprising human IgG1 hinge, CH2 and CH3 regions. A “depleted Fc-mediated effector function” or similar phrases includes a complete or essentially complete inhibition, i.e., a reduction to zero or essentially to zero.

In the context of the present disclosure, the term “induce Fc-mediated effector function to a lesser extent” used in relation to an antibody, including a multispecific antibody, means that the antibody induces Fc-mediated effector functions, such function in particular being selected from the list of IgG Fc receptor (FcgammaR, FcγR) binding, C1q binding, ADCC or CDC, to a lesser extent compared to a human IgG1 antibody comprising (i) the same CDR sequences, in particular comprising the same first and second antigen-binding regions, as said antibody and (ii) two heavy chains comprising human IgG1 hinge, CH2 and CH3 regions.

The Fc-mediated effector function may be determined by measuring binding of the binding agent to Fcγ receptors, binding to C1q, or induction of Fc-mediated cross-linking of Fcγ receptors. In particular, the Fc-mediated effector function may be determined by measuring binding of the binding agent to C1q and/or IgG FC-gamma RI.

In one embodiment relating to use of the antibody binding to PD-1, the amino acid at the position corresponding to position 236 in a human IgG1 heavy chain according to EU numbering is a basic amino acid.

The term “amino acid” and “amino acid residue” may herein be used interchangeably, and are not to be understood limiting. Amino acids are organic compounds containing amine (—NH₂) and carboxyl (—COOH) functional groups, along with a side chain (R group) specific to each amino acid. In the context of the present disclosure, amino acids may be classified based on structure and chemical characteristics.

In the present disclosure, amino acid residues are expressed by using the following abbreviations. Also, unless explicitly otherwise indicated, the amino acid sequences of peptides and proteins are identified from N-terminal to C-terminal (left terminal to right terminal), the N-terminal being identified as a first residue. Amino acids are designated by their 3-letter abbreviation, 1-letter abbreviation, or full name, as follows. Ala A: alanine; Asp D: aspartic acid; Glu: E: glutamic acid; Phe: F: phenylalanine; Gly: G glycine; His: H: histidine; Ile: I: isoleucine; Lys: K: lysine; Leu: L: leucine; Met: M: methionine; Asn: N: asparagine; Pro: P: proline; Gln: Q: glutamine; Arg: R: arginine; Ser: S: serine; Thr: T: threonine; Val: V: valine; Trp: W: tryptophan; Tyr: Y: tyrosine; Cys: C cysteine.

Naturally occurring amino acids may also be generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cysteine, serine, threonine, tyrosine) amino acids. Phenylalanine, tryptophan, and tyrosine are sometimes classified jointly as aromatic amino acids.

In one embodiment relating to use of an antibody binding to PD-1, the basic amino acid at the position corresponding to position 236 in a human IgG1 heavy chain according to EU numbering is selected from the group consisting of lysine, arginine and histidine. In one embodiment, the basic amino acid at the position corresponding to position 236 in a human IgG1 heavy chain according to EU numbering is arginine (G236R). Such an amino acid subsitition is also referred to herein as G236R. The term “G236R” indicates that at position 236 in a human IgG1 heavy chain according to EU numbering the amino acid glycine (G) is substituted by arginine (R). Within the present disclosure similar terms are used for other amino acid positions and amino acids. Unless indicated to the contrary the referenced amino acid position in these terms is the amino acid position in a human IgG1 heavy chain according to EU numbering.

In one embodiment relating to use of an antibody binding to PD-1, the amino acid at the position corresponding to position 234 in a human IgG1 heavy chain according to EU numbering is an aromatic amino acid. In one embodiment, the aromatic amino acid at this position is selected from the group consisting of phenylalanine, tryptophan and tyrosine.

In one embodiment relating to use of an antibody binding to PD-1, the amino acid at the position corresponding to position 234 in a human IgG1 heavy chain according to EU numbering is a non-polar amino acid. In one embodiment, the non-polar amino acid at this position is selected from the group consisting of alanine, valine, leucine, isoleucine, proline, phenylalanine, methionine and tryptophan. In one embodiment, the non-polar amino acid at this position is selected from the group consisting of isoleucine, proline, phenylalanine, methionine and tryptophan.

In one embodiment relating to use of an antibody binding to PD-1, the amino acid at the position corresponding to position 234 in a human IgG heavy chain according to EU numbering is phenylalanine (L234F).

Exemplary combinations of possible amino acids at the positions corresponding to positions 234 and 236 in a human IgG1 heavy chain according to EU numbering are set forth in the table below:

TABLE 22

| Amino acid position 234 | Amino acid position 236 |
|---|---|
| Phenylalanine (F) | Arginine (R) |
| Tryptophan (W) | Arginine (R) |
| Tyrosine (Y) | Arginine (R) |
| Alanine (A) | Arginine (R) |
| Valine (V) | Arginine (R) |
| Leucine (L) | Arginine (R) |
| Isoleucine (I) | Arginine (R) |
| Proline (P) | Arginine (R) |
| Methionine (M) | Arginine (R) |
| Phenylalanine (F) | Lysine (K) |
| Tryptophan (W) | Lysine (K) |
| Tyrosine (Y) | Lysine (K) |
| Alanine (A) | Lysine (K) |
| Valine (V) | Lysine (K) |
| Leucine (L) | Lysine (K) |
| Isoleucine (I) | Lysine (K) |
| Proline (P) | Lysine (K) |
| Methionine (M) | Lysine (K) |
| Phenylalanine (F) | Histidine (H) |
| Tryptophan (W) | Histidine (H) |
| Tyrosine (Y) | Histidine (H) |
| Alanine (A) | Histidine (H) |
| Valine (V) | Histidine (H) |
| Leucine (L) | Histidine (H) |
| Isoleucine (I) | Histidine (H) |
| Proline (P) | Histidine (H) |
| Methionine (M) | Histidine (H) |

For example, at the positions corresponding to the positions 234 and 236 in a human IgG1 heavy chain according to EU numbering, in particular the following amino acids may be present in the heavy chain constant region of the antibody binding to PD-1: 234F/236R, 234W/236R, 234Y/236R, 234A/236R, 234L/236R, 234F/236K, 234W/236K, 234Y/236K, 234A/236K, 234L/236K, 234F/236H, 234W/236H, 234Y/236H, 234A/236H, or 234L/236H.

The aforementioned amino acids or amino acids substitutions at positions 234 and 236 may be present only in one heavy chain of the antibody binding to PD-1 or in both heavy chains of the antibody binding to PD-1. The respective amino acids present in first and the second heavy chain of the antibody may be selected independently from each other.

For example, at least one heavy chain of the antibody binding to PD-1 can comprise the following sequence (SEQ ID NO: 38):

| | |
|---|---|
| ASTKGPSVFPLAPSSKSTSGGTAALGCLVKDYFPEPVTVSWNSGALTSGVHTFPAVLQSS | 60 |
| GLYSLSSVVTVPSSSLGTQTYICNVNHKPSNTKVDKRVEPKSCDKTHTCPPCPAPEFLRG | 120 |
| PSVFLFPPKPKDTLMISRTPEVTCVVVDVSHEDPEVKFNWYVDGVEVHNAKTKPREEQYN | 180 |
| STYRVVSVLTVLHQDWLNGKEYKCKVSNKALPAPIEKTISKAKGQPREPQVYTLPPSREE | 240 |
| MTKNQVSLTCLVKGFYPSDIAVEWESNGQPENNYKTTPPVLDSDGSFFLYSKLTVDKSRW | 300 |
| QQGNVFSCSVMHEALHNHYTQKSLSLSPG | 329 |

In one embodiment relating to the antibody binding to PD-1, the said heavy chain in which the amino acids at the position corresponding to positions 234 and 236 in a human IgG heavy chain according to EU numbering are as specified above, furthermore the amino acid at the position corresponding to position 235 in a human IgG1 heavy chain according to EU numbering is an acidic amino acid. In one embodiment, the acidic amino acid at this position is selected from aspartate or glutamate. In one embodiment, the amino acid at the position corresponding to position 235 in a human IgG1 heavy chain according to EU numbering is glutamate (L235E).

In one embodiment relating to the antibody binding to PD-1, in the heavy chain constant region the amino acids at the position corresponding to positions 234, 235 and 236 in a human IgG heavy chain according to EU numbering are a non-polar or aromatic amino acid at position 234, an acidic amino acid at position 235 and a basic amino acid at position 236.

Exemplary combinations of possible amino acids at the positions corresponding to positions 234, 235 and 236 in a human IgG1 heavy chain according to EU numbering are set forth in the table below:

TABLE 23

| Amino acid | | Amino acid |
|---|---|---|
| position 234 | Amino acid position 235 | position 236 |
| Phenylalanine (F) | Asparatate (D) or Glutamate | Arginine (R) |
| | (E) | |
| Tryptophan (W) | Asparatate (D) or Glutamate | Arginine (R) |
| | (E) | |
| Tyrosine (Y) | Asparatate (D) or Glutamate | Arginine (R) |
| | (E) | |
| Alanine (A) | Asparatate (D) or Glutamate | Arginine (R) |
| | (E) | |
| Valine (V) | Asparatate (D) or Glutamate | Arginine (R) |
| | (E) | |
| Leucine (L) | Asparatate (D) or Glutamate | Arginine (R) |
| | (E) | |
| Isoleucine (I) | Asparatate (D) or Glutamate | Arginine (R) |
| | (E) | |
| Proline (P) | Asparatate (D) or Glutamate | Arginine (R) |
| | (E) | |
| Methionine (M) | Asparatate (D) or Glutamate | Arginine (R) |
| | (E) | |
| Phenylalanine (F) | Asparatate (D) or Glutamate | Lysine (K) |
| | (E) | |
| Tryptophan (W) | Asparatate (D) or Glutamate | Lysine (K) |
| | (E) | |
| Tyrosine (Y) | Asparatate (D) or Glutamate | Lysine (K) |
| | (E) | |
| Alanine (A) | Asparatate (D) or Glutamate | Lysine (K) |
| | (E) | |
| Valine (V) | Asparatate (D) or Glutamate | Lysine (K) |
| | (E) | |
| Leucine (L) | Asparatate (D) or Glutamate | Lysine (K) |
| | (E) | |
| Isoleucine (I) | Asparatate (D) or Glutamate | Lysine (K) |
| | (E) | |
| Proline (P) | Asparatate (D) or Glutamate | Lysine (K) |
| | (E) | |
| Methionine (M) | Asparatate (D) or Glutamate | Lysine (K) |
| | (E) | |
| Phenylalanine (F) | Asparatate (D) or Glutamate | Histidine (H) |
| | (E) | |
| Tryptophan (W) | Asparatate (D) or Glutamate | Histidine (H) |
| | (E) | |
| Tyrosine (Y) | Asparatate (D) or Glutamate | Histidine (H) |
| | (E) | |
| Alanine (A) | Asparatate (D) or Glutamate | Histidine (H) |
| | (E) | |
| Valine (V) | Asparatate (D) or Glutamate | Histidine (H) |
| | (E) | |
| Leucine (L) | Asparatate (D) or Glutamate | Histidine (H) |
| | (E) | |
| Isoleucine (I) | Asparatate (D) or Glutamate | Histidine (H) |
| | (E) | |
| Proline (P) | Asparatate (D) or Glutamate | Histidine (H) |
| | (E) | |
| Methionine (M) | Asparatate (D) or Glutamate | Histidine (H) |
| | (E) | |

For example, at the positions corresponding to the positions 234, 235 and 236 in a human IgG1 heavy chain according to EU numbering, in particular the following amino acids may be present in the heavy chain constant region of the antibody binding to PD-1: 234F/235E/236R, 234W/235E/236R, 234Y/235E/236R, 234A/235E/236R, 234L/235E/236R, 234F/235D/236R, 234W/235D/236R, 234Y/235D/236R, 234A/235D/236R, 234L/235D/236R, 234F/235L/236R, 234W/235L/236R, 234Y/235L/236R, 234A/235L/236R, 234L/235L/236R, 234F/235A/236R, 234W/235A/236R, 234Y/235A/236R, 234A/235A/236R, 234L/235A/236R, 234F/235E/236K, 234W/235E/236K, 234Y/235E/236K, 234A/235E/236K, 234L/235E/236K, 234F/235D/236K, 234W/235D/236K, 234Y/235D/236K, 234A/235D/236K, 234L/235D/236K, 234F/235L/236K, 234W/235L/236K, 234Y/235L/236K, 234A/235L/236K, 234L/235L/236K, 234F/235A/236K, 234W/235A/236K, 234Y/235A/236K, 234A/235A/236K, 234L/235A/236K, 234F/235E/236H, 234W/235E/236H, 234Y/235E/236H, 234A/235E/236H, 234L/235E/236H, 234F/235D/236H, 234W/235D/236H, 234Y/235D/236H, 234A/235D/236H, 234L/235D/236H, 234F/235L/236H, 234W/235L/236H, 234Y/235L/236H, 234A/235L/236H, 234L/235L/236H, 234F/235A/236H, 234W/235A/236H, 234Y/235A/236H, 234A/235A/236H, or 234L/235A/236H.

The aforementioned amino acids or amino acids substitutions at positions 234, 235 and 236 may be present only in one heavy chain of the antibody or in both heavy chains of the antibody. The respective amino acids present in first and the second heavy chain of the antibody may be selected independently from each other.

For example, at least one heavy chain of the antibody binding to PD-1 can comprise the following sequence (SEQ ID NO: 128 or 38):

| | |
|---|---|
| ASTKGPSVFPLAPSSKSTSGGTAALGCLVKDYFPEPVTVSWNSGALTSGVHTFPAVLQSS | 60 |
| GLYSLSSVVTVPSSSLGTQTYICNVNHKPSNTKVDKRVEPKSCDKTHTCPPCPAPEFERG | 120 |
| PSVFLFPPKPKDTLMISRTPEVTCVVVDVSHEDPEVKFNWYVDGVEVHNAKTKPREEQYN | 180 |
| STYRVVSVLTVLHQDWLNGKEYKCKVSNKALPAPIEKTISKAKGQPREPQVYTLPPSREE | 240 |
| MTKNQVSLTCLVKGFYPSDIAVEWESNGQPENNYKTTPPVLDSDGSFFLYSKLTVDKSRW | 300 |
| QQGNVFSCSVMHEALHNHYTQKSLSLSPG | 329 |

Any permutations and combinations of all described amino acid substitutions at positions 234, 236 and 235, if applicable, in this application, e.g., as shown in Tables 22 and 23, should be considered disclosed by the description of the present application unless the context indicates otherwise. For example, in one embodiment of the antibody the first heavy chain comprises the amino acids FER at the position corresponding to positions 234 to 236 in a human IgG1 heavy chain according to EU numbering or the first heavy chain comprises or consists essentially of or consists of an amino acid sequence set forth in SEQ ID NO: 38, and the second heavy chain of said antibody comprises other amino acids, e.g., the amino acids AAG or LLG at the positions corresponding to positions 234 to 236 in a human IgG1 heavy chain according to EU numbering or comprises or the second heavy chain of said antibody comprises or consists essentially of or consists of an amino acid sequence set forth in SEQ ID NO: 37 or 43. In another embodiment of the antibody, the first and the second heavy chains comprise the same amino acids at the position corresponding to positions 234 to 236 in a human IgG1 heavy chain according to EU numbering, i.e., the same aromatic or non-polar amino acid at the position corresponding to position 234 in a human IgG1 heavy chain according to EU numbering, e.g. F, and the same amino acid other than glycine at the position corresponding to position 236 in a human IgG1 heavy chain according to EU numbering, e.g., R, such as the specific combination of FER or FLR.

In one embodiment, the antibody binding to PD-1 comprises at least one or two heavy chain constant regions, wherein the amino acid corresponding to position 234 is phenylalanine, the amino acid corresponding to position 235 is glutamate, and the amino acid corresponding to position 236 is arginine (L234F/L235E/G236R=FER).

In one embodiment, the antibody binding to PD-1 comprises one or more a heavy chain constant region (CH) comprising a sequence having at least 70%, at least 75%, at least 80%, at least 85%, at least 90%, at least 95%, at least 97%, at least 99%, or 100% identity to the amino acid sequence of the heavy chain constant region sequence as set forth in SEQ ID NO: 38.

In one embodiment, the antibody binding to PD-1 comprises one or more, e.g., two heavy chain constant region (CH), wherein the heavy chain constant region comprises the sequence as set forth in SEQ ID NO: 38.

In one embodiment, the antibody binding to PD-1 comprises a heavy chain having the sequence as set forth in SEQ ID NO: 139, and a light chain having the sequence as set forth in SEQ ID NO: 140.

The antibody is preferably of the IgG1 isotype.

As used herein, the term “isotype” refers to the immunoglobulin class that is encoded by heavy chain constant region genes. When the IgG1 isotype, is mentioned herein, the term is not limited to a specific isotype sequence, e.g., a particular IgG1 sequence, but is used to indicate that the antibody is closer in sequence to that isotype, e.g. IgG1, than to other isotypes. Thus, e.g., an IgG1 antibody disclosed herein may be a sequence variant of a naturally-occurring IgG1 antibody, including variations in the constant regions.

IgG1 antibodies can exist in multiple polymorphic variants termed allotypes (reviewed in Jefferis and Lefranc 2009. mAbs Vol 1 Issue 4 1-7) any of which are suitable for use in some of the embodiments herein. Common allotypic variants in human populations are those designated by the letters a, f, n, z or combinations thereof. In any of the embodiments herein, the antibody may comprise a heavy chain Fc region comprising a human IgG Fc region. In further embodiments, the human IgG Fc region comprises a human IgG1.

In mammals there are two types of light chains, i.e., lambda and kappa. The immunoglobulin chains comprise a variable region and a constant region. The constant region is essentially conserved within the different isotypes of the immunoglobulins, wherein the variable part is highly divers and accounts for antigen recognition.

For example or in an embodiment, an antibody, preferably a monoclonal antibody, used according to the present invention the present invention is a IgG1, K isotype or A isotype, preferably comprising human IgG1/K or human IgG1/A constant parts, or the antibody, preferably the monoclonal antibody, is derived from a IgG1, A (lambda) or IgG1, K (kappa) antibody, preferably from a human IgG1, A (lambda) or a human IgG1, K (kappa) antibody.

In one embodiment, the antibody binding to PD-1 comprises a light chain having a light chain constant region (LC) comprising a sequence having at least 70%, at least 75%, at least 80%, at least 85%, at least 90%, at least 95%, at least 97%, at least 99%, or 100% identity to the amino acid sequence of the LC sequence as set forth in SEQ ID NO: 42. In one embodiment, the antibody comprises a light chain having a light chain constant region (LC) comprising the sequence as set forth in SEQ ID NO: 42.

In one embodiment of the invention, the antibody binding to PD-1 is a full-length IgG1 antibody, e.g., e.g., IgG1, K. In one embodiment of the invention, the binding agent is a full-length human IgG1 antibody, e.g., IgG1, K.

In one embodiment, the antibody binding to PD-1 can be derivatized, linked to or co-expressed to other binding specificities. In another embodiment, the antibody can be derivatized, linked to or co-expressed with another functional molecule, e.g., another peptide or protein (e.g., a Fab′ fragment). For example, the can be functionally linked (e.g., by chemical coupling, genetic fusion, noncovalent association or otherwise) to one or more other molecular entities, such as another antibody (e.g., to produce a bispecific or a multispecific antibody).

The antibody binding to PD-1 may be a human antibody. The term “human antibody”, as used herein, is intended to include antibodies having variable and constant regions derived from human germline immunoglobulin sequences. The human antibody binding to PD-1 may include amino acid residues not encoded by human germline immunoglobulin sequences (e.g., mutations introduced by random or site-specific mutagenesis in vitro or by somatic mutation in vivo).

The present disclosure includes the use of bispecific and multispecific molecules comprising at least one first binding specificity for PD-1 and a second binding specificity (or further binding specifities) for a second target epitope (or for further target epitopes).

In one embodiment the first antigen-binding region of the multispecific antibody binding to PD-1 comprises the heavy chain variable region (VH) and/or the light chain variable region (VL) as set forth herein.

In one embodiment relating to the use of a multispecific antibody binding to PD-1, the antibody comprises first and second binding arms derived from full-length antibodies, such as from full-length IgG1, A (lambda) or IgG1, K (kappa) antibodies as mentioned above. In one embodiment, the first and second binding arms are derived from monoclonal antibodies.

For example or in a preferred embodiment, the first and/or second binding arm is derived from a IgG1, K isotype or A isotype, preferably comprising human IgG1/K or human IgG1/A constant parts.

The said first antigen-binding region binding to PD-1 of the multispecific or bispecific antibody used according to the present invention may comprise heavy and light chain variable regions of an antibody which competes for PD-1 binding with PD-L1 and/or PD-L2. In one embodiment relating to the use of the multispecific or bispecific antibody, the first antigen-binding region binding to PD-1 comprises the heavy chain variable region (VH) and/or the light chain variable region (VL) as set forth herein.

As used herein, the term “effector cell” refers to an immune cell which is involved in the effector phase of an immune response, as opposed to the cognitive and activation phases of an immune response. Exemplary immune cells include cells of myeloid or lymphoid origin, e.g, lymphocytes (e.g., B cells and T cells including cytolytic T cells (CTLs), killer cells, natural killer cells, macrophages, monocytes, eosinophils, neutrophils, polymorphonuclear cells, granulocytes, mast cells, and basophils. “Target cell” shall mean any undesirable cell in a subject (e.g., a human or animal) that can be targeted by an antibody. In preferred embodiments, the target cell is a tumor cell.

In another embodiment, the PD1/PD-L1 inhibitor is a multispecific antibody, such as a bispecific antibody.

In a preferred embodiment, the PD1/PD-L1 inhibitor is a PD-L1 inhibitor comprising a first binding region binding to CD137 and a second binding region binding to PD-1.

In one embodiment, PD-L1 is human PD-L1, in particular human PD-L1 comprising the sequence set forth in SEQ ID NO: 98. In one embodiment, CD137 is human CD137, in particular human CD137 comprising the sequence set forth in SEQ ID NO: 97.

In one embodiment, a) the first binding region of the PD-L1 inhibitor comprises a heavy chain variable region (VH) comprising the CDR1, CDR2, and CDR3 sequences set forth in: SEQ ID NO: 80, 81, and 82, respectively, and a light chain variable region (VL) comprising the CDR1, CDR2, and CDR3 sequences set forth in: SEQ ID NO: 84, GAS, and SEQ ID NO: 85, respectively; and b) the second binding region of the PD-L1 inhibitor comprises a heavy chain variable region (VH) comprising the CDR1, CDR2, and CDR3 sequences set forth in: SEQ ID NO: 87, 88, and 89, respectively, and a light chain variable region (VL) comprising the CDR1, CDR2, and CDR3 sequences set forth in: SEQ ID NO: 91, DDN, and SEQ ID NO: 92, respectively.

In one embodiment, a) the first binding region of the PD-L1 inhibitor comprises a heavy chain variable region (VH) comprising the amino acid sequence set forth in SEQ ID NO: 79 and a light chain variable region (VL) region comprising the amino acid sequence set forth in SEQ ID NO: 83; and b) the second binding region of the PD-L1 inhibitor comprises a heavy chain variable region (VH) comprising the amino acid sequence set forth in SEQ ID NO: 86 and a light chain variable region (VL) region comprising the amino acid sequence set forth in SEQ ID NO: 90.

In one embodiment, the PD-L1 inhibitor is a multispecific antibody, such as a bispecific antibody.

In one embodiment, the PD-L1 inhibitor is in the format of a full-length antibody or an antibody fragment.

In one embodiment the PD-L1 inhibitor is an antibody comprising a first binding arm and a second binding arm, wherein the first binding arm comprises
i) a polypeptide comprising said first heavy chain variable region (VH) and a first heavy chain constant region (CH), and
ii) a polypeptide comprising said first light chain variable region (VL) and a first light chain constant region (CL);
and the second binding arm comprises
iii) a polypeptide comprising said second heavy chain variable region (VH) and a second heavy chain constant region (CH), and
iv) a polypeptide comprising said second light chain variable region (VL) and a second light chain constant region (CL).

In one embodiment, the PD-L1 inhibitor comprises
i) a first heavy chain and light chain comprising said antigen-binding region capable of binding to CD137, the first heavy chain comprising a first heavy chain constant region and the first light chain comprising a first light chain constant region; and
ii) a second heavy chain and light chain comprising said antigen-binding region capable of binding PD-L1, the second heavy chain comprising a second heavy chain constant region and the second light chain comprising a second light chain constant region.

In one embodiment, (i) the amino acid in the position corresponding to F405 in a human IgG1 heavy chain according to EU numbering is L in said first heavy chain constant region (CH), and the amino acid in the position corresponding to K409 in a human IgG1 heavy chain according to EU numbering is R in said second heavy chain constant region (CH), or (ii) the amino acid in the position corresponding to K409 in a human IgG1 heavy chain according to EU numbering is R in said first heavy chain, and the amino acid in the position corresponding to F405 in a human IgG1 heavy chain according to EU numbering is L in said second heavy chain.

In one embodiment, the positions corresponding to positions L234 and L235 in a human IgG1 heavy chain according to EU numbering are F and E, respectively, in said first and second heavy chains.

In one embodiment, the positions corresponding to positions L234, L235, and D265 in a human IgG1 heavy chain according to EU numbering are F, E, and A, respectively, in said first and second heavy chain constant regions (HCs).

In one embodiment, the positions corresponding to positions L234 and L235 in a human IgG1 heavy chain according to EU numbering of both the first and second heavy chain constant regions in the PD-L1 inhibitor are F and E, respectively, and (i) the position corresponding to F405 in a human IgG1 heavy chain according to EU numbering of the first heavy chain constant region is L, and the position corresponding to K409 in a human IgG1 heavy chain according to EU numbering of the second heavy chain is R, or (ii) the position corresponding to K409 in a human IgG1 heavy chain according to EU numbering of the first heavy chain constant region is R, and the position corresponding to F405 in a human IgG1 heavy chain according to EU numbering of the second heavy chain is L.

In one embodiment, the positions corresponding to positions L234, L235, and D265 in a human IgG1 heavy chain according to EU numbering of both the first and second heavy chain constant regions in the PD-L1 inhibitor are F, E, and A, respectively, and wherein (i) the position corresponding to F405 in a human IgG1 heavy chain according to EU numbering of the first heavy chain constant region is L, and the position corresponding to K409 in a human IgG1 heavy chain according to EU numbering of the second heavy chain constant region is R, or (ii) the position corresponding to K409 in a human IgG1 heavy chain according to EU numbering of the first heavy chain is R, and the position corresponding to F405 in a human IgG1 heavy chain according to EU numbering of the second heavy chain is L.

In one embodiment, the constant region of said first and/or second heavy chain in the PD-L1 inhibitor, such as the second heavy chain, comprises or consists essentially of or consists of an amino acid sequence selected from the group consisting of
a) the sequence set forth in SEQ ID NO: 94 or 96 [IgG1-Fc_FEAL];
b) a subsequence of the sequence in a), such as a subsequence, wherein 1, 2, 3, 4, 5, 6, 7, 8, 9 or 10 consecutive amino acids has/have been deleted, starting from the N-terminus or C-terminus of the sequence defined in a); and
c) a sequence having at most 6 substitutions, such as at most 5 substitutions, at most 4 substitutions, at most 3, at most 2 substitutions or at most 1 substitution, compared to the amino acid sequence defined in a) or b).

In one embodiment, the constant region of said first and/or second heavy chain in the PD-L1 inhibitor, such as the first heavy chain, comprises or consists essentially of or consists of an amino acid sequence selected from the group consisting of
a) the sequence set forth in SEQ ID NO: 93 or 95 [IgG1-Fc_FEAR];
b) a subsequence of the sequence in a), such as a subsequence, wherein 1, 2, 3, 4, 5, 6, 7, 8, 9 or 10 consecutive amino acids has/have been deleted, starting from the N-terminus or C-terminus of the sequence defined in a); and
c) a sequence having at most 6 substitutions, such as at most 5 substitutions, at most 4, at most 3, at most 2 substitutions or at most 1 substitution, compared to the amino acid sequence defined in a) or b).

In one embodiment, said PD-L1 inhibitor comprises a kappa (κ) light chain constant region.

In one embodiment, said PD-L1 inhibitor comprises a lambda (A) light chain constant region.

In one embodiment, said first light chain constant region of the PD-L1 inhibitor is a kappa (κ) light chain constant region or a lambda (A) light chain constant region.

In one embodiment, said second light chain constant region of the PD-L1 inhibitor is a lambda (λ) light chain constant region or a kappa (κ) light chain constant region.

In one embodiment, said first light chain constant region of the PD-L1 inhibitor is a kappa (κ) light chain constant region and said second light chain constant region is a lambda (λ) light chain constant region or said first light chain constant region is a lambda (λ) light chain constant region and said second light chain constant region is a kappa (κ) light chain constant region.

In one embodiment, the kappa (κ) light chain of the PD-L1 inhibitor comprises an amino acid sequence selected from the group consisting of
a) the sequence set forth in SEQ ID NO: 16,
b) a subsequence of the sequence in a), such as a subsequence, wherein 1, 2, 3, 4, 5, 6, 7, 8, 9 or 10 consecutive amino acids has/have been deleted, starting from the N-terminus or C-terminus of the sequence defined in a); and
c) a sequence having at most 10 substitutions, such as at most 9 substitutions, at most 8, at most 7, at most 6, at most 5, at most 4 substitutions, at most 3, at most 2 substitutions or at most 1 substitution, compared to the amino acid sequence defined in a) or b).

In one embodiment, the lambda (λ) light chain of the PD-L1 inhibitor comprises an amino acid sequence selected from the group consisting of
a) the sequence set forth in SEQ ID NO: 17,
b) a subsequence of the sequence in a), such as a subsequence, wherein 1, 2, 3, 4, 5, 6, 7, 8, 9 or 10 consecutive amino acids has/have been deleted, starting from the N-terminus or C-terminus of the sequence defined in a); and
c) a sequence having at most 10 substitutions, such as at most 9 substitutions, at most 8, at most 7, at most 6, at most 5, at most 4 substitutions, at most 3, at most 2 substitutions or at most 1 substitution, compared to the amino acid sequence defined in a) or b).

In one embodiment, the PD-L1 inhibitor is of an isotype selected from the group consisting of IgG1, IgG2, IgG3, and IgG4.

In one embodiment, the PD-L1 inhibitor is a full-length IgG1 antibody.

In one embodiment, the PD-L1 inhibitor is an antibody of the IgG1m(f) allotype.

In one embodiment, the PD-L1 inhibitor is a bispecific antibody binding to CD137 and PD-1, the bispecific antibody having i) a first heavy chain comprising the amino acid sequence set forth in SEQ ID NO: 75 and a first light chain comprising the amino acid sequence set forth in SEQ ID NO: 76, and ii) a second heavy chain comprising the amino acid sequence set forth in SEQ ID NO: 77 and a second light chain comprising the amino acid sequence set forth in SEQ ID NO: 78.

In one embodiment, the PD-L1 inhibitor is acasunlimab or a biosimilar thereof.

### Subject and Tumor or Cancer to be Treated

The subject to be treated according to the present disclosure is preferably a human subject.

In one embodiment the tumor or cancer is a solid tumor.

In one embodiment said tumor is a PD-L1 positive tumor.

In one embodiment the tumor or cancer is head and neck squamous cell carcinoma (HNSCC), such as HNSCC of the oral cavity, pharynx or larynx.

In one embodiment the HNSCC is recurrent, unresectable or metastatic.

In one embodiment the tumor or cancer is non-small cell lung cancer (NSCLC), such as a squamous or non-squamous NSCLC.

In one embodiment the NSCLC is recurrent, unresectable or metastatic.

In one embodiment the NSCLC does not have an epidermal growth factor (EGFR)-sensitizing mutation and/or anaplastic lymphoma (ALK) translocation and/or ROS1 rearrangement.

In one embodiment the NSCLC is NTRK1/2/3 (neurotrophic receptor tyrosine kinase 1/2/3) fusion positive, and/or has a mutation in KRAS (KRAS proto-oncogene, GTPase), BRAF (B-Raf proto-oncogene, serine/threonine kinase), or MET (MET proto-oncogene, receptor tyrosine kinase) gene, and/or has RET (ret proto-oncogene) gene rearrangements, and the subject has received prior treatment with a respective targeted therapy.

In one embodiment the subject has received prior treatment with a PD-1 inhibitor or a PD-L1 inhibitor, such as anti-PD-1 antibody or an anti-PD-L1 antibody, preferably at least two doses of the PD-1 inhibitor or the PD-L1 inhibitor.

In one embodiment the subject has received prior treatment with a platinum-based therapy or an alternative chemotherapy if platinum ineligible, eg a gemcitabine-containing regimen.

In one embodiment the tumor or cancer has relapsed and/or progressed after treatment, such as systemic treatment with a checkpoint inhibitor.

In one embodiment the subject has received at least one prior line of systemic therapy, such as systemic therapy comprising a PD-1 inhibitor or a PD-L1 inhibitor, such as an anti-PD-1 antibody or an anti-PD-L1 antibody.

In one embodiment the cancer or tumor has relapsed and/or is refractory, or the subject has progressed after treatment with a PD-1 inhibitor or a PD-L1 inhibitor, such as an anti PD-1 antibody or an anti-PD-L1 antibody, the PD-1 inhibitor or PD-L1 inhibitor being administered as monotherapy or as part of a combination therapy.

In one embodiment last prior treatment was with a PD1 inhibitor or PD-L1 inhibitor, such as an anti PD-1 antibody or an anti-PD-L1 antibody, the PD-1 inhibitor or PD-L1 inhibitor being administered as monotherapy or as part of a combination therapy.

In one embodiment the time from progression on last treatment with a PD-1 inhibitor or PD-L1 inhibitor, such as an anti PD-1 antibody or an anti-PD-L1 antibody is 6 months or less.

In one embodiment the time from last dosing of a PD-1 inhibitor or PD-L1 inhibitor, such as an anti PD-1 antibody or an anti-PD-L1 antibody as part of last prior treatment is 6 months or less.

In one embodiment the cancer or tumor has relapsed and/or is refractory, or the subject has progressed during or after i) platinum doublet chemotherapy following treatment with an anti-PD-1 antibody or an anti-PD-L1 antibody, or ii) treatment with an anti-PD-1 antibody or an anti-PD-L1 antibody following platinum doublet chemotherapy.

In a second aspect, the present disclosure provides a kit comprising i) a binding agent comprising at least one binding region binding to CD27 and ii) a PD1/PD-L1 inhibitor.

In one embodiment of the kit according to the second aspect, the binding agent is as defined in any aspect or embodiment of the present disclosure.

In one embodiment of the kit according to the second aspect, the PD1/PD-L1 inhibitor is as defined in any aspect or embodiment of the present disclosure.

In one embodiment of the kit according to the second aspect, the binding agent, the PD1/PD-L1 inhibitor, and, if present, one or more additional therapeutic agents are for systemic administration, in particular for injection or infusion, such as intravenous injection or infusion.

In a third aspect, the present disclosure provides a kit for use in a method for reducing progression or preventing progression of a tumor or treating cancer in a subject, said kit comprising i) a binding agent comprising at least one binding region binding to CD27 and ii) a PD1/PD-L1 inhibitor.

In one embodiment of the kit for use according to the third aspect, the kit is as defined in any aspect or embodiment of the present disclosure.

In one embodiment of the kit for use according to the third aspect, the tumor or cancer is as defined in any aspect or embodiment of the present disclosure.

In one embodiment of the kit for use according to the third aspect, the subject is as defined in any aspect or embodiment of the present disclosure.

In one embodiment of the kit for use according to the third aspect, the method is as defined in any aspect or embodiment of the present disclosure.

In a fourth aspect, the present disclosure provides a pharmaceutical composition comprising i) a binding agent comprising at least one binding region binding to CD27; ii) a PD1/PD-L1 inhibitor; and iii) optionally a pharmaceutical acceptable carrier.

In one embodiment of the pharmaceutical composition according to the fourth aspect, the binding agent is as defined in any aspect or embodiment of the present disclosure.

In one embodiment of the pharmaceutical composition according to the fourth aspect, the PD1/PD-L1 inhibitor is as defined in any aspect or embodiment of the present disclosure.

In a fifth aspect, the present disclosure provides a pharmaceutical composition for use in a method for reducing progression or preventing progression of a tumor or treating cancer in a subject, said pharmaceutical composition comprising i) a binding agent comprising at least one binding region binding to CD27 and ii) a PD1/PD-L1 inhibitor.

In one embodiment of the pharmaceutical composition for use according to the fifth aspect, the pharmaceutical composition is as defined in any aspect or embodiment of the present disclosure.

In one embodiment of the pharmaceutical composition for use according to the fifth aspect, the tumor or cancer is as defined in any aspect or embodiment of the present disclosure.

In one embodiment of the pharmaceutical composition for use according to the fifth aspect, the subject is as defined in any aspect or embodiment of the present disclosure.

In one embodiment of the pharmaceutical composition for use according to the fifth aspect, the method is as defined in any aspect or embodiment of the present disclosure.

In a sixth aspect, the present disclosure provides a binding agent for use in a method for reducing progression or preventing progression of a tumor or treating cancer in a subject, said method comprising administering to said subject i) the binding agent comprising at least one binding region binding to CD27; and ii) a PD1/PD-L1 inhibitor.

In one embodiment of the binding agent for use according to the sixth aspect, the method is as defined in any aspect or embodiment of the present disclosure.

In one embodiment of the binding agent for use according to the sixth aspect, the binding agent is as defined in any aspect or embodiment of the present disclosure.

In one embodiment of the binding agent for use according to the sixth aspect, the PD1/PD-L1 inhibitor is as defined in any aspect or embodiment of the present disclosure.

In a seventh aspect, the present disclosure provides a PD1/PD-L1 inhibitor for use in a method for reducing progression or preventing progression of a tumor or treating cancer in a subject, said method comprising administering to said subject i) a binding agent comprising at least one binding region binding to CD27; and ii) the PD1/PD-L1 inhibitor.

In one embodiment of the PD1/PD-L1 inhibitor for use according to the seventh aspect, the method is as defined in any aspect or embodiment of the present disclosure.

In one embodiment of the PD1/PD-L1 inhibitor for use according to the seventh aspect, the binding agent is as defined in any aspect or embodiment of the present disclosure.

In one embodiment of the PD1/PD-L1 inhibitor for use according to the seventh aspect, the PD1/PD-L1 inhibitor is as defined in any aspect or embodiment of the present disclosure.

Citation of documents and studies referenced herein is not intended as an admission that any of the foregoing is pertinent prior art. All statements as to the contents of these documents are based on the information available to the applicants and do not constitute any admission as to the correctness of the contents of these documents.

The description (including the following examples) is presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown, but are to be accorded the scope consistent with the claims.

### ITEMS OF THE PRESENT DISCLOSURE

1. A method for reducing progression or preventing progression of a tumor or treating cancer in a subject, said method comprising administering to said subject i) a binding agent comprising at least one binding region binding to CD27; and ii) a PD1/PD-L1 inhibitor.
2. The method of item 1, wherein said binding agent comprises a heavy chain variable (VH) region CDR1, CDR2, and CDR3 comprising the sequences as set forth in SEQ ID NOs: 5, 6, and 7, respectively, and a light chain variable (VL) region CDR1, CDR2, and CDR3 comprising the sequences as set forth in SEQ ID NO: 9, 10 and 11, respectively.
3. The method of item 1 or 2, wherein said binding agent comprises two binding regions capable of binding to human CD27 wherein said antibody comprises the heavy chain variable (VH) region CDR1, CDR2, and CDR3 comprising the sequences as set forth in SEQ ID NOs: 5, 6, and 7, respectively, and the light chain variable (VL) region CDR1, CDR2, and CDR3 comprising the sequences as set forth in SEQ ID NO: 9, 10, and 11, respectively.
4. The method of any one of the preceding items, wherein said binding agent comprises a VH region comprising a sequence as set forth in SEQ ID NO: 4.
5. The method of any one of the preceding items, wherein said binding agent comprises a VL region comprising a sequence as set forth in SEQ ID NO: 8.
6. The method of any one of the preceding items, wherein said binding agent comprises the VH and VL regions comprising the sequences as set forth in SEQ ID NO: 4 and SEQ ID NO: 8, respectively.
7. The method of any one of the preceding items, wherein said binding agent is an antibody, preferably a human or a humanized antibody.
8. The method of any one of the preceding items, wherein the antibody is a full-length antibody further comprising a light chain constant region (CL) and a heavy chain constant region (CH).
9. The method of item 8, wherein the light chain constant region is human kappa.
10. The method of item 8, wherein the light chain constant region is human lambda.
11. The method of any one of the preceding items, wherein said binding agent further comprises a heavy chain constant region, which is of a human IgG isotype, optionally of a modified human IgG.
12. The method of item 11, wherein the human IgG or modified human IgG is selected from IgG1, IgG2, IgG3 or IgG4, such as human IgG1.
13. The method of item 11 or 12, wherein the IgG is a modified human IgG comprising one or more amino acid substitutions.
14. The method of any one of items 11 to 13, wherein the modified human IgG is a modified human IgG1 comprising one or more amino acid substitutions, such as two or more amino acid substitutions.
15. The method of any one of items 11 to 14, wherein the modified human IgG heavy chain constant region comprises at most 10 amino acid substitutions, such as at most 9, such as at most 8, such as at most 7, such as at most 6, such as at most 5, such as at most 4, such as at most 3, such as at most 2 amino acid substitutions.
16. The method of any one of items 11 to 15, wherein said substitution in the heavy chain constant region induces increased CD27 agonism compared to an identical antibody except for comprising a wild type IgG1 antibody heavy chain constant region.
17. The method of any one of items 11 to 16, wherein the amino acid residue at the position corresponding to position E345 or E430 in a human IgG1 heavy chain according to Eu numbering is selected from the group comprising: A, C, D, F, G, H, I, K, L, M, N, Q, R, S, T, V, W and Y.
18. The method of any one of items 11 to 17, wherein the amino acid residue at the position corresponding to position E345 in a human IgG1 heavy chain according to Eu numbering is R.
19. The method of any one of items 11 to 18, wherein the amino acid residue at the position corresponding to position E430 in a human IgG1 heavy chain according to Eu numbering is G.
20. The method of any one of items 11 to 19, wherein the amino acid residue at the position corresponding to position P329 in a human IgG1 heavy chain according to Eu numbering is R.
21. The method of any one of items 11 to 20, wherein the amino acid residue at the positions corresponding to position E345 and P329 in a human IgG1 heavy chain according to Eu numbering are both R.
22. The method of any one of items 11 to 21, wherein the binding agent has a pharmacokinetic profile as the parent antibody comprising a wild type IgG1 heavy chain constant region.
23. The method of any one of the preceding items, wherein the binding agent comprises the heavy chain constant region comprising a sequence selected from the group comprising: SEQ ID No 12, 13, 14, 15, 18, 19, 20, 21, 22, 23, 27, 28, 29, 30, 31, 32, 33, 34 and 36.
24. The method of any one of the preceding items, wherein the binding agent comprises the heavy chain constant region comprising the sequence as set forth in SEQ ID No 15.
25. The method of any one of the preceding items, wherein said binding agent comprises a heavy chain constant region, which is modified so that the binding agent induces one or more Fc-mediated effector functions to a lesser extent relative to a parent antibody.
26. The method of item 25, wherein the one or more Fc-mediated effector functions is decreased by at least 20%, such as by at least 30% or by at least 40%, or by at least 50% or by at least 60% or by at least 70%, or by at least 80% or by at least 90%.
27. The method of item 25 or 26, wherein the binding agent does not induce one or more Fc-mediated effector functions.
28. The method of any one of items 25 to 27, wherein the one or more Fc-mediated effector functions is selected from the following group: complement-dependent cytotoxicity (CDC), complement-dependent cell-mediated cytotoxicity (CDCC), complement activation, antibody-dependent cell-mediated cytotoxicity (ADCC), antibody-dependent cell-mediated phagocytosis (ADCP), C1q binding and FcγR binding.
29. The method of any one of items 25 to 28, wherein the binding agent does not induce C1q binding when measured by the method of Example 8.
30. The method of any one of the preceding items, wherein the binding agent is a monovalent antibody.
31. The method of any one of the preceding items, wherein the binding agent is a bivalent antibody.
32. The method of any one of the preceding items, wherein the binding agent is a monospecific antibody.
33. The method of any one of the preceding items, wherein the binding agent is a bispecific antibody comprising a first antigen binding region capable of binding human CD27 according to any one of the preceding items and comprising a second antigen binding region capable of binding to a different epitope on human CD27 or capable of binding a different target.
34. The method of any one of the preceding items, wherein CD27 is human CD27, in particular said human CD27 comprises the sequence as set forth in SEQ ID NO: 1 or the human CD27 variant as set forth in SEQ ID NO: 2.
35. The method of any one of the preceding items, wherein said binding agent comprises:
a. The VH region comprising the amino acid sequence set forth in SEQ ID No: 4;
b. The VL region comprising the amino acid sequence set forth in SEQ ID No: 8;
c. The CH region comprising the amino acid sequence set forth in SEQ ID No: 15; and
d. The CL region comprising the amino acid sequence set forth in SEQ ID No: 17.
36. The method of any one of the preceding items, wherein said binding agent comprises a heavy chain comprising the amino acid sequence set forth in SEQ ID NO: 35 and a light chain comprising the amino acid sequence set forth in SEQ ID NO: 25.
37. The method of any one of the preceding items, wherein PD-L1 is human PD-L1, in particular human PD-L1 comprising the sequence set forth in SEQ ID NO: 98.
38. The method of any one of the preceding items, wherein PD1 is human PD1, preferably the PD1 has or comprises the amino acid sequence as set forth in SEQ ID NO: 58 or SEQ ID NO: 59, or the amino acid sequence of PD1 has at least 70%, at least 75%, at least 80%, at least 85%, at least 90%, at least 95%, at least 97%, at least 99%, or 100% identity to the amino acid sequence as set forth in SEQ ID NO: 58 or SEQ ID NO: 59, or is an immunogenic fragment thereof.
39. The method of any one of the preceding items, wherein the PD1/PD-L1 inhibitor is an antibody binding to PD1 or PD-L1, preferably an antibody which is an antagonist of PD1/PD-L1 interaction and/or is a PD1 or PD-L1 blocking antibody.
40. The method of any one of the preceding items, wherein the PD1/PD-L1 inhibitor is an antibody of an isotype selected from the group consisting of IgG1, IgG2, IgG3, and IgG4, such as of the IgG1 isotype.
41. The method of any one of the preceding items, wherein the PD1/PD-L1 inhibitor is a full-length antibody or antibody fragment, such as a full-length IgG1 antibody.
42. The method of any one of the preceding items, wherein the PD1/PD-L1 inhibitor is a monospecific antibody.
43. The method of any one of the preceding items, wherein said PD1/PD-L1 inhibitor is an antibody binding to PD1 comprising a heavy chain variable region (VH) comprising the CDR1, CDR2 and CDR3 sequences set forth in SEQ ID NO: 99, 100 and 101, respectively, and a light chain variable region (VL) comprising the CDR1, CDR2, and CDR3 sequences set forth in SEQ ID NO: 102, LAS and SEQ ID NO: 103, respectively.
44. The method of any one of the preceding items, wherein said PD1/PD-L1 inhibitor is an antibody binding to PD1 comprising a VH region comprising the amino acid sequence of SEQ ID NO: 104 and a VL region comprising the amino acid sequence of SEQ ID NO: 105.
45. The method of any one of the preceding items, wherein said PD1/PD-L1 inhibitor is an antibody binding to PD1 comprising a heavy chain comprising the amino acid sequence of SEQ ID NO: 106 and a light chain comprising the amino acid sequence of SEQ ID NO: 107.
46. The method of any one of the preceding items, wherein
a) said binding agent is an antibody comprises a heavy chain comprising the amino acid sequence set forth in SEQ ID NO: 35 and a light chain comprising the amino acid sequence set forth in SEQ ID NO: 25;
b) said PD1/PD-L1 inhibitor is Pembrolizumab or a biosimilar thereof.
47. The method of any one of items 1-42, wherein
a) said binding agent is an antibody comprises a heavy chain comprising the amino acid sequence set forth in SEQ ID NO: 35 and a light chain comprising the amino acid sequence set forth in SEQ ID NO: 25;
b) said PD1/PD-L1 inhibitor is Nivolumab or a biosimilar thereof.
48. The method of any one of items 1-42, wherein
a) said binding agent is an antibody comprises a heavy chain comprising the amino acid sequence set forth in SEQ ID NO: 35 and a light chain comprising the amino acid sequence set forth in SEQ ID NO: 25;
b) said PD1/PD-L1 inhibitor is Atezolizumab or a biosimilar thereof.
49. The method of any one of items 1-42, wherein said PD1/PD-L1 inhibitor is an antibody binding to PD1, or an antigen binding fragment thereof, wherein said antibody binding to PD1 comprises a VH region CDR1, CDR2, and CDR3 comprising the sequences as set forth in SEQ ID NOs: 49, 46, and 45, respectively, and a VL region CDR1, CDR2, and CDR3 comprising the sequences as set forth in SEQ ID NO: 52, QAS and SEQ ID NO: 50, respectively.
50. The method of item 49, wherein the antibody binding to PD1 comprises a heavy chain variable region (VH) comprising a sequence having at least 70%, at least 75%, at least 80%, at least 85%, at least 90%, at least 95%, at least 97%, at least 99%, or 100% identity to the amino acid sequence of the VH sequence as set forth in SEQ ID NO: 56.
51. The method of item 50, wherein the antibody binding to PD1 comprises a heavy chain variable region (VH), wherein the VH comprises the sequence as set forth in SEQ ID NO: 56.
52. The method of any one of items 49-51, wherein the antibody binding to PD1 comprises a light chain variable region (VL) comprising a sequence having at least 70%, at least 75%, at least 80%, at least 85%, at least 90%, at least 95%, at least 97%, at least 99%, or 100% identity to the amino acid sequence of the VL sequence as set forth in SEQ ID NO: 57.
53. The method of item 52, wherein the antibody binding to PD1 comprises a light chain variable region (VL), wherein the VL comprises the sequence as set forth in SEQ ID NO: 57.
54. The method of any one of items 49-53, wherein the antibody binding to PD1 comprises a heavy chain variable region (VH) and a light chain variable region (VL), wherein the VH comprises or has the sequence as set forth in SEQ ID NO: 56 and the VL comprises or has the sequence as set forth in SEQ ID NO: 57.
55. The method of any one of items 49-54, wherein the antibody binding to PD1 comprises a heavy chain constant region, wherein the heavy chain constant region comprises an aromatic or non-polar amino acid at the position corresponding to position 234 in a human IgG1 heavy chain according to EU numbering and an amino acid other than glycine at the position corresponding to position 236 in a human IgG1 heavy chain according to EU numbering.
56. The method of item 55, wherein the amino acid at the position corresponding to position 236 is a basic amino acid.
57. The method of item 56, wherein the basic amino acid is selected from the group consisting of lysine, arginine and histidine.
58. The method of item 56 or 57, wherein the basic amino acid is arginine (G236R).
59. The method of any one of items 55-58, wherein the amino acid at the position corresponding to position 234 is an aromatic amino acid.
60. The method of item 59, wherein the aromatic amino acid is selected from the group consisting of phenylalanine, tryptophan and tyrosine.
61. The method of any one of items 55-58, wherein the amino acid at the position corresponding to position 234 is a non-polar amino acid.
62. The method of item 61, wherein the non-polar amino acid is selected from the group consisting of alanine, valine, leucine, isoleucine, proline, phenylalanine, methionine and tryptophan.
63. The method of item 61 or 62, wherein the non-polar amino acid is selected from the group consisting of isoleucine, proline, phenylalanine, methionine and tryptophan.
64. The method of any one of items 55-63, wherein the amino acid at the corresponding to position 234 is phenylalanine (L234F).
65. The method of any one of items 55-64, wherein the amino acid at the position corresponding to position 235 in a human IgG1 heavy chain according to EU numbering in said heavy chain constant region of the antibody binding to PD1 is an acidic amino acid.
66. The method of item 65, wherein the acidic amino acid is aspartate or glutamate.
67. The method of any one of items 55-66, wherein the amino acid at the position corresponding to position 235 in a human IgG1 heavy chain according to EU numbering in said heavy chain constant region of the antibody binding to PD1 is glutamate (L235E).
68. The method of any one of items 55-67, wherein the amino acids at the position corresponding to positions 234, 235 and 236 in said heavy chain constant region of the antibody binding to PD1 are a non-polar or an aromatic amino acid at position 234, an acidic amino acid at position 235 and a basic amino acid at position 236.
69. The method of any one of items 55-68, wherein the amino acid corresponding to position 234 is phenylalanine, the amino acid corresponding to position 235 is glutamate, and the amino acid corresponding to position 236 is arginine in said heavy chain constant region of the antibody binding to PD1 (L234F/L235E/G236R).
70. The method of any one of items 49-69, wherein the heavy chain constant region of the antibody binding to PD1 comprises a sequence having at least 70%, at least 75%, at least
80%, at least 85%, at least 90%, at least 95%, at least 97%, at least 99%, or 100% identity to the amino acid sequence of the HC sequence as set forth in SEQ ID NO: 38.
71. The method of any one of items 49-70, wherein the heavy chain constant region of the antibody binding to PD1 comprises the sequence as set forth in SEQ ID NO: 38.
72. The method of any one of items 49-71, wherein the isotype of the heavy chain constant region of the antibody binding to PD1 is IgG1.
73. The method of any one of items 49-72, wherein the antibody binding to PD1 comprises a heavy chain having the sequence as set forth in SEQ ID NO: 139, and a light chain having the sequence as set forth in SEQ ID NO: 140.
74. The method of any one of items 49-73, wherein the antibody binding to PD1 is a monoclonal, chimeric or humanized antibody or a fragment of such an antibody.
75. The method of any one of items 49-74, wherein the antibody binding to PD1 has a reduced or depleted Fc-mediated effector function.
76. The method of any one of items 49-75, wherein binding of complement protein C1q to the constant region of the antibody binding to PD1 is reduced compared to a wild-type antibody, preferably by at least 70%, at least 80%, at least 90%, at least 95%, at least 97% or 100%.
77. The method of any one of items 49-76, wherein binding to one or more of the IgG Fc-gamma receptors to the antibody binding to PD1 is reduced compared to a wild-type antibody, preferably by at least 70%, at least 80%, at least 90%, at least 95%, at least 97% or 100%.
78. The method of item 77, wherein the one or more IgG Fc-gamma receptors are selected from at least one of Fc-gamma RI, Fc-gamma RII and Fc-gamma RIII.
79. The method of item 77 or 78, wherein the IgG Fc-gamma receptor is Fc-gamma RI.
80. The method of any one of items 49-79, wherein the antibody binding to PD1 is not capable of inducing Fc-gamma RI-mediated effector functions or wherein the induced Fc-gamma RI-mediated effector functions are reduced compared to a wild-type antibody, preferably by at least 70%, at least 80%, at least 90%, at least 95%, at least 97% or 100%,
81. The method of any one of items 49-80, wherein the antibody binding to PD1 is not capable of inducing at least one of complement dependent cytotoxicity (CDC) mediated lysis, antibody dependent cellular cytotoxicity (ADCC) mediated lysis, apoptosis, homotypic adhesion and/or phagocytosis or wherein at least one of complement dependent cytotoxicity (CDC) mediated lysis, antibody dependent cellular cytotoxicity (ADCC) mediated lysis, apoptosis, homotypic adhesion and/or phagocytosis is induced in a reduced extent, preferably reduced by at least 70%, at least 80%, at least 90%, at least 95%, at least 97% or 100%.
82. The method of any one of items 49-81, wherein binding of neonatal Fc receptor (FcRn) to the antibody binding to PD1 is unaffected, as compared to a wild-type antibody.
83. The method of any one of items 49-82, the antibody binding to PD1 binds to a native epitope of PD1 present on the surface of living cells.
84. The method of any one of items 49-83, wherein the antibody binding to PD1 is a multispecific antibody comprising a first antigen binding region binding to PD1 and at least one further antigen binding region binding to another antigen.
85. The method of item 84, wherein the antibody binding to PD1 is a bispecific antibody comprising a first antigen binding region binding to PD1 and a second antigen binding region binding to another antigen.
86. The method of item 84 or 85, wherein the first antigen binding region binding to PD1 comprises the heavy chain variable region (VH) and/or the light chain variable region (VL) as set forth in any one of items 50 to 54.
87. The method of any one of items 49-86, wherein
a) the binding agent comprises a VH region comprising the amino acid sequence set forth in SEQ ID No: 4, a VL region comprising the amino acid sequence set forth in SEQ ID No: 8;
b) the antibody binding to PD1 comprises a VH region and a VL region, wherein the VH comprises or has the sequence as set forth in SEQ ID NO: 56 and the VL comprises or has the sequence as set forth in SEQ ID NO: 57.
88. The method of any one of items 49-87, wherein
a) said binding agent is an antibody comprising a VH region comprising the amino acid sequence set forth in SEQ ID No: 4, a VL region comprising the amino acid sequence set forth in SEQ ID No: 8, a CH region comprising the amino acid sequence set forth in SEQ ID No: 15, and a CL region comprising the amino acid sequence set forth in SEQ ID No: 17;
b) said antibody binding to PD1 comprising a VH region comprising the amino acid sequence set forth in SEQ ID No: 56, a VL region comprising the amino acid sequence set forth in SEQ ID No: 57, a CH region comprising the amino acid sequence set forth in SEQ ID No: 38, and a CL region comprising the amino acid sequence set forth in SEQ ID No: 42.
89. The method of any one of items 1-41, wherein the PD1/PD-L1 inhibitor is a multispecific antibody, such as a bispecific antibody.
90. The method of item 89, wherein the PD1/PD-L1 inhibitor is a PD-L1 inhibitor comprising a first binding region binding to CD137 and a second binding region binding to PD-1.
91. The method of item 90, wherein CD137 is human CD137, in particular human CD137 comprising the sequence set forth in SEQ ID NO: 97.
92. The method of item 90 or 91, wherein
a) the first binding region of the PD-L1 inhibitor comprises a heavy chain variable region (VH) comprising the CDR1, CDR2, and CDR3 sequences of SEQ ID NO: 79, and a light chain variable region (VL) comprising the CDR1, CDR2, and CDR3 sequences of SEQ ID NO: 83; and
b) the second binding region of the PD-L1 inhibitor comprises a heavy chain variable region (VH) comprising the CDR1, CDR2, and CDR3 sequences of SEQ ID NO: 86, and a light chain variable region (VL) comprising the CDR1, CDR2, and CDR3 sequences of SEQ ID NO: 90.
93. The method of any one of items 90-92, wherein a) the first binding region of the PD-L1 inhibitor comprises a heavy chain variable region (VH) comprising the CDR1, CDR2, and CDR3 sequences set forth in: SEQ ID NO: 80, 81, and 82, respectively, and a light chain variable region (VL) comprising the CDR1, CDR2, and CDR3 sequences set forth in: SEQ ID NO: 84, GAS, and SEQ ID NO: 85, respectively; and b) the second binding region of the PD-L1 inhibitor comprises a heavy chain variable region (VH) comprising the CDR1, CDR2, and CDR3 sequences set forth in: SEQ ID NO: 87, 88, and 89, respectively, and a light chain variable region (VL) comprising the CDR1, CDR2, and CDR3 sequences set forth in: SEQ ID NO: 91, DDN, and SEQ ID NO: 92, respectively.
94. The method of any one of items 90-93, wherein
a) the first binding region of the PD-L1 inhibitor comprises a heavy chain variable region (VH) comprising the amino acid sequence set forth in SEQ ID NO: 79 and a light chain variable region (VL) region comprising the amino acid sequence set forth in SEQ ID NO: 83; and
b) the second binding region of the PD-L1 inhibitor comprises a heavy chain variable region (VH) comprising the amino acid sequence set forth in SEQ ID NO: 86 and a light chain variable region (VL) region comprising the amino acid sequence set forth in SEQ ID NO: 90.
95. The method of any one of items 90-94, wherein the PD-L1 inhibitor is an antibody comprising a first binding arm and a second binding arm, wherein the first binding arm comprises
i) a polypeptide comprising said first heavy chain variable region (VH) and a first heavy chain constant region (CH), and
ii) a polypeptide comprising said first light chain variable region (VL) and a first light chain constant region (CL);
and the second binding arm comprises
iii) a polypeptide comprising said second heavy chain variable region (VH) and a second heavy chain constant region (CH), and
iv) a polypeptide comprising said second light chain variable region (VL) and a second light chain constant region (CL).
96. The method of any one of items 90-95, wherein said PD-L1 inhibitor comprises
i) a first heavy chain and light chain comprising said antigen binding region capable of binding to CD137, the first heavy chain comprising a first heavy chain constant region and the first light chain comprising a first light chain constant region; and
ii) a second heavy chain and light chain comprising said antigen binding region capable of binding PD-L1, the second heavy chain comprising a second heavy chain constant region and the second light chain comprising a second light chain constant region.
97. The method of item 95 or 96, wherein (i) the amino acid in the position corresponding to F405 in a human IgG1 heavy chain according to EU numbering is L in said first heavy chain constant region (CH), and the amino acid in the position corresponding to K409 in a human IgG1 heavy chain according to EU numbering is R in said second heavy chain constant region (CH), or (ii) the amino acid in the position corresponding to K409 in a human IgG1 heavy chain according to EU numbering is R in said first heavy chain, and the amino acid in the position corresponding to F405 in a human IgG1 heavy chain according to EU numbering is L in said second heavy chain.
98. The method of any one of item 95-97, wherein the positions corresponding to positions L234 and L235 in a human IgG1 heavy chain according to EU numbering are F and E, respectively, in said first and second heavy chains.
99. The method of any one of item 95-98, wherein the positions corresponding to positions L234, L235, and D265 in a human IgG1 heavy chain according to EU numbering are F, E, and A, respectively, in said first and second heavy chain constant regions (HCs).
100. The method of any one of items 95-99, wherein the positions corresponding to positions L234 and L235 in a human IgG1 heavy chain according to EU numbering of both the first and second heavy chain constant regions are F and E, respectively, and wherein (i) the position corresponding to F405 in a human IgG1 heavy chain according to EU numbering of the first heavy chain constant region is L, and the position corresponding to K409 in a human IgG1 heavy chain according to EU numbering of the second heavy chain is R, or (ii) the position corresponding to K409 in a human IgG1 heavy chain according to EU numbering of the first heavy chain constant region is R, and the position corresponding to F405 in a human IgG1 heavy chain according to EU numbering of the second heavy chain is L.
101. The method of any one of items 95-100, wherein the positions corresponding to positions L234, L235, and D265 in a human IgG1 heavy chain according to EU numbering of both the first and second heavy chain constant regions are F, E, and A, respectively, and wherein (i) the position corresponding to F405 in a human IgG1 heavy chain according to EU numbering of the first heavy chain constant region is L, and the position corresponding to K409 in a human IgG1 heavy chain according to EU numbering of the second heavy chain constant region is R, or (ii) the position corresponding to K409 in a human IgG1 heavy chain according to EU numbering of the first heavy chain is R, and the position corresponding to F405 in a human IgG1 heavy chain according to EU numbering of the second heavy chain is L.
102. The method of any one of items 95-101, wherein the constant region of said first and/or second heavy chain, such as the second heavy chain, comprises or consists essentially of or consists of an amino acid sequence selected from the group consisting of
a) the sequence set forth in SEQ ID NO: 94 or 96[IgG1-Fc_FEAL];
b) a subsequence of the sequence in a), such as a subsequence, wherein 1, 2, 3, 4, 5, 6, 7, 8, 9 or 10 consecutive amino acids has/have been deleted, starting from the N-terminus or C-terminus of the sequence defined in a); and
c) a sequence having at most 6 substitutions, such as at most 5 substitutions, at most 4 substitutions, at most 3, at most 2 substitutions or at most 1 substitution, compared to the amino acid sequence defined in a) or b).
103. The method of any one of items 95-102, wherein the constant region of said first and/or second heavy chain, such as the first heavy chain, comprises or consists essentially of or consists of an amino acid sequence selected from the group consisting of
a) the sequence set forth in SEQ ID NO: 93 or 95 [IgG1-Fc_FEAR];
b) a subsequence of the sequence in a), such as a subsequence, wherein 1, 2, 3, 4, 5, 6, 7, 8, 9 or 10 consecutive amino acids has/have been deleted, starting from the N-terminus or C-terminus of the sequence defined in a); and
c) a sequence having at most 6 substitutions, such as at most 5 substitutions, at most 4, at most 3, at most 2 substitutions or at most 1 substitution, compared to the amino acid sequence defined in a) or b).
104. The method of any one of any one of items 95-103, wherein said PD-L1 inhibitor comprises a kappa (κ) light chain constant region.
105. The method of any one of any one of items 95-104, wherein said PD-L1 inhibitor comprises a lambda (λ) light chain constant region.
106. The method of any one of any one of items 95-105, wherein said first light chain constant region is a kappa (κ) light chain constant region or a lambda (λ) light chain constant region.
107. The method of any one of any one of items 95-106, wherein said second light chain constant region is a lambda (λ) light chain constant region or a kappa (κ) light chain constant region.
108. The method of any one of any one of items 95-107, wherein said first light chain constant region is a kappa (κ) light chain constant region and said second light chain constant region is a lambda (λ) light chain constant region or said first light chain constant region is a lambda (λ) light chain constant region and said second light chain constant region is a kappa (κ) light chain constant region.
109. The method of any one of items 104-108, wherein the kappa (κ) light chain comprises an amino acid sequence selected from the group consisting of
a) the sequence set forth in SEQ ID NO: 16,
b) a subsequence of the sequence in a), such as a subsequence, wherein 1, 2, 3, 4, 5, 6, 7, 8, 9 or 10 consecutive amino acids has/have been deleted, starting from the N-terminus or C-terminus of the sequence defined in a); and
c) a sequence having at most 10 substitutions, such as at most 9 substitutions, at most
8, at most 7, at most 6, at most 5, at most 4 substitutions, at most 3, at most 2 substitutions or at most 1 substitution, compared to the amino acid sequence defined in a) or b).
110. The method of any one of items 105-109, wherein the lambda (λ) light chain comprises an amino acid sequence selected from the group consisting of
a) the sequence set forth in SEQ ID NO: 17,
b) a subsequence of the sequence in a), such as a subsequence, wherein 1, 2, 3, 4, 5, 6, 7, 8, 9 or 10 consecutive amino acids has/have been deleted, starting from the N-terminus or C-terminus of the sequence defined in a); and
c) a sequence having at most 10 substitutions, such as at most 9 substitutions, at most 8, at most 7, at most 6, at most 5, at most 4 substitutions, at most 3, at most 2 substitutions or at most 1 substitution, compared to the amino acid sequence defined in a) or b).
111. The method of any one of items 90-110, wherein the PD-L1 inhibitor is an antibody of the IgG1m(f) allotype.
112. The method of an one of items 90-111, wherein the PD-L1 inhibitor is a bispecific antibody binding to CD137 and PD-L1, the bispecific antibody having i) a first heavy chain comprising the amino acid sequence set forth in SEQ ID NO: 75 and a first light chain comprising the amino acid sequence set forth in SEQ ID NO: 76, and ii) a second heavy chain comprising the amino acid sequence set forth in SEQ ID NO: 77 and a second light chain comprising the amino acid sequence set forth in SEQ ID NO: 78.
113. The method according to any one of items 90-112, wherein the PD-L1 inhibitor is acasunlimab or a biosimilar thereof.
114. The method of any one of items 90-113, wherein
a) the binding agent comprises a heavy chain variable (VH) region CDR1, CDR2, and CDR3 comprising the sequences as set forth in SEQ ID NOs: 5, 6, and 7, respectively, and a light chain variable (VL) region CDR1, CDR2, and CDR3 comprising the sequences as set forth in SEQ ID NO: 9, 10, and 11, respectively;
b) the first binding region of the PD-L1 inhibitor comprises a heavy chain variable region (VH) comprising the CDR1, CDR2, and CDR3 sequences set forth in: SEQ ID NO: 80, 81, and
82, respectively, and a light chain variable region (VL) comprising the CDR1, CDR2, and CDR3 sequences set forth in: SEQ ID NO: 84, GAS, and SEQ ID NO: 85, respectively; and
c) the second binding region of the PD-L1 inhibitor comprises a heavy chain variable region (VH) comprising the CDR1, CDR2, and CDR3 sequences set forth in: SEQ ID NO: 87, 88, and 89, respectively, and a light chain variable region (VL) comprising the CDR1, CDR2, and CDR3 sequences set forth in: SEQ ID NO: 91, DDN, and SEQ ID NO: 92, respectively.
115. The method of any one of items 90-114, wherein
a) the binding agent comprises a VH region comprising the amino acid sequence set forth in SEQ ID No: 4, a VL region comprising the amino acid sequence set forth in SEQ ID No: 8;
b) the first binding region of the PD-L1 inhibitor comprises a heavy chain variable region (VH) comprising the amino acid sequence set forth in SEQ ID NO: 79 and a light chain variable region (VL) region comprising the amino acid sequence set forth in SEQ ID NO: 83; and
c) the second binding region of the PD-L1 inhibitor comprises a heavy chain variable region (VH) comprising the amino acid sequence set forth in SEQ ID NO: 86 and a light chain variable region (VL) region comprising the amino acid sequence set forth in SEQ ID NO: 90.
116. The method of any one of items 90-115, wherein
a) said binding agent is an antibody comprising a VH region comprising the amino acid sequence set forth in SEQ ID No: 4, a VL region comprising the amino acid sequence set forth in SEQ ID No: 8, a CH region comprising the amino acid sequence set forth in SEQ ID No: 15, and a CL region comprising the amino acid sequence set forth in SEQ ID No: 17;
b) said PD-L1 inhibitor is an antibody comprising a first binding arm and a second binding arm, the first binding arm comprising the first binding region and a second binding arm comprising the second binding region;
c) the first binding arm of the PD-L1 inhibitor comprises a VH region comprising the amino acid sequence set forth in SEQ ID No: 79, a VL region comprising the amino acid sequence set forth in SEQ ID No: 83; a CH region comprising the amino acid sequence set forth in SEQ ID No: 95, and a CL region comprising the amino acid sequence set forth in SEQ ID No: 16; and
d) the second binding arm of the PD-L1 inhibitor comprises a VH region comprising the amino acid sequence set forth in SEQ ID No: 86, a VL region comprising the amino acid sequence set forth in SEQ ID No: 90, a CH region comprising the amino acid sequence set forth in SEQ ID No: 96, and a CL region comprising the amino acid sequence set forth in SEQ ID No: 17.
117. The method of any one of items 90-116, wherein
a) said binding agent comprises a heavy chain comprising the amino acid sequence set forth in SEQ ID NO: 35 and a light chain comprising the amino acid sequence set forth in SEQ ID NO: 25;
b) said PD-L1 inhibitor is a bispecific antibody binding to CD137 and PD-L1, the bispecific antibody having i) a first heavy chain comprising the amino acid sequence set forth in SEQ ID NO: 75 and a first light chain comprising the amino acid sequence set forth in SEQ ID NO:
76, and ii) a second heavy chain comprising the amino acid sequence set forth in SEQ ID NO: 77 and a second light chain comprising the amino acid sequence set forth in SEQ ID NO: 78.
118. The method of any one of items 90-117, wherein
a) said binding agent comprises a heavy chain comprising the amino acid sequence set forth in SEQ ID NO: 35 and a light chain comprising the amino acid sequence set forth in SEQ ID NO: 25;
b) said PD-L1 inhibitor is acasunlimab or a biosimilar thereof.
119. The method of any one of items 1-38, wherein the PD1/PD-L1 inhibitor is a PD1 inhibitor selected from Pembrolizumab, Nivolumab, Cemiplimab, Dostarlimab, JTX-4014, Spartalizumab, Camrelizumab, Sintilimab, Tislelizumab, Toripalimab, INCMGA00012 (MGA012), AMP-224, AMP-514, or a respective biosimilar thereof.
120. The method of any one of items 1-38, wherein the PD1 inhibitor is selected from Pembrolizumab, Nivolumab, Cemiplimab, Dostarlimab, JTX-4014, Spartalizumab, Camrelizumab, Sintilimab, Tislelizumab, Toripalimab, INCMGA00012 (MGA012), AMP-514, or a respective biosimilar thereof.
121. The method of any one of items 1-38, wherein the PD1/PD-L1 inhibitor is a PD-L1 inhibitor selected from Atezolizumab, Avelumab, Durvalumab, KN035, CK-301, Acasunlimab, AUNP12, CA-170, BMS-986189, or a respective biosimilar thereof.
122. The method of any one of items 1-38, wherein the PD-L1 inhibitor is selected from Atezolizumab, Avelumab, Durvalumab, KN035, CK-301, Acasunlimab, or a respective biosimilar thereof.
123. The method of any one of the preceding items, wherein the subject is a human subject.
124. The method of any one of the preceding items, wherein the tumor or cancer is a solid tumor.
125. The method according to any one of the preceding items, wherein said tumor is a PD-L1 positive tumor.
126. The method of any one of the preceding items, wherein the tumor or cancer is head and neck squamous cell carcinoma (HNSCC), such as HNSCC of the oral cavity, pharynx or larynx.
127. The method of item 126, wherein the HNSCC is recurrent, unresectable or metastatic.
128. The method of any one of items 1-125, wherein the tumor or cancer is non-small cell lung cancer (NSCLC), such as a squamous or non-squamous NSCLC.
129. The method of item 128, wherein the NSCLC is recurrent, unresectable or metastatic.
130. The method of item 128 or 129, wherein the NSCLC does not have an epidermal growth factor (EGFR)-sensitizing mutation and/or anaplastic lymphoma (ALK) translocation and/or ROS1 rearrangement.
131. The method of any one of items 128-130, wherein the NSCLC is NTRK1/2/3 (neurotrophic receptor tyrosine kinase 1/2/3) fusion positive, and/or has a mutation in KRAS (KRAS proto-oncogene, GTPase), BRAF (B-Raf proto-oncogene, serine/threonine kinase), or MET (MET proto-oncogene, receptor tyrosine kinase) gene, and/or has RET (ret proto-oncogene) gene rearrangements, and the subject has received prior treatment with a respective targeted therapy.
132. The method of any one of the preceding items, wherein the subject has received prior treatment with a PD1 inhibitor or a PD-L1 inhibitor, such as anti-PD1 antibody or an anti-PD-L1 antibody, preferably at least two doses of the PD1 inhibitor or the PD-L1 inhibitor.
133. The method of any one of the preceding items, wherein the subject has received prior treatment with a platinum-based therapy or an alternative chemotherapy if platinum ineligible, eg a gemcitabine-containing regimen.
134. The method of any one of the preceding items, wherein the tumor or cancer has relapsed and/or progressed after treatment, such as systemic treatment with a checkpoint inhibitor.
135. The method of any one of the preceding items, wherein the subject has received at least one prior line of systemic therapy, such as systemic therapy comprising a PD1 inhibitor or a PD-L1 inhibitor, such as an anti-PD1 antibody or an anti-PD-L1 antibody.
136. The method of any one of the preceding items, wherein the cancer or tumor has relapsed and/or is refractory, or the subject has progressed after treatment with a PD1 inhibitor or a PD-L1 inhibitor, such as an anti PD1 antibody or an anti-PD-L1 antibody, the PD1 inhibitor or PD-L1 inhibitor being administered as monotherapy or as part of a combination therapy.
137. The method of any one of the preceding items, wherein last prior treatment was with a PD1 inhibitor or PD-L1 inhibitor, such as an anti PD1 antibody or an anti-PD-L1 antibody, the PD1 inhibitor or PD-L1 inhibitor being administered as monotherapy or as part of a combination therapy.
138. The method of any one of the preceding items, wherein the time from progression on last treatment with a PD1 inhibitor or PD-L1 inhibitor, such as an anti PD1 antibody or an anti-PD-L1 antibody is 6 months or less.
139. The method of any one of the preceding items, wherein the time from last dosing of a PD1 inhibitor or PD-L1 inhibitor, such as an anti PD1 antibody or an anti-PD-L1 antibody as part of last prior treatment is 6 months or less.
140. The method of any one of the preceding items, wherein the cancer or tumor has relapsed and/or is refractory, or the subject has progressed during or after
i) platinum doublet chemotherapy following treatment with an anti-PD1 antibody or an anti-PD-L1 antibody, or
ii) treatment with an anti-PD1 antibody or an anti-PD-L1 antibody following platinum doublet chemotherapy.
141. A kit comprising
i) a binding agent comprising at least one binding region binding to CD27 and
ii) a PD1/PD-L1 inhibitor.
142. The kit according to item 141, wherein the binding agent is as defined in any one of items 1-140 and/or the PD1/PD-L1 inhibitor is as defined in any one of items 1-140.
143. The kit according to item 141 or 142, wherein the binding agent, the PD1/PD-L1 inhibitor, and, if present, one or more additional therapeutic agents are for systemic administration, in particular for injection or infusion, such as intravenous injection or infusion.
144. The kit according to any one of items 141-143 for use in a method for reducing progression or preventing progression of a tumor or treating cancer in a subject.
145. The kit for use according to item 144, wherein the tumor or cancer is as defined in any one of items 1-140, and/or the subject is as defined in any one of items 1-140, and/or the method is/are as defined in any one of items 1-140.
146. A pharmaceutical composition comprising
i) a binding agent comprising at least one binding region binding to CD27;
ii) a PD1/PD-L1 inhibitor; and
iii) optionally a pharmaceutical acceptable carrier.
147. The pharmaceutical composition according to item 146, wherein the binding agent is as defined in any one of items 1-140 and/or the PD1/PD-L1 inhibitor is as defined in any one of items 1-140.
148. The pharmaceutical composition according to item 146 or 147 for use in a method for reducing progression or preventing progression of a tumor or treating cancer in a subject.
149. The pharmaceutical composition for use according to item 148, wherein the tumor or cancer is as defined in any one of items 1-140, and/or the subject is as defined in any one of items 1-140, and/or the method is/are as defined in any one of items 1-140.
150. A binding agent for use in a method for reducing progression or preventing progression of a tumor or treating cancer in a subject, said method comprising administering to said subject i) the binding agent comprising at least one binding region binding to CD27; and ii) a PD1/PD-L1 inhibitor
151. The binding agent for use according to item 150, wherein the method is as defined in any one of items 1-140, and/or the binding agent is as defined in any one of items 1-140, and/or the PD1/PD-L1 inhibitor is as defined in any one of items 1-140.
152. A PD1/PD-L1 inhibitor for use in a method for reducing progression or preventing progression of a tumor or treating cancer in a subject, said method comprising administering to said subject i) a binding agent comprising at least one binding region binding to CD27; and ii) the PD1/PD-L1 inhibitor.
153. The PD1/PD-L1 inhibitor for use according to item 152, wherein the method is as defined in any one of items 1-140, and/or the binding agent is as defined in any one of items 1-140, and/or the PD1/PD-L1 inhibitor is as defined in any one of items 1-140.

Further aspects of the present disclosure are disclosed herein.

### Example 1: Generation of DuoBody-PD-L1×4-1BB and Anti-Human CD27 Antibodies and Fc Variants Thereof

Generation of anti-human CD27 antibodies through immunization and hybridoma generation was performed at Aldevron GmbH (Freiburg, Germany). cDNA's encoding human CD27 (full length and ECD) were cloned into Aldevron proprietary expression plasmids. Anti-CD27 antibodies were generated by immunization of OmniRat animals (transgenic rats expressing a diversified repertoire of antibodies with fully human idiotypes; Ligand Pharmaceuticals Inc.) using intradermal application of human CD27 cDNA-coated gold-particles using a hand-held device for particle-bombardment (“gene gun”). Serum samples were collected after a series of immunizations and tested by flow cytometry on HEK cells transiently transfected with the aforementioned expression plasmid for full length human CD27 expression. Antibody-producing cells were isolated from rat spleen and fused with mouse myeloma cells (Ag8) according to standard procedures. RNA from hybridomas producing CD27-specific antibody was extracted for sequencing.

Out of a panel of 71 CD27 antibodies six antibodies were selected for further characterization based on binding to primary T cells and diversity in CD27 binding competition assays in vitro. These six antibodies are named IgG1-CD27-A, IgG1-CD27-B, IgG1-CD27-C, IgG1-CD27-D, IgG1-CD27-E and IgG1-CD27-F herein.

The variable regions, in some cases with single point mutations to remove amino acid residues that were considered a liability for manufacturing (e.g., free cysteines or glycosylation sites), of heavy and light chains of interest were gene synthesized and cloned into expression vectors containing the backbone sequences for human antibody light chains and a human IgG1 heavy chain.

Fc variants of the six different antibodies were generated by introduction of one or more of the following amino acid mutations, according to Eu numbering: E345R, E430G, P329R, G237A, K326A, E333A, see Tables 1 and 3 below. After functional characterization in vitro as described below, CD27-specific IgG1-CD27-A (VH SEQ ID NO: 4; VL SEQ ID NO: 8) was considered to have the most optimal biological properties. Sequences of the prior art CD27-targeting antibodies used herein as benchmarks have been obtained as follows: IgG1-CD27-15 (WO2012004367; SEQ ID Nos 3 and 4), IgG1-CD27-131A (WO2018/058022; SEQ ID Nos 10 and 15), IgG1-CD27-CDX1127 (WO2016145085; SEQ ID Nos: 1 and 2), and IgG1-CD27-BMS986215 (WO2019195452A1; SEQ ID Nos 8 and 9). The VH and VL sequences of a type I anti-human CD20 antibody have been described previously in WO2019/145455A1 (SEQ ID Nos 35 and 39).

DuoBody-PD-L1×4-1BB is a bispecific antibody, based on the DuoBody technology platform (WO2011131746A2), which binds PD-L1 with one arm and 4-1BB with the other arm (WO2021/156326A1). DuoBody-PD-L1×4-1BB was generated using parental clones IgG1-CD137-009-H7 (HC SEQ ID NO: 75; LC SEQ ID NO: 76; HCDR1 SEQ ID NO: 80, HCDR2 SEQ ID NO: 81, HCDR3 SEQ ID NO: 82, LCDR1 SEQ ID NO: 84, LCDR2: GAS, LCDR3 SEQ ID NO: 85) and IgG1-PD-L1-547 (HC SEQ ID NO: 77; LC SEQ ID NO: 78; HCDR1 SEQ ID NO: 87, HCDR2 SEQ ID NO: 88, HCDR3 SEQ ID NO: 89, LCDR1 SEQ ID NO: 91, LCDR2: DDN, LCDR3 SEQ ID NO: 92). As a control antibody, anti-HIV gp120 antibody IgG1-b12 was used in this application (Barbas et al., J Mol Biol 1993 230: 812-823; VH: SEQ ID NO 68, VL: SEQ ID NO 72 of this application).

TABLE 1
list of amino acid sequences

| SEQ | | |
|---|---|---|
| ID | | |
| NO: | Identifier | Amino acid sequence |
| 1 | Human CD27 | MARPHPWWLCVLGTLVGLSATPAPKSCPERHYWAQGKLCCQMCEPGTFL |
| | | VKDCDQHRKAAQCDPCIPGVSFSPDHHTRPHCESCRHCNSGLLVRNCTI |
| | | TANAECACRNGWQCRDKECTECDPLPNPSLTARSSQALSPHPQPTHLPYV |
| | | SEMLEARTAGHMQTLADFRQLPARTLSTHWPPQRSLCSSDFIRILVIFSG |
| | | MFLVFTLAGALFLHQRRKYRSNKGESPVEPAEPCHYSCPREEEGSTIPIQE |
| | | DYRKPEPACSP |
| 2 | CD27-A59T | MARPHPWWLCVLGTLVGLSATPAPKSCPERHYWAQGKLCCQMCEPGTFL |
| | variant | VKDCDQHRKTAQCDPCIPGVSFSPDHHTRPHCESCRHCNSGLLVRNCTI |
| | | TANAECACRNGWQCRDKECTECDPLPNPSLTARSSQALSPHPQPTHLPYV |
| | | SEMLEARTAGHMQTLADFRQLPARTLSTHWPPQRSLCSSDFIRILVIFSG |
| | | MFLVFTLAGALFLHQRRKYRSNKGESPVEPAEPCHYSCPREEEGSTIPIQE |
| | | DYRKPEPACSP |
| 3 | Cyno CD27 | MARPHPWWLCFLGTLVGLSATPAPKSCPERHYWAQGKLCCQMCEPGTFL |
| | | VKDCDQHRKAAQCHPCIPGVSFSPDHHTRPHCESCRHCNSGLLIRNCTIT |
| | | ANAVCACRNGWQCRDKECTECDPPPNPSLTTWPSQALGPHPQPTHLPYV |
| | | NEMLEARTAGHMQTLADFRHLPARTLSTHWPPQRSLCSSDFIRILVIFSG |
| | | MFLVFTLAGTLFLHQQRKYRSNKGESPMEPAEPCPYSCPREEEGSTIPIQE |
| | | DYRKPEPASSP |
| 4 | CD27-A VH | QVQLMQSGSELKKPGASVKVSCRASGYTFTTYAMNWVRQAPGQGPEWM |
| | | GWINTNTGNPTYAQGFTGRFVFSLDTTVTTTYLQISSLKAEDTAVYFCARE |
| | | AGSFDYWGQGTLVTVSS |
| 5 | CD27-A VH | GYTFTTYA |
| | CDR1 | |
| 6 | CD27-A VH | INTNTGNP |
| | CDR2 | |
| 7 | CD27-A VH | AREAGSFDY |
| | CDR3 | |
| 8 | CD27-A VL | QSALTQPASVSGSPGQSITISCTGTSSDVYYYNYVSWYQQHPGRAPKLVI |
| | | YDVSNRPSGVSNRFSGSKSGNTASLTISGLRAEDEADYYCSSYTVNRVW |
| | | VFGGGTKLTVL |
| 9 | CD27-A VL | SSDVYYYNY |
| | CDR1 | |
| 10 | CD27-A VL | DVS |
| | CDR2 | |
| 11 | CD27-A VL | SSYTVNRVWV |
| | CDR3 | |
| 12 | Constant | ASTKGPSVFPLAPSSKSTSGGTAALGCLVKDYFPEPVTVSWNSGALTSGV |
| | region human | HTFPAVLQSSGLYSLSSVVTVPSSSLGTQTYICNVNHKPSNTKVDKRVEPK |
| | IgG1m(f) | SCDKTHTCPPCPAPELLGGPSVFLFPPKPKDTLMISRTPEVTCVVVDVSHE |
| | | DPEVKFNWYVDGVEVHNAKTKPREEQYNSTYRVVSVLTVLHQDWLNGKE |
| | | YKCKVSNKALPAPIEKTISKAKGQPREPQVYTLPPSREEMTKNQVSLTCLV |
| | | KGFYPSDIAVEWESNGQPENNYKTTPPVLDSDGSFFLYSKLTVDKSRWQ |
| | | QGNVFSCSVMHEALHNHYTQKSLSLSPGK |
| 13 | Constant | ASTKGPSVFPLAPSSKSTSGGTAALGCLVKDYFPEPVTVSWNSGALTSGV |
| | region human | HTFPAVLQSSGLYSLSSVVTVPSSSLGTQTYICNVNHKPSNTKVDKRVEPK |
| | IgG1-E345R | SCDKTHTCPPCPAPELLGGPSVFLFPPKPKDTLMISRTPEVTCVVVDVSHE |
| | | DPEVKFNWYVDGVEVHNAKTKPREEQYNSTYRVVSVLTVLHQDWLNGKE |
| | | YKCKVSNKALPAPIEKTISKAKGQPRRPQVYTLPPSREEMTKNQVSLTCLV |
| | | KGFYPSDIAVEWESNGQPENNYKTTPPVLDSDGSFFLYSKLTVDKSRWQ |
| | | QGNVFSCSVMHEALHNHYTQKSLSLSPGK |
| 14 | Constant | ASTKGPSVFPLAPSSKSTSGGTAALGCLVKDYFPEPVTVSWNSGALTSGV |
| | region human | HTFPAVLQSSGLYSLSSVVTVPSSSLGTQTYICNVNHKPSNTKVDKRVEPK |
| | IgG1-P329R | SCDKTHTCPPCPAPELLGGPSVFLFPPKPKDTLMISRTPEVTCVVVDVSHE |
| | | DPEVKFNWYVDGVEVHNAKTKPREEQYNSTYRVVSVLTVLHQDWLNGKE |
| | | YKCKVSNKALRAPIEKTISKAKGQPREPQVYTLPPSREEMTKNQVSLTCLV |
| | | KGFYPSDIAVEWESNGQPENNYKTTPPVLDSDGSFFLYSKLTVDKSRWQ |
| | | QGNVFSCSVMHEALHNHYTQKSLSLSPGK |
| 15 | Constant | ASTKGPSVFPLAPSSKSTSGGTAALGCLVKDYFPEPVTVSWNSGALTSGV |
| | region human | HTFPAVLQSSGLYSLSSVVTVPSSSLGTQTYICNVNHKPSNTKVDKRVEPK |
| | IgG1-delK | SCDKTHTCPPCPAPELLGGPSVFLFPPKPKDTLMISRTPEVTCVVVDVSHE |
| | E345R + P329R | DPEVKFNWYVDGVEVHNAKTKPREEQYNSTYRVVSVLTVLHQDWLNGKE |
| | | YKCKVSNKALRAPIEKTISKAKGQPRRPQVYTLPPSREEMTKNQVSLTCLV |
| | | KGFYPSDIAVEWESNGQPENNYKTTPPVLDSDGSFFLYSKLTVDKSRWQ |
| | | QGNVFSCSVMHEALHNHYTQKSLSLSPG |
| 16 | Human kappa | RTVAAPSVFIFPPSDEQLKSGTASVVCLLNNFYPREAKVQWKVDNALQSG |
| | LC | NSQESVTEQDSKDSTYSLSSTLTLSKADYEKHKVYACEVTHQGLSSPVTK |
| | | SFNRGEC |
| 17 | Human | GQPKAAPSVTLFPPSSEELQANKATLVCLISDFYPGAVTVAWKADSSPVKA |
| | lambda LC | GVETTTPSKQSNNKYAASSYLSLTPEQWKSHRSYSCQVTHEGSTVEKTVA |
| | | PTECS |
| 18 | Constant | ASTKGPSVFPLAPSSKSTSGGTAALGCLVKDYFPEPVTVSWNSGALTSGV |
| | region human | HTFPAVLQSSGLYSLSSVVTVPSSSLGTQTYICNVNHKPSNTKVDKRVEPK |
| | IgG1m(f)- | SCDKTHTCPPCPAPELLGGPSVFLFPPKPKDTLMISRTPEVTCVVVDVSHE |
| | delK | DPEVKFNWYVDGVEVHNAKTKPREEQYNSTYRVVSVLTVLHQDWLNGKE |
| | | YKCKVSNKALPAPIEKTISKAKGQPREPQVYTLPPSREEMTKNQVSLTCLV |
| | | KGFYPSDIAVEWESNGQPENNYKTTPPVLDSDGSFFLYSKLTVDKSRWQ |
| | | QGNVFSCSVMHEALHNHYTQKSLSLSPG |
| 19 | Constant | ASTKGPSVFPLAPSSKSTSGGTAALGCLVKDYFPEPVTVSWNSGALTSGV |
| | region human | HTFPAVLQSSGLYSLSSVVTVPSSSLGTQTYICNVNHKPSNTKVDKPVEPK |
| | IgG1m(a) | SCDKTHTCPPCPAPELLGGPSVFLFPPKPKDTLMISRTPEVTCVVVDVSHE |
| | | DPEVKFNWYVDGVEVHNAKTKPREEQYNSTYRVVSVLTVLHQDWLNGKE |
| | | YKCKVSNKALPAPIEKTISKAKGQPREPQVYTLPPSRDELTKNQVSLTCLV |
| | | KGFYPSDIAVEWESNGQPENNYKTTPPVLDSDGSFFLYSKLTVDKSRWQ |
| | | QGNVFSCSVMHEALHNHYTQKSLSLSPGK |
| 20 | Constant | ASTKGPSVFPLAPSSKSTSGGTAALGCLVKDYFPEPVTVSWNSGALTSGV |
| | region human | HTFPAVLQSSGLYSLSSVVTVPSSSLGTQTYICNVNHKPSNTKVDKPVEPK |
| | IgG1m(x) | SCDKTHTCPPCPAPELLGGPSVFLFPPKPKDTLMISRTPEVTCVVVDVSHE |
| | | DPEVKFNWYVDGVEVHNAKTKPREEQYNSTYRVVSVLTVLHQDWLNGKE |
| | | YKCKVSNKALPAPIEKTISKAKGQPREPQVYTLPPSREEMTKNQVSLTCLV |
| | | KGFYPSDIAVEWESNGQPENNYKTTPPVLDSDGSFFLYSKLTVDKSRWQ |
| | | QGNVFSCSVMHEGLHNHYTQKSLSLSPGK |
| 21 | Constant | ASTKGPSVFPLAPCSRSTSESTAALGCLVKDYFPEPVTVSWNSGALTSGV |
| | region human | HTFPAVLQSSGLYSLSSVVTVPSSNFGTQTYTCNVDHKPSNTKVDKTVER |
| | IgG2 | KCCVECPPCPAPPVAGPSVFLFPPKPKDTLMISRTPEVTCVVVDVSHEDPE |
| | | VQFNWYVDGVEVHNAKTKPREEQFNSTFRVVSVLTVVHQDWLNGKEYK |
| | | CKVSNKGLPAPIEKTISKTKGQPREPQVYTLPPSREEMTKNQVSLTCLVKG |
| | | FYPSDIAVEWESNGQPENNYKTTPPMLDSDGSFFLYSKLTVDKSRWQQG |
| | | NVFSCSVMHEALHNHYTQKSLSLSPGK |
| 22 | Constant | ASTKGPSVFPLAPCSRSTSGGTAALGCLVKDYFPEPVTVSWNSGALTSGV |
| | region human | HTFPAVLQSSGLYSLSSVVTVPSSSLGTQTYTCNVNHKPSNTKVDKRVEL |
| | IgG3 | KTPLGDTTHTCPRCPEPKSCDTPPPCPRCPEPKSCDTPPPCPRCPEPKSCD |
| | | TPPPCPRCPAPELLGGPSVFLFPPKPKDTLMISRTPEVTCVVVDVSHEDPEV |
| | | QFKWYVDGVEVHNAKTKPREEQYNSTFRVVSVLTVLHQDWLNGKEYKCK |
| | | VSNKALPAPIEKTISKTKGQPREPQVYTLPPSREEMTKNQVSLTCLVKGFY |
| | | PSDIAVEWESSGQPENNYNTTPPMLDSDGSFFLYSKLTVDKSRWQQGNI |
| | | FSCSVMHEALHNRFTQKSLSLSPGK |
| 23 | Constant | ASTKGPSVFPLAPCSRSTSESTAALGCLVKDYFPEPVTVSWNSGALTSGV |
| | region human | HTFPAVLQSSGLYSLSSVVTVPSSSLGTKTYTCNVDHKPSNTKVDKRVES |
| | IgG4 | KYGPPCPSCPAPEFLGGPSVFLFPPKPKDTLMISRTPEVTCVVVDVSQEDP |
| | | EVQFNWYVDGVEVHNAKTKPREEQFNSTYRVVSVLTVLHQDWLNGKEYK |
| | | CKVSNKGLPSSIEKTISKAKGQPREPQVYTLPPSQEEMTKNQVSLTCLVKG |
| | | FYPSDIAVEWESNGQPENNYKTTPPVLDSDGSFFLYSRLTVDKSRWQEGN |
| | | VFSCSVMHEALHNHYTQKSLSLSLGK |
| 24 | CD27-A IgG1 | QVQLMQSGSELKKPGASVKVSCRASGYTFTTYAMNWVRQAPGQGPEWM |
| | HC with | GWINTNTGNPTYAQGFTGRFVFSLDTTVTTTYLQISSLKAEDTAVYFCARE |
| | E345R + P329R | AGSFDYWGQGTLVTVSSASTKGPSVFPLAPSSKSTSGGTAALGCLVKDYF |
| | | PEPVTVSWNSGALTSGVHTFPAVLQSSGLYSLSSVVTVPSSSLGTQTYIC |
| | | NVNHKPSNTKVDKRVEPKSCDKTHTCPPCPAPELLGGPSVFLFPPKPKDTL |
| | | MISRTPEVTCVVVDVSHEDPEVKFNWYVDGVEVHNAKTKPREEQYNSTY |
| | | RVVSVLTVLHQDWLNGKEYKCKVSNKALRAPIEKTISKAKGQPRRPQVYT |
| | | LPPSREEMTKNQVSLTCLVKGFYPSDIAVEWESNGQPENNYKTTPPVLDS |
| | | DGSFFLYSKLTVDKSRWQQGNVFSCSVMHEALHNHYTQKSLSLSPGK |
| 25 | CD27-A LC | QSALTQPASVSGSPGQSITISCTGTSSDVYYYNYVSWYQQHPGRAPKLVI |
| | | YDVSNRPSGVSNRFSGSKSGNTASLTISGLRAEDEADYYCSSYTVNRVW |
| | | VFGGGTKLTVLGQPKAAPSVTLFPPSSEELQANKATLVCLISDFYPGAVTV |
| | | AWKADSSPVKAGVETTTPSKQSNNKYAASSYLSLTPEQWKSHRSYSCQV |
| | | THEGSTVEKTVAPTECS |
| 26 | Mouse CD27 | MAWPPPYWLCMLGTLVGLSATLAPNSCPDKHYWTGGGLCCRMCEPGTFF |
| | | VKDCEQDRTAAQCDPCIPGTSFSPDYHTRPHCESCRHCNSGFLIRNCTVT |
| | | ANAECSCSKNWQCRDQECTECDPPLNPALTRQPSETPSPQPPPTHLPHGT |
| | | EKPSWPLHRQLPNSTVYSQRSSHRPLCSSDCIRIFVTFSSMFLIFVLGAILF |
| | | FHQRRNHGPNEDRQAVPEEPCPYSCPREEEGSAIPIQEDYRKPEPAFYP |
| 27 | Human IgG1- | ASTKGPSVFPLAPSSKSTSGGTAALGCLVKDYFPEPVTVSWNSGALTSGV |
| | Fc- | HTFPAVLQSSGLYSLSSVVTVPSSSLGTQTYICNVNHKPSNTKVDKRVEPK |
| | E345R + P329R | SCDKTHTCPPCPAPELLGGPSVFLFPPKPKDTLMISRTPEVTCVVVDVSHE |
| | | DPEVKFNWYVDGVEVHNAKTKPREEQYNSTYRVVSVLTVLHQDWLNGKE |
| | | YKCKVSNKALRAPIEKTISKAKGQPRRPQVYTLPPSREEMTKNQVSLTCLV |
| | | KGFYPSDIAVEWESNGQPENNYKTTPPVLDSDGSFFLYSKLTVDKSRWQ |
| | | QGNVFSCSVMHEALHNHYTQKSLSLSPGK |
| 28 | Constant | ASTKGPSVFPLAPSSKSTSGGTAALGCLVKDYFPEPVTVSWNSGALTSGV |
| | region human | HTFPAVLQSSGLYSLSSVVTVPSSSLGTQTYICNVNHKPSNTKVDKRVEPK |
| | IgG1m(f) | SCDKTHTCPPCPAPELLGGPSVFLFPPKPKDTLMISRTPEVTCVVVDVSHE |
| | (without c- | DPEVKFNWYVDGVEVHNAKTKPREEQYNSTYRVVSVLTVLHQDWLNGKE |
| | terminal | YKCKVSNKALPAPIEKTISKAKGQPREPQVYTLPPSREEMTKNQVSLTCLV |
| | lysine) | KGFYPSDIAVEWESNGQPENNYKTTPPVLDSDGSFFLYSKLTVDKSRWQ |
| | | QGNVFSCSVMHEALHNHYTQKSLSLSPG |
| 29 | Constant | ASTKGPSVFPLAPSSKSTSGGTAALGCLVKDYFPEPVTVSWNSGALTSGV |
| | region human | HTFPAVLQSSGLYSLSSVVTVPSSSLGTQTYICNVNHKPSNTKVDKRVEPK |
| | IgG1-E345R | SCDKTHTCPPCPAPELLGGPSVFLFPPKPKDTLMISRTPEVTCVVVDVSHE |
| | (without c- | DPEVKFNWYVDGVEVHNAKTKPREEQYNSTYRVVSVLTVLHQDWLNGKE |
| | terminal | YKCKVSNKALPAPIEKTISKAKGQPRRPQVYTLPPSREEMTKNQVSLTCLV |
| | lysine) | KGFYPSDIAVEWESNGQPENNYKTTPPVLDSDGSFFLYSKLTVDKSRWQ |
| | | QGNVFSCSVMHEALHNHYTQKSLSLSPG |
| 30 | Constant | ASTKGPSVFPLAPSSKSTSGGTAALGCLVKDYFPEPVTVSWNSGALTSGV |
| | region human | HTFPAVLQSSGLYSLSSVVTVPSSSLGTQTYICNVNHKPSNTKVDKRVEPK |
| | IgG1-P329R | SCDKTHTCPPCPAPELLGGPSVFLFPPKPKDTLMISRTPEVTCVVVDVSHE |
| | (without c- | DPEVKFNWYVDGVEVHNAKTKPREEQYNSTYRVVSVLTVLHQDWLNGKE |
| | terminal | YKCKVSNKALRAPIEKTISKAKGQPREPQVYTLPPSREEMTKNQVSLTCLV |
| | lysine) | KGFYPSDIAVEWESNGQPENNYKTTPPVLDSDGSFFLYSKLTVDKSRWQ |
| | | QGNVFSCSVMHEALHNHYTQKSLSLSPG |
| 31 | Constant | ASTKGPSVFPLAPSSKSTSGGTAALGCLVKDYFPEPVTVSWNSGALTSGV |
| | region human | HTFPAVLQSSGLYSLSSVVTVPSSSLGTQTYICNVNHKPSNTKVDKPVEPK |
| | IgG1m(a) | SCDKTHTCPPCPAPELLGGPSVFLFPPKPKDTLMISRTPEVTCVVVDVSHE |
| | (without c- | DPEVKFNWYVDGVEVHNAKTKPREEQYNSTYRVVSVLTVLHQDWLNGKE |
| | terminal | YKCKVSNKALPAPIEKTISKAKGQPREPQVYTLPPSRDELTKNQVSLTCLV |
| | lysine) | KGFYPSDIAVEWESNGQPENNYKTTPPVLDSDGSFFLYSKLTVDKSRWQ |
| | | QGNVFSCSVMHEALHNHYTQKSLSLSPG |
| 32 | Constant | ASTKGPSVFPLAPSSKSTSGGTAALGCLVKDYFPEPVTVSWNSGALTSGV |
| | region human | HTFPAVLQSSGLYSLSSVVTVPSSSLGTQTYICNVNHKPSNTKVDKPVEPK |
| | IgG1m(x) | SCDKTHTCPPCPAPELLGGPSVFLFPPKPKDTLMISRTPEVTCVVVDVSHE |
| | (without c- | DPEVKFNWYVDGVEVHNAKTKPREEQYNSTYRVVSVLTVLHQDWLNGKE |
| | terminal | YKCKVSNKALPAPIEKTISKAKGQPREPQVYTLPPSREEMTKNQVSLTCLV |
| | lysine) | KGFYPSDIAVEWESNGQPENNYKTTPPVLDSDGSFFLYSKLTVDKSRWQ |
| | | QGNVFSCSVMHEGLHNHYTQKSLSLSPG |
| 33 | Constant | ASTKGPSVFPLAPCSRSTSESTAALGCLVKDYFPEPVTVSWNSGALTSGV |
| | region human | HTFPAVLQSSGLYSLSSVVTVPSSNFGTQTYTCNVDHKPSNTKVDKTVER |
| | IgG2 (without | KCCVECPPCPAPPVAGPSVFLFPPKPKDTLMISRTPEVTCVVVDVSHEDPE |
| | c-terminal | VQFNWYVDGVEVHNAKTKPREEQFNSTFRVVSVLTVVHQDWLNGKEYK |
| | lysine) | CKVSNKGLPAPIEKTISKTKGQPREPQVYTLPPSREEMTKNQVSLTCLVKG |
| | | FYPSDIAVEWESNGQPENNYKTTPPMLDSDGSFFLYSKLTVDKSRWQQG |
| | | NVFSCSVMHEALHNHYTQKSLSLSPG |
| 34 | Constant | ASTKGPSVFPLAPCSRSTSGGTAALGCLVKDYFPEPVTVSWNSGALTSGV |
| | region human | HTFPAVLQSSGLYSLSSVVTVPSSSLGTQTYTCNVNHKPSNTKVDKRVEL |
| | IgG3 (without | KTPLGDTTHTCPRCPEPKSCDTPPPCPRCPEPKSCDTPPPCPRCPEPKSCD |
| | c-terminal | TPPPCPRCPAPELLGGPSVFLFPPKPKDTLMISRTPEVTCVVVDVSHEDPEV |
| | lysine) | QFKWYVDGVEVHNAKTKPREEQYNSTFRVVSVLTVLHQDWLNGKEYKCK |
| | | VSNKALPAPIEKTISKTKGQPREPQVYTLPPSREEMTKNQVSLTCLVKGFY |
| | | PSDIAVEWESSGQPENNYNTTPPMLDSDGSFFLYSKLTVDKSRWQQGNI |
| | | FSCSVMHEALHNRFTQKSLSLSPG |
| 35 | CD27-A IgG1 | QVQLMQSGSELKKPGASVKVSCRASGYTFTTYAMNWVRQAPGQGPEWM |
| | HC with | GWINTNTGNPTYAQGFTGRFVFSLDTTVTTTYLQISSLKAEDTAVYFCARE |
| | E345R + P329R | AGSFDYWGQGTLVTVSSASTKGPSVFPLAPSSKSTSGGTAALGCLVKDYF |
| | (without c- | PEPVTVSWNSGALTSGVHTFPAVLQSSGLYSLSSVVTVPSSSLGTQTYIC |
| | terminal | NVNHKPSNTKVDKRVEPKSCDKTHTCPPCPAPELLGGPSVFLFPPKPKDTL |
| | lysine) | MISRTPEVTCVVVDVSHEDPEVKFNWYVDGVEVHNAKTKPREEQYNSTY |
| | | RVVSVLTVLHQDWLNGKEYKCKVSNKALRAPIEKTISKAKGQPRRPQVYT |
| | | LPPSREEMTKNQVSLTCLVKGFYPSDIAVEWESNGQPENNYKTTPPVLDS |
| | | DGSFFLYSKLTVDKSRWQQGNVFSCSVMHEALHNHYTQKSLSLSPG |
| 36 | Human IgG1- | ASTKGPSVFPLAPSSKSTSGGTAALGCLVKDYFPEPVTVSWNSGALTSGV |
| | Fc- | HTFPAVLQSSGLYSLSSVVTVPSSSLGTQTYICNVNHKPSNTKVDKRVEPK |
| | E345R + P329R | SCDKTHTCPPCPAPELLGGPSVFLFPPKPKDTLMISRTPEVTCVVVDVSHE |
| | (without c- | DPEVKFNWYVDGVEVHNAKTKPREEQYNSTYRVVSVLTVLHQDWLNGKE |
| | terminal | YKCKVSNKALRAPIEKTISKAKGQPRRPQVYTLPPSREEMTKNQVSLTCLV |
| | lysine) | KGFYPSDIAVEWESNGQPENNYKTTPPVLDSDGSFFLYSKLTVDKSRWQ |
| | | QGNVFSCSVMHEALHNHYTQKSLSLSPG |
| 37 | constant | ASTKGPSVFPLAPSSKSTSGGTAALGCLVKDYFPEPVTVSWNSGALTSGV |
| | region human | HTFPAVLQSSGLYSLSSVVTVPSSSLGTQTYICNVNHKPSNTKVDKRVEPK |
| | HC IgG1m(f) | SCDKTHTCPPCPAPELLGGPSVFLFPPKPKDTLMISRTPEVTCVVVDVSHE |
| | | DPEVKFNWYVDGVEVHNAKTKPREEQYNSTYRVVSVLTVLHQDWLNGKE |
| | | YKCKVSNKALPAPIEKTISKAKGQPREPQVYTLPPSREEMTKNQVSLTCLV |
| | | KGFYPSDIAVEWESNGQPENNYKTTPPVLDSDGSFFLYSKLTVDKSRWQ |
| | | QGNVFSCSVMHEALHNHYTQKSLSLSPG |
| 38 | constant | ASTKGPSVFPLAPSSKSTSGGTAALGCLVKDYFPEPVTVSWNSGALTSGV |
| | region human | HTFPAVLQSSGLYSLSSVVTVPSSSLGTQTYICNVNHKPSNTKVDKRVEPK |
| | HC IgG1m(f)- | SCDKTHTCPPCPAPEX₁X₂X₃GPSVFLFPPKPKDTLMISRTPEVTCVVVDVS |
| | L234F-L235E- | HEDPEVKFNWYVDGVEVHNAKTKPREEQYNSTYRVVSVLTVLHQDWLNG |
| | G236R and | KEYKCKVSNKALPAPIEKTISKAKGQPREPQVYTLPPSREEMTKNQVSLTC |
| | variants | LVKGFYPSDIAVEWESNGQPENNYKTTPPVLDSDGSFFLYSKLTVDKSRW |
| | | QQGNVFSCSVMHEALHNHYTQKSLSLSPG |
| | | wherein: |
| | | X₁ = A, V, L, I, P, F, M or W, preferably I, P, F, |
| | | M or W, more preferably F; |
| | | X₂ = L, D or E, preferably D or E, more preferably E; |
| | | X₃ = not G, preferably K, R or H, more preferably R; |
| | | most preferably X₁X₂X₃ = FER |
| 39 | constant | ASTKGPSVFPLAPSSKSTSGGTAALGCLVKDYFPEPVTVSWNSGALTSGV |
| | region human | HTFPAVLQSSGLYSLSSVVTVPSSSLGTQTYICNVNHKPSNTKVDKRVEPK |
| | HC IgG1m(f)- | SCDKTHTCPPCPAPEFERGPSVFLFPPKPKDTLMISRTPEVTCVVVDVSHE |
| | L234F-L235E- | DPEVKFNWYVDGVEVHNAKTKPREEQYNSTYRVVSVLTVLHQDWLNGKE |
| | G236R-K409R | YKCKVSNKALPAPIEKTISKAKGQPREPQVYTLPPSREEMTKNQVSLTCLV |
| | | KGFYPSDIAVEWESNGQPENNYKTTPPVLDSDGSFFLYSRLTVDKSRWQ |
| | | QGNVFSCSVMHEALHNHYTQKSLSLSPG |
| 40 | constant | ASTKGPSVFPLAPSSKSTSGGTAALGCLVKDYFPEPVTVSWNSGALTSGV |
| | region human | HTFPAVLQSSGLYSLSSVVTVPSSSLGTQTYICNVNHKPSNTKVDKRVEPK |
| | HC IgG1m(f)- | SCDKTHTCPPCPAPELLGGPSVFLFPPKPKDTLMISRTPEVTCVVVDVSHE |
| | E430G | DPEVKFNWYVDGVEVHNAKTKPREEQYNSTYRVVSVLTVLHQDWLNGKE |
| | | YKCKVSNKALPAPIEKTISKAKGQPREPQVYTLPPSREEMTKNQVSLTCLV |
| | | KGFYPSDIAVEWESNGQPENNYKTTPPVLDSDGSFFLYSKLTVDKSRWQ |
| | | QGNVFSCSVMHGALHNHYTQKSLSLSPG |
| 41 | constant | ASTKGPSVFPLAPCSRSTSESTAALGCLVKDYFPEPVTVSWNSGALTSGV |
| | region human | HTFPAVLQSSGLYSLSSVVTVPSSSLGTKTYTCNVDHKPSNTKVDKRVES |
| | HC IgG4 | KYGPPCPSCPAPEFLGGPSVFLFPPKPKDTLMISRTPEVTCVVVDVSQEDP |
| | | EVQFNWYVDGVEVHNAKTKPREEQFNSTYRVVSVLTVLHQDWLNGKEYK |
| | | CKVSNKGLPSSIEKTISKAKGQPREPQVYTLPPSQEEMTKNQVSLTCLVKG |
| | | FYPSDIAVEWESNGQPENNYKTTPPVLDSDGSFFLYSRLTVDKSRWQEGN |
| | | VFSCSVMHEALHNHYTQKSLSLSLGK |
| 42 | constant | RTVAAPSVFIFPPSDEQLKSGTASVVCLLNNFYPREAKVQWKVDNALQSG |
| | region human | NSQESVTEQDSKDSTYSLSSTLTLSKADYEKHKVYACEVTHQGLSSPVTK |
| | kappa LC | SFNRGEC |
| 43 | Heavy chain | ASTKGPSVFPLAPSSKSTSGGTAALGCLVKDYFPEPVTVSWNSGALTSGV |
| | human IgG1- | HTFPAVLQSSGLYSLSSVVTVPSSSLGTQTYICNVNHKPSNTKVDKKVEPK |
| | LALA | SCDKTHTCPPCPAPEAAGGPSVFLFPPKPKDTLMISRTPEVTCVVVDVSHE |
| | | DPEVKFNWYVDGVEVHNAKTKPREEQYNSTYRVVSVLTVLHQDWLNGKE |
| | | YKCKVSNKALPAPIEKTISKAKGQPREPQVYTLPPSREEMTKNQVSLTCLV |
| | | KGFYPSDIAVEWESNGQPENNYKTTPPVLDSDGSFFLYSKLTVDKSRWQ |
| | | QGNVFSCSVMHEALHNHYTQKSLSLSPG |
| 44 | HCDR 3 inter- | AFYDDYDYNV |
| | section of | |
| | Kabat and | |
| | IMGT | |
| | (= Kabat) | |
| 45 | HCDR 3 IMGT | ARAFYDDYDYNV |
| 46 | HCDR 2 inter- | ISGGTIG |
| | section of | |
| | Kabat and | |
| | IMGT | |
| | (= IMGT) | |
| 47 | HCDR 2 | IISGGTIGHYASWAKG |
| | Kabat | |
| | HCDR 1 inter- | SYN |
| | section of | |
| | Kabat and | |
| | IMGT | |
| 48 | HCDR 1 | SYNMG |
| | Kabat | |
| 49 | HCDR 1 IMGT | GFSLYSYN |
| 50 | LCDR 3 inter- | AGGYSSSSDTT |
| | section = | |
| | Kabat = | |
| | IMGT | |
| | LCDR2 inter- | QAS |
| | section of | |
| | Kabat and | |
| | IMGT | |
| | (= IMGT) | |
| 51 | LCDR2 Kabat | QASKLET |
| 52 | LCDR1 inter- | QSVYGNNQ |
| | section of | |
| | Kabat and | |
| | IMGT | |
| | (= IMGT) | |
| 53 | LCDR1 Kabat | QSSQSVYGNNQLS |
| 54 | VH MAB-19- | QSVEESGGRLVTPGTPLTLTCTVSGFSLYSYNMGWVRQAPGKGLEYIGIIS |
| | 0202 | GGTIGHYASWAKGRFTISKTSSTTVDLKMTSLTTEDTATYFCARAFYDDY |
| | | DYNVWGPGTLVTVSS |
| 55 | VL MAB-19- | AAVLTQTPSPVSAAVGGTVTISCQSSQSVYGNNQLSWYQQKPGQPPKLLI |
| | 0202 | YQASKLETGVPSRFKGSGSGTQFTLTISDLESDDAATYYCAGGYSSSSDT |
| | | TFGGGTEVVVK |
| 56 | VH IgG1-PD1 | QVQLVESGGGLVQPGTSLRLSCSVSGFSLYSYNMGWVRQAPGKGLEYI |
| | (H5 derived | GIISGGTIGHYASWAKGRFTISRDTSKTTLYLQMNSLTTEDTATYFCARA |
| | from MAB- | FYDDYDYNVWGPGTLVTVSS |
| | 19-0202) | |
| 57 | VL IgG1-PD1 | AIQLTQSPSSLSASVGGTVTITCQSSQSVYGNNQLSWYQQKPGQPPKLL |
| | (L4 derived | IYQASKLETGVPSRFRGSGSGTQFTLTISSLQSEDFATYYCAGGYSSSSD |
| | from MAB- | TTFGGGTEVVVK |
| | 19-0202) | |
| 58 | Human PD-1 | MQIPQAPWPVVWAVLQLGWRPGWFLDSPDRPWNPPTFSPALLVVTEGD |
| | complete | NATFTCSFSNTSESFVLNWYRMSPSNQTDKLAAFPEDRSQPGQDCRFRV |
| | | TQLPNGRDFHMSVVRARRNDSGTYLCGAISLAPKAQIKESLRAELRVTER |
| | | RAEVPTAHPSPSPRPAGQFQTLVVGVVGGLLGSLVLLVWVLAVICSRAAR |
| | | GTIGARRTGQPLKEDPSAVPVFSVDYGELDFQWREKTPEPPVPCVPEQTE |
| | | YATIVFPSGMGTSSPARRGSADGPRSAQPLRPEDGHCSWPL |
| 59 | Human PD-1 | FLDSPDRPWNPPTFSPALLVVTEGDNATFTCSFSNTSESFVLNWYRMSPS |
| | complete | NQTDKLAAFPEDRSQPGQDCRFRVTQLPNGRDFHMSVVRARRNDSGTYL |
| | extracellular | CGAISLAPKAQIKESLRAELRVTERRAEVPTAHPSPSPRPAGQFQTLV |
| | domain | |
| 60 | Nucleic acid | agtttcccttccgctcacctccgcctgagcagtggagaaggcggcactctggtggggctgctc |
| | human PD-1 | caggcatgcagatcccacaggcgccctggccagtcgtctgggcggtgctacaactgggctg |
| | | gcggccaggatggttcttagactccccagacaggccctggaacccccccaccttctccccagc |
| | | cctgctcgtggtgaccgaaggggacaacgccaccttcacctgcagcttctccaacacatcgg |
| | | agagcttcgtgctaaactggtaccgcatgagccccagcaaccagacggacaagctggccgc |
| | | cttccccgaggaccgcagccagcccggccaggactgccgcttccgtgtcacacaactgccca |
| | | acgggcgtgacttccacatgagcgtggtcagggcccggcgcaatgacagcggcacctacct |
| | | ctgtggggccatctccctggcccccaaggcgcagatcaaagagagcctgcgggcagagctc |
| | | agggtgacagagagaagggcagaagtgcccacagcccaccccagcccctcacccaggcca |
| | | gccggccagttccaaaccctggtggttggtgtcgtgggcggcctgctgggcagcctggtgct |
| | | gctagtctgggtcctggccgtcatctgctcccgggccgcacgagggacaataggagccagg |
| | | cgcaccggccagcccctgaaggaggacccctcagccgtgcctgtgttctctgtggactatgg |
| | | ggagctggatttccagtggcgagagaagaccccggagccccccgtgccctgtgtccctgagc |
| | | agacggagtatgccaccattgtctttcctagcggaatgggcacctcatcccccgcccgcaggg |
| | | gctcagctgacggccctcggagtgcccagccactgaggcctgaggatggacactgctcttgg |
| | | cccctctgaccggcttccttggccaccagtgttctgcagaccctccaccatgagcccgggtcag |
| | | cgcatttcctcaggagaagcaggcagggtgcaggccattgcaggccgtccaggggctgagc |
| | | tgcctgggggcgaccggggctccagcctgcacctgcaccaggcacagccccaccacaggac |
| | | tcatgtctcaatgcccacagtgagcccaggcagcaggtgtcaccgtcccctacagggagggc |
| | | cagatgcagtcactgcttcaggtcctgccagcacagagctgcctgcgtccagctccctgaatc |
| | | tctgctgctgctgctgctgctgctgctgctgcctgcggcccggggctgaaggcgccgtggccc |
| | | tgcctgacgccccggagcctcctgcctgaacttgggggctggttggagatggccttggagca |
| | | gccaaggtgcccctggcagtggcatcccgaaacgccctggacgcagggcccaagactggg |
| | | cacaggagtgggaggtacatggggctggggactccccaggagttatctgctccctgcaggc |
| | | ctagagaagtttcagggaaggtcagaagagctcctggctgtggtgggcagggcaggaaac |
| | | ccctccacctttacacatgcccaggcagcacctcaggccctttgtggggcagggaagctgag |
| | | gcagtaagcgggcaggcagagctggaggcctttcaggcccagccagcactctggcctcctg |
| | | ccgccgcattccaccccagcccctcacaccactcgggagagggacatcctacggtcccaagg |
| | | tcaggagggcagggctggggttgactcaggcccctcccagctgtggccacctgggtgttggg |
| | | agggcagaagtgcaggcacctagggccccccatgtgcccaccctgggagctctccttggaa |
| | | cccattcctgaaattatttaaaggggttggccgggctcccaccagggcctgggtgggaaggt |
| | | acaggcgttcccccggggcctagtacccccgccgtggcctatccactcctcacatccacacac |
| | | tgcacccccactcctggggcagggccaccagcatccaggcggccagcaggcacctgagtgg |
| | | ctgggacaagggatcccccttccctgtggttctattatattataattataattaaatatgagagc |
| | | atgctaaggaaaa |
| 61 | VH CD52- | QVQLQESGPGLVRPSQTLSLTCTVSGFTFTDFYMNWVRQPPGRGLEWI |
| | CAMPATH-1H | GFIRDKAKGYTTEYNPSVKGRVTMLVDTSKNQFSLRLSSVTAADTAVYY |
| | | CAREGHTAAPFDYWGQGSLVTVSS |
| 62 | VH CD52- | GFTFTDFY |
| | CAMPATH-1H | |
| | CDR1 | |
| 63 | VH CD52- | IRDKAKGYTT |
| | CAMPATH-1H | |
| | CDR2 | |
| 64 | VH CD52- | AREGHTAAPFDY |
| | CAMPATH-1H | |
| | CDR3 | |
| 65 | VL CD52- | DIQMTQSPSSLSASVGDRVTITCKASQNIDKYLNWYQQKPGKAPKLLIY |
| | CAMPATH-1H | NTNNLQTGVPSRFSGSGSGTDFTFTISSLQPEDIATYYCLQHISRPRTFG |
| | | QGTKVEIK |
| 66 | VL CD52- | QNIDKY |
| | CAMPATH-1H | |
| | CDR1 | |
| | VL CD52- | NTN |
| | CAMPATH-1H | |
| | CDR2 | |
| 67 | VL CD52- | LQHISRPRT |
| | CAMPATH-1H | |
| | CDR3 | |
| 68 | VH gp120- | QVQLVQSGAEVKKPGASVKVSCQASGYRFSNFVIHWVRQAPGQRFEWM |
| | b12 | GWINPYNGNKEFSAKFQDRVTFTADTSANTAYMELRSLRSADTAVYYCAR |
| | | VGPYSWDDSPQDNYYMDVWGKGTTVIVSS |
| 69 | VH gp120- | GYRFSNFV |
| | b12 CDR1 | |
| 70 | VH gp120- | INPYNGNK |
| | b12 CDR2 | |
| 71 | VH gp120- | ARVGPYSWDDSPQDNYYMDV |
| | b12 CDR3 | |
| 72 | VL gp120- | EIVLTQSPGTLSLSPGERATFSCRSSHSIRSRRVAWYQHKPGQAPRLVIH |
| | b12 | GVSNRASGISDRFSGSGSGTDFTLTITRVEPEDFALYYCQVYGASSYTFG |
| | | QGTKLERK |
| 73 | VL gp120- | HSIRSRR |
| | b12 CDR1 | |
| | VL gp120- | GVS |
| | b12 CDR2 | |
| 74 | VL gp120- | QVYGASSYT |
| | b12-CDR3 | |
| 75 | CD137-009 | EVQLVESGGGLVQPGRSLRLSCTASGFSLNDYWMSWVRQAPGKGLEWV |
| | heavy chain | GYIDVGGSLYYAASVKGRFTISRDDSKSIAYLQMNSLKTEDTAVYYCARG |
| | | GLTYGFDLWGQGTLVTVSSASTKGPSVFPLAPSSKSTSGGTAALGCLVKD |
| | | YFPEPVTVSWNSGALTSGVHTFPAVLQSSGLYSLSSVVTVPSSSLGTQTYI |
| | | CNVNHKPSNTKVDKRVEPKSCDKTHTCPPCPAPEFEGGPSVFLFPPKPKD |
| | | TLMISRTPEVTCVVVAVSHEDPEVKFNWYVDGVEVHNAKTKPREEQYNST |
| | | YRVVSVLTVLHQDWLNGKEYKCKVSNKALPAPIEKTISKAKGQPREPQVY |
| | | TLPPSREEMTKNQVSLTCLVKGFYPSDIAVEWESNGQPENNYKTTPPVLD |
| | | SDGSFFLYSRLTVDKSRWQQGNVFSCSVMHEALHNHYTQKSLSLSPG |
| 76 | CD137-009 | DIVMTQSPSSLSASVGDRVTITCQASEDISSYLAWYQQKPGKAPKRLIYG |
| | light chain | ASDLASGVPSRFSASGSGTDYTFTISSLQPEDIATYYCHYYATISGLGVAF |
| | | GGGTKVEIKRTVAAPSVFIFPPSDEQLKSGTASVVCLLNNFYPREAKVQW |
| | | KVDNALQSGNSQESVTEQDSKDSTYSLSSTLTLSKADYEKHKVYACEVTH |
| | | QGLSSPVTKSFNRGEC |
| 77 | PD-L1-547 | EVQLLEPGGGLVQPGGSLRLSCEASGSTFSTYAMSWVRQAPGKGLEWVS |
| | heavy chain | GFSGSGGFTFYADSVRGRFTISRDSSKNTLFLQMSSLRAEDTAVYYCAIPA |
| | | RGYNYGSFQHWGQGTLVTVSSASTKGPSVFPLAPSSKSTSGGTAALGCL |
| | | VKDYFPEPVTVSWNSGALTSGVHTFPAVLQSSGLYSLSSVVTVPSSSLGT |
| | | QTYICNVNHKPSNTKVDKRVEPKSCDKTHTCPPCPAPEFEGGPSVFLFPPK |
| | | PKDTLMISRTPEVTCVVVAVSHEDPEVKFNWYVDGVEVHNAKTKPREEQY |
| | | NSTYRVVSVLTVLHQDWLNGKEYKCKVSNKALPAPIEKTISKAKGQPREP |
| | | QVYTLPPSREEMTKNQVSLTCLVKGFYPSDIAVEWESNGQPENNYKTTPP |
| | | VLDSDGSFLLYSKLTVDKSRWQQGNVFSCSVMHEALHNHYTQKSLSLSP |
| | | G |
| 78 | PD-L1-547 | SYVLTQPPSVSVAPGQTARITCGGNNIGSKSVHWYQQKPGQAPVLVVYD |
| | light chain | DNDRPSGLPERFSGSNSGNTATLTISRVEAGDEADYYCQVWDSSSDHVV |
| | | FGGGTKLTVLGQPKAAPSVTLFPPSSEELQANKATLVCLISDFYPGAVTVA |
| | | WKADSSPVKAGVETTTPSKQSNNKYAASSYLSLTPEQWKSHRSYSCQVT |
| | | HEGSTVEKTVAPTECS |
| 79 | VH_CD137- | EVQLVESGGGLVQPGRSLRLSCTASGFSLNDYWMSWVRQAPGKGLEWV |
| | 009-H7 | GYIDVGGSLYYAASVKGRFTISRDDSKSIAYLQMNSLKTEDTAVYYCARG |
| | | GLTYGFDLWGQGTLVTVSS |
| 80 | VH_CD137- | GFSLNDYW |
| | 009- | |
| | H7_CDR1 | |
| 81 | VH_CD137- | IDVGGSL |
| | 009- | |
| | H7_CDR2 | |
| 82 | VH_CD137- | ARGGLTYGFDL |
| | 009- | |
| | H7_CDR3 | |
| 83 | VL_CD137- | DIVMTQSPSSLSASVGDRVTITCQASEDISSYLAWYQQKPGKAPKRLIYG |
| | 009-L2 | ASDLASGVPSRFSASGSGTDYTFTISSLQPEDIATYYCHYYATISGLGVAF |
| | | GGGTKVEIK |
| 84 | VL_CD137- | EDISSY |
| | 009-L2_CDR1 | |
| | VL_CD137- | GAS |
| | 009-L2_CDR2 | |
| 85 | VL_CD137- | HYYATISGLGVA |
| | 009-L2_CDR3 | |
| 86 | VH-PD-L1- | EVQLLEPGGGLVQPGGSLRLSCEASGSTFSTYAMSWVRQAPGKGLEWVS |
| | 547 | GFSGSGGFTFYADSVRGRFTISRDSSKNTLFLQMSSLRAEDTAVYYCAIPA |
| | | RGYNYGSFQHWGQGTLVTVSS |
| 87 | VH-PD-L1- | GSTFSTYA |
| | 547-CDR1 | |
| 88 | VH-PD-L1- | FSGSGGFT |
| | 547-CDR2 | |
| 89 | VH-PD-L1- | AIPARGYNYGSFQH |
| | 547-CDR3 | |
| 90 | VL-PD-L1- | SYVLTQPPSVSVAPGQTARITCGGNNIGSKSVHWYQQKPGQAPVLVVYD |
| | 547 | DNDRPSGLPERFSGSNSGNTATLTISRVEAGDEADYYCQVWDSSSDHVV |
| | | FGGGTKLTVL |
| 91 | VL-PD-L1- | NIGSKS |
| | 547-CDR1 | |
| | VL-PD-L1- | DDN |
| | 547-CDR2 | |
| 92 | VL-PD-L1- | QVWDSSSDHVV |
| | 547-CDR3 | |
| 93 | IgG1-FEAR-Fc | ASTKGPSVFPLAPSSKSTSGGTAALGCLVKDYFPEPVTVSWNSGALTSGV |
| | | HTFPAVLQSSGLYSLSSVVTVPSSSLGTQTYICNVNHKPSNTKVDKRVEPK |
| | | SCDKTHTCPPCPAPEFEGGPSVFLFPPKPKDTLMISRTPEVTCVVVAVSHE |
| | | DPEVKFNWYVDGVEVHNAKTKPREEQYNSTYRVVSVLTVLHQDWLNGKE |
| | | YKCKVSNKALPAPIEKTISKAKGQPREPQVYTLPPSREEMTKNQVSLTCLV |
| | | KGFYPSDIAVEWESNGQPENNYKTTPPVLDSDGSFFLYSRLTVDKSRWQ |
| | | QGNVFSCSVMHEALHNHYTQKSLSLSPGK |
| 94 | IgG1-FEAL-Fc | ASTKGPSVFPLAPSSKSTSGGTAALGCLVKDYFPEPVTVSWNSGALTSGV |
| | | HTFPAVLQSSGLYSLSSVVTVPSSSLGTQTYICNVNHKPSNTKVDKRVEPK |
| | | SCDKTHTCPPCPAPEFEGGPSVFLFPPKPKDTLMISRTPEVTCVVVAVSHE |
| | | DPEVKFNWYVDGVEVHNAKTKPREEQYNSTYRVVSVLTVLHQDWLNGKE |
| | | YKCKVSNKALPAPIEKTISKAKGQPREPQVYTLPPSREEMTKNQVSLTCLV |
| | | KGFYPSDIAVEWESNGQPENNYKTTPPVLDSDGSFLLYSKLTVDKSRWQ |
| | | QGNVFSCSVMHEALHNHYTQKSLSLSPGK |
| 95 | IgG1-FEAR-Fc | ASTKGPSVFPLAPSSKSTSGGTAALGCLVKDYFPEPVTVSWNSGALTSGV |
| | without C- | HTFPAVLQSSGLYSLSSVVTVPSSSLGTQTYICNVNHKPSNTKVDKRVEPK |
| | terminal | SCDKTHTCPPCPAPEFEGGPSVFLFPPKPKDTLMISRTPEVTCVVVAVSHE |
| | Lysine | DPEVKFNWYVDGVEVHNAKTKPREEQYNSTYRVVSVLTVLHQDWLNGKE |
| | | YKCKVSNKALPAPIEKTISKAKGQPREPQVYTLPPSREEMTKNQVSLTCLV |
| | | KGFYPSDIAVEWESNGQPENNYKTTPPVLDSDGSFFLYSRLTVDKSRWQ |
| | | QGNVFSCSVMHEALHNHYTQKSLSLSPG |
| 96 | IgG1-FEAL-Fc | ASTKGPSVFPLAPSSKSTSGGTAALGCLVKDYFPEPVTVSWNSGALTSGV |
| | without C- | HTFPAVLQSSGLYSLSSVVTVPSSSLGTQTYICNVNHKPSNTKVDKRVEPK |
| | terminal | SCDKTHTCPPCPAPEFEGGPSVFLFPPKPKDTLMISRTPEVTCVVVAVSHE |
| | Lysine | DPEVKFNWYVDGVEVHNAKTKPREEQYNSTYRVVSVLTVLHQDWLNGKE |
| | | YKCKVSNKALPAPIEKTISKAKGQPREPQVYTLPPSREEMTKNQVSLTCLV |
| | | KGFYPSDIAVEWESNGQPENNYKTTPPVLDSDGSFLLYSKLTVDKSRWQ |
| | | QGNVFSCSVMHEALHNHYTQKSLSLSPG |
| 97 | Human | LQDPCSNCPAGTFCDNNRNQICSPCPPNSFSSAGGQRTCDICRQCKGVF |
| | CD137 | RTRKECSSTSNAECDCTPGFHCLGAGCSMCEQDCKQGQELTKKGCKDC |
| | (UniProtKB- | CFGTFNDQKRGICRPWTNCSLDGKSVLVNGTKERDVVCGPSPADLSPGA |
| | Q07011; | SSVTPPAPAREPGHSPQIISFFLALTSTALLFLLFFLTLRFSVVKRGRKKLLYI |
| | mature | FKQPFMRPVQTTQEEDGCSCRFPEEEEGGCEL |
| | sequence) | |
| 98 | Human PD-L1 | FTVTVPKDLYVVEYGSNMTIECKFPVEKQLDLAALIVYWEMEDKNIIQFVH |
| | (UniProtKB- | GEEDLKVQHSSYRQRARLLKDQLSLGNAALQITDVKLQDAGVYRCMISYG |
| | Q9NZQ7; | GADYKRITVKVNAPYNKINQRILVVDPVTSEHELTCQAEGYPKAEVIWTSS |
| | mature | DHQVLSGKTTTTNSKREEKLFNVTSTLRINTTTNEIFYCTFRRLDPEENHTA |
| | sequence) | ELVIPELPLAHPPNERTHLVILGAILLCLGVALTFIFRLRKGRMMDVKKCGIQ |
| | | DTNSKKQSDTHLEET |
| 99 | Pembrolizumab | GYTFTNYY |
| | VH CDR1 | |
| 100 | Pembrolizumab | INPSNGGT |
| | VH CDR2 | |
| 101 | Pembrolizumab | ARRDYRFDMGFDY |
| | VH CDR3 | |
| 102 | Pembrolizumab | KGVSTSGYSY |
| | VL CDR1 | |
| | Pembrolizumab | LAS |
| | VL CDR2 | |
| 103 | Pembrolizumab | QHSRDLPLT |
| | VL CDR3 | |
| 104 | Pembrolizumab | QVQLVQSGVEVKKPGASVKVSCKASGYTFTNYYMYWVRQAPGQGLEWM |
| | VH | GGINPSNGGTNFNEKFKNRVTLTTDSSTTTAYMELKSLQFDDTAVYYCAR |
| | | RDYRFDMGFDYWGQGTTVTVSS |
| 105 | Pembrolizumab | EIVLTQSPATLSLSPGERATLSCRASKGVSTSGYSYLHWYQQKPGQAPRL |
| | VL | LIYLASYLESGVPARFSGSGSGTDFTLTISSLEPEDFAVYYCQHSRDLPLTF |
| | | GGGTKVEIK |
| 106 | Pembrolizumab | QVQLVQSGVEVKKPGASVKVSCKASGYTFTNYYMYWVRQAPGQGLEWM |
| | Heavy chain | GGINPSNGGTNFNEKFKNRVTLTTDSSTTTAYMELKSLQFDDTAVYYCAR |
| | | RDYRFDMGFDYWGQGTTVTVSSASTKGPSVFPLAPCSRSTSESTAALGCL |
| | | VKDYFPEPVTVSWNSGLTSGVHTFPAVLQSSGLYSLSSVVTVPSSSLGTK |
| | | TYTCNVDHKPSNTKVDKRVESKYGPPCPPCPAPEFLGGPSVFLFPPKPKDT |
| | | LMISRTPEVTCVVVDVSQEDPEVQFNWYVDGVEVHNAKTKPREEQFNST |
| | | YRVVSVLTVLHQDWLNGKEYKCKVSNKGLPSSIEKTISKAKGQPREPQVY |
| | | TLPPSQEEMTKNQVSLTCLVKGFYPSDIAVEWESNGQPENNYKTTPPVLD |
| | | SDGSFFLYSRLTVDKSRWQEGNVFSCSVMHEALHNHYTQKSLSLSLGK |
| 107 | Pembrolizumab | EIVLTQSPATLSLSPGERATLSCRASKGVSTSGYSYLHWYQQKPGQAPRL |
| | Light chain | LIYLASYLESGVPARFSGSGSGTDFTLTISSLEPEDFAVYYCQHSRDLPLTF |
| | | GGGTKVEIKRTVAAPSVFIFPPSDEQLKSGTASVVCLLNNFYPREAKVQW |
| | | KVDNALQSGNSQESVTEQDSKDSTYSLSSTLTLSKADYEKHKVYACEVTH |
| | | QGLSSPVTKSFNRGEC |
| 108 | Nivolumab | QVQLVESGGGVVQPGRSLRLDCKASGITFSNSGMHWVRQAPGKGLEWV |
| | Heavy chain | AVIWYDGSKRYYADSVKGRFTISRDNSKNTLFLQMNSLRAEDTAVYYCAT |
| | | NDDYWGQGTLVTVSSASTKGPSVFPLAPCSRSTSESTAALGCLVKDYFPE |
| | | PVTVSWNSGALTSGVHTFPAVLQSSGLYSLSSVVTVPSSSLGTKTYTCNV |
| | | DHKPSNTKVDKRVESKYGPPCPPCPAPEFLGGPSVFLFPPKPKDTLMISRT |
| | | PEVTCVVVDVSQEDPEVQFNWYVDGVEVHNAKTKPREEQFNSTYRVVSV |
| | | LTVLHQDWLNGKEYKCKVSNKGLPSSIEKTISKAKGQPREPQVYTLPPSQ |
| | | EEMTKNQVSLTCLVKGFYPSDIAVEWESNGQPENNYKTTPPVLDSDGSFF |
| | | LYSRLTVDKSRWQEGNVFSCSVMHEALHNHYTQKSLSLSLGK |
| 109 | Nivolumab | EIVLTQSPATLSLSPGERATLSCRASQSVSSYLAWYQQKPGQAPRLLIYDA |
| | Light chain | SNRATGIPARFSGSGSGTDFTLTISSLEPEDFAVYYCQQSSNWPRTFGQG |
| | | TKVEIKRTVAAPSVFIFPPSDEQLKSGTASVVCLLNNFYPREAKVQWKVDN |
| | | ALQSGNSQESVTEQDSKDSTYSLSSTLTLSKADYEKHKVYACEVTHQGLS |
| | | SPVTKSFNRGEC |
| 110 | Atezolizumab | EVQLVESGGGLVQPGGSLRLSCAASGFTFSDSWIHWVRQAPGKGLEWV |
| | Heavy chain | AWISPYGGSTYYADSVKGRFTISADTSKNTAYLQMNSLRAEDTAVYYCAR |
| | | RHWPGGFDYWGQGTLVTVSSASTKGPSVFPLAPSSKSTSGGTAALGCLV |
| | | KDYFPEPVTVSWNSGALTSGVHTFPAVLQSSGLYSLSSVVTVPSSSLGTQ |
| | | TYICNVNHKPSNTKVDKKVEPKSCDKTHTCPPCPAPELLGGPSVFLFPPKP |
| | | KDTLMISRTPEVTCVVVDVSHEDPEVKFNWYVDGVEVHNAKTKPREEQY |
| | | ASTYRVVSVLTVLHQDWLNGKEYKCKVSNKALPAPIEKTISKAKGQPREP |
| | | QVYTLPPSREEMTKNQVSLTCLVKGFYPSDIAVEWESNGQPENNYKTTPP |
| | | VLDSDGSFFLYSKLTVDKSRWQQGNVFSCSVMHEALHNHYTQKSLSLSP |
| | | GK |
| 111 | Atezolizumab | DIQMTQSPSSLSASVGDRVTITCRASQDVSTAVAWYQQKPGKAPKLLIYS |
| | Light chain | ASFLYSGVPSRFSGSGSGTDFTLTISSLQPEDFATYYCQQYLYHPATFGQG |
| | | TKVEIKRTVAAPSVFIFPPSDEQLKSGTASVVCLLNNFYPREAKVQWKVDN |
| | | ALQSGNSQESVTEQDSKDSTYSLSSTLTLSKADYEKHKVYACEVTHQGLS |
| | | SPVTKSFNRGEC |
| 112 | MAB-19- | QSVEESGGRLVTPGTPLTLTCTVSGFSLY |
| | 0202-HC FR1 | |
| 113 | MAB-19- | WVRQAPGKGLEYIG |
| | 0202-HC FR2 | |
| 114 | MAB-19- | RFTISKTSSTTVDLKMTSLTTEDTATYFCAR |
| | 0202-HC FR3 | |
| 115 | MAB-19- | WGPGTLVTVSS |
| | 0202-HC FR4 | |
| 116 | MAB-19- | AAVLTQTPSPVSAAVGGTVTISC |
| | 0202-LC FR1 | |
| 117 | MAB-19- | WYQQKPGQPPKLLIY |
| | 0202-LC FR2 | |
| 118 | MAB-19- | GVPSRFKGSGSGTQFTLTISDLESDDAATYYC |
| | 0202-LC FR3 | |
| 119 | MAB-19- | FGGGTEVVVK |
| | 0202-LC FR4 | |
| 120 | MAB-19- | QVQLVESGGGLVQPGTSLRLSCSVSGFSLY |
| | 0202-H5 FR1 | |
| 121 | MAB-19- | WVRQAPGKGLEYIG |
| | 0202-H5 FR2 | |
| 122 | MAB-19- | RFTISRDTSKTTLYLQMNSLTTEDTATYFCAR |
| | 0202-H5 FR3 | |
| 123 | MAB-19- | WGPGTLVTVSS |
| | 0202-H5 FR4 | |
| 124 | MAB-19- | AIQLTQSPSSLSASVGGTVTITC |
| | 0202-L4 FR1 | |
| 125 | MAB-19- | WYQQKPGQPPKLLIY |
| | 0202-L4 FR2 | |
| 126 | MAB-19- | GVPSRFRGSGSGTQFTLTISSLQSEDFATYYC |
| | 0202-L4 FR3 | |
| 127 | MAB-19- | FGGGTEVVVK |
| | 0202-L4 FR4 | |
| 128 | constant | ASTKGPSVFPLAPSSKSTSGGTAALGCLVKDYFPEPVTVSWNSGALTSGV |
| | region human | HTFPAVLQSSGLYSLSSVVTVPSSSLGTQTYICNVNHKPSNTKVDKRVEPK |
| | HC IgG1m(f)- | SCDKTHTCPPCPAPEFERGPSVFLFPPKPKDTLMISRTPEVTCVVVDVSHE |
| | L234F-L235E- | DPEVKFNWYVDGVEVHNAKTKPREEQYNSTYRVVSVLTVLHQDWLNGKE |
| | G236R | YKCKVSNKALPAPIEKTISKAKGQPREPQVYTLPPSREEMTKNQVSLTCLV |
| | | KGFYPSDIAVEWESNGQPENNYKTTPPVLDSDGSFFLYSKLTVDKSRWQ |
| | | QGNVFSCSVMHEALHNHYTQKSLSLSPG |
| 129 | Human PD-L1 | MRIFAVFIFMTYWHLLNAFTVTVPKDLYVVEYGSNMTIECFPVEKQLDLAA |
| | (UniProtKB- | LIVYWEMEDKNIIQFVHGEEDLKVQHSSYRQRARLLKDQLSLGNAALQIT |
| | Q9NZQ7; | DVKLQDAGVYRCMISYGGADYKRITVKVNAPYNKINQRILVVDPVTSEHE |
| | incl. signal | LTCQAEGYPKAEVIWTSSDHQVLSGKTTTTNSKREEKLFNVTSTLRINTTT |
| | peptide | NEIFYCTFRRLDPEENHTAELVIPELPLAHPPNERTHLVILGAILLCLGVALTF |
| | sequence: aa | IFRLRKGRMMDVKKCGIQDTNSKKQSDTHLEET |
| | 1-18) | |
| 130 | Human | MGNSCYNIVATLLLVLNFERTRSLQDPCSNCPAGTFCDNNRNQICSPCPP |
| | CD137 | NSFSSAGGQRTCDICRQCKGVFRTRKECSSTSNAECDCTPGFHCLGAGC |
| | (UniProtKB- | SMCEQDCKQGQELTKKGCKDCCFGTFNDQKRGICRPWTNCSLDGKSVL |
| | Q07011; incl. | VNGTKERDVVCGPSPADLSPGASSVTPPAPAREPGHSPQIISFFLALTSTA |
| | signal peptide | LLFLLFFLTLRFSVVKRGRKKLLYIFKQPFMRPVQTTQEEDGCSCRFPEEEE |
| | sequence: aa | GGCEL |
| | 1-23) | |
| 131 | IgG1-CD27- | QSALTQPASVSGSPGQSITISCTGTSSDVYYYNYVSWYQQHPGRAPKLVI |
| | A-P329R- | YDVSNRPSGVSNRFSGSKSGNTASLTISGLRAEDEADYYCSSYTVNRVW |
| | E345R-LNluc | VFGGGTKLTVLGQPKAAPSVTLFPPSSEELQANKATLVCLISDFYPGAVTV |
| | | AWKADSSPVKAGVETTTPSKQSNNKYAASSYLSLTPEQWKSHRSYSCQV |
| | | THEGSTVEKTVAPTECSSLGSSGVFTLEDFVGDWRQTAGYNLDQVLE |
| | | QGGVSSLFQNLGVSVTPIQRIVLSGENGLKIDIHVIIPYEGLSGDQ |
| | | MGQIEKIFKVVYPVDDHHFKVILHYGTLVIDGVTPNMIDYFGRPY |
| | | EGIAVFDGKKITVTGTLWNGNKIIDERLINPDGSLLFRVTINGVTG |
| | | WRLCERILA |
| 132 | IgG1-CD27- | QSALTQPASVSGSPGQSITISCTGTSSDVYYYNYVSWYQQHPGRAPKLVI |
| | A-P329R- | YDVSNRPSGVSNRFSGSKSGNTASLTISGLRAEDEADYYCSSYTVNRVW |
| | E345R-LHalo | VFGGGTKLTVLGQPKAAPSVTLFPPSSEELQANKATLVCLISDFYPGAVTV |
| | | AWKADSSPVKAGVETTTPSKQSNNKYAASSYLSLTPEQWKSHRSYSCQV |
| | | THEGSTVEKTVAPTECSSLEPTTEDLYFQSDNDGSEIGTGFPFDPHY |
| | | VEVLGERMHYVDVGPRDGTPVLFLHGNPTSSYVWRNIIPHVAPT |
| | | HRCIAPDLIGMGKSDKPDLGYFFDDHVRFMDAFIEALGLEEVVLVI |
| | | HDWGSALGFHWAKRNPERVKGIAFMEFIRPIPTWDEWPEFARET |
| | | FQAFRTTDVGRKLIIDQNVFIEGTLPMGVVRPLTEVEMDHYREPFL |
| | | NPVDREPLWRFPNELPIAGEPANIVALVEEYMDWLHQSPVPKLLF |
| | | WGTPGVLIPPAEAARLAKSLPNCKAVDIGPGLNLLQEDNPDLIGS |
| | | EIARWLSTLEISG |
| 133 | IgG1-b12- | EIVLTQSPGTLSLSPGERATFSCRSSHSIRSRRVAWYQHKPGQAPRLVIH |
| | P329R- | GVSNRASGISDRFSGSGSGTDFTLTITRVEPEDFALYYCQVYGASSYTFG |
| | E345R-LNLuc | QGTKLERKRTVAAPSVFIFPPSDEQLKSGTASVVCLLNNFYPREAKVQWK |
| | | VDNALQSGNSQESVTEQDSKDSTYSLSSTLTLSKADYEKHKVYACEVTH |
| | | QGLSSPVTKSFNRGECSLGSSGVFTLEDFVGDWRQTAGYNLDQVLE |
| | | QGGVSSLFQNLGVSVTPIQRIVLSGENGLKIDIHVIIPYEGLSGDQ |
| | | MGQIEKIFKVVYPVDDHHFKVILHYGTLVIDGVTPNMIDYFGRPY |
| | | EGIAVFDGKKITVTGTLWNGNKIIDERLINPDGSLLFRVTINGVTG |
| | | WRLCERILA |
| 134 | IgG1-b12- | EIVLTQSPGTLSLSPGERATFSCRSSHSIRSRRVAWYQHKPGQAPRLVIH |
| | P329R- | GVSNRASGISDRFSGSGSGTDFTLTITRVEPEDFALYYCQVYGASSYTFG |
| | E345R-LHalo | QGTKLERKRTVAAPSVFIFPPSDEQLKSGTASVVCLLNNFYPREAKVQWK |
| | | VDNALQSGNSQESVTEQDSKDSTYSLSSTLTLSKADYEKHKVYACEVTH |
| | | QGLSSPVTKSFNRGECSLEPTTEDLYFQSDNDGSEIGTGFPFDPHYV |
| | | EVLGERMHYVDVGPRDGTPVLFLHGNPTSSYVWRNIIPHVAPTH |
| | | RCIAPDLIGMGKSDKPDLGYFFDDHVRFMDAFIEALGLEEVVLVIH |
| | | DWGSALGFHWAKRNPERVKGIAFMEFIRPIPTWDEWPEFARETF |
| | | QAFRTTDVGRKLIIDQNVFIEGTLPMGVVRPLTEVEMDHYREPFL |
| | | NPVDREPLWRFPNELPIAGEPANIVALVEEYMDWLHQSPVPKLLF |
| | | WGTPGVLIPPAEAARLAKSLPNCKAVDIGPGLNLLQEDNPDLIGS |
| | | EIARWLSTLEISG |
| 135 | IgG1-CD27- | QSALTQPASVSGSPGQSITISCTGTSSDVYYYNYVSWYQQHPGRAPKLVI |
| | A-LNLuc | YDVSNRPSGVSNRFSGSKSGNTASLTISGLRAEDEADYYCSSYTVNRVW |
| | | VFGGGTKLTVLGQPKAAPSVTLFPPSSEELQANKATLVCLISDFYPGAVTV |
| | | AWKADSSPVKAGVETTTPSKQSNNKYAASSYLSLTPEQWKSHRSYSCQV |
| | | THEGSTVEKTVAPTECSSLGSSGVFTLEDFVGDWRQTAGYNLDQVLE |
| | | QGGVSSLFQNLGVSVTPIQRIVLSGENGLKIDIHVIIPYEGLSGDQ |
| | | MGQIEKIFKVVYPVDDHHFKVILHYGTLVIDGVTPNMIDYFGRPY |
| | | EGIAVFDGKKITVTGTLWNGNKIIDERLINPDGSLLFRVTINGVTG |
| | | WRLCERILA |
| 136 | IgG1-CD27- | QSALTQPASVSGSPGQSITISCTGTSSDVYYYNYVSWYQQHPGRAPKLVI |
| | A-LHalo | YDVSNRPSGVSNRFSGSKSGNTASLTISGLRAEDEADYYCSSYTVNRVW |
| | | VFGGGTKLTVLGQPKAAPSVTLFPPSSEELQANKATLVCLISDFYPGAVTV |
| | | AWKADSSPVKAGVETTTPSKQSNNKYAASSYLSLTPEQWKSHRSYSCQV |
| | | THEGSTVEKTVAPTECSSLEPTTEDLYFQSDNDGSEIGTGFPFDPHY |
| | | VEVLGERMHYVDVGPRDGTPVLFLHGNPTSSYVWRNIIPHVAPT |
| | | HRCIAPDLIGMGKSDKPDLGYFFDDHVRFMDAFIEALGLEEVVLVI |
| | | HDWGSALGFHWAKRNPERVKGIAFMEFIRPIPTWDEWPEFARET |
| | | FQAFRTTDVGRKLIIDQNVFIEGTLPMGVVRPLTEVEMDHYREPFL |
| | | NPVDREPLWRFPNELPIAGEPANIVALVEEYMDWLHQSPVPKLLF |
| | | WGTPGVLIPPAEAARLAKSLPNCKAVDIGPGLNLLQEDNPDLIGS |
| | | EIARWLSTLEISG |
| 137 | IgG1-CD20- | EIVLTQSPATLSLSPGERATLSCRASQSVSSYLAWYQQKPGQAPRLLIYDA |
| | 11B8-E430G- | SNRATGIPARFSGSGSGTDFTLTISSLEPEDFAVYYCQQRSDWPLTFGGG |
| | LNLuc | TKVEIKRTVAAPSVFIFPPSDEQLKSGTASVVCLLNNFYPREAKVQWKVDN |
| | | ALQSGNSQESVTEQDSKDSTYSLSSTLTLSKADYEKHKVYACEVTHQGLS |
| | | SPVTKSFNRGECSLGSSGVFTLEDFVGDWRQTAGYNLDQVLEQGG |
| | | VSSLFQNLGVSVTPIQRIVLSGENGLKIDIHVIIPYEGLSGDQMGQ |
| | | IEKIFKVVYPVDDHHFKVILHYGTLVIDGVTPNMIDYFGRPYEGIA |
| | | VFDGKKITVTGTLWNGNKIIDERLINPDGSLLFRVTINGVTGWRL |
| | | CERILA |
| 138 | IgG1-CD37- | DIQMTQSPASLSVSVGETVTITCRASENIRSNLAWYQQKQGKSPQLLVNV |
| | 37.3-E430G- | ATNLADGVPSRFSGSGSGTQYSLKINSLQSEDFGTYYCQHYWGTTWTFG |
| | LHalo | GGTKLEIKRTVAAPSVFIFPPSDEQLKSGTASVVCLLNNFYPREAKVQWKV |
| | | DNALQSGNSQESVTEQDSKDSTYSLSSTLTLSKADYEKHKVYACEVTHQ |
| | | GLSSPVTKSFNRGECSLEPTTEDLYFQSDNDGSEIGTGFPFDPHYVE |
| | | VLGERMHYVDVGPRDGTPVLFLHGNPTSSYVWRNIIPHVAPTHR |
| | | CIAPDLIGMGKSDKPDLGYFFDDHVRFMDAFIEALGLEEVVLVIHD |
| | | WGSALGFHWAKRNPERVKGIAFMEFIRPIPTWDEWPEFARETFQ |
| | | AFRTTDVGRKLIIDQNVFIEGTLPMGVVRPLTEVEMDHYREPFLNP |
| | | VDREPLWRFPNELPIAGEPANIVALVEEYMDWLHQSPVPKLLFW |
| | | GTPGVLIPPAEAARLAKSLPNCKAVDIGPGLNLLQEDNPDLIGSEI |
| | | ARWLSTLEISG |
| 139 | IgG1-PD1 | QVQLVESGGGLVQPGTSLRLSCSVSGFSLYSYNMGWVRQAPGKGLEYI |
| | heavy chain | GIISGGTIGHYASWAKGRFTISRDTSKTTLYLQMNSLTTEDTATYFCARA |
| | | FYDDYDYNVWGPGTLVTVSSASTKGPSVFPLAPSSKSTSGGTAALGCLV |
| | | KDYFPEPVTVSWNSGALTSGVHTFPAVLQSSGLYSLSSVVTVPSSSLGTQ |
| | | TYICNVNHKPSNTKVDKRVEPKSCDKTHTCPPCPAPEFERGPSVFLFPPKP |
| | | KDTLMISRTPEVTCVVVDVSHEDPEVKFNWYVDGVEVHNAKTKPREEQY |
| | | NSTYRVVSVLTVLHQDWLNGKEYKCKVSNKALPAPIEKTISKAKGQPREP |
| | | QVYTLPPSREEMTKNQVSLTCLVKGFYPSDIAVEWESNGQPENNYKTTPP |
| | | VLDSDGSFFLYSKLTVDKSRWQQGNVFSCSVMHEALHNHYTQKSLSLSP |
| | | G |
| 140 | IgG1-PD1 | AIQLTQSPSSLSASVGGTVTITCQSSQSVYGNNQLSWYQQKPGQPPKLL |
| | light chain | IYQASKLETGVPSRFRGSGSGTQFTLTISSLQSEDFATYYCAGGYSSSSD |
| | | TTFGGGTEVVVKRTVAAPSVFIFPPSDEQLKSGTASVVCLLNNFYPREAKV |
| | | QWKVDNALQSGNSQESVTEQDSKDSTYSLSSTLTLSKADYEKHKVYACE |
| | | VTHQGLSSPVTKSFNRGEC |

### Example 2: Agonist Activity of Anti-CD27 Antibodies in a CD27 Activation Reporter Cell Assay

CD27 agonist activity of the different anti-CD27 antibodies with and without an E345R or an E430G hexamerization-enhancing Fc mutation was measured using the CD27 Thaw and Use Bioassay kit (Promega, Custom Assay Services, CAS #CS1979A25). The kit contains NF-κB Reporter-Jurkat recombinant cells expressing the firefly luciferase gene under the control of NF-κB response elements with constitutive expression of human CD27 and was used essentially according to the manufacturer's instructions. Briefly, Thaw-and-Use GloResponse NFκB-luc2/CD27 Jurkat cells were thawed and incubated in 96-well flat bottom culture plates (PerkinElmer, Cat #6005680) with antibody dilution series (final concentration range 0.04-20 μg/mL) in Bio-Glo Luciferase Assay Buffer for 6 h at 37° C., 5% C02. The anti-CD27 antibodies were wild-type (WT*) IgG1-CD27-A, IgG1-CD27-B, IgG1-CD27-C, IgG1-CD27-D, IgG1-CD27-E, IgG1-CD27-F, and variants of each one harboring the E430G or E345R mutation. Anti-CD27 benchmark antibodies were IgG1-CD27-131A (WT and E430G variant) and a non-hexamerizing IgG1-CD27-15 (IgG1-CD27-15-P329R-E345R-K439E, that carries a combination of Fc mutations that prevents hexamerization and thus the mutations are functionally irrelevant in the context of this experiment and is therefore referred to as WT in the figure) and a hexamerizing variant of IgG1-CD27-15 comprising a E345R mutation. An anti-HIV gp120 human antibody, IgG1-b12-E345R, was used as a non-binding negative control antibody (ctrl). After the antibody incubation, Bio-Glo Luciferase Assay Reagent (equilibrated to RT) was added to each well and incubated at RT for 5-10 min. Luminescence was measured using an EnVision Multilabel Reader (PerkinElmer) and presented as relative luminescence units (RLU) in bar diagrams generated using GraphPad Prism software.

Introduction of a hexamerization-enhancing Fc mutation (E345R or E430G) resulted in enhanced CD27 agonism compared to the corresponding WT antibody for antibody clones IgG1-CD27-A to -E and for the benchmark antibodies IgG1-CD27-131A (tested with E430G) and IgG1-CD27-15 (tested with E345R; FIG. 1).

Whereas IgG-CD27-A, B and C demonstrated enhanced CD27 agonist activity after introduction of E430G or E345R at all concentrations tested, IgG1-CD27-D and E variant containing hexamerization-enhancing mutations did not show increased agonism at the lowest antibody concentrations. IgG1-CD27-F variants with the E430G or E345R mutations only showed enhanced CD27 agonism at the highest antibody concentration tested. For variants IgG1-CD27-A to -E, introduction of the E345R mutation resulted in stronger CD27 activation than the E430G mutation. Antibodies IgG1-CD27-A to -E having the E345R mutation showed higher or similar CD27 activation levels compared to IgG1-CD27-131A having the E430G mutation or CD27-15 having the E345R mutation, respectively.

**The WT antibodies for IgG1-CD27-B and IgG1-CD27-F carried a F405L mutation in the IgG Fc domain, which is functionally irrelevant in the context of this experiment.

### Example 3 Binding Affinities of Anti-Human CD27 Antibodies for Recombinant Human, Mouse and Cynomolgus Monkey CD27

The binding affinities of five anti-human CD27 IgG1 antibodies (IgG1-CD27-A, -B, -C, -D and -E) for recombinant human, cynomolgus monkey and mouse CD27 protein were determined using label-free biolayer interferometry on an Octet HTX instrument (FortéBio, Portsmouth, UK). Experiments were performed using bispecific antibodies comprising one CD27-specific Fab-arm and a non-binding Fab-arm, so that the antibody is monovalent for CD27. These bispecific antibodies were generated by controlled Fab-arm exchange between the CD27 antibodies and non-binding antibodies (as described in Labrijn A F et al., Nat Protoc. 2014 October; 9(10):2450-63).

To determine the affinity of the CD27 antibodies for human and mouse CD27, 100 nM recombinant His-tagged mouse or human CD27 protein (Sino Biological, Cat #10039-H08B1 [human], Cat #50110-M08H [mouse]) was loaded to pre-conditioned anti-Penta-HIS (HIS1K) biosensors (FortéBio, Cat #18-5120) for 600 sec.

To assess the affinity of the CD27 antibodies for cynomolgus monkey CD27, 5 μg/mL of recombinant cynomolgus monkey CD27-Fc fusion protein (R&D systems, Cat #9904-CD-100) was loaded to activated Amine Reactive 2nd Generation (AR2G) biosensors (FortéBio, Cat #18-5092).

After baseline measurements in Sample Diluent (FortéBio, Cat #18-1104) for 300 sec, the association (200 sec) and dissociation (1,000 sec) of CD27 antibodies was determined for antibody concentration series of 0.78-800 nM with two-fold dilution steps in Sample Diluent. An antibody molecular mass of 150 kDa was used for calculations. Reference sensors were incubated with Sample Diluent.

Data were acquired using Data Acquisition Software v11.1.1.19 (FortéBio) and analyzed with Data Analysis Software v9.0.0.14 (FortéBio). Data traces were corrected per antibody by subtraction of the reference sensor. The Y-axis was aligned to the last 10 sec of the baseline and Interstep Correction alignment to dissociation and Savitzky-Golay filtering were applied. Data traces were excluded from analysis when the response was <0.05 nm and calculated equilibrium was near to saturation (Req/Rmax>95% using a dissociation time of 50 sec). The data was fitted with the 1:1 model using a window of interest for the association set at 200 sec and dissociation time set at 50 sec. The dissociation time was chosen based on the coefficient of determination (R²), which is an estimate of the goodness of the curve fit (preferentially >0.98), visual inspection of the curve, and at least 5% signal decay during the association step.

Affinities for human CD27 could be accurately determined for three CD27 antibodies (IgG1-CD27-A, -B, -C) with K_{D} values in the nanomolar range (Table 2). For IgG1-CD27-D, and -E, BioLayer Interferometry experiments confirmed binding to human CD27 with affinities in a similar range, although suboptimal curve fitting did not allow calculation of accurate K_{D} values (as indicated in Table 2).

IgG1-CD27-A and -B also showed binding to recombinant cynomolgus monkey CD27, with K_{D} values in the same range as for human CD27. Results obtained with IgG1-CD27-C, -D and -E also confirmed binding to cynomolgus monkey CD27 with affinities in a similar range, although suboptimal curve fitting did not allow calculation of accurate K_{D} values (as indicated in Table 2).

Binding to recombinant mouse CD27 was only observed for antibody IgG1-CD27-C.

TABLE 2
Binding affinities of IgG1-CD27-A to -E antibodies
to CD27 from the indicated species.

| | Loading | | | |
|---|---|---|---|---|
| Sample | sample | K_{D} (M) | kₒₙ (1/Ms) | k_{dis} (1/s) |
| IgG1- | Human CD27-His | 1.3E−07 | 1.3E+05 | 1.8E−02 |
| CD27-A | Mouse CD27-His | n.b. | | |
| | Cyno CD27-Fc | 1.2E−07 | 1.7E+05 | 2.0E−02 |
| IgG1- | Human CD27-His | 5.4E−08 | 3.3E+05 | 1.8E−02 |
| CD27-B | Mouse CD27-His | n.b. | | |
| | Cyno CD27-Fc | 3.5E−08 | 6.5E+05 | 2.3E−02 |
| IgG1- | Human CD27-His | 7.0E−08 | 1.4E+05 | 9.8E−03 |
| CD27-C | Mouse CD27-His | 4.3E−07 | 5.0E+04 | 2.2E−02 |
| | Cyno CD27-Fc | 5.1E−08* | 1.2E+05* | 6.4E−03* |
| IgG1- | Human CD27-His | 4.5E−08* | 1.4+05* | 6.4E−03* |
| CD27-D | Mouse CD27-His | n.b. | n.b. | n.b. |
| | Cyno CD27-Fc | 2.1E−08* | 2.4E+05* | 5.0E−03* |
| IgG1- | Human CD27-His | 4.9E−08* | 1.3+05* | 6.4E−03* |
| CD27-E | Mouse CD27-His | n.b. | n.b. | n.b. |
| | Cyno CD27-Fc | 3.5E−08* | 1.6E+05* | 5.5E−03* |

*Binding was observed but KD, kₒₙ and k_{dis} are less reliable values due to suboptimal curve fitting, resulting in unreliable interpretation using the 1:1 model.
n.b.: no binding observed.

### Example 4: Binding of Anti-CD27 Antibodies to Cell Surface-Expressed Human and Cynomolgus Monkey CD27

Binding of anti-CD27 antibodies IgG1-CD27-A to -E* and prior art IgG1-CD27-131A* to cell surface-expressed human and cynomolgus monkey CD27 was analyzed by flow cytometry using transiently transfected HEK293F cells and primary T cells, which endogenously express CD27. Non-binding control antibody IgG1-b12-FEAR was used as negative control antibody.

FreeStyle 293-F suspension cells (HEK293F; ThermoFisher, Cat #R79007) were transiently transfected with mammalian expression vector pSB encoding full length human or cynomolgus monkey CD27 using 293fectin Transfection Reagent (ThermoFisher, Cat #12347019) according to the manufacturer's instructions.

Human and cynomolgus monkey PBMC were purified from buffy coats obtained from human healthy donors (Sanquin Blood Bank, the Netherlands) or from a cynomolgus monkey (BPRC, the Netherlands, Cat #S-1135) by low density gradient centrifugation using Lymphocyte Separation Medium (LSM; Corning, Cat #25-072CV) according to the manufacturer's instructions.

Cells were seeded in 96-wells plates (100,000 cells per well; Greiner Bio-one, Cat #650180) for sequential incubations, with washing steps in between with FACS buffer, consisting of PBS (Lonza, Cat #BE17-517Q)+1% BSA (Roche, Cat #10735086001)+0.02% Sodium Azide (Bio-World, Cat #41920044-3). The following incubations were applied: antibody concentration series (0.0001-10 μg/mL final concentration) for 30 min at 4° C.; live/dead marker FVS510 (BD, Cat #564406, diluted 1:1,000 in PBS) for 20 min at RT; PE-labeled polyclonal goat anti-human IgG (Jackson Immuno Research, Cat #109-116-098, diluted 1:500) for 30 min at 4° C.; and anti-CD3 antibody for T-cell identification (anti-human CD3: BD, Cat #555335, diluted 1:10; anti-cyno CD3: Miltenyi, Cat #130-091-998, diluted 1:10) for 30 min at 4° C. All samples were analyzed on a FACSCelesta flow cytometer (BD) and FlowJo software. Data were processed and visualized using GraphPad Prism.

All tested antibodies showed dose-dependent binding to human CD27, both on human T cells and transfected HEK293F cells (FIG. 2 A,B). Highest maximal binding was observed for IgG1-CD27-B and IgG1-CD27-C compared to intermediate binding for IgG1-CD27-A and IgG1-CD27-131A, and low binding for IgG1-CD27-D and IgG1-CD27-E, with the differences being most pronounced using human T cells. For binding to cynomolgus money CD27 T cells, highest binding was observed for IgG1-CD27-B, followed by Ig1-CD27-131A and IgG1-CD27-A. Lower binding was observed for IgG1-CD27-D and -E, whereas IgG1-CD27-C showed minimal binding to cynomolgus monkey T cells. All CD27 antibodies showed dose-dependent binding to HEK cells transfected with cynomolgus monkey CD27. Highest maximal binding was observed for IgG1-CD27-B and IgG1-CD27-131-A, somewhat lower binding was observed for IgG1-CD27-A, -D and -E. IgG1-CD27-C showed the lowest binding to HEK cells transfected with cynomolgus monkey CD27 (FIG. 2 C,D).

In conclusion, IgG1-CD27-A and IgG1-CD27-B showed dose-dependent binding to human and cynomolgus monkey CD27 expressed endogenously on human or cynomolgus monkey T cells, and transiently expressed in transfected HEK cells. IgG1-CD27-A and IgG-CD27-131A showed comparable binding to human T cells, whereas IgG1-CD27-B showed higher maximal binding.

**N.B. IgG1-CD27-A, -B, -C, -D and -E carried mutations F405L-L234F-L235E-D265A in the IgG Fc domain, which are functionally irrelevant in the context of this experiment. IgG1-CD27-131A carried a functionally irrelevant F405L mutation in the IgG1 Fc domain.

### Example 5: Binding of Anti-CD27 Antibodies to a Natural Human CD27-A59T Variant

Approximately 19% of the human population expresses a natural CD27 variant harboring an A59T mutation in the extracellular domain (SEQ ID NO. 2). Binding to human CD27-A59T was tested by flow cytometry for anti-CD27 antibodies IgG1-CD27-A, IgG1-CD27-B, IgG1-CD27-C* and benchmark IgG1-CD27-131A. Non-binding antibody IgG1-b12-FEAL was used as a negative control antibody. Transiently transfected HEK293F cells expressing human CD27-A59T (15,000 cells per well) were incubated with concentration series (0.0001-10 μg/mL using 10-fold dilution steps) of primary test antibodies IgG1-CD27-A to -C, non-binding control antibody IgG1-b12 (ctrl), and the prior art benchmark IgG-CD27-131A, which has been described previously to bind to CD27-A59T (WO2018/058022). After incubation, antibodies were PE-labeled with polyclonal goat anti-human IgG. Binding was analyzed on a FACSCelesta flow cytometer (BD) and FlowJo software. Data were processed and visualized using GraphPad Prism v.8.

The tested anti-CD27 antibodies IgG1-CD27-A, IgG1-CD27-B, IgG1-CD27-C, and IgG1-CD27-131A showed dose-dependent binding to CD27-A59T-transfected HEK293F cells with similar binding curves among the different antibodies (FIG. 3).

*N.B. IgG1-CD27-A, -B and -C carried mutations F405L-L234F-L235E-D265A in the IgG Fc domain, which are functionally irrelevant in the context of this experiment. IgG1-CD27-131A carried a functionally irrelevant F405L mutation in the IgG1 Fc domain.

### Example 6: Induction of Human T Cell Proliferation by Anti-CD27 Antibodies

As enhanced IgG hexamerization through Fc-Fc interactions upon introduction of the E345R or E430G mutation enhanced CD27 agonist activity of anti CD27 antibodies (Example 2), the capacity of IgG1-CD27-A, IgG1-CD27-B, and IgG1-CD27-C antibody variants carrying the E430G or E345R mutations to increase proliferation of TCR activated T cells was tested in vitro.

Additionally, Fc mutations that were reported to reduce binding to C1q and FcγR (G237A or P329R) or that enhance binding to C1q (K326A/E333A double mutation) were introduced to test their potential effect on CD27 agonist activity of CD27 antibodies carrying the E345R or E430G mutations. The K326A/E333A double mutation was previously shown to enhance C1q binding and to contribute to enhanced agonistic activity of DR5-specific humanized IgG1 antibodies comprising an Fc-Fc interaction enhancing mutation (WO2018/146317A1). The mutations G237A, P329R, or K326A/E333A were introduced, in addition to E430G or E345R, to IgG1-CD27-A, IgG1-CD27-B and IgG1-C (Table 3) and their effect on T-cell proliferation was determined using human PBMCs obtained from healthy donors (Sanquin Blood Bank, the Netherlands).

TABLE 3
Mutations in the Fc domain of antibodies IgG1-CD27-A,
IgG1-CD27-B, or IgG1-CD27-C and their biological effect

| | Fc mutation | | | | |
|---|---|---|---|---|---|
| | E430G | E345R | P329R | G237A | K326/E333A |
| | Described effect | | | | |
| | | | Reduced | Reduced | Enhanced |
| | Enhanced | Enhanced | C1q/FcγR | C1q/FcγR | C1q |
| Antibody* | hexamerization | hexamerization | binding | binding | binding |
| IgG1-CD27-X-E430G | + | | | | |
| IgG1-CD27-X-P329R- | + | | + | | |
| E430G | | | | | |
| IgG1-CD27-X-G237A- | + | | | + | |
| E430G | | | | | |
| IgG1-CD27-X-K326A- | + | | | | + |
| E333A-E430G | | | | | |
| IgG1-CD27-X-E345R | | + | | | |
| IgG1-CD27-X-P329R- | | + | + | | |
| E345R | | | | | |
| IgG1-CD27-X-G237A- | | + | | + | |
| E345R | | | | | |
| IgG1-CD27-X-K326A- | | + | | | + |
| E333A-E345R | | | | | |

*X in IgG1-CD27-X, refers to IgG1-CD27 clones IgG1-CD27-A, IgG1-CD27-B, or IgG1-CD27-C.

PBMCs were resuspended in PBS at a density of 5×10⁶ cells/mL and labeled with CFSE using CellTrace CFSE Cell Proliferation Kit (Invitrogen, Cat #C34564; 1:10,000), according to the manufacturer's instructions. CFSE-labeled PBMCs (100,000 cells/well) were incubated in 96-well round-bottom plates (Greiner Bio-one, Cat #650180) with 0.1 μg/mL anti-CD3 antibody clone UCHT1 (Stemcell Technologies, Cat #60011) to activate T cells, and CD27 antibodies (1 μg/mL final concentration) in T-cell Activation Medium (ATCC, Cat #80528190) supplemented with 5% Normal Human Serum (NHS; Sanquin, Product #B0625) for 96 h at 37° C./5% C02. For identification of viable cells in CD4⁺ and CD8⁺ T-cell subsets by flow cytometry, cells were sequentially incubated with live/dead marker FVS510 (1:1,000) for 20 min at RT and a staining mix for lymphocyte markers, containing APC-eFluor780-labeled anti-human CD4 antibody (Invitrogen, Cat #47-0048-42, 1:50), AlexaFluor700-labeled anti-human CD8a antibody (BioLegend, Cat #301028; 1:100), PE-Cy7-labeled mouse anti-human CD14 antibody (BD Biosciences, Cat #557742; 1:50) and BV785-labeled anti-human CD19 antibody (BioLegend, Cat #363028; 1:50) for 30 min at 4° C. in the dark. Samples were measured on a FACSCelesta (BD Biosciences) flow cytometer and CFSE dilution peaks in the viable CD4⁺ and CD8⁺ T-cell subsets (FVS510⁻CD14⁻CD19⁻CD4⁺ and FVS510⁻CD14⁻CD19⁻CD8⁺) were analyzed using FlowJo 10 software as a readout for T-cell proliferation. T-cell proliferation was expressed as the percentage of proliferated cells or the division index both calculated by using the FlowJo software (version 10). Percentage of proliferated (divided) cells was determined by gating for the cells that have gone through CFSE dilution (CFSE^{low peaks}) The division index is the average number of divisions that the cells underwent. Heatmaps were generated using GraphPad Prism version 8. Proliferation assays were performed using PBMCs from four different healthy donors.

Variants of IgG1-CD27-A, -B and -C carrying an E430G or E345R mutation induced a small increase in proliferation of CD8⁺ T cells compared to control antibody in two out of the four donors tested. The introduction of additional mutations (P329R, G237A or K326A/E333A) into IgG1-CD27-A, -B or -C variants carrying an E430G mutation showed variable effects on CD8⁺ T cell proliferation across the four PBMC donors. In contrast, introduction of the P329R mutation into IgG1-CD27-A and IgG1-CD27-C variants carrying an E345R mutation consistently increased their capacity to enhance proliferation of activated CD8⁺ T cells. This particularly applied to IgG1-CD27-A: whereas the measured CD8⁺ T cell proliferation was comparable for IgG-CD27-A-E345R, IgG1-CD27-B-E345R and IgG1-CD27-C-E345R in each of the donors, introduction of an additional P329R mutation consistently led to a higher increase in CD8⁺ T cell proliferation for clone IgG1-CD27-A-E345R compared to IgG1-CD27-B-E345R or IgG1-CD27-C-E345R. Thus, the effect of the E345R mutation in combination with the P329R mutation on proliferation of TCR activated CD8⁺ T cells was consistently larger for clone IgG1-CD27-A than for IgG1-CD27-B and IgG1-CD27-C. Across all antibody variants tested, IgG1-CD27-A-E345R-P329R induced the largest increase in CD8⁺ T cell proliferation in all donors (FIG. 4A).

The addition of the mutations G237A or K326A-E333A into CD27 antibody variants carrying the E345R mutation did not or only minimally increase the proliferation of CD8⁺ T cells in any of the clones tested, as compared to antibodies comprising the single mutations E345R (FIG. 4A).

Also in CD4⁺ T cells, the highest and most consistent increase in T cell proliferation was observed in presence of IgG1-CD27-A-E345R-P329R (FIG. 4B). Whereas CD4⁺ T cell proliferation was generally comparable between IgG1-CD27-A, -B and -C variants carrying only the E430G or E345R mutations, introduction of an additional P329R mutation led to a larger increase in CD4⁺ T cell proliferation for the IgG1-CD27-A variant carrying the E345R variant compared to IgG1-CD27-A-E430G or IgG1-CD27-B or -C variants carrying either the E430G or the E345R mutation. This effect was observed in three out of four donors tested. In donor 1, the effect of additional mutations in addition to E430G or E345R on CD4⁺ T cell proliferation was generally small, and effects observed in this donor were not reproduced in the other three donors.

The combination of the E345R with the P329R mutations also consistently increased CD4⁺ T cell proliferation for IgG1-CD27-C, although the difference between the E345R mutation alone and the combination of E345R and P329R was smaller for clone IgG1-CD27-C than for clone-A. For clone IgG1-CD27-B, a modest increase in CD4⁺ T cell proliferation was observed for IgG1-CD27-B-E345R-P329R compared to IgG1-CD27-B-E345R in two out of the four donors.

Introduction of the P329R, G327A or K326A/E333A mutations into IgG1-CD27-A, -B, or -C variants carrying the E430G mutation did not, or not consistently, induce effects on CD4⁺ T cell proliferation. Similarly, no or inconsistent effects were observed after introduction of the G327A or K326A/E333A in IgG1-CD27-A, -B or -C variants carrying the E345R mutation.

In summary, IgG1-CD27-A-E345R-P329R consistently induced the highest increase in proliferation of activated CD8⁺ and CD4⁺ T cells, demonstrating that IgG1-CD27-A-E345R-P329R induces most efficient CD27 agonism. DR5-specific, hexamerization-enhanced antibodies with the P329R mutation previously showed reduced capacity to induce DR5 agonism compared to DR5-specific hexamerization-enhanced antibodies without the P329R mutation (Overdijk et al, Mol Canc Ther 2020). It was thus considered surprising that introduction of the P329R mutation in addition to the E345R mutation in IgG1-CD27-A enhanced CD27 agonist activity. Moreover, it is not known why the combined effect of the E345R+P329R mutations was consistently larger for IgG1-CD27-A than for IgG1-CD27-B or IgG1-CD27-C.

### Example 7: Induction of Human T-Cell Proliferation by Anti-CD27 Antibody IgG1-CD27-A-P329R-E345R

The capacity of IgG1-CD27-A-P329R-E345R to increase proliferation of TCR stimulated human CD4⁺ and CD8⁺ T-cells was analyzed in CSFE dilution assays using human healthy donor PBMCs, and compared to prior art anti-CD27 clones IgG1-CD27-131A*, IgG1-CD27-CDX1127, and IgG1-CD27-BMS986215*. The T-cell proliferation assays were performed as described in Example 6, with minor deviations (75,000 cells/well; concentration range 0.002-10 μg/mL). Samples using T cells without anti-CD3 stimulation were included to test potential CD27 agonist activity of the antibodies in absence of T-cell receptor activation (FIGS. 5A and 5B). Such activity is unwanted as it would pose a safety risk if the antibody was able to induce proliferation of resting T cells.

Percentage of proliferated T cells (FIG. 5A, B, C, D) was calculated as the percentage of cells with reduced CFSE fluorescence, indicating cell divisions using FlowJo software. Expansion index (FIGS. 5E and 5F) identifies the fold increase of cells in the wells and was calculated using the Proliferation Modeling tool in FlowJo version 10. Manual adjustments to the peaks were made where necessary to define the number of the peaks present more consistently.

None of the CD27 antibodies of the invention and the prior art antibodies tested here induced proliferation of unstimulated T cells (i.e. in absence of CD3 crosslinking (FIGS. 5A and B).

Most of the CD27 antibodies induced some proliferation of activated CD4⁺ and CD8⁺ T-cells at the highest antibody concentrations tested (FIGS. 5C and D). Based on this, an expansion index was calculated (FIGS. 5E and F). Antibody IgG1-CD27-A-P329R-E345R of the invention more profoundly enhanced proliferation of CD4⁺ and CD8⁺ T cells in vitro compared to the prior art anti-CD27 clones IgG1-CD27-131A, IgG1-CD27-CDX1127 and IgG1-CD27-BMS986215.

*For IgG1-CD27-131A and IgG1-CD27-BMS986215, variants carrying a F405L mutation, that is functionally irrelevant in the context of this experiments, were used.

### Example 8: C1q Binding to Membrane-Bound CD27 Antibodies

The P329R mutation was previously described to reduce interaction of IgG1 antibodies with C1q and FcγR (Overdijk et al, Molecular Cancer Therapeutics 2020). The effect of the P329R mutation on C1q binding of IgG1-CD27-A comprising the E345R mutation was tested in cellular C1q binding assays in vitro using human healthy donor T cells. Anti-HIV gp120 antibody IgG1-b12-F405L was used as non-binding isotype control antibody (ctrl). T cells were enriched from human healthy donor PBMCs using RosetteSep Human T cell Enrichment cocktail (Stemcell, Cat #15061) and resuspended in culture medium (RPMI 1640 [Gibco, Cat #A10491-01] supplemented with 0.1% BSA and 1% Pen/Strep [Lonza, Cat #DE17-603E]). T cells (2×10⁶ cells/well) were pre-incubated in polystyrene 96-well round-bottom plates with antibody dilution series (8× five-fold dilution starting at 15 μg/mL final assay concentration) for 15 min at 37° C. to allow the antibodies to bind to the T cells. Then, cells were cooled on ice, supplemented with NHS as a source of human C1q (20% NHS final assay concentration) and incubated on ice for 45 min. Cells were subsequently incubated on ice with FITC-labeled Rabbit anti-human C1q antibody (DAKO, Cat #F0254; 20 μg/mL) for 30 min and resuspended in FACS buffer with TO-PRO-3 (ThermoFisher, Cat #T3605; 1:5,000 dilution). C1q binding was determined by flow cytometry measuring the FITC signal on live cells.

Membrane bound WT IgG1-CD27-A antibody did not show C1q binding (FIG. 6). Introduction of the hexamerization-enhancing mutation E430G or E345R (IgG1-CD27-A-E430G and IgG1-CD27-A-E345R) resulted in binding of C1q to CD27 antibodies on the T-cell surface, in line with the increased binding avidity of the hexameric C1q protein to hexameric antibody ring structures on the cell surface (FIG. 6). Introduction of the P329R mutation in IgG1-CD27-A-E345R (IgG1-CD27-A-P329R-E345R) resulted in loss of C1q binding (FIG. 6), demonstrating that IgG1-CD27-A-P329R-E345R was unable to bind C1q.

These data show that IgG1-CD27-A-P329R-E345R is unable to bind C1q upon binding to CD27 on the cell surface of T cells. This indicates that C1q binding does not contribute to antibody-induced CD27 agonist activity of IgG1-CD27-A-P329R-E345R. This is in contrast to what was previously described for other hexamerization-enhanced agonistic antibodies. Moreover, lack of C1q binding indicates that IgG1-CD27-A-P329R-E345R is unable to activate the classical pathway of complement activation. Thus, IgG1-CD27-A-P329R-E345R is not expected to induce complement activation and CDC on T cells which activity would be unwanted.

### Example 9: Binding of Anti-CD27 Antibodies to Human Fc Receptors

Binding of IgG1-CD27-A-P329R-E345R to human FcγR variants was analyzed using a Biacore surface plasmon resonance (SPR) system and compared to an anti-HIV gp120 antibody IgG1-b12 (ctrl). Biacore Series S Sensor Chips CM5 (Cytiva, Cat #29104988) were covalently coated with anti-His antibody using amine-coupling and His capture kits (Cytiva, Cat #BR100050 and Cat #29234602) according to the manufacturer's instructions. Next, 125 nM Fcγ-receptor FcγRIa, FcγRIIa (167-His [H] and 167-Arg [R]), FcγRIIb or FcγRIIIa (176-Phe [F] and 176-Val [V]) (Sino Biological, Cat #10256-H08S-B, Cat #10374-H27H, Cat #10374-H27H1-B, Cat #10259-H27H-B, Cat #10389-H27H-B and Cat #10389-H27H1-B) in HBS-P+ (Cytiva, Cat #BR100827) were captured onto the surface. After three cycles of buffer, antibody samples were injected for 36 cycles to generate binding curves using antibody ranges of 0-3,000 nM for FcγRI and 0-10,000 nM for the other FcγRs. Each sample that was analyzed on an FcR-coated surface (Active Surface) was also analyzed on a parallel flow-cell without FcR (Reference Surface), which was used for background correction. Dissociation from the anti-His-coated surface was performed by regeneration of the surface using 10 mM Glycine-HCl pH 1.5 (Cytiva, Cat #BR100354). Sensograms were generated using Biacore Insight Evaluation software (Cytiva) and a four-parameter logistic (4PL) fit was applied to calculate relative binding of IgG1-CD27-A-P329R-E345R against the reference sample (ctrl).

Binding of IgG1-CD27-A-P329R-E345R to high affinity receptor FcγRIa was strongly reduced compared to the ctrl antibody, although some binding was observed at higher antibody concentrations (FIG. 7A). IgG1-CD27-A-P329R-E345R did not bind to the human low affinity receptors FcγRIIa (FIGS. 7B and C), FcγRIIb (FIG. 7D) and FcγRIIIa (FIGS. 7E and F).

In conclusion, IgG1-CD27A-P329R-E345R shows minimal (FcγRIa) or no (FcγRIIa, FcγRIIb, and FcγRIIIa) binding to human IgG Fc receptors.

### Example 10: Binding of Anti-CD27 Antibody IgG1-CD27-A-E345R-P329R to Human T Cells

Binding of IgG1-CD27-A-P329R-E345R to CD27 on human healthy donor T cells was characterized in more detail using flow cytometry. Anti-HIV gp120 antibody variant IgG1-b12-P329R-E345R was used as non-binding control antibody (ctrl). Human PBMCs were isolated from buffy coats obtained from human healthy donors. PBMCs (1×10⁵ cells/well) in FACS buffer were added to polystyrene 96-well round-bottom plates (Greiner bio-one, Cat #650101) and pelleted by centrifugation at 300×g for 3 min at 4° C. The cells were resuspended in 50 μL/well serial antibody dilutions in FACS buffer (range 0.0015 to 10 μg/mL in 3-fold dilution steps) and incubated for 30 min at 4° C. Cells were pelleted, washed twice with FACS buffer and incubated in 50 μL/well with FITC-conjugated secondary antibody (FITC AffiniPure F(ab′)₂ fragment goat anti-human IgG, F(ab′)₂ fragment specific, Jackson ImmunoResearch, Cat #109-096-097, diluted 1:100) for 30 min at 4° C. in the dark. Cells were pelleted again, washed twice with FACS buffer and incubated for 30 min at 4° C. in the dark in 50 μL/well of a staining mix for lymphocyte markers, containing BV711-labeled anti-human CD19 antibody (BioLegend, Cat #302246, 1:50), AlexaFluor700-labeled anti-human CD8a antibody (BioLegend, Cat #301028, 1:100), APC-eFluor780-labeled anti-human CD4 antibody (Invitrogen, Cat #47-0048-42, 1:50), PE-CF594-labeled mouse anti-human CD56 antibody (BD Biosciences, Cat #564849, 1:100), PE-Cy7-labeled mouse anti-human CD14 antibody (BD Biosciences, Cat #557742, 1:50) and eFluor450-labeled anti-human CD3 antibody (Invitrogen, Cat #48-0037-42, 1:200). Cells were pelleted again, washed twice using FACS buffer, and resuspended in 80 μL FACS buffer containing death cell marker 7-Amino-Actinomycin D (7-AAD; BD Biosciences, Cat #51-68981E, 1:240 diluted). The samples were measured by flow cytometry on an LSRFortessa (BD) flow cytometer and analyzed using FlowJo software. Binding curves were analyzed using non-linear regression (sigmoidal dose-response with variable slope) using GraphPad Prism 8 software.

Anti-CD27 antibody IgG1-CD27-A-P329R-E345R showed dose-dependent binding to healthy donor T cells, with similar binding characteristics for CD4⁺ and CD8⁺ T cells (FIG. 8).

### Example 11: FcγR-Independent Induction of CD27 Cell Signaling by Anti-CD27 Antibody IgG1-CD27-A-P329R-E345R

A CD27-specific monoclonal antibody that can induce CD27 signaling independent of secondary FcγR-mediated cross-linking may be immunostimulatory in the absence of FcγR-positive cells, which would be an advantage in tumors where the frequency of FcγR-bearing cells is low.

CD27 agonist activity of IgG1-CD27-A-P329R-E345R was tested in the presence or absence of FcγR-bearing cells and compared to the corresponding WT antibody IgG1-CD27-A and prior art antibodies IgG1-CD27-131A*, IgG1-CD27-CDX1127, and IgG1-CD27-BMS986215*. Non-binding antibody IgG1-b12-P329R-E345R was used as a negative control (ctrl). CD27 reporter assays were performed, essentially as described in Example 2, with the exception that in the current example, Thaw-and-Use GloResponse NFκB-luc2/CD27 Jurkat cells were cultured in the presence of human FcγRIIb-expressing cells that can facilitate FcγR-mediated crosslinking of membrane-bound antibodies.

Thaw-and-Use effector FcγRIIb CHO-K1 cells (Promega, Cat #JA2251) were plated in 96-well flat bottom culture plates (PerkinElmer, Cat #0815), undiluted or at three increasing dilutions (1/3, 1/9. 1/27) and incubated overnight at 37° C./5% C02. Supernatants of the adherent FcγRIIb-expressing cells was replaced by a Thaw-and-Use NFκB-luc2/CD27 Jurkat cell suspension of a fixed cell concentration in Bio-Glo Luciferase Assay Buffer (starting at a NFκB-luc2/CD27 Jurkat:FcγRIIb CHO-K1 ratio of 1:1 for undiluted FcγRIIb CHO-K1 cells), containing serial dilutions of antibody (final concentration range 0.0002-10 μg/mL). After 6 h incubation at 37° C./5% CO₂, plates were equilibrated to RT and bioluminescence was measured and presented as RLU as described in Example 2.

IgG1-CD27-A-P329R-E345R induced dose-dependent CD27 activation, which was independent of FcγRIIb-expressing cells (FIG. 9A). In contrast, the corresponding WT antibody IgG1-CD27-A, without the E345R hexamerization-enhancing mutation and the P329R mutation, only showed CD27 agonism in the presence of FcγRIIb-expressing cells (FIG. 9A-E). Similarly, CD27 activation by the prior art antibodies IgG1-CD27-131A, IgG1-CD27-CDX1127 and IgG1-CD27-BMS986215 was also dependent on the presence of FcγRIIb-expressing cells and decreased gradually with decreasing NFκB-luc2/CD27 Jurkat:FcγRIIb CHO-K1 ratios (FIG. 9 F-J).

In conclusion, these data indicate that IgG1-CD27-A-P329R-E345R can induce CD27 agonism independent of secondary FcγR-mediated cross-linking. This is in contrast to prior art anti-CD27 antibodies that were dependent on the presence of FcγR-bearing cells to induce CD27 agonism.

*For IgG1-CD27-131A and IgG1-CD27-BMS986215, variants carrying a F405L mutation, that is functionally irrelevant in the context of this experiment, were used.

### Example 12: Pharmacokinetic (PK) Analysis of Anti-CD27 Antibody IgG1-CD27-A-P329R-E345R in Absence of Target Binding, Studied in Mice

The pharmacokinetic characteristics of anti-CD27 antibody IgG1-CD27-A-P329R-E345R*, in absence of target binding, was analyzed in mice and compared to the corresponding WT antibody IgG1-CD27-A*. IgG1-CD27-A does not bind to mouse CD27 (Example 3, Table 2), and thus the experiment was designed to test pharmacokinetic behaviour of IgG1-CD27-A and IgG1-CD27-A-P329R-E345R in vivo, in absence of target binding. The study was carried out by Crown Bioscience (China) by qualified personnel, in accordance with the approved IACUC protocol and Crown Bioscience, Inc. Standard Operating Procedures. 11-12 weeks old female SCID mice (C.B-17, Vital River Laboratory Animal Technology Co., Ltd. (VR, Beijing, China; 3 mice per group) were injected intravenously with 500 μg antibody (25 mg/kg) in a 200 μL injection volume. 40 μL blood samples were collected at 10 min, 4 h, 1 d, 2 d, 7 d, 14 d and 21 d after antibody administration, plasma was collected from blood samples and stored at −80° C. until determination of total human IgG concentrations by ELISA. 96-well ELISA plates (Greiner, Cat #655092) were coated overnight at 4° C. with 2 μg/mL anti-human IgG (Sanquin, The Netherlands, Article #M9105, Lot #8000260395) and subsequently blocked for 1 h with PBSA (PBS supplemented with 0.2% bovine serum albumin [BSA, Roche, Cat #10735086001]). Next, with washing steps in between, the anti-human IgG-coated plates were sequentially incubated on a plate shaker for 1 h at RT with the plasma samples that were serially diluted in ELISA Buffer (PBSA supplemented with 0.05% Tween 20 [Sigma-Aldrich, Cat #P1379]), for 1 h at RT with polyclonal peroxidase-conjugated goat anti-human IgG secondary antibody (Jackson, Cat #109-035-098), and finally with 2,2′-azino-bis(3-ethylbenzthiazoline-6-sulfonic acid) (ABTS; Roche, Cat #11112422001). The reaction was stopped by adding 2% Oxalic Acid (Riedel de Haen, Cat #33506). Dilution series of the respective materials used for injection were used to generate reference curves. Absorbance was measured in an EL808 Microtiter plate reader (BioSPX) at 405 nm and total human IgG concentrations (in μg/mL) were plotted.

There was no substantial difference between the PK profile of IgG1-CD27-A-P329R-E345R and the counterpart WT antibody IgG1-CD27-A (FIG. 10), as determined by measuring plasma IgG levels at different timepoints after intravenous injection in mice.

Although a steeper decline in the initial (distribution) phase was observed for IgG1-CD27-A-P329R-E345R and its WT counterpart (IgG1-CD27-A) compared to predictions for human IgG1 in mice, the terminal elimination of both antibodies was in line with predictions rates for human wild-type IgG1 based on a 2-compartment model (Bleeker W K, Teeling J L, Hack C E. Blood. 2001 Nov. 15; 98(10):3136-42).

Together, this demonstrates that introduction of the P329R and E345R mutations did not affect the pharmacokinetics properties of IgG1-CD27-A in absence of target binding.

N.B. the experiment described in this example used variants of IgG1-CD27-A and IgG1-CD27-A-P329R-E345R carrying a F405L mutation, which is functionally irrelevant in the context of this experiment.

### Example 13: Induction of Antibody-Dependent Cellular Phagocytosis by Anti-CD27 Antibody IgG1-CD27-A-P329R-E345R

Antibody-dependent cellular cytotoxicity (ADCC) is mediated primarily through FcγRIIIa expressed on NK cells, whereas antibody-dependent cellular phagocytosis (ADCP) can be mediated by monocytes, macrophages, neutrophils, and dendritic cells via FcγRI, FcγRIIa, and FcγRIII (Hayes, J. M et al 2016). To understand the effect of residual binding of anti-CD27 antibody IgG1-CD27-A-P329R-E345R to FcγRIa (Example 9) on effector functions of FcγRIa-expressing immune cells, the capacity of IgG1-CD27-A-P329R-E345R to induce ADCP was analyzed in vitro using CTV-labeled CD27⁺ Burkitt's lymphoma Daudi cells as target cells, and human monocyte-derived macrophages (hMDM) as effector cells (E:T=2:1). hMDMs were isolated from PBMCs by positive selection using CD14 MicroBeads (Miltenyi Biotec, cat. no. 130-050-201), according to the manufacturer's instruction. PBMCs were centrifuged (1,200 RPM, 5 min, RT) and resuspended in ice-cold monocyte isolation buffer (PBS, 0.5% BSA, 2 mM EDTA) at a density of 1.25×10⁷ PBMCs/mL. 20 μL CD14 MicroBeads were added per 80 μL of PBMC suspension and incubated with agitation at 4° C. for 15 min on a rollerbank. 30 mL of ice-cold monocyte isolation buffer was added, PBMC/CD14 MicroBeads mixtures centrifuged (300×g, 10 min, 4° C.) and resuspended in 6 mL ice-cold monocyte isolation buffer. LS columns (Miltenyi Biotec, cat. no. 130-042-401) were rinsed with 3 mL ice-cold monocyte isolation buffer and each column loaded with 3 mL PBMC/CD14 MicroBeads mixtures. After flow through of the CD14⁻ cells and three washes of the column in ice-cold monocyte isolation buffer, CD14⁺ monocytes were recovered in 3 mL of ice-cold monocyte isolation buffer by using a plunger. The CD14⁺ cells were counted on a Cellometer Auto 2000 Cell Viability Counter (Nexcelom Bioscience) using ViaStain™ Viability Dye acridine orange/propidium iodide (AOPI; Nexcelom Bioscience, cat. no. CS2-0106), and resuspended at a density of 0.8×10⁶ cells/mL in Celgene® GMP DC medium (CellGenix, cat. no. 20801-0500) supplemented with macrophage colony-stimulating factor (M-CSF; Gibco, cat. no. PH9501; 50 ng/mL final concentration) and 3 mL of monocyte suspension (i.e., 2.4×10⁶ monocytes) in 100 mm² Nunc™ dishes with UpCell™ Surface, which allows cell harvesting by leaving plates at RT (Thermo Fisher Scientific, cat. no. 174902). After three days of incubation, 2 mL of fresh medium containing 5×M-CSF was added to the plates. After incubation for seven days (37° C., 5% CO₂), macrophages were detached from the surface by leaving plates at RT for 1 to 1.5 h. Detached macrophages were pelleted by centrifugation, counted using AOPI, and resuspended at a density of 1×10⁶ cells/mL in culture medium (RPMI 1640 with 10% DBSI).

Human Burkitt's lymphoma Daudi cells (ATCC® CCL-213™) were labeled using the CellTrace™ Violet Cell Proliferation Kit (Thermo Fisher Scientific, cat. no. C34557), according to the manufacturer's instructions. Briefly, Cell Trace Violet (CTV) was added to a final concentration of 0.2 μM to 1×10⁶ Daudi cells/mL in PBS and incubated in the dark at 37° C. for 20 min (15 mL incubation volume). 10 mL DBSI was added to inactivate unbound dye. Cells were pelleted by centrifugation (300×g, 5 min), washed in PBS, and counted with AOPI. CTV-labeled Daudi cells were resuspended at a density of 0.5×10⁶ cells/mL in culture medium.

For the ADCP assay, hMDM (50,000 cells/well) and CTV-labeled Daudi cells (25,000 cells/well) were seeded together (E:T=2:1) on ice in 96-well plates in a final volume of 150 μL culture medium and incubated with anti-CD27 antibody IgG1-CD27-A-P329R-E345R or anti-CD20 antibody IgG1-CD20 (0.000001 to 10 μg/mL concentration range in 10-fold dilutions), for 4 h (37° C., 5% C02). After incubation, 100 μL Human BD Fc Block™ (BD Biosciences, cat. no. 564220; 1:100 in FACS buffer) was added and incubated at 4° C. for 10 min. Cells were pelleted by centrifugation (300×g, 5 min), resuspended in FACS buffer containing PE-Cy7 conjugated antihuman CD11b antibody (BioLegend, cat. no. 301322; 1:80) and TO-PRO-3 (Thermo Fisher Scientific, cat. no. T3605; 1:25,000) and incubated at 4° C. for 30 min. Cells were washed, resuspended in FACS buffer and collected and analyzed on a FACSymphony™ A3 Cell Analyzer (BD Biosciences). Data were analyzed using FlowJo software to measure viable target cell numbers and phagocytic hMDM and processed and visualized using GraphPad Prism software.

The percentage of viable Daudi cells for each condition was calculated according to the following formula:

$$\% viable Daudi cells = ( \frac{\% TO - PRO - {3}^{-} CD 11 {b}^{-} {CTV}^{+} cells incubated with test antibody}{\% TO - PRO - {3}^{-} CD 11 {b}^{-} {CTV}^{+} cells incubated without antibody} ) \times 100$$

The quantity of phagocytic hMDM for each condition was determined as % TO-PRO-3⁻CD11b⁺CTV⁺ cells.

IgG1-CD27-A-P329R-E345R did not increase the percentage of phagocytic hMDM or reduce the percentage of viable Daudi cells in the phagocytosis assay, using hMDM from four different human healthy donors. This demonstrates that residual FcγRIa binding did not result in FcγRIa-mediated effector functions for IgG1-CD27-A-P329R-E345R (data from representative human healthy donor shown in FIG. 11). The positive control antibody IgG1-CD20 efficiently induced phagocytosis of Daudi cells, that express high levels of CD20, as demonstrated by an increase in the percentage of phagocytic hMDM and a decrease in the percentage of viable Daudi cells.

In conclusion, residual binding to FcγRIa was not sufficient to induce IgG1-CD27-A-P329R-E345R-dependent ADCP of CD27⁺ cells.

### Example 14: Fluid-Phase, Target-Independent, Complement Activation by Anti-CD27 Antibody IgG1-CD27-A-P329R-E345R as Determined by Measurement of C4d

Deposition Fc-Fc interaction-enhanced antibodies generally exist as monomeric IgG1 molecules in solution, and hexamerize on the cell surface upon target binding to form a C1q docking place in case of an active Fc region (Diebolder, C. A et al 2014; de Jong, R. N et al, 2016). The IgG Fc domain of anti-CD27 antibody IgG1-CD27-A-P329R-E345R is silenced by introduction of the P329R mutation, which results in lack of C1q binding to membrane-bound IgG1-CD27-A-P329R-E345R (FIG. 6). To confirm that IgG1-CD27-A-P329R-E345R is unable to activate complement in solution in the absence of target binding, fluid phase, target-independent, complement activation was investigated by determination of C4d deposition, which is considered a measure for activation of the classical complement pathway. Fluid phase C4d fragment deposition by IgG1-CD27-A-P329R-E345R was analyzed by an enzyme-linked immunosorbent assay (ELISA) using the MicroVue™ C4d Enzyme Immunoassay (EIA; Quidel, cat. no. A008) and was performed according to the manufacturer's protocol. Heat Aggregated Gamma Globulin (HAGG; Complement Activator; Quidel, cat. no. A114) was used as a positive control for the assay. IgG1-b12 and IgG1-b12-RGY (WO2014006217A1)) were included as control antibodies. Introduction of E345R/E430G/S440Y (RGY) Fc mutations in an IgG1 antibody has been described to induce the formation of hexamers in solution, resulting in fluid phase complement activation (Diebolder, C. A et al, 2014; Wang, G., R. N et al, 2016; de Jong, R. N et al, 2016). IgG1-b12-P329R-E345R was included as isotype control antibody.

Antibody dilutions were prepared in phosphate-buffered saline (PBS) to a concentration of 1 mg/mL, except for HAGG, which was diluted to a concentration of 10 mg/mL. Then, the test samples were further diluted to a concentration of 100 μg/mL (for monoclonal IgG) or 1,000 μg/mL (for HAGG) in 90% (final concentration) normal human serum (NHS) (CompTech, Lot. no. 42a) and incubated at 37° C. for 1 h. In parallel, ‘No antibody’ samples (no antibody, 90% NHS) and ‘PBS only’ samples (no antibody, no NHS) were included as negative controls. Next, the samples were diluted 1:250 in cold kit-provided Complement Specimen Diluent. In the meantime, the strips coated with mouse anti-human C4d antibody were placed in a 96-wells plate and the assay wells were washed three times with 250 to 300 μL Wash Buffer with a 1-min waiting step after the first wash. The test samples were added to the wells (100 μL/well) and as a negative control, Complement Specimen Diluent only (blank) was used in the ELISA. In parallel, 100 μL of the standards (Standard A-E) and internal controls provided by the kit were added to separate wells. The plates were incubated for 30 min at RT. Then, the plates were washed five times with Wash Buffer as described above. 50 μL of C4d Conjugate (peroxidase-conjugated goat anti-human C4d) was added to the wells and the plates were incubated for 30 min at RT. After five washing steps with Wash Buffer as described above, 100 μL of C4d Substrate [0.7% 2-2′-Azino-di-(3-ethylbenzthiazoline sulfonic acid diammonium salt] was added and again the plates were incubated for 30 min at RT. Finally, 50 μL kit-provided Stop Solution was added and within 1 h, the optical density was measured at 405 nm using an ELISA Plate Reader (EL808 BioSPX, BioTek).

IgG1-CD27-A-P329R-E345R and the control antibody IgG1-b12-P329R-E345R (having the same Fc backbone as IgG1-CD27-A-P329R-E345R) did not induce fluid phase C4d deposition at the tested concentration of 100 μg/mL; the measured C4d levels were similar to background levels of the control antibody with a wild-type Fc domain (IgG1-b12) and the no antibody control (FIG. 12). In contrast, the positive control antibody IgG1-b12-RGY, that is known to form hexamers in solution, induced C4d deposition to the same level as HAGG.

These data show that IgG1-CD27-A-P329R-E345R did not induce target-independent, fluid phase complement activation in vitro.

### Example 15: Capacity of Anti-CD27 Antibody IgG1-CD27-A-P329R-E345R to Compete for Ligand-Binding with CD70

To determine if anti-CD27 antibody IgG1-CD27-A-P329R-E345R interferes with the interaction of CD27 with its natural ligand CD70, binding of a saturating concentration of biotinylated recombinant human CD70 extracellular domain (ECD) to CD27, endogenously expressed on human Burkitt's lymphoma cell line Daudi, was studied in the presence and absence of excess IgG1-CD27-A-P329R-E345R.

Daudi cells (ATCC® CCL-213™) cultured in RPMI 1640 medium (Gibco, cat. no. A10491-01) supplemented with 10% donor bovine serum with iron (DBSI; Gibco, cat. no. 20731-030) were seeded at 50,000 cells/well in round bottom 96-well plates (Greiner Bio One, cat. no. 650261). Cells were pelleted by centrifugation (300×g, 3 min at 4° C.) and resuspended in FACS buffer (PBS, 1% BSA [Roche, cat. no. 1073508600]) containing anti-CD27 or control antibodies (50 μg/mL final concentration). Biotinylated recombinant human CD70 ECD (Abcam, cat. no. ab271443) was added at a saturating concentration (6 μg/mL) and cells were incubated at 4° C. for 30 min.

Cells were washed twice and resuspendend in FACS buffer containing Brilliant Violet (BV) 421™ labeled streptavidin (BioLegend, cat. no. 405225; 0.0025 μg/mL final concentration) and R phycoerythrin (PE) labeled polyclonal AffiniPure F(ab′)2 fragment goat-anti-human IgG Fc (Jackson ImmunoResearch, cat. no. 109 116098; 0.0025 μg/mL final concentration) at 4° C. for 30 min. Cells were washed twice, resuspended in FACS buffer containing TO-PRO-3 iodide (Thermo Fisher Scientific, cat. no. T3605; 1:25,000) and analyzed. Data were collected on a BD FACSymphony™ A3 flow cytometer (BD Biosciences) and analyzed using FlowJo software. For compensation, one drop of UltraComp eBeads™ Compensation Beads (Life Technologies, cat. no. 01-2222-42) was added to each well. 2 μL of each antibody was added and mix was incubated for 20 min. Plates were spun down and beads were resuspended in FACS buffer and measured. For viability compensation, cells were treated at 65° C. for 10 min and mixed 1:1 with viable cells. Cells were spun down and resuspended in TO-PRO-3 diluted in FACS buffer. Data were processed and visualized using GraphPad Prism.

IgG1-CD27-A-P329R-E345R or IgG1-CD27-A did not block binding of the CD70 ECD to CD27⁺ Daudi cells, as CD70 binding levels were comparable to those for Daudi cells incubated with the nonbinding isotype control antibodies IgG1-b12-P329R-E345R or IgG1-b12, or cells without antibody (FIG. 13). Also, prior art anti-CD27 antibodies IgG1-CD27-BMS986215 and IgG1-CD27-131A showed a weak blocking effect on CD27 binding to CD70 ECD. In contrast, CD70 was unable to bind to surface CD27 on Daudi cells in presence of prior art anti-CD27 antibody IgG1-CD27-CDX1127 (FIG. 13) that was previously reported to block ligand-binding (Vitale et al, 2012).

In conclusion, IgG1-CD27-A-P329R-E345R binding does not block CD27 binding by its natural ligand CD70 on Daudi cells.

### Example 16: T-Cell Activation Marker Expression Upon Incubation of Polyclonally Stimulated Human PBMCs with Anti-CD27 Antibodies

The effect of IgG1-CD27-A-P329R-E345R on expression of T-cell activation markers in polyclonally activated T cells was studied using PBMCs obtained from three different healthy human donors. Expression of HLA-DR, CD25, CD107a, and 4-1BB were analyzed after incubating PBMCs with IgG1-CD27-A-P329R-E345R or prior art anti-CD27 antibodies for two and five days.

Freshly isolated 75,000 PBMCs/well were seeded in 96-well U bottom plates (Greiner Bio-One) in cell culture medium. Duplicate wells were incubated simultaneously with anti-CD3 antibody (UCHT1 clone; Stemcell; 0.1 μg/mL); and IgG1-CD27-A-P329R-E345R (0.0005 to 30 μg/mL in threefold dilutions); or prior art anti-CD27 antibodies IgG1-CD27-CDX1127, IgG1-CD27-131A, and IgG1-CD27-BMS986215 (30 μg/mL); or nonbinding control antibody IgG1-b12-P329R-E345R (10 μg/mL). To determine expression of each activation marker in absence of treatment, duplicate control wells with untreated (no anti-CD3 or anti-CD27 antibodies) cells were supplemented with culture medium alone. To set the gates for identifying activation marker positive cells, fluorescence minus one (FMO) controls were used. For the FMO controls, all the antibodies used in the experiment except for one corresponding to an activation marker in duplicate wells was added to 75,000 PBMCss/well from one donor activated with anti-CD3 antibody. Untreated cells from each donor in single wells with no staining antibody were included as negative controls. To detect viable cells, untreated cells from each donor were stained with 4′,6-diamidino-2-phenylindole (DAPI) alone in single wells.

After incubation for two or five days (37° C., 5% CO₂), plates were washed once with FACS buffer and resuspended in an antibody mixture in FACS buffer containing antibodies for T-cell activation markers 4-1BB, CD25, CD107a, human leukocyte antigen (HLA)-DR; and antibodies for gating CD4⁺ and CD8⁺ T-cell subsets in flow cytometry. After incubation at 4° C. for 30 min, all plates were washed twice with FACS buffer and cells were resuspended in FACS buffer. The samples were analyzed on a BD LSRFortessa Cell Analyzer using FlowJo software to determine the median fluorescence intensity (MFI) and percentage of positive cells for each T-cell activation marker on CD4⁺ and CD8⁺ T cells. Anti-CD27 antibody induced changes in the expression levels of the T-cell activation markers were presented as the fold change in MFI of the anti-CD27 antibody sample relative to the nonbinding control antibody IgG1-b12-P329R-E345R. The samples were analyzed on a BD LSRFortessa™ Cell Analyzer (BD Biosciences) using FlowJo software.

IgG1-CD27-A-P329R-E345R increased expression of CD25, CD107a and 4-1BB on activated CD4⁺ T cells (FIG. 14A). These effects were more pronounced after 2 days of incubation than after 5 days of incubation. On CD8⁺ T cells, incubation with IgG1-CD27-A-P329R-E345R resulted in an increased expression of HLA-DR, CD107a and 4-1BB both after 2 and 5 days of incubation (FIG. 14B).

The expression of T-cell activation markers was also assessed upon incubation for 2 and 5 days with three prior art antibodies. IgG1-CD27-131A and IgG1-CD27-BMS986215 induced a comparable increase in expression of HLA-DR, 4-1BB, CD25, and CD107a on CD4⁺ and CD8⁺ T cells, while the effect of incubation for 2 or 5 days with IgG1-CD27-CDX1127 on T-cell activation marker expression was less pronounced.

In conclusion, incubation of polyclonally activated PBMCs with IgG1-CD27-A-P329R-E345R resulted in an increased expression of activation markers HLA-DR, CD25, CD107a and 4-1BB on CD4⁺ and CD8⁺ T cells.

### Example 17: Percentages of OVA-Specific CD8⁺ T Cells in OVA Protein-Immunized Mice after Injection of Anti-CD27 Antibodies in a Human CD27-KI Mouse Model

The effect of IgG1-CD27-A-P329R-E345R treatment on expansion of antigen-specific T cells in the hCD27 KI OVA model in splenocytes was analyzed by flow cytometry.

Homozygous human CD27 (hCD27)-KI mice on a C57BL/6 background (hCD27 KI mice) were obtained from Beijing Biocytogen Co., Ltd. (strain name C57BL/6-Cd27tm1(CD27)/Bcgen, Stock no. 110006). This strain was developed in collaboration with the HuGEMM™ platform of Crown Bioscience, featuring a humanized drug target (CD27 in this case) within mice with a functional immune system. In hCD27 KI mice, exons 1-5 of the mouse CD27 gene encoding the extracellular domain were replaced by human CD27 exons 1-5. OVA-specific T cells were induced in vivo by subcutaneous (s.c.) injection of the immunogen ovalbumin (OVA) in hCD27-KI mice and the agonist effect of IgG1-CD27-A-P329R-E345R was tested by simultaneously treating the mice intravenously (i.v.) with the antibody.

On day 0, the mice were injected s.c. with 5 mg OVA (InvivoGen, cat. no. vac-pova-100, lot. no. EFP-42-04) and treated by i.v. injection into the tail vain with IgG1-CD27-A-P329R-E345R (30 mg/kg), IgG1-CD27-CDX1127 (30 mg/kg) or IgG1-b12-P329R-E345R (30 mg/kg). On day 12 and day 21, mice were boosted with OVA and treated with antibody as on day 0. On day 10, day 19 and day 24, blood was collected via cheek pouch or saphena in BD Microtainer® blood collection tubes containing di-potassium ethylenediaminetetraacetic acid (K2-EDTA; BD, cat. no. 365974) and immediately used in further analysis. On day 28, mice were euthanized and spleens were resected under sterile conditions.

Resected spleen tissue in RPMI1640 medium (Thermo Fisher Scientific, cat. no. C22400500BT) was transferred to gentleMACs™ C Tubes (Miltenyi Biotec, cat. no. 130-093-237) and mechanically dissociated to a single cell suspension using the gentleMACS™ Dissociator (Miltenyi, cat. no. 130-093-235), according to the manufacturer's instructions. After dissociation, the cell suspension was filtered through a 70 μm cell strainer (Falcon, cat. no. 352350). Next, samples were washed twice by resuspension in 3 mL wash buffer (sterile PBS [Hyclone, SH0256.01B] supplemented with 4% FBS [Gibco, cat. no. 10099 141]). Cells were counted on a Cellometer Auto T4 (Nexcelom Bioscience) and the number of cells was adjusted to 2×10⁶ splenocytes per tube.

2×10⁶ splenocytes were transferred to FACS tubes (Falcon, cat. no. 352052) and resuspended in wash buffer (sterile PBS [Hyclone, SH0256.01B] supplemented with 4% FBS [Gibco, cat. no. 10099 141]) supplemented with 1 μg/mL purified rat anti-mouse CD16/CD32 (Mouse BD Fc Block™, BD Biosciences, cat. no. 553141). After a preincubation at 2-8° C. for 10 min in the dark, 10 μL PE-labeled OVA-tetramer (MBL Life science, cat. no. TS 5001 1C) was added, and the samples were gently vortexed before further incubating at 2-8° C. for 30-60 min in the dark. Without washing, labeled antibodies and compounds used for flow cytometry gating of T-cell subsets were added. The samples were gently vortexed and incubated at 2-8° C. for an additional 30 min in the dark. Next, samples were washed twice by resuspension in 2 mL wash buffer and centrifuged at 300×g for 5 min. Finally, the cells were resuspended in 250 μL wash buffer and analyzed on a BD LSRFortessa™ X-20 Cell Analyzer (BD Biosciences). Data were processed using Kaluza Analysis Software (Beckman Coulter).

IgG1-CD27-A-P329R-E345R increased the percentages of OVA-specific CD8⁺ T cells in the spleen of mice simultaneously injected with OVA protein vaccination. The percentages of OVA-specific CD8⁺ T cells in mice treated with 30 mg/kg IgG1-CD27-CDX1127 were lower than the IgG1-CD27-A-P329R-E345R-treated group and comparable to the IgG1-b12-P329R-E345R-treated group (FIG. 15). Similar observations were made in peripheral blood samples.

### Example 18: IFNγ Secretion by OVA-Specific CD8⁺ T Cells from Spleens of OVA-Immunized Mice Injected with Anti-CD27 Antibodies

Resected spleen tissue in RPMI1640 medium (see Example 17) was gently mashed over a 70 μm cell strainer (Falcon, cat. no. 352350), pelleted by centrifugation (1,500 rpm, 5 min), and resuspended in 10 mL Ammonium-Chloride-Potassium (ACK) Lysing Buffer (Invitrogen, cat. no. A1049201). After 3-5 min incubation at RT, samples were washed twice with 10-20 mL PBS and resuspended in 5 mL Cellular Technology Limited (CTL) Test™ Medium (ImmunoSpot, cat. no. CTLT-005) supplemented with 50 U/mL penicillin and 50 μg/mL streptomycin (pen/strep, Gibco, cat. no. 15070-063). The collected splenocytes were filtered again through a 70 μm cell strainer and counted on a Vi-CELL™ XR Cell Viability Analyzer (Beckman Coulter) to adjust the concentration to 3.125×10⁶ cells/mL with CTL-Test Medium containing pen/strep.

IFNγ production by splenocytes was analyzed using the Mouse IFN-γ ELISpotPLUS kit (Mabtech, cat. no. 3321-4HPW-2), essentially as described by the manufacturer. Pre-coated MultiScreenHTS IP Filter (MSIP) white plates (mAb AN18) were washed four times with 200 μL sterile PBS per well and conditioned with 200 μL CTL-Test Medium containing pen/strep (RT, 30 min). Medium was removed and 5×10⁵ splenocytes/well were incubated in duplicate with 2 μg/mL OVA257-264 peptide SIINFEKL (Invivogen, cat. no. vac-sin), or scrambled control peptide FILKSINE (SB-PEPTIDE, cat. no. SB073-1MG) in a total volume of 180 μL/well for 20 h in a humidified incubator (37° C., 5% C02). As a positive control for IFNγ production, splenocytes were incubated in parallel with a cell stimulation cocktail consisting of 500 ng/mL phorbol myristate acetate (PMA) and 10 μg/mL ionomycin (PMA+Ionomycin, Dakewe Biotech, cat. no. DKW ST PI). Cultures of splenocytes without peptide were included as a negative control. After incubation, the cells were removed and the plates were washed five times with PBS. Next, plates were sequentially incubated, with five wash steps with PBS in between, with Biotinylated detection mAb (R4-6A2; RT, 2 h), Streptavidin-horseradish peroxidase (HRP; RT, 1 h), and finally 3,3′,5,5′-tetramethylbenzidine (TMB) substrate solution (all provided by the kit). When distinct spots emerged, the reaction was stopped by washing extensively in deionized water. Spots were counted on an AID iSpot ELISpot Reader (Autoimmun Diagnostika [AID] GMBH, ELR08IFL) using spotAID V8 software (AID). ELISpot data were analyzed and presented in bar diagrams using GraphPad Prism software and presented as the mean number of spots per well±SEM from all mice per treatment group (n=5).

Splenocytes from all IgG1-CD27-A-P329R-E345R-treated animal groups showed increased IFNγ production in response to treatment with OVA peptide, as demonstrated by ELISpot analysis (FIG. 16). Stimulation of the splenocytes with a scrambled control peptide induced no or minimal IFNγ production, suggesting that IFNγ was produced by OVA-specific T cells. In contrast, no IFNγ production was observed in splenocytes from mice treated with 30 mg/kg IgG1-CD27-CDX1127.

### Example 19: Effect of IgG1-CD27-A-P329R-E345R Treatment on T-Cell Activation in OVA-Immunized Mice In Vivo

The effect of IgG1-CD27-A-P329R-E345R treatment on CD8⁺ T-cell activation was studied in vivo by analyzing the expression of PD-1 on CD8⁺ T cells derived from OVA-treated hCD27-KI mice. Mice were treated as described in Example 17. Also, methods to obtain and analyze splenocytes by FACS are described in Example 17.

IgG1-CD27-A-P329R-E345R induced an increase in the percentage of CD8⁺ T cells expressing activation marker PD-1 on day 28. CD8⁺PD-1⁺ T-cell percentages were low in animals treated with IgG1-CD27-CDX1127 or control antibody IgG1-b12-P329R-E345R (FIG. 17).

### Example 20: Effect of IgG1-CD27-A-P329R-E345R Treatment on In Vivo Induction of T-Cell Subsets in OVA-Immunized Mice

The effect of IgG1-CD27-A-P329R-E345R on the expansion of T-cell subsets was studied by analyzing the expression of CD44 and CD62L in splenocyte samples from OVA-treated hCD27-KI mice. Memory CD8⁺ T cells derived from spleens of IgG1-CD27-A-P329R-E345R-treated, OVA-immunized, hCD27-KI mice were quantified by flow cytometry. Memory T cells were classified as effector memory (CD44⁺CD62L⁻) and pre-effector T cells (CD44⁻CD62L⁻; Nakajima, Y., K et al 2018). Mice were treated as described in Example 17. Also, methods to obtain and analyze splenocytes by FACS are described in Example 17.

IgG1-CD27-A-P329R-E345R (30 mg/kg) induced increased percentages of pre-effector T cells and effector memory CD8⁺ T cells in the spleen on day 28 when compared to splenocytes of mice treated with IgG1-b12-P329R-E345R (FIG. 18). Within the CD45⁺ population, IgG1-CD27-A-P329R-E345R induced higher percentages of pre-effector T cells and effector memory T cells than IgG1-CD27-CDX1127 (30 mg/kg), while comparable mean percentages of these T-cell populations were induced by both anti-CD27 antibodies in the CD8⁺ fraction of splenocytes.

### Example 21: Effect of IgG1-CD27-A-P329R-E345R Treatment on In Vivo Expansion of T Cells in OVA-Immunized Mice

The effect of IgG1-CD27-A-P329R-E345R on expansion of T cells was studied by analyzing the expression of CD3 in splenocyte and blood samples from OVA-treated hCD27-KI mice. Mice were treated as described in Example 17. Also, methods to obtain and analyze splenocytes and blood samples by flow cytometry are described in Example 17.

Treatment of OVA-immunized hCD27-KI mice with 30 mg/kg IgG1-CD27-A-P329R-E345R did not increase the percentage of CD3⁺ T cells in the spleen, compared to treatment with the non-binding control antibody IgG1-b12-P329R-E345R (FIG. 19). In contrast, treatment with benchmark antibody IgG1-CD27-CDX1127 (30 mg/kg) resulted in a decrease of CD3⁺ T cells in the spleen. Similar observations were made in peripheral blood samples.

### Example 22: Effect of IgG1-CD27-A-P329R-E345R on T-Cell Cytokine Production in Antigen-Specific Studies

The capacity of IgG1-CD27-A-P329R-E345R to increase cytokine production was studied using T cells that had been stimulated by their cognate antigen. PBMC were isolated from buffy coats obtained from healthy human donors by Ficoll-Paque density gradient separation (GE Healthcare, cat. no. 17 1440 03) according to the manufacturer's instructions.

Human magnetic CD14 and CD8 MicroBeads (Miltenyi Biotec, cat. no. 130 050 201 and 130 045 201, respectively) were used for positive selection of CD14⁺ monocytes and negative selection of CD14⁻ PBL from human PBMC, and positive selection of CD8⁺ T cells from frozen PBL. Cell suspensions were centrifuged and resuspended in magnetic-activated cell sorting (MACS) buffer (Dulbecco's phosphate-buffered saline [DPBS] with 5 mM EDTA and 1% human albumin) at 1×10⁷ live cells per 80 μL MACS buffer. Per 1×10⁷ cells, 12 μL CD14 or CD8 MicroBeads were added. Subsequent MACS separation was performed using an automated magnetic cell separation instrument or by manual separation. Automated MACS separation was performed using an autoMACS® Pro Separator (Miltenyi Biotec), according to the manufacturer's instructions. Eluted CD14⁺ monocytes and CD8⁺ T cells were centrifuged (8 min, 300×g at RT) resuspended in X-VIVO 15 medium (Lonza), and counted with erythrosine B solution for further use; i.e., monocyte differentiation into iDC or electroporation of CD8⁺ T cells with PD-1 and/or CLDN6-specific T-cell receptor (TCR) mRNA.

For the generation of monocyte-derived iDC, up to 40×10⁶ PBMC-derived CD14⁺ monocytes were cultured (37° C., 5% CO₂) for five days in T175 flasks in DC medium (RPMI 1640, 5% pooled human serum [PHS; One Lambda, cat. no. A25761], 1× minimum essential medium non-essential amino acid solution [MEM NEAA, Life Technologies, cat. no. 11140 035], 1 mM sodium pyruvate [Life Technologies, cat. no. 11360 039]) supplemented with 100 ng/mL human granulocyte/macrophage colony-stimulating factor (GM-CSF; Miltenyi Biotec, cat. no. 130-093-868) and 50 ng/mL human IL-4 (Miltenyi Biotec, cat. no. 130093 924). After three days in culture, half of the medium per flask was replaced. Since the medium taken from the flask contained non-adherent monocytes, it was centrifuged (8 min, 300×g, RT), the supernatant discarded, the cell pellet resuspended in fresh DC medium and then returned into the originator flask together with 200 ng/mL GM-CSF and 200 ng/mL IL-4 (final concentration). After the five days of incubation, the iDC which adhered to the culture flask were detached using 10 mL DPBS containing 2 mM EDTA (37° C., 10 min). The isolated iDC were washed, pelleted (8 min, 300×g at RT) and used for electroporation with CLDN6 mRNA.

Human CD8⁺ T cells were electroporated with RNA encoding the alpha and beta chains of a mouse TCR specific for human CLDN6, either alone or together with RNA encoding PD-1, and human monocyte-derived iDC were electroporated with RNA encoding human CLDN6. Up to 5×10⁶ iDC or 15×10⁶ CD8⁺ T cells were electroporated in 250 μL X-VIVO 15 medium at RT using an ECM 830 Square Wave Electroporation System (BTX®). Cells were mixed with RNA, pulsed (500 V, 3 ms for T cells or 300 V, 12 ms for iDC), and immediately diluted with 750 μL pre-warmed assay medium (IMDM GlutaMAX [Life technologies, cat. no. 31980030] with 5% PHS). Electroporated iDC were transferred to 6- or 12-well plates and cultured O/N (37° C., 5% C02). After O/N incubation, electroporated CD8⁺ T cells and iDC were evaluated by flow cytometry to evaluate cell purity, expression of transfected RNA (PD-1 and CLDN6-TCR on CD8⁺ T cells and CLDN6 on iDC), and baseline expression of CD27 and PD-1 on CD8⁺ T cells and PD-L1 on iDC. Approximately 78% to 93%, 78% to 92%, and 36% to 98% of electroporated CD8⁺ T cells expressed CLDN6-TCR, PD-1, and endogenous CD27, respectively. Approximately 47% to 91% and 94% to 99% of electroporated iDC expressed CLDN6 and endogenous PD-L1, respectively (not shown).

CD8⁺ T cells and iDC were seeded at a 10:1 ratio (7.5×10⁴ T cells and 7.5×10³ iDC per well) in a 96-well round-bottom plate. IgG1-CD27-A-P329R-E345R was diluted in assay medium and 25 μL of diluted IgG1-CD27-A-P329R-E345R was added to the wells, to reach a final concentration of 10 μg/mL. Similarly, the control antibodies IgG1-CD27-131A and IgG1-b12-P329R-E345R were added to reach final concentrations of 10 μg/mL. Antigen-specific T-cell activity upon antibody treatment was analyzed in vitro by measuring cytokines in the supernatant of T cells transduced to express CLDN6-TCR, which were co-cultured with iDC transduced to express and present CLDN6. Supernatants were collected after two days, and concentrations of multiple proinflammatory cytokines and chemokines were determined by multiplex electrochemiluminescence assays (ECLIA) using the 10-spot U-PLEX ImmunoOncology Group 1 (human) kit (MSD; cat. no. K151AEL 2) following the manufacturer's instructions.

For the 10-spot U-PLEX Immuno-Oncology Group 1 kit, biotinylated capture antibodies were pre-incubated at RT with the assigned linkers, which have a biotin-binding domain, for 30 min, followed by 30 min incubation with Stop Solution. Plates were coated with a mix of the linker coupled capture antibodies by incubating at RT with shaking for 1 hr. Plates were washed three times with 1×MSD Wash Buffer. Supernatant samples or kit standards were diluted 1:2 in Assay Diluent, added to the wells and incubated at RT for 2 h with constant shaking. The plates were washed three times with Wash Buffer, and incubated with SULFO-TAG-conjugated detection antibodies from the kit at RT for 1 h with constant shaking. The plates were washed three times with Wash Buffer before adding Read Buffer B to catalyze the electrochemiluminescent reaction. The plates were immediately analyzed by measuring light intensity on a MESO QuickPlex SQ 120 imager (MSD).

IgG1-CD27-A-P329R-E345R-induced changes in cytokine production were assessed by multiplex ECLIA in supernatants from the CD8⁺ T cell/iDC co-cultures after two days of incubation (n=4 different donors). IgG1-CD27-A-P329R-E345R induced a significant increase in the production of GM-CSF and IFNγ in CD8⁺ T cell/iDC co-cultures with CD8⁺ T cells expressing endogenous levels of PD-1 (FIG. 20A), while also an increase in IL-13 and TNFα production was observed. A considerable increase for the same cytokines was observed in cultures containing PD-1-overexpressing T cells (FIG. 20B). While cytokine levels were generally decreased when T cells overexpressed PD-1, the relative increase (fold increase) in cytokine production in presence of IgG1-CD27-A-P329R-E345R was generally higher this setting (FIGS. 20A and B). In contrast, prior art anti-CD27 antibody IgG1-CD27-131A showed minimal effect on cytokine production compared to the nonbinding control antibody IgG1-b12-P329R-E345R (FIGS. 20A and B).

### Example 23: Expression of Cytotoxicity-Associated Molecules by Antigen-Specific CD8⁺ T Cells Incubated with IgG1-CD27-A-P329R-E345R

The induction of T-cell mediated cytotoxicity upon antibody treatment was studied by analyzing the expression of cytotoxicity-associated molecules on the antigen-specific T cells by flow cytometry in co-cultures of human healthy donor T cells transduced to express a CLDN6-TCR and MDA-MB-231_hCLDN6 target cells.

MDA-MB-231_hCLDN6 cells were generated by lentiviral transduction. To this end, 2×10⁵ MDA-MB-231 cells in 250 μL Dulbecco's modified eagle medium (DMEM, Thermo Fisher Scientific, cat. no. 31966-047) supplemented with 10% FBS (non-heat-inactivated) were seeded per well in a 12-well tissue culture plate. The cells were incubated for 1-2 h at 37° C. (7.5% CO₂). Supernatants containing lentiviral vectors encoding human CLDN6 (pL64b42E(EF1a-hClaudin6)Hygro-T2A-GFP) were thawed on ice and diluted in a total volume of 750 μL DMEM/10% FBS to obtain titers of 2×10⁵, 8×10⁴, and 3.2×10⁴ TU/mL. These titers corresponded to MOI of 1, 0.4, and 0.16, respectively. The supernatants were then added to the MDA-MB-231 cells, and the cells were incubated for 72 h at 37° C. (5% CO₂) without disturbance. For the experiments described in the current Example, MDA-MB-231-hCLDN6 cells were cultured in DMEM/10% FBS. Cells were passaged or harvested for experiments at 70% to 90% confluence. Cells were detached by treatment with Accutase (Thermo Fisher Scientific, cat. no. A11105010) for 5 min (37° C., 7.5% CO₂), and resuspended by addition of culture medium. Cells were centrifuged (300×g, 4 min at RT) and counted. MDA-MB-231_hCLDN6 cells were not cultured for more than 20 passages.

MDA-MB-231_hCLDN6 cells were seeded at 1.2 to 1.5×10⁴ cells/well, in 96-well flat-bottom plates (for flow cytometry analysis) and xCELLigence E-plates (Agilent, cat. no. 05232368001; for impedance measurement) and allowed to settle at RT for 30 min. Next, plates were incubated for one day in the incubator and the xCELLigence real-time cell analysis (RTCA) instrument (ACEA Biosciences), respectively (37° C., 5% CO₂).

Isolated CD8⁺ T cells (see Example 22) were electroporated with CLDN6-specific TCR mRNA and incubated O/N. After CD8⁺ T-cell isolation and electroporation, T-cell cultures contained 49% to 99% CD8⁺ T cells. Of these electroporated CD8⁺ T cells, approximately 78% to 93% expressed CLDN6-TCR and 59% to 98% of CLDN6-TCR⁺ CD8⁺ cells were CD27⁺. Cells were centrifuged (8 min, 300×g at RT), resuspended in DMEM/10% FBS and counted. The cells were centrifuged again, resuspended at 3×10⁶ cells/mL in DMEM/10% FBS, and added to the wells containing the previously seeded MDA-MB-231_hCLDN6 cells (1.5×10⁵ CD8⁺ T cells/well; T cell:tumor cell, effector:target, ratio of 10:1). IgG1-CD27-A-P329R-E345R, IgG1-CD27-131A, and the nonbinding control antibody IgG1-b12-P329R-E345R were added to the co-cultures at 10 μg/mL. CD107a and GzmB expression were determined by flow cytometry.

After two days of incubation in the presence of 10 μg/mL IgG1-CD27-A-P329R-E345R, the percentage of GzmB⁺CD107a⁺CD8⁺ T cells was significantly enhanced compared to treatment with the nonbinding control antibody or prior art anti-CD27 antibody IgG1-CD27-131A (FIG. 21).

In conclusion, these data show that IgG1-CD27-A-P329R-E345R was able to induce cytotoxicity-associated molecules on activated antigen-specific T cells.

### Example 24: Capacity of IgG1-CD27-A-P329R-E345R to Induce T-Cell Mediated Tumor Cytotoxicity

To evaluate T-cell mediated cytotoxicity, CLDN6-TCR-electroporated CD8⁺ T cells were co-cultured with MDA-MB-231_hCLDN6 cells in the presence of IgG1-CD27-A-P329R-E345R, prior art anti-CD27 antibody IgG1-CD27-131A, or nonbinding control antibody IgG1-b12-P329R-E345R for five days in an xCELLigence real-time cell analysis instrument (Acea Biosciences), with impedance measurements at two-hour intervals, as described in Example 23. Cell index values were derived from impedance measurements conducted at two-hour intervals. Area-under-the-curve (AUC) were obtained from cell index data over five days of co-culture. AUC were normalized to co-cultures treated with IgG1-b12-P329R-E345R. The magnitude of impedance is dependent on cell number, cell morphology, and cell size and on the strength of cell attachment to the plate, which altogether is used in this particular case as an indirect readout of tumor cell mass. Decrease in impedance in this experimental setting is considered a surrogate of tumor-cell killing by CD8⁺ T cells. It should be noted that impedance may underestimate tumor cell killing due to proliferation of T cells.

IgG1-CD27-A-P329R-E345R induced a decrease in cell index, indicative of tumor-cell killing. IgG1-CD27-131A did not have a visible effect on cell index, indicating minimal capacity to increase tumor-cell killing (FIG. 22).

### Example 25: Capacity of IgG1-CD27-A-P329R-E345R to Induce Expansion of Tumor-Infiltrating Lymphocytes

The capacity of IgG1-CD27-A-P329R-E345R to induce expansion of tumor-infiltrating lymphocyte (TIL) subsets (CD4⁺ and CD8⁺ T cells, NK cells, and regulatory T cells [Treg]) was evaluated ex vivo using cryopreserved tumors that had been surgically resected from NSCLC patients.

Surgically resected human NSCLC tissues were received in transport medium (HypoThermosol® FRS Preservation Solution [BioLife Solutions, cat. no. 101104], 7.5 μg/mL Amphotericin B [Thermo Fisher Scientific, cat. no. 15290026], and 300 units/mL (U/mL) pen/strep [Thermo Fisher Scientific, cat. no. 15140-122]). Samples were washed three times in wash medium (5 mL X-VIVO 15 [Lonza], 2.5 μg/mL Amphotericin B, [Thermo Fisher Scientific] and 100 U/mL pen/strep [Thermo Fisher Scientific]) and transferred to a cell culture dish. Fatty tissue and necrotic areas were removed with a scalpel, and the tissue was cut into fragments of approximately 5 mm³. Each fragment was placed in an individual cryovial, and 1 mL freezing medium (FBS, 10% DMSO) was added to each vial. The vials were transferred into a controlled freeze-chamber (Mr. Frosty freezing container), which was placed in a −80° C. freezer. After at least 16 h at −80° C., the vials were transferred to liquid nitrogen for long-term storage.

4 to 6 cryopreserved vials containing tumor fragments of approximately 5 mm³ from one tumor specimen were thawed per experiment in a 37° C. water bath for approximately 2 min and washed five times with wash medium and transferred to a cell culture dish. The tumor fragments were further dissected with a scalpel into fragments of approximately 1 mm³. Most of the fragments were used for TIL expansion upon culturing with IL-2 and treatment antibody and remaining fragments were used to determine expression of specific cell surface markers at baseline, without any treatment.

Two tumor fragments per well (on average) were seeded in 24-well plates (2 mL/well total volume capacity used in assay) in 0.1 mL prewarmed TIL cultivation medium (X-VIVO 15 [Lonza] with 2% human serum albumin [HSA; CSL Behring, cat. no. PZN-00504775], 100 U/mL pen/strep [Thermo Fisher Scientific], and 2.5 μg/mL Amphotericin B [Thermo Fisher Scientific]) containing 45 to 50 U/mL IL-2 (Proleukin S; Novartis Pharma, cat. no. PZN-02238131). IgG1-CD27-A-P329R-E345R was diluted in TIL cultivation medium containing 45 to 50 U/mL IL-2 and 900 μL of this dilution was added to the wells as appropriate. Final IgG1-CD27-A-P329R-E345R concentrations in the wells were 1 or 10 μg/mL. As a control, medium containing 45 to 50 U/mL IL-2 without antibodies was added to tumor fragments in separate wells. A total of 8 to 16 wells were incubated for each experimental condition per donor (37° C., 5% CO₂).

After three days of culture, fresh TIL cultivation medium containing 45 to 50 U/mL IL-2 and IgG1-CD27-A-P329R-E345R was added to the wells (1 mL/well, same antibody concentrations as above). Between day 5 and 14/17 after assay initiation, the cultures were regularly monitored with a microscope for proliferation of TIL that migrated from the tissue fragments and the formation of TIL microclusters. If >25 TIL microclusters were observed in one well after seven or eight culture days, cells and tissue fragments from two identically treated original wells were resuspended and pooled into one well of a 6-well plate (5 to 6 mL/well total volume capacity used in assay) with the culture medium and fresh IL 2 containing TIL cultivation medium was added (estimated 33 U/mL IL-2 final concentration).

Every two to three days, cultures were supplemented with fresh IL-2-containing TIL cultivation medium. IL-2 concentrations in the medium added to cultures were reduced to 10 U/mL, or first reduced to 25 U/mL and then to 10 U/mL thereafter after supplementing the wells with medium throughout the assay. On day 14 or 17, the cells were harvested for flow cytometry analysis.

IgG1-CD27-A-P329R-E345R enhanced expansion of TIL subtypes compared to control cultures treated with IL-2 alone, with the largest relative increase in cell count observed for CD8⁺ T cells and Tregs, followed by CD4⁺ T cells, and NK cells. For all TIL subsets, expansion was more pronounced with IgG1-CD27-A-P329R-E345R at 1 μg/mL than 10 μg/mL (Table 4 and FIG. 23).

TABLE 4
Fold-expansion of IgG1-CD27-A-P329R-E345R-treated TIL
Tumor tissues derived from human NSCLC specimens were cultured
with low-dose IL-2 in the presence or absence of IgG1-CD27-A-P329R-E345R.
Absolute cell counts of the indicated cell subsets were determined
by flow cytometry after 14 to 17 days of treatment. Fold differences
in cell numbers for IgG1-CD27-A-P329R-E345R-treated cultures relative
to cultures treated with IL-2 are shown. Data shown are from five
tumor tissues from individual patients tested in five independent
experiments. P = 0.0236, 1 μg/mL vs. 10 μg/mL IgG1-CD27-A-P329R-E345R
(two-way ANOVA).

| IgG1-CD27- | | | | | | | | | | |
|---|---|---|---|---|---|---|---|---|---|---|
| A-P329R- | | | | | | | | | | |
| E345R | Cell population | | | | | | | | | |
| concentration | All TIL | | CD4⁺ T cells | | CD8⁺ T cells | | Treg | | NK cells | |
| (μg/mL) | 1 | 10 | 1 | 10 | 1 | 10 | 1 | 10 | 1 | 10 |
| Patient #578 | 14.9 | 2.1 | 19.3 | 2.3 | 19.6 | 2.9 | 86.9 | 2.1 | 11.3 | 1.1 |
| Patient #507 | 27.9 | 5.1 | 33.7 | 4.4 | 107.1 | 17.4 | 32.2 | 11.9 | 14.4 | 6.0 |
| Patient #594 | 0.6 | 1.5 | 0.4 | 1.0 | 1.8 | 2.2 | 0.4 | 2.3 | 0.8 | 2.6 |
| Patient #592 | 0.9 | 0.8 | 0.4 | 0.2 | 2.9 | 1.7 | 4.8 | 2.6 | 2.3 | 1.2 |
| Patient #561 | 0.8 | 1.6 | 0.2 | 2.9 | 0.8 | 1.0 | n.d. | n.d. | 1.1 | 1.2 |
| Average ± SD ^{a} | 11.1 ± 11.3 | 2.4 ± 1.6 | 13.5 ± 14.0 | 2.0 ± 1.6 | 32.9 ± 43.4 | 6.1 ± 6.6 | 31.1 ± 34.5 | 4.9 ± 4.1 | 7.2 ± 5.8 | 2.7 ± 2.0 |

^{a} Average and SD calculations exclude patient #561 for better comparability between cell populations.
Abbreviations: ANOVA = analysis of variance; n.d. = not determined; NK = natural killer; NSCLC = non-small cell lung cancer; SD = standard deviation; TIL = tumor-infiltrating lymphocyte; Treg = regulatory T cell.

### Example 26: BRET Analysis to Assess Intermolecular Interactions of IgG1-CD27-A-P329R-E345R Molecules on the Cell Surface

The capacity of CD27 antibodies harboring the hexamerization-enhancing mutation (E345R) to increase intermolecular Fc-Fc interactions after binding to CD27 on the cell surface was determined using bioluminescence resonance energy transfer (BRET) analysis. This molecular proximity-based assay detects protein interactions by measuring energy transfer from a bioluminescent protein donor to a fluorescent protein acceptor. Energy transfer occurs only when the donor and acceptor are in close proximity (<10 nm [Wu and Brand, 1994; Dacres et al, 2012]).

First, cell surface expression of CD27, as well as CD20 and CD37 (as positive control molecules), was determined on huCD27-K562, a human chronic myelogenous leukemia cell line genetically modified to stably express human CD27, and on Daudi cells, using an indirect immunofluorescence assay (QIFIKIT, Agilent Technologies, cat no. K0078). Cells were seeded at 100,000 cells/well and incubated with 10 μg/mL primary antibody (CD27: IgG1-7730-143-C102S-FEAL; CD20: IgG1-11B8-FEAR; CD37: IgG1-3009-010-FEAR). This was followed by incubation with a FITC-labeled polyclonal goat anti-human IgG (Jackson Immuno Research, cat. no. 109-096-097), in parallel with QIFIKIT beads coated with a defined number of antibody molecules. The number of antibody molecules per cell was determined by interpolating the measured mean fluorescence intensity (MFI) of a test sample on the calibration curve generated by plotting the MFI of the individual bead populations against the known number of antibody molecules per bead. Samples were measured on an LSRFortessa Cell Analyzer flow cytometer (BD Biosciences) and analyzed using FlowJo software.

QiFi analysis showed moderate CD27 expression and high CD20 and CD37 expression on Daudi cells, whereas huCD27-K562 cells expressed high levels of CD27, but no CD20 and CD37 (Table 5).

TABLE 5
Cell surface expression in antibody molecules per cell

| | huCD27-K562 | Daudi |
|---|---|---|
| CD27 | 390,373 | 15,484 |
| CD20 | — | 180,217 |
| CD37 | — | 219,663 |

BRET assay (NanoBRET™ System, Promega, cat no. N1661) was performed essentially according to the manufacturer's instructions. To generate NanoLuc (donor) and HaloTag (acceptor) tagged antibodies, variable light chain sequences with either NanoLuc or HaloTag (Table 1, sequences 131-138) were prepared by gene synthesis, cloned into appropriate expression vectors and full-length antibodies produced as described in Example 1. For analysis, 0.5×10⁵ huCD27-K562 or Daudi cells were seeded in 96-well round-bottom plates (Greiner Bio-One, cat. no. 650101) in a total volume of 100 μL. Cells were pelleted by centrifugation (3 min at 300×g) and resuspended in 50 μL assay medium (Opti-MEM I [Gibco, cat. no. 11058-021]+4% FBS [ATCC, cat. no. 30-2020]) containing mixtures of NanoLuc- or HaloTag-tagged antibody pairs each at a concentration of 5 μg/mL. Next, 50 μL HaloTag NanoBret 618 ligand (Promega, cat. no. G980A, 1:1000 dilution in assay medium) was added. For each antibody mixture, a no-ligand control sample was prepared in parallel, by adding 50 μL medium without HaloTag NanoBret 618 ligand. Cells were incubated for 30 min at 37° C. in the dark, washed twice with medium and resuspended in 100 μL assay medium without FBS. 25 μL NanoBRET NanoGLO substrate (Promega, cat. no. N1571, 1:200 dilution in assay medium without FBS) was added to each well. Plates were shaken for 30 s and 120 μL of each sample was transferred to an OptiPlate (Perkin Elmer, cat. no. 6005299). An EnVision Multilabel Reader (Perkin Elmer) was used to measure donor emission at 460 nm and acceptor emission at 618 nm.

$$BRET was calculated in milliBRET units ( mBU ) = ( 618 {nm}_{e m} / 460 {nm}_{e m} ) \times 1000.$$

Results are reported as Corrected BRET, which is corrected for donor-contributed background or bleedthrough, and calculated as: mBU ligand−mBU no-ligand control.

The proximity of NanoLuc- and HaloTag-labeled IgG1-CD27-A-P329R-E345R antibodies after binding CD27 on the cell surface was compared to WT IgG1-CD27-A antibodies carrying the same tags. IgG1-CD20-11B8-E430G-LNLuc and IgG1-CD37-37.3-E430G-LHalo antibodies, containing an E430G mutation that induces hexamerization (WO2019243636A1), were used as a positive control for proximity-induced BRET. IgG1-CD20-11B8-E430G and IgG1-CD37-37.3-E430G were previously shown to form heterohexamers upon binding to cells expressing CD20 and CD37, using molecular proximity assays (Oostindie, S. C. et al, Haematologica, 2019). Nonbinding antibody IgG1-b12-P329R-E345R was used as a negative control.

As positive and negative controls for BRET signal induction, Daudi cells (high CD20 and CD37 expression) and huCD27-K562 cells (no CD20 and CD37 expression) were opsonized with antibody pair IgG1-CD20-11B8-E430G-LNLuc and IgG1-CD37-37.3-E430G-LHalo. BRET induction was detected only on Daudi cells, and not on huCD27-K562 cells lacking CD20 and CD37 (FIG. 24). Similarly, a non-binding control antibody pair (IgG1-b12-P329R-E345R-LNLuc+IgG1-b12-P329R-E345R-LHalo) did not induce BRET on either cell line. When huCD27-K562 cells were opsonized with a mixture of NanoLuc- and HaloTag-labeled CD27 antibodies bearing the hexamerization-enhancing mutation (IgG1-CD27-A-P329R-E345R-LNLuc+IgG1-CD27-A-P329R-E345R-LHalo), high BRET was detected, while BRET on Daudi cells did not exceed background levels (FIG. 24). A mixture of IgG1-CD27-A-LNLuc and IgG1-CD27-A-LHalo (WT) antibodies induced considerably lower BRET on huCD27-K562 cells compared to CD27 antibodies carrying the P329R and E345R mutations, and no BRET on Daudi cells. These results indicate that BRET signal was associated with higher target expression. CD27 expression on huCD27-K562 cells was found to be ~26 fold higher than on Daudi cells, while BRET levels for CD27-binding IgG1-CD27-A-P329R-E345R on huCD27-K562 cells were ~24 fold higher than on Daudi cells. Mixtures of NanoLuc- and HaloTag-labeled nonbinding and CD27-binding antibody pairs (IgG1-b12-P329R-E345R-LNLuc+IgG1-CD27-A-P329R-E345R-LHalo, and IgG1-CD27-A-P329R-E345R-LNLuc+IgG1-b12-P329R-E345R-LHalo respectively), did not induce BRET on either cell line. This confirms that observed BRET was dependent on simultaneous interaction of donor and acceptor antibodies bound to the cell-surface target.

In summary, IgG1-CD27-A-P329R-E345R induced high BRET on huCD27-K562 cells compared to its WT variant. This finding confirms enhanced proximity between membrane-bound IgG1-CD27-A-P329R-E345R molecules, compared to its WT variant, consistent with E345R-enhanced Fc-Fc interactions between cell surface-bound antibodies.

N.B. the experiment described in this example used a variant of IgG1-CD27-A carrying a F405L mutation, which is functionally irrelevant in the context of this experiment.

### Example 27: Binding of IgG1-CD27-A-P329R-E345R to FcγRIa⁺ M0 and M1 Macrophages

Example 9 assessed binding of IgG1-CD27-A-P329R-E345R to human FcγR variants using surface plasmon resonance (SPR), showing minimal (FcγRIa) or no (FcγRIIa, FcγRIIb, and FcγRIIIa) binding to recombinant human IgG Fc receptor molecules. This residual FcγRIa binding was not sufficient to induce IgG1-CD27-A-P329R-E345R-dependent ADCP of CD27⁺ cells (see Example 13). To further exclude interactions of IgG1-CD27-A-P329R-E345R with FcγRIa-positive macrophages, Fc-mediated binding of IgG1-CD27-A-P329R-E345R to M0 and M1 macrophages was determined.

Human CD14⁺ monocytes were isolated from PBMCs from two healthy donors as described in Example 13, and differentiated into monocyte-derived macrophages by culturing the cells in medium (CellGenix, cat. no. 20801-0500) supplemented with 50 ng/mL M-CSF (Gibco, cat. no. PHC9501) to obtain M0 macrophages, or 50 ng/mL GM-CSF (Immunotools, cat. no. 11343125) for differentiation into M1 macrophages. After 6 days of culture, M0 and M1 phenotypes were confirmed by FACS analysis according to expression of markers as defined in Table 6. Additionally, both macrophage subtypes were confirmed to express human Fc receptors FcγRIa, FcγRII and FcγRIIIa (Table 6).

TABLE 6

| Phenotype markers | M0 | M1 |
|---|---|---|
| CD40 (BD Pharmingen, cat. no. 561211, 1:50 dilution) | + | + |
| CD86 (MACS, cat. no. 30-097-877, 1:50 dilution) | + | ++ |
| CD163 (Biolegend, cat. no. 333612, 1:200 dilution) | +/− | − |
| CD206 (Biolegend, cat. no. 321136, 1:200 dilution) | +/− | + |
| Fc receptors | | |
| FcγRIa (Biolegend, cat. no. 305006, 1:25 dilution) | ++ | ++ |
| FcγRII (BD Pharmingen, cat. no. 552883, 1:50 dilution) | ++ | ++ |
| FcγRIIIa (BD Pharmingen, cat. no. 555407, 1:50 dilution) | + | +/− |

Binding of IgG1-CD27-A-P329R-E345R to M0 and M1 macrophages was compared to binding of a WT IgG1 antibody (IgG1-b12) with an irrelevant antigen-binding region as a positive control for FcγRIa binding, and a variant of the same antibody also carrying the P329R mutation previously described to reduce interaction with FcγR (IgG1-b12-P329R-E345R). Since macrophages should not express CD27, any binding observed is hypothesized to occur via FcγRIa, which is the only FcγR that binds monovalent IgG. The differentiated macrophages were incubated with IgG1-CD27-A-P329R-E345R or control antibodies (30 μg/mL in DC medium) for 15 min, and PE-labeled polyclonal goat anti-human IgG (Jackson Immuno Research, cat. no. 109-116-097, dilution 1:200, 30 min at 4° C.). After incubation, cells were washed and resuspended in 100 μL FACS buffer containing nucleus-staining DAPI (BD Pharmingen, cat. no. 564907, 1:5000 dilution). Samples were measured on a FACSymphony flow cytometer (BD Biosciences) and analyzed using FlowJo software.

No binding above background (secondary antibody only) to M0 or M1 macrophages isolated from two independent donors was observed with either IgG1-CD27-A-P329R-E345R or control IgG1-b12-P329R-E345R (FIG. 25). WT IgG1-b12, which contains an active Fc region, consistently bound to both M0 and M1 macrophages.

In conclusion, the IgG1-CD27-A-P329R-E345R and control IgG1-b12-P329R-E345R do not bind M0 or M1 macrophages expressing FcγRIa, FcγRII and FcγRIIIa.

### Example 28: Generation of IgG1-PD1 and Screening Materials

### PD-1 and FcγR Constructs

Plasmids encoding various full-length PD-1 variants were generated: human (Homo sapiens; UniProtKB ID: Q15116), cynomolgus monkey (Macaca fascicularis; UniProtKB ID: BOLAJ3), dog (Canis familiaris; UniProtKB ID: E2RPS2), rabbit (Oryctolagus cuniculus; UniProtKB ID: G1SUF0), pig (Sus scrofa; UniProtKB ID: A0A287A1C3), rat (Rattus norvegicus; UniProtKB ID: D3ZIN8), and mouse (Mus musculus; UniProtKB ID: Q02242), as well as a plasmid encoding human FcγRIa (UniProt KB ID: P12314).

### Generation of CHO-S Cell Lines Transiently Expressing Full-Length PD-1 or FcγR Variants

CHO-S cells (a subclone of CHO cells adapted to suspension growth; ThermoFisher Scientific, cat. no. R800-07) were transfected with PD-1 or FcγR plasmids using FreeStyle™ MAX Reagent (ThermoFisher Scientific, cat. no. 16447100) and OptiPRO™ serum-free medium (ThermoFisher Scientific, cat. no. 12309019), according to the manufacturer's instructions.

### Production of Antibody Variants

### IgG1-PD1

Three New Zealand White rabbits were immunized with recombinant human His-tagged PD-1 protein (R&D Systems, cat. no. 8986-PD). Single B cells from blood were sorted and supernatants screened for production of PD-1 specific antibodies by human PD-1 enzyme-linked immunosorbent assay (ELISA), cellular human PD-1 binding assay and by human PD-1/PD-L1 blockade bioassay. From screening-positive B cells, RNA was extracted, and sequencing was performed. The variable regions of heavy and light chain were gene synthesized and cloned N-terminal of human immunoglobulin constant parts (IgG1/K) containing mutations L234A and L235A (LALA; Labrijn et al. Sci Rep 2017, 7:2476) wherein the amino acid position number is according to Eu numbering (SEQ ID NO: 43) to minimize interactions with Fcγ receptors.

Transient transfections of HEK293-FreeStyle cells using 293-free transfection reagent (Novagen/Merck) were executed by Tecan Freedom Evo device. Produced chimeric antibodies were purified from cell supernatant using protein-A affinity chromatography on a Dionex Ultimate 3000 HPLC with plate autosampler. Purified antibodies were used for further analysis in particular retesting by human PD-1 ELISA, cellular human PD-1 binding assay, human PD-1/PD-L1 blockade bioassay, and T-cell proliferation assay. The chimeric rabbit antibody MAB-19-0202 (SEQ ID NO: 54 and 55) was identified as best performing clone and subsequently humanized.

The variable region sequences of the chimeric PD-1 antibody MAB-19-0202 are shown in the following tables. Table 7 shows the variable regions of the heavy chain, while Table 8 shows the variable regions of the light chain. In both cases the framing regions (FRs) as well as the complementarity determining regions (CDRs) according to Kabat numbering are defined. The underlined amino acids indicate the CDRs according to the IMGT numbering. The bold letters indicate the intersection of Kabat and IMGT numbering.

TABLE 7
HEAVY CHAIN

| Sequence ID | FR1 | CDR1 | FR2 | CDR2 | FR3 | CDR3 | FR4 |
|---|---|---|---|---|---|---|---|
| MAB-19-0202-HC | QSVEES | SYNMG | WVRQA | IISGGT | RFTISK | AFYDD | WGPGTL |
| SEQ ID NO: 54 | GGRLVT | | PGKGL | IGHYAS | TSSTTV | YDYNV | VTVSS |
| | PGTPLT | | EYIG | WAKG | DLKMTS | | |
| | LTCTVS | | | | LTTEDT | | |
| | GFSLY | | | | ATYFCA | | |
| | | | | | R | | |

TABLE 8
LIGHT CHAIN

| Sequence ID | FR1 | CDR1 | FR2 | CDR2 | FR3 | CDR3 | FR4 |
|---|---|---|---|---|---|---|---|
| MAB-19-0202-LC | AAVLTQ | QSSQS | WYQQK | QASKLE | GVPSRF | AGGYS | FGGGTE |
| SEQ ID NO: 55 | TPSPVS | VYGNN | PGQPP | T | KGSGSG | SSSDT | VVVK |
| | AAVGGT | QLS | KLLIY | | TQFTLT | I | |
| | VTISC | | | | ISDLES | | |
| | | | | | DDAATY | | |
| | | | | | YC | | |

Humanized heavy and light chain variable region antibody sequences were generated by structural modelling-assisted CDR grafting, gene synthesized and cloned N-terminal of human immunoglobulin constant parts (IgG/K with LALA mutations). Humanized antibodies were used for further analysis in particular retesting by human PD-1 ELISA, cellular human PD-1 binding assay, human PD-1/PD-L1 blockade bioassay, and the T-cell proliferation assay. The humanized antibody MAB-19-0618 (SEQ ID NO: 56 and 57) was identified as best performing clone.

The allocation of the humanized light and heavy chains to antibody ID of the recombinant humanized sequences are listed in Table 9. The variable region sequences of the humanized light and heavy chains are shown in Table 10 and 11. Table 10 shows the variable regions of the heavy chain, while Table 11 shows the variable regions of the light chain. In both cases the framing regions (FRs) as well as the complementarity determining regions (CDRs) according to Kabat numbering are defined. The underlined amino acids indicate the CDRs according to the IMGT numbering.

TABLE 9

| | light chain | | heavy chain | |
|---|---|---|---|---|
| antibody | humanized | Light chain | humanized | Heavy chain |
| ID | variant | SEQ ID NO: | variant | SEQ ID NO: |
| MAB-19- | MAB-19-0202- | 57 | MAB-19-0202- | 56 |
| 0618 | L4 | | H5 | |

TABLE 10
HEAVY CHAIN

| Sequence ID | FR1 | CDR1 | FR2 | CDR2 | FR3 | CDR3 | FR4 |
|---|---|---|---|---|---|---|---|
| MAB-19-0202-H5 | QVQLVE | SYNMG | WVRQA | IISGGT | RFTISR | AFYDD | WGPGT |
| SEQ ID NO: 56 | SGGGLV | | PGKGL | IGHYAS | DTSKTT | YDYNV | LVTVS |
| | QPGTSL | | EYIG | WAKG | LYLQMN | | S |
| | RLSCSV | | | | SLTTED | | |
| | SGFSLY | | | | TATYFC | | |
| | | | | | AR | | |

TABLE 11
LIGHT CHAIN

| Sequence ID | FR1 | CDR1 | FR2 | CDR2 | FR3 | CDR3 | FR4 |
|---|---|---|---|---|---|---|---|
| MAB-19-0202-L4 | AIQLTQ | QSSQS | WYQQK | QASKLE | GVPSRF | AGGYSS | FGGGTE |
| SEQ ID NO: 57 | SPSSLS | VYGNN | PGQPP | T | RGSGSG | SSDTT | VVVK |
| | ASVGGT | QLS | KLLIY | | TQFTLT | | |
| | VTITC | | | | ISSLQS | | |
| | | | | | EDFATY | | |
| | | | | | YC | | |

The sequences of the variable regions of the heavy and light chains of MAB-19-0618 were gene synthesized and cloned by ligation-independent cloning (LIC) into expression vectors with codon-optimized sequences encoding the human IgG1m(f) heavy chain constant domain containing the Fc-silencing mutations L234F, L235E and G236R (FER) wherein the amino acid position number is according to Eu numbering (SEQ ID NO: 38) and the human kappa light chain constant domain (SEQ ID NO: 42). The resulting antibody was designated IgG1-PD1.

The GS Xceed® Expression System (Lonza) was used to generate a stable cell line expressing IgG1-PD1. The sequences encoding the heavy and light chain of IgG1-PD1 were cloned into the expression vectors pXC-18.4 and pXC-Kappa (containing the glutamine synthetase [GS] gene), respectively, by Lonza Biologics plc. Next, a double gene vector (DGV) encoding both the heavy and light chain of IgG1-PD1 was constructed by ligating the complete expression cassette from the heavy chain vector into the light chain vector. The DNA of this DGV was linearized with the restriction enzyme PvuI-HF (New England Biolabs, R3150L) and used for stable transfection of CHOK1SV® GS-KO® cells. IgG1-PD1 was purified for functional characterization.

### IgG1-CD52-E430G

A human IgG1 antibody with an E430G hexamerization-enhancing mutation (WO2013/004842 A2) in the Fc domain (SEQ ID NO: 40) and antigen-binding domains identical to CAMPATH-1H, a CD52-specific antibody, was used as a positive control in C1q binding and FcγR signaling experiments experiments (Crowe et al., 1992 Clin Exp Immunol. 87(1):105-110) (SEQ ID NO. 61 and 65).

### Control Antibodies

Human IgG1 antibodies with antigen-binding domains identical to b12, an HIV1 gp120-specific antibody, were used as negative controls in several experiments (Barbas et al., J Mol Biol. 1993 Apr. 5; 230(3):812-2). VH and VL domains of b12 (SEQ ID NO. 68 and 72) were prepared by de novo gene synthesis (GeneArt Gene Synthesis; ThermoFisher Scientific, Germany) and cloned into expression vectors containing a human IgG1 heavy chain constant region (i.e. CH1, hinge, CH2 and CH3 region) of the human IgG1m(f) allotype (SEQ ID NO: 37) or a variant thereof (containing the L234F/L235E/G236R mutations and an additional, in the context of this study functionally irrelevant, K409R mutation in the Fc domain, abbreviated as the FERR mutations) (SEQ ID NO:39) or containing a human IgG4 heavy chain constant region (SEQ ID NO: 41); or the constant region of the human kappa light chain (LC) (SEQ ID NO: 42), as appropriate for the selected binding domains. Antibodies were obtained by transfection of heavy and light chain expression vectors in production cell lines and purified for functional characterization.

### Example 29: Binding of IgG1-PD1 to PD-1 from Various Species

Binding of IgG1-PD1 to PD-1 of species commonly used for nonclinical toxicology studies was assessed by flow cytometry using CHO-S cells transiently expressing PD-1 from different animal species.

CHO-S cells (5×10⁴ cells/well) were seeded in round-bottom 96-well plates. Antibody dilutions (1.7×10⁻⁴-30 μg/mL or 5.6×10⁻⁵-10 μg/mL, 3fold dilutions) of IgG1-PD1, IgG1-ctrl-FERR, and pembrolizumab were prepared in Genmab (GMB) fluorescence-activated cell sorting (FACS) buffer (phosphate-buffered saline [PBS; Lonza, cat. no. BE17-517Q, diluted to 1×PBS in distilled water] supplemented with 0.1% [w/v] bovine serum albumin [BSA; Roche, cat. no. 10735086001] and 0.02% [w/v] sodium azide [NaN₃; bioWORLD, cat. no. 41920044-3]). An IgG4 isotype control (BioLegend, cat. no. 403702) for pembrolizumab was included only at the highest concentration tested (30 μg/mL or 10 μg/mL). Cells were centrifuged, supernatant was removed, and cells were incubated in 50 μL of the antibody dilutions for 30 min at 4° C. Cells were washed twice with GMB FACS buffer and incubated with 50 μL secondary antibody R-phycoerythrin (PE)-conjugated goat-anti-human IgG F(ab′)2 (Jackson ImmunoResearch, cat. no. 109-116-098; diluted 1:500 in GMB FACS buffer) for 30 min at 4° C., protected from light. Cells were washed twice with GMB FACS buffer, resuspended in GMB FACS buffer supplemented with 2 mM ethylenediaminetetraacetic acid (EDTA; Sigma-Aldrich, cat. no. 03690) and 4′,6-diamidino-2-phenylindole (DAPI) viability marker (1:5,000; BD Pharmingen, cat. no. 564907). Antibody binding to viable cells (as identified by DAPI exclusion) was analyzed by flow cytometry on an Intellicyt® iQue PLUS Screener (Intellicyt Corporation) using FlowJo software. Binding curves were analyzed using non-linear regression analysis (four-parameter dose-response curve fits) in GraphPad Prism.

Binding of IgG1-PD1 to PD-1 of different species was evaluated by flow cytometry using CHO-S cells transiently transfected to express human, cynomolgus monkey, dog, rabbit, pig, rat, or mouse PD-1 protein on the cell surface. Dose-dependent binding of IgG1-PD1 was observed for human and cynomolgus monkey PD-1 (FIG. 26A-B). Pembrolizumab demonstrated comparable binding. Substantially reduced cross-reactivity of IgG1-PD1, and only at the highest concentrations, was observed to rodent PD-1 (mouse, rat; FIGS. 26C-D) and no binding was observed to PD-1 of other species frequently used in toxicology studies (rabbit, dog, pig; FIG. 26E). No IgG1-PD1 binding was observed to non-transfected control cells (FIG. 26E), nor was binding of IgG1-ctrl-FERR, included as a negative control, observed to PD-1 of any of the tested species (FIG. 26).

In conclusion, IgG1-PD1 showed comparable binding to membrane-expressed human and cynomolgus monkey PD-1 and significantly lower or no binding to mouse, rat, rabbit, dog, and pig PD-1.

### Example 30: Binding to Human and Cynomolgus Monkey PD-1 Determined by Surface Plasmon Resonance

Binding of immobilized IgG1-PD1, pembrolizumab, and nivolumab to human and cynomolgus monkey PD-1 was analyzed by surface plasmon resonance (SPR) using a Biacore 8K SPR system. Recombinant human and cynomolgus monkey PD-1 extracellular domain (ECD) with a C-terminal His-tag were obtained from Sino Biological (cat. no. HPLC-10377-H08H and 90311-C08H, respectively).

Biacore Series S Sensor Chips CM5 (Cytiva, cat. no. 29149603) were covalently coated with anti-Fc antibody using amine coupling and the Human Antibody Capture Kit, Type 2 (Cytiva, cat. no. BR100050 and BR100839) according to the manufacturer's instructions.

Subsequently, IgG1-PD1 (2 nM), nivolumab (Bristol-Myers Squibb, lot no. ABP6534; 1.25 nM), and pembrolizumab (Merck Sharp & Dohme, lot. no. T019263; 1.25 nM), diluted in HBS-EP+ buffer (Cytiva, cat. no. BR100669; diluted to 1× in distilled water [B Braun, cat. no. 00182479E]), were captured onto the surface at 25° C., with a flow rate of 10 μL/min and a contact time of 60 seconds. This resulted in captured levels of approximately 50 resonance units (RU).

After three start-up cycles of HBS-EP+ buffer, human or cynomolgus monkey PD-1 ECD samples (0.19-200 nM; 2-fold dilution in HBS-EP+ buffer; 12 cycles) were injected to generate binding curves. Each sample that was analyzed on an antibody coated surface (active surface) was also analyzed on a parallel flow cell without antibody (reference surface), which was used for background correction.

At the end of each cycle, the surface was regenerated using 10 mM Glycine-HCl pH 1.5 (Cytiva, cat. no. BR100354). The data were analyzed using the predefined “Multi-cycle kinetics using capture” evaluation method in the Biacore Insight Evaluation software (Cytiva). The sample with the highest concentration of human or cynomolgus monkey PD-1 (200 nM) was omitted from analysis to allow better curve fits of the data.

Immobilized IgG1-PD1 bound to human PD-1 ECD with a binding affinity (K_{D}) of 1.45±0.05 nM (Table 10). Nivolumab and pembrolizumab bound human PD-1 ECD with a binding affinity comparable to the K_{D} of IgG1-PD1, ie, with K_{D} values in the low nanomolar range (4.43±0.08 nM and 3.59±0.10 nM, respectively) (Table 12).

Immobilized IgG1-PD1 bound to cynomolgus monkey PD-1 ECD with a K_{D} of 2.74±0.58 nM (Table 11), comparable to the affinity of IgG1-PD1 for human PD-1. Nivolumab and pembrolizumab bound cynomolgus monkey PD-1 ECD with a binding affinity comparable to the K_{D} of IgG1-PD1 for cynomolgus monkey PD-1 ECD and comparable to the K_{D} of nivolumab and pembrolizumab for human PD-1 ECD, ie, with K_{D} values in the low nanomolar range (2.93±0.58 nM and 0.90±0.06 nM, respectively) (Table 13).

TABLE 12
Binding affinities of PD-1 antibodies to the extracellular domain of human PD-1 as determined
by surface plasmon resonance. The association rate constant kₐ (1/Ms), dissociation rate constant
k_{d} (1/s) and equilibrium dissociation constant K_{D} (M) of IgG1-PD1, nivolumab, and pembrolizumab
for the ECD of human PD-1 were determined by SPR.

| | K_{D} (M) | | kₐ (1/M × s) | | k_{d} (1/s) | |
|---|---|---|---|---|---|---|
| Antibody | Average | SD | Average | SD | Average | SD |
| IgG1-PD1^{a} | 1.45 × 10⁻⁹ | 4.51 × 10⁻¹¹ | 5.17 × 10⁵ | 2.03 × 10⁴ | 7.51 × 10⁻⁴ | 9.61 × 10⁻⁶ |
| Nivolumab^{b} | 4.43 × 10⁻⁹ | 8.49 × 10⁻¹¹ | 4.06 × 10⁵ | 3.54 × 10³ | 1.80 × 10⁻³ | 2.12 × 10⁻⁵ |
| Pembrolizumab^{b} | 3.59 × 10⁻⁹ | 9.90 × 10⁻¹¹ | 2.12 × 10⁶ | 1.36 × 10⁶ | 7.57 × 10⁻³ | 4.71 × 10⁻³ |

^{a}Average and SD from three independent experiments.
^{b}Average and SD from two independent experiments.
Abbreviations: K_{D} = equilibrium dissociation constant; kₐ = association rate constant; k_{d} = dissociation rate constant or off-rate; SD = standard deviation.

TABLE 13
Binding affinities of PD-1 antibodies to the extracellular domain of cynomolgus monkey PD-1
as determined by surface plasmon resonance. The association rate constant kₐ (1/Ms), dissociation rate
constant k_{d} (1/s) and equilibrium dissociation constant K_{D} (M) of IgG1-PD1, nivolumab, and
pembrolizumab for the ECD of cynomolgus monkey PD-1 were determined by SPR.

| | K_{D} (M) | | kₐ (1/M × s) | | k_{d} (1/s) | |
|---|---|---|---|---|---|---|
| Antibody | Average | SD | Average | SD | Average | SD |
| IgG1-PD1^{a} | 2.74 × 10⁻⁹ | 5.79 × 10⁻¹⁰ | 5.34 × 10⁵ | 9.39 × 10⁴ | 1.43 × 10⁻³ | 7.23 × 10⁻⁵ |
| Nivolumab^{a} | 2.93 × 10⁻⁹ | 5.81 × 10⁻¹⁰ | 3.28 × 10⁵ | 5.07 × 10⁴ | 9.43 × 10⁻⁴ | 9.07 × 10⁻⁵ |
| Pembrolizumab^{b} | 8.99 × 10⁻¹⁰ | 5.73 × 10⁻¹¹ | 7.14 × 10⁵ | 3.04 × 10⁴ | 6.42 × 10⁻⁴ | 6.86 × 10⁻⁵ |

^{a}Average and SD from three independent experiments.
^{b}Average and SD from two independent experiments.
Abbreviations: K_{D} = equilibrium dissociation constant; kₐ = association rate constant; k_{d} = dissociation rate constantor off-rate; SD = standard deviation.

### Example 31: Effect of IgG1-PD1 on PD-1 Ligand Binding and PD-1/PD-L1 Signaling

To confirm that IgG1-PD1 functions as a classical immune checkpoint inhibitor, the capacity of IgG1-PD1 to disrupt PD-1 ligand binding and PD-1 checkpoint function was assessed in vitro.

Competitive binding of IgG1-PD1 with recombinant human PD-L1 and PD-L2 to membrane-expressed human PD-1 was assessed by flow cytometry. CHO-S cells transiently transfected with human PD-1 (see Example 26; 5×10⁴ cells/well) were added to the wells of a round-bottom 96-well plate (Greiner, cat. no. 650180), pelleted, and placed on ice. Biotinylated recombinant human PD-L1 (R&D Systems, cat. no. AVI156) or PD-L2 (R&D Systems, cat. no. AVI1224), diluted in PBS (Cytiva, cat. no. SH3A3830.03), was added to the cells (final concentration: 1 μg/mL), immediately after which a concentration range of IgG1-PD1, pembrolizumab (MSD, lot no. T019263 and T036998), or IgG1-ctrl-FERR, diluted in PBS, was added (final concentrations: 30 μg/mL-0.5 ng/mL in three-fold dilution steps). Cells were then incubated for 45 min at RT. Cells were washed twice with PBS and incubated with 50 μL streptavidin-allophycocyanin (R&D Systems, cat. no. F0050; diluted 1:20 in PBS) for 30 min at 4° C., protected from light. Cells were washed twice with PBS and resuspended in 20 μL GMB FACS buffer. Streptavidin-allophycocyanin binding was analyzed by flow cytometry on an Intellicyt® iQue Screener PLUS (Sartorius) using FlowJo software.

The effect of IgG1-PD1 on the functional interaction of PD-1 and PD-L1 was determined using a bioluminescent cell-based PD-1/PD-L1 blockade reporter assay (Promega, cat. no. J1255), essentially as described by the manufacturer. Briefly, cocultures of PD-L1 aAPC/CHO-K1 Cells and PD-1 Effector Cells were incubated with serially diluted IgG1-PD1, pembrolizumab (MSD, lot no. 10749880 or T019263), nivolumab (Bristol-Myers Squibb, lot no. 11024601), or IgG1-ctrl-FERR (final assay concentrations: 15-0.0008 μg/mL in 3-fold dilutions or 10-0.0032 μg/mL in 5-fold dilutions) for 6 h at 37° C., 5% CO₂. Cells were then incubated at RT with reconstituted Bio-Glo™ for 5-30 min, after which luminescence (in relative light units [RLU]) was measured using an Infinite® F200 PRO Reader (Tecan) or an EnVision Multilabel Plate Reader (PerkinElmer).

Dose-response curves were analyzed by non-linear regression analysis (four-parameter dose-response curve fits) using GraphPad Prism software, and the concentrations at which 50% of the maximal (inhibitory) effect was observed (EC₅₀/IC₅₀) were derived from the fitted curves.

IgG1-PD1 disrupted binding of human PD-L1 and PD-L2 to membrane-expressed human PD-1 in a dose-dependent manner (FIG. 27), with IC₅₀ values of 2.059±0.653 μg/mL (13.9±4.4 nM) for PD-L1 binding inhibition and 1.659±0.721 μg/mL (11.2±4.9 nM) for PD-L2 binding inhibition, ie, in the nanomolar range (Table 14). Pembrolizumab showed PD-L1 and PD-L2 binding inhibition with comparable potency, i.e., with IC₅₀ values in the nanomolar range.

Functional blockade of the PD-1/PD-L1 axis was tested using a cell-based bioluminescent PD-1/PD-L1 blockade reporter assay. Cocultures of reporter Jurkat T cells expressing human PD-1 and harboring an NFAT-RE-driven luciferase, and PD-L1 aAPC/CHOK1 cells expressing human PD-L1 and an antigen-independent TCR activator, were incubated in absence and presence of concentration dilution series of IgG1-PD1, pembrolizumab, or nivolumab. IgG1-ctrl-FERR was included as a negative control. Blockade of the PD-1/PD-L1 interaction results in the release of the PD1/PDL1 mediated inhibitory signal, leading to TCR activation and NFAT-RE-mediated luciferase expression (luminescence measured). IgG1-PD1 induced a dose-dependent increase of TCR signaling in PD-1⁺ reporter T cells (FIG. 28). The EC₅₀ was 0.165±0.056 μg/mL (1.12±0.38 nM; Table 15). Pembrolizumab similarly alleviated PD-1 mediated inhibition of TCR signaling, with an EC₅₀ of 0.129±0.051 μg/mL (0.86±0.34 nM), ie, with comparable potency. Nivolumab alleviated the inhibition of TCR signaling with an EC₅₀ of 0.479±0.198 μg/mL (3.28±1.36 nM), i.e., with slightly lower potency.

In summary, IgG1-PD1 acts as a classical immune checkpoint inhibitor in vitro, by blocking PD-1 ligand binding and disrupting PD-1 immune checkpoint function.

TABLE 14
IC₅₀ values of IgGI-PDI-mediated inhibition of PD-1 ligand binding.
IC₅₀ values were calculated from the competition binding curves.

| Competitive binding with human PD-L1 | | | | Competitive binding with human PD-L2 | | | |
|---|---|---|---|---|---|---|---|
| (average IC₅₀ [±SD]) | | | | (average IC₅₀ [±SD]) | | | |
| IgG1-PD1 | | pembrolizumab | | IgG1-PD1 | | pembrolizumab | |
| μg/mL | nM | μg/mL | nM | μg/mL | nM | μg/mL | nM |
| 2.059 | 13.9 | 1.134 | 7.6 | 1.659 | 11.2 | 1.186 | 8.0 |
| [±0.653] | [±4.4] | [±0.493] | [±3.3] | [±0.721] | [±4.9] | [±0.770] | [±5.2] |

Abbreviations: IC₅₀ = concentration at which 50% of the inhibitory effect was observed; PD-1 = programmed cell death protein 1; PD-L1 = programmed cell death 1 ligand 1; PD-L2 = programmed cell death 1 ligand 2; SD = standard deviation.

TABLE 15
EC₅₀ of PD-1/PD-L1 checkpoint blockade. Cocultures of PD-1⁺
reporter T cells and PD-L1 aAPC/CHO-K cells were incubated with
concentration series of IgG1-PD1, pembrolizumab, or nivolumab in
PD-1/PD-L1 blockade reporter assays. Inhibition of PD-1/PD-L1
checkpoint function, resulting in downstream TCR signaling and
luciferase expression in the reporter T cells, was determined
by measuring luminescence. From the resulting dose-response
curves, EC₅₀ values were calculated.
Average EC₅₀ [± SD]

| IgG1-PD1 | | Pembrolizumab | | Nivolumab | |
|---|---|---|---|---|---|
| μg/mL | nM | μg/mL | nM | μg/mL | nM |
| 0.165 [± | 1.12 | 0.129 [± | 0.86 | 0.479 [± | 3.28 |
| 0.056] | [± 0.38] | 0.051] | [± 0.34] | 0.198] | [± 1.36] |

Abbreviations: aAPC = artificial antigen-presenting cell; CHO = Chinese hamster ovary; EC₅₀ = concentration at which 50% of the maximal effect is observed; PD-1 = programmed cell death protein 1; PD-L1 = programmed cell death 1 ligand 1; SD = standard deviation; TCR = T-cell receptor.

### Example 32: Antigen-Specific Proliferation Assay to Determine the Capacity of IgG1-PD1 to Enhance Proliferation of Activated T Cells

To determine the capacity of IgG1-PD1 to enhance T-cell proliferation, an antigen-specific proliferation assay was conducted using PD-1-overexpressing human CD8⁺ T cells.

HLA-A*02+ peripheral blood mononuclear cells (PBMCs) were obtained from healthy donors (Transfusionszentrale, University Hospital, Mainz, Germany). Monocytes were isolated from PBMCs by magnetic-activated cell sorting (MACS) technology using anti-CD14 MicroBeads (Miltenyi; cat. no. 130-050-201), according to the manufacturer's instructions. The peripheral blood lymphocytes (PBLs, CD14-negative fraction) were cryopreserved in RPMI 1640 containing 10% DMSO (AppliChem GmbH, cat. no A3672,0050) and 10% human albumin (CSL Behring, PZN 00504775) for T-cell isolation. For differentiation into immature DCs (iDCs), 1×106 monocytes/mL were cultured for five days in RPMI 1640 (Life Technologies GmbH, cat. no. 61870-010) containing 5% pooled human serum (One Lambda Inc., cat. no. A25761), 1 mM sodium pyruvate (Life technologies GmbH, cat. no. 11360-039), 1× non-essential amino acids (Life Technologies GmbH, cat. no. 11140-035), 200 ng/mL granulocyte-macrophage colony-stimulating factor (GM-CSF; Miltenyi, cat. no. 130-093-868) and 200 ng/mL interleukin-4 (IL-4; Miltenyi, cat. no. 130-093-924). After three days in culture days, half of the medium was replaced with fresh medium. On day 5, iDCs were harvested by collecting non-adherent cells and adherent cells were detached by incubation with Dulbecco's phosphate-buffered saline (DPBS) containing 2 mM EDTA for 10 min at 37°. After washing with DPBS iDCs were cryopreserved in in fetal bovine serum (FBS; Sigma-Aldrich, cat. no. F7524) containing 10% DMSO for future use in antigen-specific T cell assays.

One day prior to the start of an antigen-specific CD8⁺ T cell proliferation assay, frozen PBLs and iDCs from the same donor were thawed. CD8⁺ T cells were isolated from PBLs by MACS technology using anti-CD8 MicroBeads (Miltenyi, cat. no. 130-045-201), according to the manufacturer's instructions. About 10×10⁶ to 15×10⁶ CD8⁺ T cells were electroporated with each 10 μg of in vitro transcribed (IVT)-RNA encoding the alpha and beta chains of a murine TCR specific for human claudin-6 (CLDN6; HLA-A*02-restricted; described in WO 2015150327 A1) plus 10 μg IVT-RNA encoding PD-1 (UniProt Q15116) in 250 μL X-Vivo15 medium (Lonza, cat. no. BE02-060Q). The cells were transferred to a 4-mm electroporation cuvette (VWR International GmbH, cat. no. 732-0023) and electroporated using the BTX ECM® 830 Electroporation System (BTX; 500 V, 1×3 ms pulse). Immediately after electroporation, cells were transferred into fresh IMDM GlutaMAX medium (Life Technologies GmbH, cat. no. 319800-030) containing 5% pooled human serum and rested at 37° C., 5% CO₂ for at least 1 hour. T cells were labeled using 1.6 μM carboxyfluorescein succinimidyl ester (CFSE; Life Technologies GmbH, cat. No V12883) in PBS according to the manufacturer's instructions and incubated in IMDM medium supplemented with 5% pooled human serum overnight.

Up to 5×10⁶ thawed iDCs were electroporated with 2 μg IVT-RNA encoding full-length human CLDN6 (WO 2015150327 A1), in 250 μL X-Vivo15 medium, using the electroporation system as described above (300 V, 1×12 ms pulse) and incubated in IMDM medium supplemented with 5% pooled human serum overnight.

The next day, cells were harvested. Cell-surface expression of CLDN6 on iDCs, as well as cell-surface expression of the CLDN6-specific TCR and PD-1 on T cells was confirmed by flow cytometry. To this end, iDCs were stained with a DyLight650-conjugated CLDN6-specific antibody (non-commercially available; in-house production). T cells were stained with a brilliant violet (BV)421-conjugated anti-mouse TCR-β chain antibody (Becton Dickinson GmbH, cat. no. 562839) and an allophycocyanin (APC)-conjugated anti-human PD-1 antibody (Thermo Fisher Scientific, cat. no. 17-2799-42).

Electroporated iDCs were incubated with electroporated, CFSE-labeled T cells at a ratio of 1:10 in the presence of IgG1-PD1, pembrolizumab (Keytruda®, MSD Sharp & Dohme GmbH, PZN 10749897), or nivolumab (Opdivo®, Bristol-Myers Squibb, PZN 11024601) at 4-fold serial dilutions (range 0.00005 to 0.8 μg/mL) in IMDM medium containing 5% pooled human serum in a 96-well round-bottom plate. The negative control antibody IgG1-ctrl-FERR was used at a single concentration of 0.8 μg/mL. After 4 d of culture, the cells were stained with an APC-conjugated anti-human CD8 antibody. T-cell proliferation was evaluated by flow cytometry analysis of CFSE dilution in CD8⁺ T cells using a BD FACSCelesta™ flow cytometer (Becton Dickinson GmbH).

Flow cytometry data was analyzed using FlowJo software version 10.7.1. CFSE label dilution of CD8⁺ T cells was assessed using the proliferation modeling tool in FlowJo, and expansion indices calculated using the integrated formula. Dose-response curves were generated in GraphPad Prism version 9 (GraphPad Software, Inc.) using a 4-parameter logarithmic fit. Statistical significance was determined by Friedman's test and Dunn's multiple comparisons test using GraphPad Prism version 9.

Antigen-specific proliferation of CD8⁺ T cells was enhanced by IgG1-PD1 in a dose-dependent manner (FIG. 29), with EC₅₀ values in the picomolar range (Table 16). Treatment with pembrolizumab or nivolumab also enhanced T-cell proliferation in a dose-dependent manner.

The average EC₅₀ of pembrolizumab was comparable to IgG1-PD1, whereas the EC₅₀ of nivolumab was significantly (P=0.0267) higher than that of IgG1-PD1.

TABLE 16
EC₅₀ values in the antigen-specific proliferation assay. EC₅₀ values of
IgG1-PD1, pembrolizumab, and nivolumab were determined using the
CD8⁺ T-cell expansion indices as measured by an antigen-
specific T-cell proliferation assay. Data shown are the values
calculated based on the 4-parameter logarithmic fit.
Average EC₅₀ [± SD]

| IgG1-PD1 | | Pembrolizumab | | Nivolumab | |
|---|---|---|---|---|---|
| μg/mL | nM | μg/mL | nM | μg/mL | nM |
| 0.0124 | 0.0837 | 0.0152 | 0.1018 | 0.0701 | 0.4802 |
| [± 0.0018] | [± 0.0123] | [± 0.0049] | [± 0.0333] | [± 0.0238] | [± 0.1632] |

Abbreviations: EC₅₀ = half-maximal effective concentration; FERR = L234F/L235E/G236R-K409R; PD1 = programmed cell death protein 1; SD = standard deviation.

### Example 33: Effect of IgG1-PD1 on Cytokine Secretion in an Allogeneic MLR Assay

To investigate the capacity of IgG1-PD1 to enhance cytokine secretion in a mixed lymphocyte reaction (MLR) assay, three unique, allogeneic pairs of human mature dendritic cells (mDCs) and CD8⁺ T cells were cocultured in the presence of IgG1-PD1. The levels of IFNγ were measured using an IFNγ-specific immunoassay, while the levels of monocyte chemoattractant protein-1 (MCP-1), GM-CSF, interleukin (IL)-1β, IL-2, IL-4, IL-5, IL-6, IL-8, IL-10, IL12-p40, IL-15, IL-17a, and tumor necrosis factor (TNFα) were determined using a customized Luminex multiplex immunoassay.

Human CD14⁺ monocytes were obtained from healthy donors (BioIVT). For differentiation into immature dendritic cells (iDCs), monocytes were cultured for 6 d in RPMI-1640 complete medium (ATCC modification formula; Thermo Fisher, cat. no. A1049101) supplemented with 10% heat-inactivated fetal bovine serum (FBS; Gibco, cat. no. 16140071), 100 ng/mL GM-CSF and 300 ng/mL IL-4 (BioLegend, cat. no. 766206) at 37° C. On day 4, the medium was replaced with fresh medium with supplements. To mature the iDCs, the cells were incubated in RPMI-1640 complete medium supplemented with 10% FBS, 100 ng/mL GM-CSF, 300 ng/mL IL-4, and 5 μg/mL lipopolysaccharide (LPS; Thermo Fisher Scientific, cat. no. 00 4976 93) at 37° C. for 24 h prior to start of the MLR assay. In parallel, purified CD8⁺ T cells obtained from allogeneic healthy donors (BioIVT) were thawed and incubated in RPMI-1640 complete medium supplemented with 10% FBS and 10 ng/mL IL-2 (BioLegend, cat. no. 589106) at 37° C. O/N.

The next day, the LPS-matured dendritic cells (mDCs) and allogeneic CD8⁺ T cells were harvested and resuspended in prewarmed AIM-V medium (Thermo Fisher Scientific, cat. no. 12055091) at 4×10⁵ cells/mL and 4×10⁶ cells/mL, respectively. The mDCs (20,000 cells/well) were incubated with allogeneic naïve CD8⁺ T cells (200,000 cells/well) in the presence of an antibody concentration range (0.001-30 μg/mL) of IgG1-PD1, IgG1-ctrl-FERR, or pembrolizumab (MSD, cat. no. T019263) or in the presence of 30 μg/mL IgG4 isotype control (BioLegend, cat. no. 403702) in AIM-V medium in a 96-well round-bottom plate at 37° C.

After 5 d, cell-free supernatant was transferred from each well to a new 96-well plate and stored at −80° C. until further analysis of cytokine concentrations.

The IFNγ levels were determined using an IFNγ-specific immunoassay (Alpha Lisa IFNγ kit; Perkin Elmer, cat. no. AL217) on an Envision instrument, according to the manufacturer's instructions.

The levels of MCP-1, GM-CSF, IL-1p, IL-2, IL-4, IL-5, IL-6, IL-8, IL-10, IL12-p40, IL-15, IL-17α and TNFα were determined using a customized Luminex® multiplex immunoassay (Millipore, order no. SPR1526) based on the Human TH17 Magnetic Bead Panel (MILLIPLEX®). Briefly, cell-free supernatants were thawed and 10 μL of each sample was added to 10 μL Assay Buffer in wells of a 384-well plate (Greiner Bio-One, cat. no. 781096) prewashed with 1× Wash Buffer. In parallel, 10 μL of Standard or Control in Assay Buffer was added to the wells, after which 10 μL of assay medium was added. Magnetic beads against the different cytokines were mixed and diluted to 1× concentrations in Bead Diluent, after which 10 μL of the mixed beads was added to each well. The plate was sealed and incubated at 4° C., shaking, O/N. Wells were washed three times with 60 μL 1× Wash Buffer. Subsequently, 10 μL of Custom Detection Antibodies was added to each well, and the plate was sealed and incubated at RT, shaking, for 1 h. Next, 10 μL of streptavidin-PE was added to each well, and the plate was sealed and incubated at RT, shaking, for 30 min. Wells were washed three times with 60 μL 1× Wash Buffer as described above, after which beads were resuspended in 75 μL Luminex Sheath Fluid by shaking at RT for 5 min. Samples were run on a Luminex FlexMap 3D system.

At the start and at the end of the MLR assay, expression of PD-1 on the CD8⁺ T cells and expression of PD-L1 on the mDCs was confirmed by flow cytometry using PE-Cy7-conjugated anti-PD-1 (BioLegend, cat. no. 329918; 1:20), allophycocyanin-conjugated anti-PD-L1 (BioLegend, cat. no. 329708; 1:80), BUV496-conjugated anti-CD3 (BD Biosciences, cat. no. 612940; 1:20), and BUV395-conjugated anti-CD8 (BD Biosciences, cat. no. 563795; 1:20). IgG1-PD1 consistently enhanced secretion of IFNγ (FIG. 30) in a dose-dependent manner. IgG1-PD1 also enhanced secretion of MCP-1, GM-CSF, IL-2, IL-6, IL-12p40, IL-17a, IL-10, and TNFα (FIG. 31). Pembrolizumab had a comparable effect on cytokine secretion.

### Example 34: Evaluation of C1q Binding to IgG1-PD1

Binding of complement protein C1q to IgG1-PD1 harboring the FER Fc-silencing mutations in the constant heavy chain region was assessed using activated human CD8⁺ T cells. As a positive control, IgG1-CD52-E430G was included, which has VH and VL domains based on the CD52 antibody CAMPATH-1H and which has an Fc-enhanced backbone that is known to efficiently bind C1q when bound to the cell surface. As non-binding negative control antibodies, IgG1-ctrl-FERR and IgG1-ctrl were included.

Human CD8⁺ T cells were purified (enriched) from buffy coats obtained from healthy volunteers (Sanquin) by negative selection using the RosetteSep™ Human CD8⁺ T Cell Enrichment Cocktail (Stemcell Technologies, cat. no. 15023C.2) or by positive selection via magnetic activated cell sorting (MACS), using CD8 MicroBeads (Miltenyi Biotec, cat. no. 130-045-201) and LS columns (Miltenyi Biotec, cat. no. 130-042-401), all according to the manufacturer's instructions. Purified T cells were resuspended in T-cell medium (Roswell Park Memorial Institute [RPMI]-1640 medium with 25 mM HEPES and L-glutamine [Lonza, cat. no. BE12-115F], supplemented with 10% heat-inactivated donor bovine serum with iron [DBSI; Gibco, cat. no. 20731-030] and penicillin/streptomycin [pen/strep; Lonza, cat. no. DE17-603E]).

Anti-CD3/CD28 beads (Dynabeads™ Human T-Activator CD3/CD28; ThermoFisher Scientific, cat. no. 11132D) were washed with PBS and resuspended in T-cell medium. The beads were added to the enriched human CD8⁺ T cells at a 1:1 ratio and incubated at 37° C., 5% CO₂ for 48 h. Next, the beads were removed using a magnet, and the cells were washed twice in PBS and counted again.

PD-1 expression on the activated CD8⁺ T cells was confirmed by flow cytometry, using IgG1-PD1 (30 μg/mL) and R-phycoerythrin (PE)-conjugated goat-anti-human IgG F(ab′)2 (diluted 1:200 in GMB FACS buffer; Jackson ImmunoResearch, cat. no. 109-116-098), or a commercial PE-conjugated PD-1 antibody (BioLegend, cat. no. 329906; diluted 1:50).

Activated CD8⁺ T cells were seeded in a round-bottom 96-well plate (30,000 or 50,000 cells/well), pelleted, and resuspended in 30 μL assay medium (RPMI-1640 with 25 mM HEPES and L-glutamine, supplemented with 0.1% [w/v] bovine serum albumin fraction V [BSA; Roche, cat. no. 10735086001] and penicillin/streptomycin). Subsequently, 50 μL of IgG1-PD1, IgG1-ctrl-FERR, IgG1-CD52-E430G, or IgG1-ctrl (final concentrations of 1.7×10⁻⁴-30 μg/mL in 3-fold dilution steps in assay medium) was added to each of the wells and incubated at 37° C. for 15 min to allow the antibodies to bind to the cells.

Human serum (20 μL/well; Sanquin, lot 20L15-02), as a source of C1q, was added to a final concentration of 20%. Cells were incubated on ice for 45 min, followed by two washes with cold GMB FACS buffer and incubation with 50 μL fluorescein isothiocyanate (FITC)-conjugated rabbit anti-human C1q (final concentration of 20 μg/mL [DAKO, cat no. F0254]; diluted 1:75 in GMB FACS buffer) in the presence or absence of allophycocyanin-conjugated mouse-anti-CD8 (BD Biosciences, cat. no. 555369; diluted 1:50 in GMB FACS buffer) in the dark at 4° C. for 30 min. Cells were washed twice with cold GMB FACS buffer, resuspended in 20 μL of GMB FACS buffer supplemented with 2 mM ethylenediaminetetraacetic acid (EDTA; Sigma-Aldrich, cat. no. 03690) and 4′,6-diamidino-2-phenylindole (DAPI) viability dye (1:5,000; BD Pharmingen, cat. no. 564907). C1q binding to viable cells (as identified by DAPI exclusion) was analyzed by flow cytometry on an IntelliCyt® iQue Screener PLUS (Sartorius) or iQue3 (Sartorius). Binding curves were analyzed using non-linear regression analysis (sigmoidal dose-response with variable slope) using GraphPad Prism software.

Whereas dose-dependent C1q binding was observed to membrane-bound IgG1-CD52-E430G, no C1q binding was observed to membrane-bound IgG1-PD1 or to the non-binding control antibodies (FIG. 32).

These results indicate that the functionally inert backbone of IgG1-PD1 does not bind C1q.

### Example 35: Binding of IgG1-PD1 to Fcγ Receptors as Determined by SPR

The binding of IgG1-PD1 to immobilized FcγRs (FcγRIa, FcγRIIa, FcγRIIb and FcγRIIIa) was assessed in vitro by SPR. Both polymorphic variants were included for FcγRIIa (H131 and R131) and FcγRIIIa (V158 and F158). As a positive control for FcγR binding, IgG1-ctrl with a wild-type Fc region was included.

In a first experiment, binding of IgG1-PD1, or IgG1-ctrl to immobilized human recombinant FcγR variants (FcγRIa, FcγRIIa, FcγRIIb, and FcγRIIIa) was analyzed using a Biacore 8K SPR system. In a second set of experiments, using the same method, binding of IgG1-PD1, nivolumab (Bristol-Meyers Squibb, lot no. ABP6534), pembrolizumab (Merck Sharp & Dohme, lot no. U013442), dostarlimab (GlaxoSmithKline, lot. no. 1822049), cemiplimab (Regeneron, lot no. 1F006A), IgG1-ctrl, or IgG4-ctrl was analyzed.

Biacore Series S Sensor Chips CM5 (Cytiva, cat. no. 29104988) were covalently coated with anti-Histidine (His) antibody using amine-coupling and His capture kits (Cytiva, cat. no. BR100050 and cat. no. 29234602) according to the manufacturer's instructions. FcγRIa, FcγRIIa (H131 and R131), FcγRIIb and FcγRIIIa (V158 and F158) (SinoBiological, cat. no. 10256-H08S-B, 10374-H08H1, 10374-H27H, 10259-H27H, 10389-H27H1, and 10389-H27H, respectively) diluted in HBS-EP+(Cytiva, cat. no. BR100669) were captured onto the surface of the anti-His coated sensor chip with a flow rate of 10 μL/min and a contact time of 60 seconds to result in captured levels of approximately 350-600 resonance units (RU).

After three start-up cycles of HBS-EP+ buffer, test antibodies (IgG1-PD1, nivolumab, pembrolizumab, dostarlimab, cemiplimab, IgG1-ctrl, or IgG4-ctrl) were injected to generate binding curves, using antibody ranges as indicated in Table 17. Each sample that was analyzed on a surface with captured FcγRs (active surface) was also analyzed on a parallel flow cell without captured FcγRs (reference surface), which was used for background correction. The third start-up cycle containing HBS-EP+ as a (mock) analyte was subtracted from other sensorgrams to yield double-referenced data.

At the end of each cycle, the surface was regenerated using 10 mM Glycine-HCl pH 1.5 (Cytiva, cat. no. BR100354). Sensorgrams were generated using Biacore Insight Evaluation software (Cytiva) and a four-parameter logistic fit was applied on end-point measurements (binding plateau versus post-capture baseline). Data of the first experiment (n=1; qualified SPR assay) is shown in FIG. 33; data of the second set of experiments (n=3) is shown in FIG. 34.

TABLE 17
Test conditions for binding to individual FcγRs

| | Anti-PD-1 antibody concentration range tested | | |
|---|---|---|---|
| | | | Lowest |
| | Start concentration | | concentration |
| FcγR | (nM) | Fold dilution | (nM) |
| FcγRIa | 3,000 | 1:3 | 0.02 |
| FcγRIIa-H131 | 10,000 | 1:2.5 | 0.42 |
| FcγRIIa-R131 | 10,000 | 1:2.5 | 0.42 |
| FcγRIIb | 10,000 | 1:2 | 4.9 |
| FcγRIIIa-V158 | 10,000 | 1:3 | 0.06 |
| FcγRIIIa-F158 | 10,000 | 1:2.5 | 0.42 |

Results from the first experiment showed binding of IgG1-ctrl to all FcγRs, while no binding was observed for IgG1-PD1 to FcγRIa, FcγRIIa (H131 and R131), FcγRIIb, and FcγRIIIa (V158 and F158) (FIG. 31).

Results from the second set of experiments confirmed lack of FcγR binding for IgG1-PD1 (FIG. 32). IgG4-ctrl and the other anti-PD-1 antibodies tested (nivolumab, pembrolizumab, dostarlimab, and cemiplimab; all of the IgG4 subclass) demonstrated clear binding to FcγRIa, FcγRIIa-H131, FcγRIIa-R131, and FcγRIIb, and minimal to very minimal binding to FcγRIIIa-F158 and FcγRIIIa-V158.

These data confirm lack of FcγR binding for the Fc domain of IgG1-PD1 and demonstrate FcγR binding to nivolumab, pembrolizumab, dostarlimab, and cemiplimab. Taken together, these data suggest that the Fc domain of IgG1-PD1 is unable to induce FcγR-mediated effector functions (ADCC, ADCP).

### Example 36: Binding of IgG1-PD1 to Cell Surface Expressed FcγRIa as Determined by Flow Cytometry

Binding of IgG1-PD1, nivolumab, pembrolizumab, dostarlimab, and cemiplimab to human cell surface expressed FcγRIa was analyzed using flow cytometry.

FcγRIa was expressed on transiently transfected CHO-S cells, and cell surface expression was confirmed by flow cytometry using FITC-conjugated anti-FcγRI antibody (BioLegend, cat. no. 305006; 1:25). Binding of anti-PD-1 antibodies to transfected_CHO-S cells was assessed as described in Example 27. Briefly, antibody dilutions (final concentrations: 1.69×10⁻⁴-10 μg/mL, 3-fold dilutions) of IgG1-PD1, nivolumab (Bristol-Meyers Squibb, lot no. ABP6534), pembrolizumab (Merck Sharp & Dohme, lot no. U013442), dostarlimab (GlaxoSmithKline, lot. no. 1822049), cemiplimab (Regeneron, lot no. 1F006A), IgG1-ctrl, and IgG1-ctrl-FERR were prepared in GMB FACS buffer. Cells were centrifuged, supernatant was removed, and cells (30,000 cells in 50 μL) were incubated with 50 μL of the antibody dilutions for 30 min at 4° C. Cells were washed twice with GMB FACS buffer and incubated with 50 μL secondary antibody (PE-conjugated goat-anti-human IgG F(ab′)₂; 1:500) for 30 min at 4° C., protected from light. Cells were washed twice with GMB FACS buffer and resuspended in GMB FACS buffer supplemented with 2 mM EDTA and DAPI viability marker (1:5,000).

Antibody binding to viable cells was analyzed by flow cytometry on an Intellicyt iQue PLUS Screener (Intellicyt Corporation) using FlowJo software by gating on PE-positive, DAPI-negative cells. Binding curves were analyzed using non-linear regression analysis (four-parameter dose-response curve fits) in GraphPad Prism.

In the flow cytometry binding assays, the positive control antibody IgG1-ctrl (with a wild-type Fc region) showed binding to cells transiently expressing FcγRIa, while no binding was observed for the negative control antibody IgG1-ctrl-FERR (with an Fc region containing the FER inertness mutations and an additional, in the context of this study functionally irrelevant, K409R mutation) (FIG. 35). No binding was observed for IgG1-PD1, while concentration-dependent binding was observed for pembrolizumab, nivolumab, cemiplimab, and dostarlimab.

These data confirm lack of FcγRIa binding for the Fc domain of IgG1-PD1 and demonstrate FcγRIa binding to nivolumab, pembrolizumab, dostarlimab, and cemiplimab. Taken together, these data suggest that the Fc domain of IgG1-PD1 is unable to induce FcγRIa-mediated effector functions.

### Example 37: Binding to Neonatal Fc Receptor by IgG1-PD1

The neonatal Fc receptor (FcRn) is responsible for the long plasma half-life of IgG by protecting IgG from degradation. IgG binds to FcRn in an acidic (pH 6.0) endosomal environment but dissociates from FcRn at neutral pH (pH 7.4). This pH-dependent binding of antibodies to FcRn causes recycling of the antibody together with FcRn, preventing intracellular antibody degradation, and therefore is an indicator for the in vivo pharmacokinetics of that antibody. The binding of IgG1-PD1 to immobilized FcRn was assessed in vitro at pH 6.0 and pH 7.4 by means of surface plasmon resonance (SPR).

Binding of IgG1-PD1 to immobilized human FcRn was analyzed using a Biacore 8K SPR system. Biacore Series S Sensor Chips CM5 (Cytiva, cat. no. 29104988) were covalently coated with anti-histidine (His) antibody using amine coupling and His capture kits (Cytiva, cat. no. BR100050 and cat. no. 29234602) according to the manufacturer's instructions. FcRn (SinoBiological, cat. no. CT071-H27H-B) diluted to a 5 nM coating concentration in PBS-P+ buffer pH 7.4 (Cytiva, cat. no. 28995084) or in PBS-P+ buffer with the pH adjusted to 6.0 (by addition of hydrochloric acid [Sigma-Aldrich, cat. no. 07102]) was captured onto the surface of the anti-His coated sensor chip with a flow rate of 10 μL/min and a contact time of 60 seconds. This resulted in captured levels of approximately 50 RU. After three start-up cycles of pH 6.0 or pH 7.4 PBS-P+ buffer, test antibodies (6.25-100 nM two-fold dilution series of IgG1-PD1, pembrolizumab (MSD, lot. no. T019263), or nivolumab (Bristol-Myers Squibb, lot. no. ABP6534) in pH 6.0 or pH 7.4 PBS-P+ buffer) were injected to generate binding curves. Each sample that was analyzed on a surface with captured FcRn (active surface) was also analyzed on a parallel flow cell without captured FcRn (reference surface), which was used for background correction. The third start-up cycle containing HBS-EP+ as a (mock) analyte was subtracted from other sensorgrams to yield double-referenced data. At the end of each cycle, the surface was regenerated using 10 mM Glycine HCl pH 1.5 (Cytiva, cat. no. BR100354). The data were analyzed using the predefined “Multi-cycle kinetics using capture” evaluation method in the Biacore Insight Evaluation software (Cytiva). Data is based on three separate experiments with technical duplicates.

At pH 6.0, IgG1-PD1 bound FcRn with an average affinity (K_{D}) of 50 nM (Table 18), which is comparable to an IgG1-ctrl antibody with a wild-type Fc region (a broad range of affinities is reported for wild-type IgG1 molecules in literature; in previous in-house experiments with the same assay set-up, an average K_{D} of 34 nM was measured for IgG1-ctrl across 12 data points). The affinity of pembrolizumab and nivolumab was approximately two-fold lower (K_{D} of 116 nM and 133 nM, respectively). No FcRn binding was observed at pH 7.4 (not shown). Taken together, these results demonstrate that the FER inertness mutations in the IgG1-PD1 Fc region do not affect FcRn binding and suggest that IgG1-PD1 will retain typical IgG pharmacokinetic properties in vivo.

TABLE 18
Affinity for FcRn as determined by SPR. Binding of IgG1-PD1, pembrolizumab, and nivolumab
to sensor chips coated with human FcRn was analyzed by SPR. The average affinity and
SD are based on three independent measurements with technical duplicates.

| | K_{D} (M) | | kₐ (1/M × s) | | k_{d} (1/s) | |
|---|---|---|---|---|---|---|
| Antibody | Average | SD | Average | SD | Average | SD |
| IgG1-PD1 | 4.99 × 10⁻⁸ | 6.85 × 10⁻⁹ | 8.65 × 10⁵ | 6.79 × 10⁴ | 4.29 × 10⁻² | 4.88 × 10⁻³ |
| Nivolumab | 1.33 × 10⁻⁷ | 2.01 × 10⁻⁸ | 5.18 × 10⁵ | 4.70 × 10⁴ | 6.89 × 10⁻² | 1.19 × 10⁻² |
| Pembrolizumab | 1.16 × 10⁻⁷ | 1.69 × 10⁻⁸ | 5.46 × 10⁵ | 4.42 × 10⁴ | 6.36 × 10⁻² | 1.15 × 10⁻² |

Abbreviations: K_{D} = equilibrium dissociation constant; kₐ = association rate constant; k_{d} = dissociation rate constant or off-rate; SD = standard deviation.

### Example 38: Pharmacokinetic Analysis of IgG1-PD1 in Absence of Target Binding

The pharmacokinetic properties of IgG1-PD1 were analyzed in mice. PD-1 is expressed mainly on activated B and T cells, and as such, its expression is expected to be limited in non-tumor bearing SCID mice, which lack mature B and T cells. Furthermore, IgG1-PD1 shows substantially reduced cross-reactivity to cells transiently overexpressing mouse PD-1 (Example 27). Therefore, the pharmacokinetic (PK) properties of IgG1-PD1 in non-tumor bearing SCID mice are expected to reflect the PK properties of IgG1-PD1 in absence of target binding.

The mice in this study were housed in the Central Laboratory Animal Facility (Utrecht, the Netherlands). All mice were kept in individually ventilated cages with food and water provided ad libitum. All experiments were in compliance with the Dutch animal protection law (WoD) translated from the directives (2010/63/EU) and were approved by the Dutch Central Commission for animal experiments and by the local Ethical committee). SCID mice (C.B-17/IcrHan®Hsd-Prkdc^{scid}, Envigo) were injected intravenously with 1 or 10 mg/kg IgG1-PD1, using 3 mice per group. Blood samples (40 μL) were collected from the saphenous vein or the cheek veins at 10 min, 4 h, 1 day, 2 days, 8 days, 14 days, and 21 days after antibody administration. Blood was collected into vials containing K₂-ethylenediaminetetraacetic acid and stored at −65° C. until determination of antibody concentrations.

By a total human IgG (hIgG) electrochemiluminescence immunoassay (ECLIA), specific hIgG concentrations were determined. Meso Scale Discovery (MSD) standard plates (96-well MULTI-ARRAY plate, cat. no. L15XA-3) were coated with mouse anti-hIgG capture antibody (IgG2amm-1015-6A05) diluted in PBS (Lonza, cat. no. BE17-156Q) for 16-24 h at 2-8° C. After washing the plate with PBS-Tween (PBS-T; PBS supplemented with 0.05% (w/v) Tween-20 [Sigma, cat. no. P1379]) to remove non-bound antibody, the unoccupied surfaces were blocked for 60±5 min at RT (PBS-T supplemented with 3% (w/v) Blocker-A [MSD, cat. no. R93AA-1]) followed by washing with PBS-T. Mouse plasma samples were initially diluted 50-fold (2% mouse plasma) in assay buffer (PBS-T supplemented with 1% (w/v) Blocker-A). To create a reference curve, IgG1-PD1 (same batch as the material used for injection) was diluted (measuring range: 0.156-20.0 μg/mL; anchor points: 0.0781 and 40.0 μg/mL) in Calibrator Diluent (2% mouse plasma [K₂EDTA, pooled plasma, BIOIVT, cat. no. MSE00PLK2PNN] in assay buffer). To accommodate for the expected wide range of antibody concentrations present in the samples, samples were additionally diluted 1:10 or 1:50 in Sample Diluent (2% mouse plasma in assay buffer). The coated and blocked plates were incubated with 50 μL diluted mouse samples, the reference curve, and appropriate quality control samples (pooled mouse plasma spiked with IgG1-PD1, covering the range of the reference curve) at RT for 90±5 min. After washing with PBS-T, the plates were incubated with SULFO-TAG-conjugated mouse anti-hIgG detection antibody IgG2amm-1015-4A01 at RT for 90±5 min. After washing with PBS-T, immobilized antibodies were visualized by adding Read Buffer (MSD GOLD Read Buffer, cat. no. R92TG-2) and measuring light emission at ~620 nm using an MSD Sector S600 plate reader. Processing of analytical data was performed using SoftMax Pro GxP Software v7.1. Extrapolation below the run lower limit of quantitation (LLOQ) or above the upper limit of quantitation (ULOQ) was not allowed.

The plasma clearance profile of IgG1-PD1 in absence of target binding was comparable to the clearance profile of a wild-type human IgG1 antibody in SCID mice predicted by a two-compartment model based on IgG1 clearance in humans (Bleeker et al., 2001, Blood. 98(10):3136-42) (FIG. 36). No clinical observations were noted, and no body weight loss was observed.

In conclusion, these data indicate that the PK properties of IgG1-PD1 are comparable to those of normal human IgG antibodies in absence of target binding.

### Example 39: Antitumor Activity of IgG1-PD1 in Human PD-1 Knock-In Mice

IgG1-PD1 shows only limited binding to cells transiently overexpressing mouse PD-1 (Example 27). Therefore, to assess antitumor activity of IgG1-PD1 in vivo, C57BL/6 mice engineered to express the human PD-1 extracellular domain (ECD) in the mouse PD-1 gene locus (hPD-1 knock-in [KI] mice) were used.

All animal experiments were performed at Crown Bioscience Inc. and approved by their Institutional Animal Care and Use Committee (IACUC) prior to execution. Animals were housed and handled in accordance with good animal practice as defined by the regulations of the Association for Assessment and Accreditation of Laboratory Animal Care (AAALAC). Female homozygous human PD-1 knock-in mice on a C57BL/6 background (hPD-1 KI mice; Beijing Biocytogen Co., Ltd; C57BL/6-Pdcd1^{tm1(PDCD1)}/Bcgen, stock no. 110003), 7-9 weeks old, were injected subcutaneously (SC) with syngeneic MC38 colon cancer cells (1×10⁶ cells) in the right lower flank. Tumor growth was evaluated using a caliper (three times per week after randomization), and tumor volumes (mm³) were calculated from caliper measurements as: tumor volume=0.5×(length×width²), where the length is the longest tumor dimension, and the width is the longest tumor dimension perpendicular to the length. Mice were randomized (9 mice per group) based on tumor volume and body weight when tumors had reached an average volume of approximately 60 mm³ (denoted as day 0). At the start of treatment, mice were injected intravenously (IV; dosing volume 10 mL/kg in PBS) with 0.5, 2, or 10 mg/kg IgG1-PD1 or pembrolizumab (obtained from Merck by Crown Bioscience Inc., lot no. T042260), or with 10 mg/kg isotype control antibody IgG1-ctrl-FERR. Subsequent doses were administered intraperitoneally (IP). A dosing regimen of two doses weekly for three weeks (2QW×3) was used. Animals were monitored daily for morbidity and mortality and monitored routinely for other clinical observations. The experiment ended for individual mice when the tumor volume exceeded 1,500 mm³ or when the animals reached other humane endpoints.

To compare progression-free survival between the groups, curve fits were applied to the individual tumor growth graphs to establish the day of progression beyond a tumor volume of 500 mm³ for each mouse. These day values were plotted in a Kaplan-Meier survival curve and used to perform a Mantel-Cox analysis between individual curves using SPSS software. The difference in tumor volumes between the groups was compared using a nonparametric Mann-Whitney analysis (in GraphPad Prism) on the last day that all groups were still intact (ie, until the first tumor-related death in the study, ie, Day 11). P-values are presented accompanied by median values (per group) including the 95% confidence interval of the difference in median (Hodges Lehmann).

The mice showed no signs of illness, but two mice were found dead (one in the 2 mg/kg IgG1-PD1 group and one in the 2 mg/kg pembrolizumab treatment group). The cause of these deaths was undetermined.

Treatment with IgG1-PD1 and pembrolizumab inhibited tumor growth at all doses tested (FIG. 37A). On day 11, the last day that all treatment groups were complete, tumors in mice treated with IgG1-PD1 or pembrolizumab were significantly smaller at all doses tested than tumors in mice treated with 10 mg/kg IgG1-ctrl-FERR (FIG. 37B). In addition, at 10 mg/kg, tumor volumes in mice treated with IgG1-PD1 were significantly smaller than in mice treated with an equivalent dose of pembrolizumab (Mann-Whitney test, p=0.0188).

Treatment with IgG1-PD1 or pembrolizumab significantly increased progression-free survival (PFS) at all doses tested compared to mice treated with 10 mg/kg IgG1-ctrl-FERR (FIG. 37C). At 10 mg/kg, progression-free survival in mice treated with IgG1-PD1 was significantly extended as compared to mice treated with pembrolizumab (median PFS 10 mg/kg IgG1-PD1: 20.56 days, median PFS 10 mg/kg pembrolizumab: 13.94 days; P-value=0.0021).

In conclusion, IgG1-PD1 exhibited potent antitumor activity in MC38 tumor-bearing hPD-1 KI mice.

### Example 40. PD Activity of IgG1-PD1 in Human PD-1 Knock-In Mice

IgG1-PD1 showed potent antitumor activity in MC38 tumor-bearing hPD-1 KI mice (Example 39). To explore the pharmacodynamic effects of IgG1-PD1 treatment, MC38 tumor-bearing hPD-1 KI mice were treated with IgG1-PD1, and blood, spleen, and tumor samples were collected at predetermined timepoints. The effect of IgG1-PD1 treatment on immune cells was determined using flow cytometry and immunohistochemistry (IHC).

The MC38 tumor-bearing hPD-1 KI mouse model was established as described in Example 39. Mice were randomized (12 mice per group) based on tumor volume when tumors had reached an average volume of approximately 60 mm³ (denoted as Day 0). At the start of treatment, mice were injected IV (dosing volume 10 mL/kg in PBS) with 0.5 or 10 mg/kg IgG1-PD1, with 10 mg/kg pembrolizumab (obtained from Merck by Crown Bioscience Inc., lot no. U036695), or with 10 mg/kg isotype control antibody IgG1-ctrl-FERR on Day 0, 3, and 7. Animals were monitored daily for morbidity and mortality and monitored routinely for other clinical observations. The mice showed no signs of illness. On Day 2, 4, and 8, animals were euthanized, and blood was collected through cardiac puncture (4 mice per treatment group at each time point) for the immunophenotyping of peripheral blood cells. In addition, the spleens and tumors were harvested. The tumors were formalin-fixed and paraffin-embedded for IHC analysis.

The spleens were enzymatically dissociated using the gentleMACS™ Dissociator (130-096-427, Miltenyi) according to the manufacturer's instructions. The resulting cell suspension was filtered through a 70 μm cell strainer (Falcon, cat. no. 352350), washed with 5 mL of FACS wash buffer (10% FBS [Gibco, cat. no. 10099-141], 40 mM EDTA [Boston BioProducts, cat. no. BM-711-K], in PBS). Red blood cells were lysed using RBC Lysing Buffer (Bio-gems, cat. no. 64010-00-100). Cells were washed twice with FACS wash buffer and resuspended in PBS for cell counting.

The blood samples and dissociated spleen samples were incubated with Mouse BD Fc Block™ (BD Biosciences, cat. no. 553141) in the dark at 4° C. for 10 min, after which cells were stained with the antibody panel described in Table 19 diluted in Fc blocking buffer at 4° C. for 30 min. Subsequently, the blood samples were incubated with RBC Lysing buffer for an additional 10 min incubation at RT. Next, cells from both the blood and dissociated spleen samples were washed with wash buffer three times. To each sample, 100 μL of 123count eBeads (eBioscience, cat. no. 01-1234-42) was added, after which the samples were analyzed by flow cytometry. Flow cytometry data were analyzed using Kaluza Analysis Software.

TABLE 19
Immunophenotyping antibody panel

| Marker | Fluorophore | Clone | Vendor | Cat. no. | Isotype | Dilution |
|---|---|---|---|---|---|---|
| CD45 | PerCP-Cy5.5 | 30-F11 | BioLegend | 103132 | Rat IgG2b, κ | 1:50 |
| CD3 | BUV395 | 17A2 | BD Biosciences | 740268 | Rat IgG2b, κ | 1:50 |
| CD4 | BV510 | GK1.5 | BioLegend | 100449 | Rat IgG2b, κ | 1:40 |
| CD8 | PE-eF610 | 53-6.7 | eBioscience | 61-0081-82 | Rat IgG2a, κ | 1:50 |
| CD44 | AF700 | IM7 | BioLegend | 103026 | Rat IgG2b, κ | 1:50 |
| CD62L | BV785 | MEL-14 | BioLegend | 104440 | Rat IgG2a, κ | 1:50 |
| CD19 ^{a} | FITC | 6D5 | BioLegend | 115506 | Rat IgG2a, κ | 1:50 |
| CD11b ^{a} | FITC | M1/70 | BioLegend | 101206 | Rat IgG2b, κ | 1:50 |
| I-A/I-E | BV711 | M5/114.15.2 | BioLegend | 107643 | Rat IgG2b, κ | 1:50 |
| (MHC II) | | | | | | |
| Fixable | eF780 | N.A. | eBioscience | 65-0865-14 | N.A. | 1:100 |
| Viability Dye | | | | | | |

^{a} CD19 and CD11b were combined in a single channel to exclude cells expressing CD19 and/or CD11b.
Abbreviations: BUV = Brilliant Ultra Violet; BV = Brilliant Violet; CD = cluster of differentiation; Cy = cyanine; eF = eFluor; FITC = fluorescein isothiocyanate; IgG = immunoglobulin G; MHC = major histocompatibility complex; N.A. = not applicable; PE = phycoerythrin; PerCP = peridinin-chlorophyll-protein.

Expression of CD3, CD4, CD8, and granzyme B (GZMB) in xenograft tumor tissues was assessed in IHC using rabbit anti-CD3E (Ventana, clone 2GV6, cat. no. 790-4341; final concentration 0.4 μg/mL), rabbit anti-CD4 (Abcam, clone EPR19514, cat. no. ab183685; final concentration 5 μg/mL), rabbit anti-CD8 (Cell Signaling, clone D4W2Z, cat. no. 98941; diluted 1:200), and rabbit anti-GZMB antibody (Abcam, clone EPR22645-206, cat. no. ab255598; final concentration 5 μg/mL) followed by an anti-rabbit specific detection protocol (OmniMap DAB anti-Rb detection kit, Roche, cat. no. 05269679001, for the CD8 IHC assay combined with HQ signal amplification kit, Roche, cat. no. 06472320001) to avoid binding to potential remaining mouse IgG. Cellular quantitation within viable tumor regions was performed on digital images with tailored image analysis algorithms using HALO software (Indica Labs). Cellular quantitation readouts were generated by calculating the percentage of marker-positive cells of all nucleated cells within viable (non-necrotic) tumor areas.

Compared to the non-binding control antibody IgG1-ctrl-FERR, treatment with 10 mg/kg IgG1-PD1 resulted in a clear trend of an increased number of T cells (CD3⁺, CD4⁺, and CD8⁺) in peripheral blood on Day 8, while treatment with 10 mg/kg pembrolizumab led to a statistically significant decrease in the number of peripheral T cells (FIG. 38). In parallel, the percentage of effector memory (CD44⁺CD62L⁻) CD8⁺ T cells was significantly increased on Day 8 in the spleens of mice treated with 10 mg/kg IgG1-PD1, but not in the spleens of mice treated with 10 mg/kg pembrolizumab (FIG. 39A), when compared to control treatment. A concomitant significant decrease in the percentage of naïve (CD44⁻CD62L⁺) CD8⁺ T cells was observed in the spleens of mice treated with 10 mg/kg IgG1-PD1 when compared to control mice and mice treated with 10 mg/kg pembrolizumab. Treatment with 10 mg/kg IgG1-PD1 significantly increased the percentage of MHC class II⁺ CD8⁺ T cells in the spleen on Day 8, suggestive of increased T-cell activation (FIG. 39B). A comparable increase in the percentage of MHC class II⁺ CD8⁺ T cells was observed in the spleens of mice treated with 10 mg/kg pembrolizumab.

On Day 8, the number of intratumoral CD3⁺ and CD8⁺ T cells was significantly lower in mice treated with 10 mg/kg pembrolizumab than in the control mice (FIG. 40A-C). Furthermore, on Day 8, the percentage of intratumoral cells expressing the cytotoxic effector molecule GZMB was significantly higher in mice treated with 10 mg/kg IgG1-PD1 than in mice treated with 10 mg/kg pembrolizumab and in control mice (FIG. 40D).

In conclusion, in vivo antitumor activity of IgG1-PD1 was associated with an increase in the number of peripheral blood and intratumoral T cells, an increased percentage of effector memory and activated (MHC class II⁺) CD8⁺ T cells in the spleen, and an increased percentage of intratumoral GZMB⁺ cells. In comparison, pembrolizumab-treated mice showed limited pharmacodynamic changes.

### Example 41. Macrophage Binding by IgG1-PD1

IgG1-PD1 showed no binding to FcγRs, while nivolumab, pembrolizumab, dostarlimab, and cemiplimab did (Example 35). The capacity of these antibodies to bind FcγR-expressing M2c-like macrophages was assessed in vitro using flow cytometry.

Human peripheral blood mononuclear cells (PBMCs) were purified from buffy coats of three healthy human donors (Sanquin blood supply foundation, the Netherlands) in LeucoSep™ tubes (Greiner, cat. no. 227290) by density gradient centrifugation (20 min at 800×g, with low brake) over Lymphocyte Separation Medium (Promocell, cat. no. C-44010), according to the manufacturer's instructions. Human monocytes were purified from PBMCs by MACS technology using anti-CD14 MicroBeads (Miltenyi; cat. no. 130-050-201), according to the manufacturer's instructions. CD14⁺ monocytes were resuspended at a density of 1.0×10⁶ cells/mL in CellGenix® GMP DC medium (CellGenix, cat. no. 20801-0500) supplemented with 50 ng/mL M-CSF (Gibco, cat. no. PHC9501). For the polarization of monocytes towards M2c-like macrophages, purified monocytes were plated in 100 mm² Nunc™ dishes with UpCell™ Surface (8×10⁶ cells/dish at a density of 1.0×10⁶ cells/mL; Thermo Fisher Scientific, cat. no. 174902) in M-CSF-supplemented CellGenix GMP DC medium and cultured (37° C., 5% CO₂) for 7 d, followed by 3 d of culture in CellGenix GMP DC medium supplemented with 50 ng/mL M-CSF, 50 ng/mL IL-4 (R&D Systems, cat. no. 204-IL), and 50 ng/mL IL-10 (R&D Systems, cat. no. 1064-IL/CF). Next, macrophages were detached from the culture dish surface by leaving the dish at RT for 40 to 60 min. Detached macrophages were pelleted by centrifugation (5 min at 300×g), counted, and resuspended at a density of 1.5×10⁶ cells/mL in CellGenix GMP DC medium. The M2c-like phenotype of the monocyte-derived macrophages was confirmed by flow cytometry using a mixture of Brilliant Violet (BV)421-conjugated anti-human CD163 (BioLegend, cat. no. 333612; diluted 1:200) and BV711-conjugated anti-human CD206 (BioLegend, cat. no. 321136; diluted 1:200). FcγR and PD-1 expression on M2c-like macrophages was assessed using FITC-conjugated anti-human CD64 (FcγRIa; BioLegend, cat. no. 305006; diluted 1:25), FITC-conjugated anti-human CD32 (FcγRII; BD Pharmingen, cat. no. 552883; diluted 1:50), PE-conjugated anti-human CD16a (FcγRIIIa; BD Pharmingen, cat. no. 555407; diluted 1:50), PE-conjugated anti-PD-1 antibody (BioLegend, cat. no. 329906, diluted 1:50), a FITC-conjugated IgG1 isotype control (BioLegend, cat. no. 400108; diluted 1:25), and a PE-conjugated IgG1 isotype control (BD Pharmingen, cat. no. 555749; diluted 1:50). The M2c-like macrophages were incubated with IgG1-PD1, pembrolizumab (MSD, lot no. U013442), nivolumab (Bristol-Myers Squibb, lot no. ABP6534), IgG4 isotype control (BioLegend, cat. no. 403702), IgG1-ctrl, and IgG1-ctrl-FERR for 24 h, and washed twice with FACS buffer. Cells were incubated with PE-conjugated goat anti-human IgG F(ab′)2 (Jackson ImmunoResearch, cat. no. 109-116-097; diluted 1:200) diluted in FACS buffer at 4° C. for 30 min. After two washes with FACS buffer, cells were resuspended in FACS buffer supplemented with the viability dye 4′,6-diamidino-2-phenylindole (DAPI; BD Pharmingen, cat. no. 564907; diluted 1:5,000) and subsequently measured on a BD LSRFortessa™ Cell Analyzer and analyzed in FlowJo.

Binding of IgG1-PD1, pembrolizumab, and nivolumab to FcγRs expressed on the cell membrane was evaluated using human monocyte-derived M2c-like macrophages (from three healthy donors). Expression of FcγRIa, FcγRII, and FcγRIIIa and absence of PD-1 expression were first confirmed by flow cytometry (FIG. 41A). IgG1-ctrl, which has a wild-type IgG1 Fc domain, Fc-inert IgG1-ctrl-FERR, and an IgG4 isotype control were included as controls. Whereas IgG1-ctrl showed efficient binding to the M2c-like macrophages after 24 h of incubation, no binding was observed for IgG1-PD1 or IgG1-ctrl-FERR for any of the donors tested (FIG. 41B). Conversely, pembrolizumab, nivolumab, and the IgG4 isotype control antibody all showed binding above that of the background control. Together, these data demonstrate that IgG1-PD1 does not bind to FcγR-expressing M2c-like macrophages, while pembrolizumab and nivolumab do.

### Example 42. FcγR Signaling Induced by IgG1-PD1

IgG1-PD1 showed no binding to FcγRs or to M2c-like macrophages, while anti-PD-1 antibodies with an IgG4 backbone did (Examples 35 and 41). The capacity of these antibodies to induce FcγR signaling was assessed in vitro using cell-based Fc effector activity reporter assays.

FcγRI, FcγRIIa-H131, FcγRIIa-R131, and FcγRIIb signaling induced by IgG1-PD1, nivolumab, pembrolizumab, dostarlimab, and cemiplimab was evaluated using bioluminescent cell-based reporter assays (Promega, cat. no. GA1341, G988A, CS178B11, and G988ACS1781E01, respectively), essentially as described by the manufacturer. Briefly, CHO cells transfected with PD-1 (generated in-house; Example 28) were preincubated with serially diluted IgG1-PD1, pembrolizumab (MSD, lot no. W003098), nivolumab (Bristol-Myers Squibb, lot no. 8006768), dostarlimab (GlaxoSmithKline, lot. no. 1822049), cemiplimab (Regeneron, lot no. 1F006A), IgG1-ctrl-FERR, or IgG4 isotype (BioLegend, cat. no. 403702) (final in-assay concentrations in FcγRI assay: 30-1.23×10⁻⁷ μg/mL in 25-fold dilutions; final assay concentrations in other FcγR assays: 30-0.00192 μg/mL in 5-fold dilutions) for 15 min at 37° C., 5% C02. IgG1-CD52-E430G, with the hexamerization-enhancing E430G mutation, was included as a positive control. Genetically engineered FcγRI, FcγRIIa-H (FcγRIIa-H131), FcγRIIa-R (FcγRIIa-R131), and FcγRIIb effector cells were added to the cultures at a 1:1 ratio, after which the samples were incubated for 5 h at 37° C., 5% CO₂. Next, the samples were incubated at RT with reconstituted Bio-Glo™ for 10 min at RT, after which luminescence (in RLU) was measured using an EnVision Multilabel Plate Reader (PerkinElmer).

Membrane-bound IgG1-CD52-E430G, with the E430G hexamerization-enhancing mutation, induced strong FcγRI, FcγRIIa-R131, FcγRIIa-H131, and FcγRIIb signaling. Membrane-bound pembrolizumab, nivolumab, cemiplimab, and dostarlimab (all of the IgG4 subclass) also induced FcγRI, FcγRIIa-R131, FcγRIIa-H131, and FcγRIIb signaling, but to a lesser extent, while membrane-bound IgG1-PD1 and the non-binding control antibodies (IgG1-ctrl-FERR, IgG4 isotype) did not (FIG. 42).

Taken together, these data demonstrate that membrane-bound nivolumab, pembrolizumab, dostarlimab, and cemiplimab induce FcγRI, FcγRIIa-R131, FcγRIIa-H131, and FcγRIIb signaling. In contrast, membrane-bound IgG1-PD1 is unable to induce FcγR-mediated signaling, confirming the functional inertness of the Fc domain of IgG1-PD1.

### Example 43: Induction of Proliferation of Polyclonally Activated Human T Cells by IgG1-CD27-A-P329R-E345R in Combination with DuoBody-PD-L1×4-1BB

The effect of IgG1-CD27-A-P329R-E345R in combination with DuoBody-PD-L1×4-1BB on activated human T cells was analyzed by flow cytometry using freshly isolated human healthy donor PBMC, in which T cells were polyclonally stimulated with a CD3 antibody. Human peripheral blood mononuclear cells (PBMCs) were freshly isolated from human healthy donor buffy coats by low density gradient centrifugation using lymphocyte separation medium (PromoCell, cat. no. C-44010) and LeucoSep tubes (Greiner Bio-One, cat. no. 227290) according to the manufacturers' instructions. PBMCs were resuspended in PBS at a density of 10×10⁶ cells/mL and labeled with CTV using CellTrace™ Violet Cell Proliferation Kit (Thermo Fisher Scientific, cat. no. C34557), according to the manufacturer's instructions. CTV-labeled PBMCs (75,000 cells/well) were seeded in 96-well U bottom plates (Greiner Bio-One, cat. no. 650180) and incubated with 0.1 μg/mL CD3 antibody clone UCHT1 (Stemcell, cat. no. 60011) with IgG1-CD27-A-P329R-E345R (0.016 to 10 μg/mL in five-fold dilutions) and DuoBody-PD-L1×4-1BB (0.000064 to 5 μg/mL, in five-fold dilutions) in culture medium (RPMI 1640 [Lonza, cat. no. 12-115F], 10% donor bovine serum with iron [DBSI; Gibco, cat. no. 20731-030], 1% Pen/Strep [Lonza, cat no. DE17-603E]; or IMDM with Hepes and L-Glutamine [Lonza, cat. no. 12-722F]; 5% human serum [Sigma Aldrich, cat. no. H4522, heat inactivated at 65° C. for 30 min], 1% Pen/Strep [Lonza]) for four days. The cell suspensions were pelleted, washed once with FACS buffer (PBS [Lonza, cat. no. BE17517Q], 0.02% sodium azide [bioWorld, cat. no. 41920044 3], 0.1% BSA [Roche, cat. no. 43279213], 2 mM EDTA [Sigma, cat. no. BCCD3789]) and incubated with FACS buffer containing the lymphocyte markers FITC-labeled anti-human CD4 (BD Biosciences, cat. no. 345768; 1:50) and APC-labeled anti-human CD8 (BD Biosciences, cat. no. 555369; 1:50) at 4° C. for 30 min. Cells were washed twice and resuspended in FACS buffer containing viability dye 7-aminoactinomycin D (7-AAD; BD Biosciences, cat. no. 51 68981E; 1:240). Flow cytometry data were acquired on an iQue+ (BioRad).

CTV dilution peaks in the viable CD4⁺ and CD8⁺ T-cell subsets (FITC-CD4⁺APC-CD8⁻⁷-AAD⁻ and FITC-CD4⁻APC-CD8⁺7-AAD⁻, respectively) were analyzed using the proliferation modeling tool in FlowJo software (v10.7.1) and expansion indices were determined according to the following formula:

$$Number of cells at start of culture = ( G 0 ) + ( G 1 ) / 2 + ( G 2 ) / 4 + ( G 3 ) / 8 + ( G 4 ) / 16 + … ( GN / 2 N )$$

$$Expansion index = Total cell number ( sum G 0 to GN ) / Number of cells at start$$

G0 to GN are single proliferation peaks, with G0 representing the undivided cell fraction and GN the cell fraction that divided N times.

A dose-dependent increase in CD4⁺ (FIG. 43A) and CD8⁺ (FIG. 43B) T-cell proliferation was observed in the PBMC samples treated with IgG1-CD27-A-P329R-E345R alone over the whole antibody concentration range. The dose-response curves of the samples treated with DuoBody-PD-L1×4-1BB alone showed a bell-shaped curve, reaching the highest expansion indices at the intermediate concentrations. The combination of IgG1-CD27-A-P329R-E345R with DuoBody-PD-L1×4-1BB increased CD4⁺ and CD8⁺ T-cell proliferation more potently than each antibody alone, and the largest effects were reached at the highest tested IgG1-CD27-A-P329R-E345R concentrations (2 to 10 μg/mL) in combination with the intermediate to high concentrations that were tested for DuoBody-PD-L1×4-1BB (0.04 to 5 μg/mL).

These data indicate that the combination of IgG1-CD27-A-P329R-E345R with DuoBody-PD-L1×4-1BB induced a higher increase in proliferation of activated T-cells compared to each antibody alone.

### Example 44: Antigen-Specific Stimulation Assay to Determine the Capacity of IgG1-CD27-A-P329R-E345R in Combination with DuoBody-PD-L1×4-1BB or PD-1/PD-L1 Inhibitors to Enhance T-Cell Proliferation and Cytokine Secretion

To determine the combinatorial effect of IgG1-CD27-A-P329R-E345R with a bispecific antibody targeting PD-L1 and 4-1BB or PD-1/PD-L1 inhibitors on T-cell proliferation and cytokine production compared to single-agent activity, an antigen-specific stimulation assay was conducted using co-cultures of PD-1-overexpressing healthy human CD8⁺ T cells and cognate antigen-expressing immature dendritic cells (iDCs).

HLA-A*02⁺ peripheral blood mononuclear cells (PBMCs) were obtained from healthy donors (Transfusionszentrale, University Hospital, Mainz, Germany). Monocytes were isolated from PBMCs by magnetic-activated cell sorting (MACS) technology using anti-CD14 MicroBeads (Miltenyi; cat. no. 130-050-201), according to the manufacturer's instructions. The peripheral blood lymphocytes (PBLs, CD14-negative fraction) were cryopreserved in RPMI 1640 containing 10% DMSO (AppliChem GmbH, cat. no. A3672,0050) and 10% human albumin (CSL Behring, PZN 00504775) for T-cell isolation. For differentiation into iDCs, 40×10⁶ monocytes/mL were cultured for five days in RPMI 1640 (Life Technologies GmbH, cat. no. 61870-010) containing 5% pooled human serum (One Lambda Inc., cat. no. A25761), 1 mM sodium pyruvate (Life technologies GmbH, cat. no. 11360-039), 1× non-essential amino acids (Life Technologies GmbH, cat. no. 11140-035), 200 ng/mL granulocyte-macrophage colony-stimulating factor (GM-CSF; Miltenyi, cat. no. 130-093-868) and 200 ng/mL human interleukin-4 (IL-4; Miltenyi, cat. no. 130-093-924). On day 3, half of the medium was replaced with fresh medium containing supplements. On day 5, iDCs were harvested by collecting non-adherent cells and adherent cells were detached by incubation with Dulbecco's phosphate-buffered saline (DPBS) containing 2 mM EDTA for 10 min at 37° C. After washing with DPBS iDCs were cryopreserved in FBS (Sigma-Aldrich, cat. no. F7524) containing 10% DMSO (AppliChem GmbH, cat. no A3672,0050) for future use in antigen-specific T-cell assays.

One day prior to the start of an antigen-specific CD8⁺ T-cell stimulation assay, frozen PBLs and iDCs from the same donor were thawed. CD8⁺ T cells were isolated from PBLs by MACS technology using anti-CD8 MicroBeads (Miltenyi, cat. no. 130-045-201), according to the manufacturer's instructions. About 10×10⁶ to 15×10⁶ CD8⁺ T cells were electroporated with each 10 μg of in vitro transcribed (IVT)-RNA encoding the alpha and beta chains of a murine TCR specific for human claudin-6 (CLDN6; HLA-A*02-restricted; described in WO 2015150327 A1) and 10 μg IVT-RNA encoding human PD-1 (UniProt Q15116) in 250 μL X-VIVO™ 15 medium (Lonza, cat. no. BE02-060Q). The cells were transferred to a 4-mm electroporation cuvette (VWR International GmbH, cat. no. 732-0023) and electroporated using the BTX ECM® 830 Electroporation System (BTX; 500 V, 3 ms pulse). Immediately after electroporation, cells were transferred into fresh IMDM GlutaMAX medium (Life Technologies GmbH, cat. no. 319800-030) containing 5% pooled human serum and rested at 37° C., 5% CO₂ for at least 1 hour. T cells were labeled using 0.8 μM carboxyfluorescein succinimidyl ester (CFSE; Life Technologies GmbH, cat. No V12883) in PBS according to the manufacturer's instructions and incubated in IMDM medium supplemented with 5% human serum overnight.

Up to 5×10⁶ thawed iDCs were electroporated with 2 μg IVT-RNA encoding full-length human CLDN6 (WO 2015150327 A1), in 250 μL X VIVO 15 medium, using the electroporation system as described above (300 V, 12 ms pulse) and incubated in IMDM medium supplemented with 5% pooled human serum overnight.

Electroporated iDCs were incubated with electroporated, CFSE-labeled T cells at a ratio of 1:10 (DC:T cells) in the presence of anti-CD27 antibodies IgG1-CD27-A-P329R-E345R (0.1, 1, or 10 μg/mL) or IgG1-CD27-131A (10 μg/mL), or in the presence of DuoBody-PD-L1×4-1BB (0.2 μg/mL), IgG1-PD1 (0.8 μg/mL), pembrolizumab (Keytruda®, Merck Sharp & Dohme GmbH, PZN 10749897; 0.8 μg/mL), nivolumab (Opdivo®, Bristol-Myers-Squibb 11024601; 1.6 μg/mL), atezolizumab (Tecentriq©, Roche PZN 11306050; 0.4 μg/mL), either alone or in combination with IgG1-CD27-A-P329R-E345R (0.1, 1, or 10 μg/mL) or IgG1-CD27-131A (10 μg/mL), in IMDM medium containing 5% pooled human serum in a 96-well round-bottom plate. After 4 days of culture, the cells were stained with an APC-conjugated anti-human CD8 antibody. T-cell proliferation was evaluated by flow cytometry analysis of CFSE dilution in CD8⁺ T cells using a BD FACSCelesta™ flow cytometer (Becton Dickinson GmbH).

Flow cytometry data was analyzed using FlowJo software version 10.7.1. CFSE label dilution of CD8⁺ T cells was assessed using the proliferation modeling tool in FlowJo, and expansion indices calculated using the following formula.

$$Number of cells at start of culture = ( G 0 ) + ( G 1 ) / 2 + ( G 2 ) / 4 + ( G 3 ) / 8 + ( G 4 ) / 16 + … ( GN / 2 N )$$

$$Expansion index = Total cell number ( sum G 0 to GN ) / Number of cells at start$$

G0 to GN are single proliferation peaks, with G0 representing the undivided cell fraction and GN the cell fraction that divided N times.

Cytokine concentrations in cell culture supernatants were determined by multiplexed electrochemiluminescence immunoassay (ECLIA) using a custom-made U-Plex biomarker group 1 (human) assay for the detection of a panel of 10 human cytokines (GM-CSF, IL-2, IL-8, IL-10, IL-12p70, IL-13, interferon [IFN]γ, IFNγ-inducible protein [IP]-10 [also known as C-X-C motif chemokine ligand 10], macrophage chemoattractant protein [MCP]1, and tumor necrosis factor [TNF]-α; Meso Scale Discovery, cat. No. K15067L-2) or a U-Plex Immuno-Oncology Group 1 (human) assay for the detection of a panel of 10 human cytokines (GM-CSF, IL-2, IL-12p70, IL-13, interferon [IFN]γ, IFNγ-inducible protein [IP]-10 [also known as C-X-C motif chemokine ligand 10], macrophage chemoattractant protein [MCP]1, macrophage inflammatory protein [MIP]-1β, sCD27 and tumor necrosis factor [TNF]α; Meso Scale Discovery, cat. No. K151AEL-2) following the manufacturer's protocol.

Single-agent treatment with IgG1-CD27-A-P329R-E345R or DuoBody-PD-L1×4-1BB enhanced proliferation of PD-1-overexpressing CD8⁺ T cells (FIG. 44A). Treatment with a combination of IgG1-CD27-A-P329R-E345R and DuoBody-PD-L1×4-1BB led to higher proliferation than treatment with each compound individually.

A combination of 1 or 10 μg/mL IgG1-CD27-A-P329R-E345R and IgG1-PD1, pembrolizumab, nivolumab or atezolizumab enhanced proliferation of PD-1-overexpressing CD8⁺ T cells compared to IgG1-CD27-A-P329R-E345R and anti-PD-(L)1 antibodies as single-agents (FIG. 44B). In contrast, the combination of 10 μg/mL IgG1-CD27-131A with IgG1-PD1 or nivolumab only led to a minor increase in proliferation and the combination of 10 μg/mL IgG1-CD27-131A with pembrolizumab or atezolizumab did not increase proliferation compared to the respective anti-PD-(L)1 single-agent treatments.

Single-agent treatment with IgG1-CD27-A-P329R-E345R modestly enhanced secretion of the proinflammatory cytokines IFN-γ and GM-CSF, and did not enhance secretion of TNFα and IL-2 in co-cultures of PD-1-overexpressing CD8⁺ T cells and iDCs, compared to untreated co-cultures (FIG. 45A). DuoBody-PD-L1×4-1BB single agent treatment markedly enhanced secretion of these cytokines whereas the combination treatment with IgG1-CD27-A-P329R-E345R and DuoBody-PD-L1×4-1BB further potentiated it.

Combination treatment with 1 or 10 μg/mL IgG1-CD27-A-P329R-E345R and IgG1-PD1, pembrolizumab, nivolumab or atezolizumab enhanced secretion of IFNγ compared to single-agent treatments (FIG. 45B). At concentrations of 1 and 10 μg/mL IgG1-CD27-A-P329R-E345R with IgG1-PD1 or nivolumab or 10 μg/mL IgG1-CD27-A-P329R-E345R with pembrolizumab or atezolizumab, the total IFNγ levels measured in the supernatant were higher than the sum of IFNγ levels observed with the single agents. In contrast, a combination of 10 μg/mL IgG1-CD27-131A and IgG1-PD1, pembrolizumab, nivolumab or atezolizumab led to no or only a minor increase in IFNγ-secretion.

These data indicate that the combination of IgG1-CD27-A-P329R-E345R with PD-1/PD-L1 inhibitors or with DuoBody-PD-L1×4-1BB induced a stronger increase in CD8⁺ T-cell proliferation and cytokine secretion compared to each antibody alone.

### Example 45: Effect of a Combination of IgG1-CD27-A-P329R-E345R and DuoBody-PD-L1×4-1BB on CLDN6-Specific T-Cell Mediated Cytotoxicity

Induction of T-cell mediated cytotoxicity upon combined IgG1-CD27-A-P329R-E345R and DuoBody-PD-L1×4-1BB treatment was analyzed by cell impedance measurement in co-cultures of human healthy donor T cells expressing CLDN6-TCR and MDA-MB-231_hCLDN6 target cells.

MDA-MB-231_hCLDN6 cells were generated by lentiviral transduction. To this end, 2×10⁵ MDA-MB-231 human breast cancer cells in 250 μL Dulbecco's modified eagle medium (DMEM, Thermo Fisher Scientific, cat. no. 31966-047) supplemented with 10% FBS (Biochrom, cat. no. S0115; non-heat-inactivated) were seeded per well in a 12-well tissue culture plate. The cells were incubated for 1-2 h at 37° C. (7.5% CO₂). Supernatants containing lentiviral vectors encoding human CLDN6 (pL64b42E(EF1a-hClaudin6)Hygro-T2A-GFP) were thawed on ice and diluted in a total volume of 750 μL DMEM/10% FBS to obtain titers of 2×10⁵, 8×10⁴, and 3.2×10⁴ TU/mL. These titers corresponded to MOI of 1, 0.4, and 0.16, respectively. The supernatants were then added to the MDA-MB-231 cells, and the cells were incubated for 72 h at 37° C. (5% CO₂) without disturbance. For the experiments described in the current Example, MDA-MB-231-hCLDN6 cells were cultured in DMEM/10% FBS. Cells were passaged or harvested for experiments at 70% to 90% confluence. Cells were detached by treatment with Accutase (Thermo Fisher Scientific, cat. no. A11105010) for 5 min (37° C., 7.5% CO₂), and resuspended by addition of culture medium. Cells were centrifuged (300×g, 4 min at RT) and counted. MDA-MB-231_hCLDN6 cells were not cultured for more than 20 passages.

Human magnetic CD8 MicroBeads (Miltenyi Biotec, cat. no. 130-045-201) were used for positive selection of CD8⁺ T cells from frozen peripheral blood leukocytes, according to the manufacturer's instructions. Cell suspensions were centrifuged and resuspended in magnetic-activated cell sorting (MACS) buffer (Dulbecco's PBS [Thermo Fisher, cat. no. 14190250] with 5 mM EDTA [Sigma-Aldrich, cat. no. 03690] and 1% human albumin [CSL Behring, cat. no. PZN-00504775]) at 1×10⁷ live cells per 80 μL MACS buffer. Per 1×10⁷ cells, 12 μL CD8 MicroBeads were added. Mixtures of cells and MicroBeads were incubated at 2 to 8° C. for 15 min. After washing with MACS buffer, the mixtures were pelleted by centrifugation (8 min, 300×g at room temperature [RT]), resuspended in MACS buffer and filtered through 30 μm cell strainers (BD Biosciences, cat. no. 340626). Subsequent MACS separation was performed using an automated magnetic cell separation instrument or by manual separation, depending on availability. Automated MACS separation was performed using an autoMACS® Pro Separator (Miltenyi Biotec). The magnetic separation columns were rinsed with MACS buffer according to the manufacturer's instruction before loading cells/MicroBeads mixtures. The preset positive selection program ‘POSSEL_s’ was used. For manual MACS separation, LS Columns (Miltenyi Biotec, cat. no. 130-042-401) were placed in a MidiMACS™ or QuadroMACS™ Separator and equilibrated with MACS buffer. Columns were loaded with cells labeled with CD8 MicroBeads and the cells were allowed to flow through by gravity flow. After washing three times with MACS buffer, the columns were removed from the magnet and the magnetic bead-labeled labeled cells were eluted in two steps with MACS buffer using the supplied plunger.

The isolated CD8⁺ T cells were electroporated with RNA encoding the alpha and beta chains of a mouse TCR specific for human CLDN6. Up to 10-15×10⁶ CD8⁺ T cells were electroporated in 250 μL X-VIVO™ 15 SF medium (Lonza, cat. no. BE02-060Q) at RT using an ECM 830 Square Wave Electroporation System (BTX®). Cells were mixed with RNA, pulsed (500 V, 3 ms), and immediately diluted with 750 μL prewarmed assay medium (IMDM GlutaMAX [Life technologies, cat. no. 31980030] with 5% PHS). After overnight incubation, electroporated CD8⁺ T cells were evaluated by flow cytometry to evaluate cell purity, expression of transfected RNA and baseline expression of CD27 on CD8⁺ T cells. To this end, single-cell suspensions were first stained for CD8, CD27 and CLDN6 with titrated amounts of BV605-labeled anti-CD8, DyLight650-labeled anti-CLDN6 and BV480-labeled anti-CD27, diluted 1:600, 1:100 and 1:50, respectively, in 30 to 50 μL staining buffer (DPBS, 2% FBS, 2 mM EDTA). Fixable viability dye eFluor780 (Thermo Fisher Scientific, cat. no. 65-0865-14; 1:2,000) was added during staining. The staining procedure was carried out at 2 to 8° C. protected from light for 15 to 20 min. Cells were washed twice with staining buffer (5 min, 450×g at RT) and resuspended in staining buffer for flow cytometry analysis. Flow cytometry data were acquired on a BD FACSCelesta flow cytometer. Approximately 78% to 93%, 78% to 92%, and 36% to 98% of electroporated CD8⁺ T cells expressed CLDN6-TCR and endogenous CD27, respectively.

Real-time cell analysis of tumor-cell killing was performed by impedance measurements on an xCELLigence real-time cell analysis (RTCA) instrument (ACEA Biosciences). A decrease in impedance in this experimental setting is considered a surrogate of tumor-cell killing by CD8⁺ T cells. It should be noted that impedance may underestimate tumor cell killing due to proliferation of T cells. MDA-MB-231_hCLDN6 cells were seeded at 1.2 to 1.5×10⁴ cells/well in an xCELLigence E-plate 96 (Agilent, cat. no. 05232368001) and allowed to settle at RT for 30 min. Next, plates were incubated for one day in the xCELLigence RTCA instrument (37° C., 5% CO₂).

T cells expressing CLDN6-TCR were added at 1.5×10⁵ CD8⁺ T cells/well to the seeded MDA-MB-231_hCLDN6 cells resulting in a T cell:tumor cell (effector:target) ratio of 10:1. IgG1-CD27-A-P329R-E345R (1 or 10 μg/mL), DuoBody-PD-L1×4-1BB (0.2 μg/mL), or non-binding control antibody IgG1-b12-P329R-E345R (10 μg/mL) were added to the co-cultures. The co-cultures in the E-plate 96 were incubated in presence of antibody in the xCELLigence RTCA instrument for five days without disturbance, with impedance measurements at two-hour intervals as a readout for total cell mass (cell index). Graphs presenting cell index values in time were graphically displayed using GraphPad Prism software and used to determine the area under the curve (AUC), which was normalized for each co-culture treated with test antibody to the co-cultures treated with non-binding control antibody IgG1-b12-P329R-E345R.

Real-time cell analysis showed that IgG1-CD27-A-P329R-E345R and DuoBody-PD-L1×4-1BB alone substantially enhanced CD8⁺ T-cell-mediated tumor cell kill, compared to the negative control antibody IgG1-b12-P329R-E345R (FIG. 46A). Tumor cell killing was most pronounced when both compounds were used in combination. In pooled analyses, normalized AUC of cultures treated with a combination of IgG1-CD27-A-P329R-E345R and DuoBody-PD-L1×4-1BB was significantly lower than the AUC of those treated with IgG1-CD27-A-P329R-E345R alone (FIG. 46B). While not statistically significant, the AUC of combination-treated cultures were also lower than those treated with DuoBody-PD-L1×4-1BB alone.

### Example 46: Effect of a Combination of IgG1-CD27-A-P329R-E345R and DuoBody-PD-L1×4-1BB on the Expression of T-Cell Cytotoxicity-Associated Molecules in an Antigen-Specific Cytotoxicity Assay

The effect of a combination of IgG1-CD27-A-P329R-E345R and DuoBody-PD-L1×4-1BB on the expression of degranulation marker CD107a and the cytotoxic mediator granzyme B (GzmB) on antigen-specific CD8⁺ T cells was assessed by flow cytometry using co-cultures of CLDN6-TCR-expressing CD8⁺ T cells with hCLDN6-MDA-MB-231 cells that were incubated with antibodies, as described in Example 40.

The co-cultures were incubated in presence of antibody for two days and subsequently stained for CD8, CD107a (lysosomal-associated membrane protein-1 [LAMP-1]), and GzmB for analysis by flow cytometry, except for the CD107a antibody that was already added to all treatment conditions at the start of the co-cultures, given that CD107a is expressed on cytotoxic granules and therewith reinternalized upon T-cell degranulation. For flow cytometry, the procedures as essentially described in Example 40 were performed with the following deviations. After two days of incubation, a small volume of the assay medium (20 μL/well) containing Golgi-Plug (Brefeldin A; final dilution in 220 μL: 1:1,000) was added to the cells, followed by incubation for an additional 4 h. Afterwards the cells were harvested, and expression of intracellular GzmB and CD107a in CD8⁺ T cells was analyzed by flow cytometry. For intracellular staining, cells were first washed twice with staining buffer (5 min, 450×g at RT) and resuspended in 200 μL Histofix 2% (Carl Roth; cat. no. P087.4, 1:2 diluted with DPBS), followed by incubation at 2 to 8° C. for at least 20 min, protected from light. Cells were then centrifuged (5 min, 600×g at 2 to 8° C.), washed with 1× permeabilization buffer (Thermo Fisher Scientific, cat. no. 00-8333-56) and stained for intracellular markers using titrated amounts of PE-labeled anti GzmB (BD, cat. no. 561142; diluted 1:2300) and AF647-labeled anti-CD107a antibodies (Biolegend, cat. no. 328611; diluted 1:2,500) in 1× permeabilization buffer. The staining procedure for intracellular markers was carried out at 2 to 8° C. for 20 to 60 min protected from light. The cells were then washed twice with 1× permeabilization buffer (5 min, 600×g at RT), and resuspended in staining buffer for flow cytometry analysis. Flow cytometry data were acquired on a BD FACSCelesta flow cytometer. The mean fluorescence intensities (MFI) measured for GzmB and CD107a using cells from six healthy donors were normalized to the MFI of control antibody IgG1-b12-P329R-E345R (10 μg/mL).

The increased cytotoxic capacity of IgG1-CD27-A-P329R-E345R and DuoBody-PD-L1×4-1BB combination treatment was associated with significantly increased expression levels of GzmB compared with treatment with DuoBody-PD-L1×4-1BB alone (FIG. 47A). In addition, expression levels of CD107a were increased by IgG1-CD27-A-P329R-E345R and DuoBody-PD-L1×4-1BB combination treatment compared with treatment with IgG1-CD27-A-P329R-E345R only (FIG. 47B). The combination of 10 μg/mL IgG1-CD27-A-P329R-E345R and DuoBody-PD-L1×4-1BB significantly increased the percentage of CD8⁺ T cells expressing both GzmB and CD107a as compared with DuoBody-PD-L1×4-1BB treatment alone (FIG. 40C). Single-agent treatment with 1 or 10 μg/mL IgG1-CD27-A-P329R-E345R or DuoBody-PD-L1×4-1BB alone increased the percentage of CD8⁺ T cells expressing both GzmB and CD107a compared with treatment with non-binding control antibody IgG1-b12-P329R-E345R.

In conclusion, the increase in cytotoxic capacity by IgG1-CD27-A-P329R-E345R and DuoBody-PD-L1×4-1BB combination treatment was associated with increased expression of degranulation marker CD107a and cytotoxic mediator GzmB in CD8+ T-cells.

### Example 47: Expansion of Tumor-Infiltrating Lymphocytes in Cell Cultures from NSCLC Tumor Fragments Treated with IgG1-CD27-A-P329R-E345R in Combination with DuoBody-PD-L1×4-1BB Ex Vivo

The effect of a IgG1-CD27-A-P329R-E345R and DuoBody-PD-L1×4-1BB combination treatment on the expansion of different subsets of tumor-infiltrating lymphocytes (TIL) was assessed. Ex vivo studies using TIL were performed using cryopreserved tumor tissue that had been surgically resected from three NSCLC patients at University Hospital Mainz, Germany.

Surgically resected human NSCLC tissues were received in transport medium (HypoThermosol® FRS Preservation Solution [BioLife Solutions, cat. no. 101104], 7.5 μg/mL Amphotericin B [Thermo Fisher Scientific, cat. no. 15290026], and 300 units/mL (U/mL) penicillin/streptomycin [Thermo Fisher Scientific, cat. no. 15140-122]). Samples were washed three times in wash medium (5 mL X VIVO 15 [Lonza], 2.5 μg/mL Amphotericin B [Thermo Fisher Scientific, and 100 U/mL penicillin/streptomycin [Thermo Fisher Scientific]) and transferred to a cell culture dish. Fatty tissue and necrotic areas were removed with a scalpel, and the tissue was cut into fragments of approximately 5 mm³. Each fragment was placed in an individual cryovial, and 1 mL freezing medium (FBS [Biochrom, cat. no. S0115], 10% DMSO [AppliChem, cat. no. A3672,0100]) was added to each vial. The vials were transferred into a controlled freeze-chamber (Mr. Frosty freezing container; Thermo Fisher Scientific), which was placed in a −80° C. freezer. After at least 16 h at −80° C., the vials were transferred to liquid nitrogen for long-term storage.

Four to six cryopreserved vials each containing tumor fragments of approximately 5 mm³ from one tumor specimen were thawed per experiment in a 37° C. water bath for approximately 2 min and washed five times with wash medium and transferred to a cell culture dish. The tumor fragments were further dissected with a scalpel into fragments of approximately 1 mm³. Most of the fragments were used for TIL expansion upon culturing with IL-2 and treatment antibodies and remaining fragments were used to determine expression of specific cell-surface markers at baseline, without any treatment.

Two tumor fragments per well (on average) were seeded in 24-well plates (2 mL/well total volume capacity used in assay) in 100 μL prewarmed TIL cultivation medium (X-VIVO 15 [Lonza, cat. no. BE02-060Q] with 2% human serum albumin [HSA; CSL Behring, cat. no. PZN-00504775], 100 U/mL penicillin/streptomycin, and 2.5 μg/mL Amphotericin B) containing 45 to 50 U/mL IL-2 (Proleukin S; Novartis Pharma, cat. no. PZN-02238131).

Antibodies were diluted in TIL cultivation medium containing 45 to 50 U/mL IL-2 and 0.9 mL of these dilutions were added to the wells as appropriate. Final IgG1-CD27-A-P329R-E345R and DuoBody-PD-L1×4-1BB concentrations in the wells were 1 or 10 μg/mL and 0.2 μg/mL, respectively. As a control, IL-2-containing medium without antibodies was added to separate wells. A total of 8 to 16 wells were used for each experimental condition per donor. The plates were incubated at 37° C., 5% C02.

After three days of culture, fresh TIL cultivation medium containing 45 to 50 U/mL IL-2, IgG1-CD27-A-P329R-E345R, and DuoBody-PD-L1×4-1BB was added to the wells (1 mL/well, same antibody concentrations as above). Between day 5 and 14 after assay initiation, the cultures were regularly monitored with a microscope for proliferation of TIL that migrated from the tissue fragments and the formation of TIL microclusters. If >25 TIL microclusters were observed in one well after seven or eight culture days, cells and tissue fragments from two identically treated original wells were resuspended and pooled into one well of a 6-well plate (5 to 6 mL/well total volume capacity used in assay) with the culture medium and fresh IL 2 containing TIL cultivation medium was added (33 U/mL IL-2). Every two to three days, cultures were supplemented with fresh IL-2-containing TIL cultivation medium. IL-2 concentrations in the medium added to the cultures were reduced to 10 U/mL, or first reduced to 25 U/mL and then to 10 U/mL thereafter after supplementing the wells with medium throughout the assay. Cultures were analyzed by flow cytometry as described below. Cryopreserved tumor fragments were thawed and further dissected as described above. Single-cell suspensions were generated by mechanical dissociation of the tumor fragments with a plunger and a cell strainer. The cells were centrifuged (8 min, 300×g at RT) and resuspended in staining buffer for flow cytometry analysis.

For flow cytometry analysis, single-cell suspensions were first stained for cell-surface markers with titrated amounts of antibodies (Table 20), diluted in 30 to 50 μL staining buffer (Dulbecco's PBS [DPBS; Thermo Fisher, cat. no. 14190250], 2% FBS, and 2 mM EDTA [Sigma, cat. no. BCCD3789]).

TABLE 20
Fluorescently labeled antibodies used for flow cytometry.

| Target | Label | Reference | Company | Dilution |
|---|---|---|---|---|
| CD3 | Pacific Blue | 558117 | BD Biosciences | 1:100 |
| CD4 | PerCP-eF710 | 46-0047-42 | Thermo Fisher | 1:100- |
| | | | Scientific | 1:200 |
| CD8a | BV605 | 564116 | BD Biosciences | 1:400 |
| CD25 | APC-eF780 | 47-0259-42 | Thermo Fisher | 1:100 |
| | | | Scientific | |
| CD56 | BV786 | 740979 | BD Biosciences | 1:100 |

Brilliant Stain Buffer Plus (BD Biosciences, cat. no. 566385) was added to the antibody mix at a final dilution of 1:5. Fixable viability stain 700 (BD Biosciences, cat. no. 564997; 1:1,000 to 1:1,500) was added during cell-surface staining. The staining procedure was carried out at 2 to 8° C. protected from light for 15 to 20 min. Cells were washed twice with staining buffer (5 min, 450×g at RT) and resuspended in staining buffer for flow cytometry analysis.

Flow cytometry data were acquired on a BD FAC Symphony or BD FACSCelesta flow cytometer. Prior to acquisition, 30 μL of CountBright Absolute counting beads (Thermo Fisher Scientific, cat. no. C36950) were added to each sample for absolute cell counts. The following TIL populations were identified and quantified by flow cytometry: CD4⁺ and CD8⁺ T cells, and natural killer (NK) cells. Within the live gate of single cells, CD56⁺ NK cells and CD3⁺ were gated. Within the CD3⁺ gate, further gating was performed on CD4⁺ and CD8⁺ cells. Flow cytometry data were analyzed using FlowJo Software version 10.7.1. Absolute cell numbers were determined using the formula:

$$Cell number = \frac{Event count ( cell )}{Event count ( beads )} \times bead concentration \times sample volume$$

IgG1-CD27-A-P329R-E345R and DuoBody-PD-L1×4-1BB as single treatments induced proliferation of CD8⁺ T cells and NK cells in two out of three and three out of three specimens, respectively (FIG. 48 and Table 21). CD8⁺ T-cell and NK-cell expansion was further enhanced by IgG1-CD27-A-P329R-E345R and DuoBody-PD-L1×4-1BB combination treatment. Importantly, strong combination effects on CD8⁺ T cells and NK cells were detectable in specimens where either IgG1-CD27-A-P329R-E345R (specimen #592) or DuoBody-PD-L1×4-1BB (specimen #578) had modest single-agent effects. DuoBody-PD-L1×4-1BB alone had marginal effects on CD4⁺ T cells, and combination treatment did not substantially enhance CD4⁺ T-cell expansion compared to single-agent IgG1-CD27-A-P329R-E345R.

TABLE 21
Fold-expansion of TIL subsets treated with a combination of IgG1-CD27-A-P329R-E345R and DuoBody-PD-L1x4-
1BB compared to IL-2 only. Tumor fragments derived from human NSCLC specimens were cultured with low-dose
IL-2 only, or in the presence of 1 μg/mL IgG1-CD27-A-P329R-E345R, 0.2 μg/mL DuoBody-PD-L1x4-1BB,
or a combination of both. Absolute cell counts of the indicated cell subsets were determined by flow
cytometry after 14 d. Fold-differences in numbers of antibody-treated cultures relative to cultures treated
with IL-2 only are shown. Data shown are from three independent experiments. NK, natural killer; n.d.,
not determined; n.t., not tested; PD-L1, programmed cell death 1 ligand 1; SD, standard deviation.

| | Fold difference in cell number of samples treated with antibody | | | | | | | | |
|---|---|---|---|---|---|---|---|---|---|
| | relative to negative control (without antibody) | | | | | | | | |
| Cell population | CD4⁺ T cells | | | CD8⁺ T cells | | | NK cells | | |
| IgG1- | + | − | + | + | − | + | + | − | + |
| CD27-A- | | | | | | | | | |
| P329R- | | | | | | | | | |
| E345R | | | | | | | | | |
| DuoBody- | − | + | + | − | + | + | − | + | + |
| PD-L1x4- | | | | | | | | | |
| 1BB | | | | | | | | | |
| Patient | 0.8 | 1.0 | 1.2 | 0.2 | 3.2 | 1.5 | 1.1 | 11.2 | 13.4 |
| #561 | | | | | | | | | |
| Patient | 19.3 | 1.9 | 9.8 | 19.6 | 4.3 | 31.3 | 11.3 | 5.0 | 51.3 |
| #578 | | | | | | | | | |
| Patient#592 | 0.4 | 0.3 | 1.8 | 2.3 | 14.6 | 41.5 | 2.3 | 26.9 | 54.6 |
| Average ± | 9.9 ± 9.5 | 1.1 ± 0.7 | 6.8 ± 4.5 | 7.4 ± 8.7 | 7.4 ± 5.1 | 24.8 ± 17.0 | 4.9 ± 4.6 | 14.4 ± 9.2 | 39.8 ± 18.7 |
| SD | | | | | | | | | |

### Example 48: Combination of IgG1-CD27-A-P329R-E345R and PD111/PD11-L1-Checkpoint Inhibitors Shows Potentiation of IFNγ Production in a CD8 Mixed-Lymphocyte Reaction Assay

To analyze if a combination of IgG1-CD27-A-P329R-E345R with IgG1-PD1 or pembrolizumab could result in enhanced immune activation over treatment with each antibody alone, a mixed-lymphocyte reaction (MLR) assay was performed. Allogeneic dendritic cells (DCs) and T cells were co-cultured for 5 days in the presence of IgG1-CD27-A-P329R-E345R combined with IgG1-PD1 or pembrolizumab, or either of the antibodies alone in the same concentration range.

CD14⁺ monocytes and CD8⁺ T cells from allogeneic donor pairs that were used for the MLR assays were obtained from BioIVT (Table 22).

TABLE 22
Allogeneic donor pairs of monocyte
and T cells used in MLR assays

| Donor | Human CD14⁺ monocytes | | Human CD8⁺ T cells | |
|---|---|---|---|---|
| pair | Lot no. | Donor ID | Lot no. | Donor ID |
| 1 | LS112418861 | 58355 | LS1170885 | CC00481 |
| 2 | | | LS1169965 | M7281 |

To differentiate CD14⁺ monocytes into immature DCs (iDCs), 1.5×10⁶ monocytes/mL were incubated in T25 culture flasks (Falcon, cat. no. 353108) in RPMI-1640 medium (ATCC modification; Thermo Fisher Scientific, cat. no. A1049101) supplemented with 10% heat-inactivated FBS (Thermo Fisher Scientific, cat. no. 16140071), 100 ng/mL GM-CSF (BioLegend, cat. no. 766106) and 300 ng/mL IL-4 (BioLegend, cat. no. 766206) at 37° C., 5% CO₂ for six days. On day 4, the old medium was aspirated and fresh medium with supplements was added. On day 6, iDCs were harvested by collecting non-adherent cells. To mature the iDCs, 1-1.5×10⁶ cells/mL were incubated in RPMI-1640 medium (ATCC modification) supplemented with 10% FBS, 100 ng/mL GM-CSF, 300 ng/mL IL-4, and 5 μg/mL lipopolysaccharide (LPS; Thermo Fisher Scientific, cat. no. 00-4976-93) at 37° C., 5% CO₂ for 24 h prior to start of the MLR assay.

One day prior to the start of an MLR assay, human CD8⁺ T cells were thawed and incubated at 1×10⁶ cells/mL in RPMI-1640 medium (ATCC modification) supplemented with 10% FBS and 10 ng/mL IL-2 (BioLegend, cat. no. 589106) in a T75 culture flask (Falcon, cat. no. 353136) at 37° C., 5% CO₂ O/N. The next day, the CD8⁺ T cells and LPS-matured DCs were harvested and resuspended in pre-warmed AIM-V medium (Thermo Fisher Scientific, cat. no. 12055091) at 4×10⁶ cells/mL and 4×10⁵ cells/mL, respectively. 20,000 DCs and 200,000 CD8⁺ T cells from allogeneic donor pairs were co-cultured (DC:T cell ratio of 1:10) in AIM-V medium in a round-bottom 96-well plate in the presence of serial dilutions of IgG1-CD27-A-P329R-E345R (final concentration range 0.001-30 μg/mL), IgG1-PD1 (final concentration range 0.001-100 μg/mL), and/or pembrolizumab (final concentration: 1 μg/mL) at 37° C., 5% CO₂ for five days. In parallel, to confirm the responsiveness of the donors, T cells were incubated with ImmunoCult™ Human CD3/CD28 T Cell Activator (Stemcell, cat. no. 10971) at 37° C. for five days. After five days, the plates were centrifuged at 500×g for 5 min and the cell-free supernatant was transferred from each well to a new round-bottom 96-well plate and stored at −80° C. until further analysis of cytokine concentrations.

To assess cytokine secretion, cytokine levels in the supernatants of MLR assays on day 5 were determined by immunoassays. The IFNγ levels were determined using the AlphaLISA Human IFNγ Detection Kit (PerkinElmer, cat. no. AL217) on an Envision instrument, according to the manufacturer's instructions. The levels of GM-CSF were determined using a customized Luminex® multiplex immunoassay (Millipore, order no. SPR1526) based on the MILLIPLEX® MAP Human TH17 Magnetic Bead Panel (HTH17MAG-14K), essentially as described by the manufacturer. Briefly, frozen supernatants from the MLR assays were thawed and 10 μL of each sample was added to 10 μL Assay Buffer per well of a 384-well plate (Greiner Bio-One, cat. no. 781096) that was prewashed with 1× kit-provided Wash Buffer. In parallel control wells, 10 μL of AIM-V medium was added to 10 μL of Standard or Control in Assay Buffer. Color-coded Magnetic Beads coated with antibodies against GM-CSF were mixed and diluted to 1× concentrations in Bead Diluent, after which 10 μL of the mixed beads was added to each well. The plate was centrifuged briefly by a pulse spin to 1,000 RPM, sealed, and incubated while shaking at 4° C., O/N. Beads were washed three times with 60 μL 1× Wash Buffer per well using a Flick and Blot Magnetic Separation Plate for 384-well plates (Thermo Fisher Scientific, cat. no. VP 771HHG4). Subsequently, 10 μL of the cocktail of biotinylated Custom Detection Antibodies was added to each well, and the plate was centrifuged briefly by a pulse spin to 1,000 RPM, sealed, and incubated while shaking at RT, for 1 h. Next, 10 μL of PE-conjugated streptavidin was added to each well, and the plate was briefly centrifuged by a pulse spin to 1,000 RPM, sealed, and incubated while shaking at RT for 30 min. Wells were washed three times with 60 μL 1× Wash Buffer as described above, after which beads were resuspended in 75 μL Luminex Sheath Fluid by shaking at RT for 5 min. From each well, 50 μL samples were collected and run on a Luminex FLEXMAP 3D® system that was calibrated using the FLEXMAP 3D Calibration Kit (Millipore, cat. no. F3D-CAL-K25). Curve-fitting of the median fluorescence intensities (MFI) was performed in Belysa™ Immunoassay Curve-Fitting Software (Merck), using a 5-parameter logistic curve-fitting method.

While treatment with IgG1-CD27-A-P329R-E345R (10 μg/mL) alone did not induce secretion of IFNγ or GM-CSF, treatment with 1 μg/mL of either IgG1-PD1 or pembrolizumab alone induced secretion of both IFNγ and GM-CSF (FIG. 49). The secretion of IFNγ and GM-CSF was potentiated by combining either IgG1-PD1 (1 μg/mL) or pembrolizumab (1 μg/mL) treatment with 10 μg/mL IgG1-CD27-A-P329R-E345R.

To assess whether a combination treatment of IgG1-CD27-A-P329R-E345R and an anti-PD-1 inhibitor antibody synergistically increased immune activation, the IFNγ secretion data obtained from the combination of IgG1-CD27-A-P329R-E345R with IgG1-PD1 were processed for each donor pair separately to perform synergy analyses. The concentrations of IFNγ (μg/ml) in each treatment condition were normalized by subtracting the control values (no treatment control wells) and expressed as percentage of the maximal value in the assay (IFNγ induction). The interaction between the two antibodies combined was analyzed using the SynergyFinder package (v3.2.2; Zheng, S et al. 2021. SynergyFinder Plus: towards a better interpretation and annotation of drug combination screening datasets. bioRxiv, 10.1101/2021.06.01.446564) in R (v4.1.0). The synergistic effect was defined as the excess of observed effect over expected effect as calculated by two reference models (synergy scoring models): Highest Single Agent (HAS; Berenbaum, M. C. (1989) What is synergy? Pharmacol Rev, 41, 93-141) and Bliss (Bliss, C. I. (1939) The Toxicity of Poisons Applied Jointly1. Annals of Applied Biology, 26, 585-615). Each of the models makes different assumptions regarding the expected effect (for details see respective references).

The combination treatment of IgG1-CD27-A-P329R-E345R and IgG1-PD1 showed synergy in both models across a range of IgG1-CD27-A-P329R-E345R and IgG1-PD1 concentrations (FIG. 50). In donor pair 1, the strongest synergy was observed when 0.01 μg/mL IgG1-PD1 was combined with a range of IgG1-CD27-A-P329R-E345R concentrations (0.1-30 μg/mL; FIG. 50A). In donor pair 2, the strongest synergy was observed when 1 μg/mL IgG1-PD1 was combined with a range of IgG1-CD27-A-P329R-E345R concentrations (0.01-10 μg/mL; FIG. 50B).

Collectively, these data show that a combination treatment of IgG1-CD27-A-P329R-E345R and anti-PD1 antibodies can enhance cytokine secretion as compared to the single treatments in a CD8 MLR assay. IFNγ secretion was shown to be synergistically enhanced by a combination treatment of IgG1-CD27-A-P329R-E345R and IgG1-PD1.

### Example 49: Effect of a Combination of IgG1-CD27-A-P329R-E345R and PD-1/PD-L1 Inhibitors on Antigen-Specific T-Cell Mediated Cytotoxicity

Induction of T-cell mediated cytotoxicity upon combined IgG1-CD27-A-P329R-E345R and PD-1/PD-L1 inhibitor treatment was analyzed by real-time cell analysis via impedance measurement in co-cultures of PD-1-overexpressing and CLDN6-TCR-expressing human healthy donor CD8⁺ T cells and a human PD-L1- and CLDN6-expressing breast cancer cell line as target.

The human PD-L1⁺ breast cancer cell line MDA-MB-231 (ATCC®, HTB-26TM) was stably transduced with the model antigen claudin-6 by lentiviral transduction. Magnetic anti-human CD8 MicroBeads (Miltenyi Biotec, cat. no. 130-045-201) were used for positive selection of CD8⁺ T cells from thawed HLA-A*02-positive PBMCs, as described in Example 45. Purity of CD8⁺ T cells was above 91%. The purified CD8⁺ T cells were electroporated with RNA encoding PD-1 as well as with RNA encoding the alpha and beta chains of a mouse TCR specific for human CLDN6, as described in Example 45.

After overnight incubation, cell surface expression of CLDN6-TCR and PD-1 on electroporated CD8⁺ T cells was confirmed by flow cytometry. To this end, single-cell suspensions were stained for viability, CD8, PD-1 and murine TCRβ with 7-Aminoactinomycin D (7-AAD; BD Biosciences, cat. no. 51-68981E; diluted 1:100), titrated amounts of BV605-labeled anti-CD8, Alexa Fluor 488-labeled anti-PD-1 and BV421-labeled anti-TCRβ antibodies, diluted 1:400, 1:50 and 1:33, respectively, in 50 μL staining buffer (DPBS, 2% FBS, 2 mM EDTA). The staining procedure was carried out at 2-8° C. protected from light for 15 min. Cells were washed twice with staining buffer (5 min, 460×g at RT) and resuspended in staining buffer for flow cytometric analyses. Flow cytometric data were acquired on a BD FACSCelesta flow cytometer. Approximately 48% to 95% and 52% to 92% of electroporated CD8⁺ T cells expressed PD-1 and CLDN6-TCR, respectively.

Real-time cell analysis of tumor-cell killing was performed by impedance measurements on an xCELLigence real-time cell analysis (RTCA) instrument (ACEA Biosciences), as described in Example 45. T cells expressing CLDN6-TCR and PD-1 were added at 7.5×10⁴ CD8⁺ T cells/well to 1.5×10⁴ seeded MDA-MB-231_hCLDN6 cells resulting in a T-cell:tumor-cell (effector:target) ratio of 5:1. IgG1-CD27-A-P329R-E345R (10 μg/mL), IgG1-PD1 (0.8 μg/mL), pembrolizumab (0.8 μg/mL), nivolumab (1.6 μg/mL), or atezolizumab (0.4 μg/mL) were added to the co-cultures, either as single agent or as a combination of IgG1-CD27-A-P329R-E345R and a PD-1/PD-L1 inhibitor. Non-binding antibody IgG1-b12-P329R-E345R (10 μg/mL) was used as a negative control. The co-cultures were incubated in presence of antibodies in the xCELLigence RTCA instrument for five to six days without disturbance, with impedance measurements at two to three-hour intervals as a readout for total cell mass. Data were normalized to the timepoint of initiation of CD8⁺ T-cell/tumor-cell co-culture, which was set to 1 (cell index). Graphs presenting cell index values in time were graphically displayed using GraphPad Prism software and used to determine the area under the curve (AUC), which was normalized for each co-culture treated with test antibodies to the co-cultures treated with non-binding control antibody IgG1-b12-P329R-E345R.

Real-time cell analysis showed that IgG1-CD27-A-P329R-E345R and all tested PD-1/PD-L1 inhibitors as single agents enhanced CD8⁺ T-cell mediated tumor cell kill, compared to non-binding control antibody IgG1-b12-P329R-E345R (FIG. 51A). Tumor cell killing was further enhanced when the PD-1/PD-L1 inhibitors were used in combination with IgG1-CD27-A-P329R-E345R. In pooled analyses from 11 to 14 donors, cultures treated with a combination of IgG1-CD27-A-P329R-E345R and IgG1-PD1, pembrolizumab, nivolumab or atezolizumab showed significantly lower normalized AUC values compared to single-agent treatments (FIG. 51B).

### Example 50: Effect of a Combination of IgG1-CD27-A-P329R-E345R and PD-1/PD-L1 Inhibitors on the Expression of T-Cell Cytotoxicity-Associated Molecules in an Antigen-Specific Cytotoxicity Assay

The effect of a combination of IgG1-CD27-A-P329R-E345R and PD-1/PD-L1 inhibitors on the expression of degranulation marker CD107a (lysosomal-associated membrane protein-1 [LAMP-1]) and the cytotoxic mediator protein granzyme B (GzmB) on antigen-specific CD8⁺ T cells was assessed by flow cytometry using co-cultures of PD-1-overexpressing and CLDN6-TCR-expressing CD8⁺ T cells with MDA-MB-231_hCLDN6 cells, as described in Example 49.

The co-cultures were incubated in the presence of antibodies for two days and subsequently analyzed for viability as well as CD8, CD107a, and GzmB expression by flow cytometry. An AF647-labeled anti-CD107a antibody (Biolegend, cat. no. 328611; diluted 1:3,333) was already added to all treatment conditions at the start of the co-cultures, given that CD107a is expressed on cytotoxic granules and therefore reinternalized upon T-cell degranulation. After two days of incubation, a small volume of the assay medium (20 μL/well) containing Golgi-Plug (Brefeldin A; final dilution in 220 μL: 1:1,000) was added to the cells, followed by incubation at 37° C. for 4 h. For extracellular staining, the cells were centrifuged and resuspended in 50 μL staining buffer containing BV605-labeled anti-CD8 antibody (1:600) and fixable viability dye eFluor780 (Thermo Fisher Scientific, cat. no. 65-0865-14; 1:2,000). The extracellular staining procedure was carried out at 2 to 8° C. for 20 min protected from light. For subsequent intracellular staining, the cells were washed twice with staining buffer (5 min, 460×g at RT) and resuspended in 200 μL Histofix 2% (Carl Roth; cat. no. P087.4, 1:2 diluted with DPBS), followed by incubation at RT for 15 min, protected from light. Cells were centrifuged (5 min, 460×g at 2 to 8° C.), washed with permeabilization buffer (Thermo Fisher Scientific, cat. no. 00-8333-56) and stained for GzmB using a PE-labeled anti-GzmB antibody (BD, cat. no. 561142; diluted 1:300) in permeabilization buffer. The intracellular staining procedure was carried out at 2 to 8° C. for 20 min protected from light. The cells were then washed twice with permeabilization buffer (5 min, 460×g at RT), and resuspended in staining buffer for flow cytometric analyses. Flow cytometric data were acquired on a BD FACSCelesta flow cytometer.

Single-agent treatment with IgG1-CD27-A-P329R-E345R or a PD-1/PD-L1 inhibitor increased the percentage of CD8⁺ T cells expressing both GzmB and CD107a compared to treatment with non-binding control antibody IgG1-b12-P329R-E345R (FIG. 52). The combination of IgG1-CD27-A-P329R-E345R and a PD-1/PD-L1 inhibitor significantly increased the percentage of CD8⁺ T cells expressing both GzmB and CD107a compared to single-agent treatments.

In conclusion, the increase in cytotoxic capacity of CD8⁺ T cells by a IgG1-CD27-A-P329R-E345R and PD-1/PD-L1 combination treatment (Example 49) was accompanied with increased expression of degranulation marker CD107a and cytotoxic mediator protein GzmB by CD8⁺ T cells.

### LEGENDS TO THE FIGURES

FIG. 1 shows CD27 agonist activity of anti-CD27 antibodies and hexamerization-enhanced Fc variants thereof as determined in a CD27 Jurkat Reporter BioAssay. Thaw-and-Use GloResponse NFκB-luc2/CD27 Jurkat reporter cells were incubated for 6 h with antibody concentration series (from left to right: 0.04 μg/mL, 0.30 μg/mL, 2.50 μg/mL, and 20 μg/mL) of the indicated antibodies. Luciferase activity, as a read-out for CD27 intracellular signaling, was quantified by determining the luminescence (RLU: relative luminescence units). The following antibodies were included as WT IgG1 and/or variants with an E430G or E345R mutation, as indicated: non-binding anti-HIV-gp120 control antibody comprising the E345R mutation (IgG1-b12-E345R, ctrl), anti-CD27 antibodies IgG1-CD27-A, IgG1-CD27-B, IgG1-CD27-C, IgG1-CD27-D, IgG1-CD27-E, and IgG1-CD27-F, and prior art anti-CD27 benchmark antibodies IgG1-CD27-131A and IgG1-CD27-15.

FIG. 2 shows binding of anti-CD27 antibodies to (A,B) human and (C,D) cynomolgus monkey CD27 expressed on (A,C) T cells in PBMC or (B,D) CD27-transfected HEK293F cells, as determined by flow cytometry. Antibody binding is presented as the median fluorescence intensity (MFI). The anti-HIV-gp120 antibody IgG1-b12-FEAR (ctrl) was included as non-binding negative control antibody.

FIG. 3 shows binding of anti-CD27 antibodies IgG1-CD27-A, IgG1-CD27-B, and IgG1-CD27-C to human CD27-A59T variant expressed on HEK293F cells, as determined by flow cytometry. Antibody binding is presented as the median MFI. The anti-HIV-gp120 antibody IgG1-b12-FEAL (ctrl) was included as non-binding negative control antibody.

FIGS. 4A-4B show heatmaps of the proliferation of TCR stimulated (FIG. 4A) CD8⁺ and (FIG. 4B) CD4⁺ T cells in the presence of 1 μg/mL CD27-specific antibody variants IgG1-CD27-A, -B, or -C harboring the Fc mutations E430R or E345R in combination with the Fc mutations P329R, G237A, or K326A-E33A, as determined by flow cytometry in a CSFE dilution assay. PMBC from four human healthy donors were used as a source of T cells. T-cell proliferation was expressed as the T-cell division index or the percentage of proliferated T cells, that was calculated by gating for the cells that have gone through CFSE dilution (CFSE^{low peaks}) by using the FlowJo software.

FIG. 5 shows the (A-D) percentage of proliferated T cells, (E, F) the expansion index of (A, B) unstimulated or (C-F) TCR stimulated (A, C, E) CD4⁺ or (B, D, F) CD8⁺ T cells after incubation of human healthy donor PBMC with IgG1-CD27-A, IgG1-CD27-A-P329R-E345R or prior art anti-CD27 clones IgG1-CD27-131A, IgG1-CD27-CDX1127, and IgG1-CD27-BMS986215, as determined by flow cytometry. The anti-HIV-gp120 antibody variant IgG1-b12-E345R-P329R (ctrl) was included as non-binding negative control antibody. % Proliferated cells were calculated by gating for the cells that have gone through CFSE dilution (CFSE^{low peaks}). Expansion index identifies the fold increase of cells in the wells and was calculated using the Proliferation Modeling tool in FlowJo version 10. Manual adjustments to the peaks were made where necessary to define the number of the peaks present more consistently.

FIG. 6 shows binding of C1q to membrane-bound CD27 antibodies of the invention, as determined by FACS. IgG1-CD27-A variants containing a E430G or E345R hexamerization-enhancing mutation (IgG1-CD27-A-E430G and IgG1-CD27-A-E345R) and the P329R mutation (IgG1-CD27-A-P329R-E345R) were tested for their capacity to bind to C1q. The anti-HIV-gp120 antibody IgG1-b12-F405L (ctrl) was included as non-binding negative control antibody.

FIG. 7 shows binding of IgG1-CD27-A-P329R-E345R to human Fc receptors as determined by surface plasmon resonance (SPR). Biacore surface chips were covalently linked with anti-His antibody and coated with recombinant His-tagged Fc receptors (A) FcγRIa, (B) FcγRIIa-H, (C) FcγRIIa-R, (D) FcγRIIb, (E) FcγRIIIa-F, or (F) FcγRIIIa-V. The anti-HIV-gp120 antibody IgG1-b12 (ctrl) was included as a reference. Shown are absolute resonance units as determined by Biacore SPR after background subtraction (no Fc receptor flow-cell).

FIG. 8 shows binding of IgG1-CD27-A-P329R-E345R to human (A) CD4⁺ and (B) CD8⁺ T-cell subsets in human healthy donor PBMC samples, as determined by flow cytometry. Negative control antibody IgG1-b12-P329R-E345R (ctrl) is an anti-HIV gp120 non-binding isotype control antibody comprising the P329R and E345R mutations. Data presented is the mean MFI+/−SD of duplicate samples.

FIG. 9 shows CD27 agonist activity of anti-CD27 antibodies in presence and absence of FcγR-mediated crosslinking, as determined in a reporter assay. A fixed number of NFκB-luc2/CD27 Jurkat reporter cells was cultured with (A-E) IgG1-CD27-A-P329R-E345R or IgG1-CD27-A, (F-J) IgG1-CD27-131A, IgG1-CD27-CDX1127 or IgG1-CD27-BMS986215, in (A,F) absence or (B-J) presence of FcγRIIb-CHO-K1 cells, at a NFκB-luc2/CD27 Jurkat:FcγRIIb CHO-K1 ratio of (B,G) 1:1, (C,H) 1:1/3, (D,I) 1:1/9, or (E,J) 1:1/27. IgG1-b12-P329R-E345R and IgG1-b12 are anti-HIV gp120 non-binding control antibodies (ctrl). Luminescence was measured as a readout for CD27 activation and presented as relative luminescence units (RLU).

FIG. 10 shows the human IgG levels in plasma of SCID mice, after intravenous injection of 25 mg/kg IgG-CD27-A or IgG-CD27-A-P329R-E345R antibodies. Total human IgG plasma concentrations were determined by sandwich ELISA and plotted against time after injection. Data shown are mean plasma concentrations+/−SEM of blood samples per group (n=3 mice).

FIG. 11 shows the percentage of viable CD27⁺ Daudi cells after co-culturing for 4 h with hMDM (E:T=2:1) in the presence of IgG1-CD27-A-P329R-E345R or wild-type CD20 antibody IgG1-CD20. Daudi cells were labeled with CellTrace™ Violet and cell viability was measured by flow cytometry. Data shown are the mean of duplicates±SD percentage of viable Daudi cells (TO-PRO-3⁻CTV⁺CD11b⁻) normalized to the no antibody controls for one donor out of four tested in two experiments.

FIG. 12 shows C4d deposition upon incubation of IgG1-CD27-A-P329R-E345R in NHS as determined by ELISA. IgG1-b12-P329R-E345R is an isotype control antibody and IgG1-b12 is a control antibody with a WT Fc domain; IgG1-b12-RGY is a positive control antibody for C4d deposition (hexameric antibody in solution). Shown is mean±SD of triplicates of one representative experiment out of three performed.

FIG. 13 shows the inhibition of CD70 binding on Daudi cells by anti-CD27 antibodies. CD27⁺ Daudi cells were incubated with 6 μg/mL biotinylated recombinant human CD70 ECD in the presence or absence of 50 μg/mL of the non-binding control antibodies (IgG1-b12-P329E-E345R or IgG1-b12) or CD27 antibodies (IgG1-CD27-A, IgG1-CD27-A-P329R-E345R, IgG1-CD27-CDX1127, IgG1-CD27-BMS986215, or IgG1-CD27-131A). Binding of the biotinylated CD70 fragment to the Daudi cells was detected by flow cytometry using BV421-labeled streptavidin. Data shown are the gMFI±SD from duplicate wells of one representative experiment out of three performed.

FIGS. 14A-14B show expression levels of T-cell activation markers in polyclonally activated CD4⁺ and CD8⁺ T cells upon treatment with anti-CD27 antibodies. Human healthy donor PBMC were incubated with 0.1 μg/mL CD3 antibody and 30 μg/mL of IgG1-CD27-A-P329R-E345R, CD27 antibody benchmarks or non-binding control antibody IgG1-b12-P329R-E345R for two or five days. The expression levels of T-cell activation markers HLA-DR, CD69, GITR, CD25, CD107a, and 4-1BB on the surface of (FIG. 14A) CD4⁺ and (FIG. 14B) CD8⁺ T cells in antibody-treated samples were quantified by flow cytometry and presented as mean fold change in MFI (+SD) relative to the nonbinding control sample of the same donor. Dotted lines indicate the fold change for cells treated with IgG1-b12-P329R-E345R, which was used as a nonbinding control and set to 1. Data shown are from three donors tested in duplicate in one experiment.

FIG. 15 shows percentages of OVA-specific CD8⁺ T cells in spleen of hCD27-KI mice after immunization with OVA and treatment with anti-CD27 antibodies. hCD27-KI mice were injected s.c. with 5 mg OVA on days 0, 12 and 21, and simultaneously treated i.v. with 30 mg/kg IgG1-CD27-A-P329R-E345R, IgG1-CD27-CDX1127 or non-binding control antibody IgG1-b12-P329R-E345R. On day 28, mice were euthanized, spleens were resected, and processed as single cell suspensions. Expansion of OVA specific CD8⁺ T cells was evaluated by flow cytometry. Data shown are the mean of % OVA⁺ of CD8⁺ cells±SD per treatment group (5 mice per group) from one experiment performed.

FIG. 16 shows the number of IFNγ-producing splenocytes on day 28 after immunization with OVA and treatment with anti-CD27 antibodies as measured by IFNγ-ELISpot. hCD27-KI mice were injected s.c. with 5 mg OVA on days 0, 12 and 21, and simultaneously treated i.v. with 30 mg/kg IgG1-CD27-A-P329R-E345R, IgG1-CD27-CDX1127, or non-binding control antibody IgG1-b12-P329R-E345R. On day 28, spleens were resected, processed as single cell suspensions and IFNγ-producing splenocytes were detected using IFNγ-ELISpot. Data shown are the mean number of spots per well±SEM of each treatment group from one experiment performed (5 mice per group).

FIG. 17 shows the percentage of activated CD8⁺ T cells in the spleen of hCD27-KI mice after immunization with OVA and treatment with anti-CD27 antibodies. hCD27-KI mice were injected s.c. with 5 mg OVA on days 0, 12 and 21, and simultaneously treated i.v. with 30 mg/kg IgG1-CD27-A-P329R-E345R, IgG1-CD27-CDX1127, or non-binding control antibody IgG1-b12-P329R-E345R. On day 28, mice were euthanized, spleens were resected, and processed as single cell suspensions. Activation of CD8⁺ T cells was evaluated in spleen samples by measuring the percentage PD-1⁺ of CD8⁺ cells in spleen by flow cytometry. Data shown are the mean±SD per treatment group (5 mice per group) from one experiment performed.

FIG. 18 shows percentages of effector CD8⁺ T cells in the spleen of hCD27-KI mice after immunization with OVA and treatment with anti-CD27 antibodies. hCD27-KI mice were injected s.c. with 5 mg OVA on days 0, 12 and 21, and simultaneously treated i.v. with 30 mg/kg IgG1-CD27-A-P329R-E345R, IgG1-CD27-CDX1127, or non-binding control antibody IgG1-b12-P329R-E345R. On day 28 mice, were euthanized, spleens were resected, and processed as single cell suspensions. Expansion of memory T cells was evaluated by expression of CD44 and CD62L by flow cytometry. Data shown are the mean±SD per treatment group (5 mice per group) from one experiment performed. (A) Percentage CD8⁺CD44⁺CD62L⁻ effector memory of CD45⁺ cells. (B) Percentage CD44⁺CD62L⁻ effector memory of CD8⁺ T cells. (C) Percentage CD8⁺CD44⁻CD62L⁻ pre-effector of CD45⁺ cells. (D) Percentage CD44⁻CD62L⁻ pre-effector of CD8⁺ T cells.

FIG. 19 shows percentage of T cells in the spleen of hCD27-KI mice after immunization with OVA and treatment with anti-CD27 antibodies. hCD27-KI mice were injected s.c. with 5 mg OVA on days 0, 12 and 21, and simultaneously treated i.v. with 30 mg/kg IgG1-CD27-A-P329R-E345R, IgG1-CD27-CDX1127, or non-binding control antibody IgG1-b12-P329R-E345R. On day 28, mice were euthanized, spleens were resected, and processed as single cell suspensions. CD3⁺ cells in the blood and spleens were evaluated by flow cytometry. Data shown are the mean±SD per treatment group (5 mice per group) from one experiment performed.

FIG. 20 shows the effect of IgG1-CD27-A-P329R-E345R on T-cell cytokine production in antigen-specific studies. Cocultures of CLDN6-TCR-expressing CD8⁺ T cells that (A) express endogenous PD-1 or (B) overexpress PD-1 and autologous CLDN6-expressing iDC were incubated with 10 μg/mL IgG1-CD27-A-P329R-E345R, CD27 benchmark antibody IgG1-CD27-131A, or nonbinding control antibody IgG1-b12-P329R-E345R for two days. Cytokine levels in coculture supernatants were analyzed by multiplex ECLIA. Data shown are mean concentrations+SD of triplicate wells from one representative donor out of seven tested in two experiments performed. Abbreviations: CLDN6=claudin 6; ECLIA=electrochemiluminescence assay; iDC=immature dendritic cell; PD-1=programmed cell death protein 1; SD=standard deviation; TCR=T cell receptor.

FIG. 21 shows expression of cytotoxicity-associated molecules in antigen-specific CD8⁺ T cells incubated with IgG1-CD27-A-P329R-E345R. CLDN6-TCR-electroporated CD8⁺ T cells were cocultured with hCLDN6-MDA-MB-231 cells in the presence of IgG1-CD27-A-P329R-E345R, CD27 benchmark IgG1-CD27-131A, or nonbinding control antibody IgG1-b12-P329R-E345R for two days. Intracellular expression of GzmB and CD107a was determined by flow cytometry. The percentage of CD8+ T cells expressing both GzmB and CD107a, as well as expression levels of GzmB and CD107a (MFI normalized to IgG1-b12-P329R-E345R) in CD8+ T cells is shown. Data shown are mean±SD of six donors tested in single replicate in experiments two experiments. **, P<0.01; *, P<0.05; Friedman-test with Dunn's multiple comparisons test. Abbreviations: CLDN6=claudin 6; GzmB=granzyme B; MFI=mean fluorescence intensity; SD=standard deviation; TCR=T-cell receptor.

FIG. 22 shows antigen-specific CD8+ T-cell mediated tumor cell kill in the presence of IgG1-CD27-A-P329R-E345R. CD8+ T-cell mediated kill of hCLDN6-MDA-MB-231 cells was evaluated by real-time cell analysis. CLDN6 TCR electroporated CD8+ T cells were cocultured with hCLDN6-MDA-MB-231 cells in the presence of IgG1-CD27-A-P329R-E345R, CD27 benchmark IgG1-CD27-131A, or nonbinding control antibody IgG1-b12-P329R-E345R for five days. Cell index values were derived from impedance measurements conducted at two-hour intervals. AUC were obtained from cell index data over five days of coculture. The AUC of each treatment condition was normalized to IgG1-b12-P329R-E345R-treated cultures from the same donor. Data shown are mean±SD from six donors tested in duplicate in experiments in two experiments. **, P<0.01; Friedman-test with Dunn's multiple comparisons test. Abbreviations: AUC=area under the curve; CLDN6=claudin 6; SD=standard deviation; TCR=T-cell receptor.

FIG. 23 shows absolute cell numbers of CD4+ and CD8+ T cells and NK cells in primary tumor cultures after treatment with IgG1-CD27-A-P329R-E345R. Human NSCLC tumor tissues were cultured with low-dose IL-2 (45 to 50 U/mL) in the presence or absence of 10 μg/mL IgG1-CD27-A-P329R-E345R. Absolute cell counts of the TIL subsets were determined by flow cytometry after 14 days of treatment. Data shown are average±SD of four replicate wells from one out of five tumor tissues tested in one experiment out of four performed. Abbreviations: IL=interleukin; NK=natural killer; NSCLC=non-small cell lung cancer; SD=standard deviation; U/mL=units per mL.

FIG. 24 shows molecular proximity determined by bioluminescence resonance energy transfer (BRET) analysis between IgG1-CD27-A-P329R-E345R antibodies on the cell surface of Daudi and huCD27-K562 cells. Cells were incubated with mixtures of NanoLuc- (donor) and HaloTag- (acceptor) tagged antibodies (5 μg/mL each): IgG1-CD27-A-P329R-E345R, WT IgG1-CD27-A or nonbinding control IgG1-b12-P329R-E345R as indicated. The antibody pair IgG1-CD20-11B8-E430G-LNLuc and IgG1-CD37-37.3-E430G-LHalo was used as positive control. BRET was calculated in milliBRET units (mBU)=(618 nmem/460 nmem)×1000, and corrected for donor bleed-through by subtracting no-ligand control values. Data shown are the corrected BRET from duplicate wells of one representative experiment out of three performed.

FIG. 25 shows binding of IgG1-CD27-A-P329R-E345R to M0 and M1 macrophages compared to a WT IgG1 antibody (IgG1-b12) with an irrelevant antigen-binding region as a positive control for FcγRIa binding, and a variant of the same antibody carrying the P329R and E345R mutations (IgG1-b12-P329R-E345R). Binding of the antibodies to the macrophages was detected by flow cytometry using PE-labeled goat anti-human secondary antibody. Data shown are mean±SD of two donors tested.

FIG. 26 shows binding of IgG1-PD1 to PD-1 of different species. CHO-S cells transiently transfected with PD-1 of different species were incubated with IgG1-PD1, pembrolizumab, or non-binding control antibodies IgG1-ctrl-FERR and IgG4-ctrl and binding analyzed using flow cytometry. Non-transfected CHO-S cells incubated with IgG1-PD1 were included as a negative control. A-B. Data shown are the geometric mean fluorescence intensities (gMFI)±SD of duplicate wells from one representative experiment out of four experiments. C-D. Data shown are the gMFI±SD of duplicate wells from one representative experiment out of two experiments. E. Data shown are the geometric mean fluorescence intensities (gMFI)±SD of duplicate wells from one representative experiment out of four experiments. Abbreviations: gMFI=geometric mean fluorescence intensity; PD-1=programmed cell death protein 1; PE=R-Phycoerythrin.

FIG. 27 shows competitive binding of IgG1-PD1 with PD-L1 and PD-L2 to human PD-1. CHO-S cells transiently transfected with human PD-1 were incubated with 1 μg/mL biotinylated recombinant human PD-L1 (A) or PD-L2 (B) in the presence of IgG1-PD1 or pembrolizumab. IgG1-ctrl-FERR was included as a negative control. Cells were stained with streptavidin-allophycocyanin, and the percentage of cells binding biotinylated PD-L1 or PD-L2 was determined by measuring the percentage of streptavidin-allophycocyanin⁺ cells using flow cytometry. The percentage of streptavidin-allophycocyanin⁺ cells in the no antibody control and in a non-transfected sample are indicated with dashed lines. Data shown are from single replicates from one representative experiment out of three separate experiments. Abbreviations: Ab=antibody; CHO-S=Chinese hamster ovary, suspension; ctrl=control; FERR=L234F/L235E/G236R-K409R; PD-1=programmed cell death protein 1; PD-L1=programmed cell death 1 ligand 1; PD-L2=programmed cell death 1 ligand 2.

FIG. 28 shows functional inhibition of the PD-1/PD-L1 checkpoint by IgG1-PD1. Blockade of the PD-1/PD-L1 axis was tested using a cell-based bioluminescent PD-1/PD-L1 blockade reporter assay. Data shown are mean luminescence±SD of duplicate wells in one representative experiment out of five (pembrolizumab and IgG1-PD1), three (IgG1-ctrl-FERR) or two (nivolumab) experiments. Abbreviations: FERR=L234F/L235E/G236R-K409R; PD1=programmed cell death protein 1; PD-L1=programmed cell death 1 ligand 1; RLU=relative light units; SD=standard deviation.

FIG. 29 shows the enhancement of CD8⁺ T-cell proliferation by IgG1-PD1 in an antigen-specific T-cell proliferation assay. Human CD8⁺ T cells were electroporated with RNA encoding a CLDN6-specific TCR and RNA encoding PD-1 and labeled with CFSE. The T cells were then co-cultured with iDCs electroporated with CLDN6-encoding RNA, in the presence of IgG1-PD1, pembrolizumab, nivolumab, or IgG1-ctrl-FERR. CFSE dilution in T cells was analyzed by flow cytometry after 4 d and used to calculate the expansion index. Data from one representative donor (26268_B) out of four donors evaluated in three independent experiments are shown. Error bars represent SD of duplicate wells. Curves were fitted by 4-parameter logarithmic fit using GraphPad Prism. Abbreviations: CFSE=carboxyfluorescein succinimidyl ester; FERR=L234F/L235E/G236R-K409R; PD1=programmed cell death protein 1; SD=standard deviation.

FIG. 30 shows IgG1-PD1-induced IFNγ secretion in an allogeneic MLR assay. Three unique donor pairs of allogeneic human mDCs and CD8⁺ T cells were cocultured in the presence of IgG1-PD1 or pembrolizumab for 5 d. IgG1-ctrl-FERR and an IgG4 isotype control were included as negative controls. IFNγ secretion was analyzed in the supernatant using an IFNγ-specific immunoassay. Data shown are mean±standard error of the mean (SEM) concentration for three unique allogeneic donor pairs. Abbreviations: FERR=L234F/L235E/G236R-K409R; IFN=interferon; IgG=immunoglobulin G; mDC=mature dendritic cell; MLR=mixed lymphocyte reaction; SEM=standard error of the mean.

FIGS. 31A-31B show IgG1-PD1-induced cytokine secretion in an allogeneic MLR assay. Three unique donor pairs of allogeneic human mDCs and CD8⁺ T cells were cocultured in the presence of 1 μg/mL IgG1-PD1 or pembrolizumab for 5 d. IgG1-ctrl-FERR was included as a negative control. Cytokine secretion was analyzed in the supernatant using Luminex. (FIG. 31A) Cytokine levels are represented as the average fold change over the cytokine levels measured in untreated cocultures. (FIG. 31B) Shown are the levels of cytokine production of three unique allogeneic donor pairs, with horizontal lines indicating the mean, upper, and lower limits. Abbreviations: FC=fold change; FERR=L234F/L235E/G236R-K409R; GM-CSF=granulocyte macrophage colony-stimulating factor; IgG=immunoglobulin G; IL=interleukin; MCP-1=monocyte chemoattractant protein 1; mDC=mature dendritic cell; MLR=mixed lymphocyte reaction; TNF=tumor necrosis factor.

FIG. 32 shows C1q binding to membrane-bound IgG1-PD1. Binding of C1q to IgG1-PD1 was analyzed using stimulated human CD8⁺ T cells. After incubation with IgG1-PD1, IgG1-ctrl-FERR, IgG1-ctrl, or positive control antibody IgG1-CD52-E430G (without inertness mutations and with a hexamerization-enhancing mutation), cells were incubated with human serum as a source of C1q. Binding of C1q was detected with a FITC-conjugated rabbit anti-C1q antibody. Data shown are the geometric mean fluorescence intensities (gMFI)±standard deviation (SD) from duplicate wells from one representative donor out of seven donors across three comparable experiments. Abbreviations: FITC=fluorescein isothiocyanate; gMFI=geometric mean fluorescence intensity; PE=R-phycoerythrocyanin.

FIG. 33 shows FcγR binding of IgG1-PD1. The binding of IgG1-PD1 to immobilized human recombinant FcγR constructs was analyzed by SPR in a qualified assay (n=1). FcγRIa (A), FcγRIIa-H131 (B), FcγRIIa-R131 (C), FcγRIIb (D), FcγRIIIa-F158 (E), and FcγRIIIa-V158 (F) binding of IgG1-PD1. The antibody IgG1-ctrl (without the FER inertness mutations) was included as a positive control for binding. Abbreviations: ctrl=control; FcγR=Fc gamma receptor; IgG=immunoglobulin G; PD-1=programmed cell death protein 1; RU=resonance units.

FIG. 34 shows FcγR binding of IgG1-PD1 and several other anti-PD-1 antibodies. The binding of IgG1-PD1, nivolumab, pembrolizumab, dostarlimab, and cemiplimab to immobilized human recombinant FcγR constructs was analyzed by SPR (n=3). FcγRIa (A), FcγRIIa-H131 (B), FcγRIIa-R131 (C), FcγRIIb (D), FcγRIIIa-F158 (E), and FcγRIIIa-V158 (F) binding of the test antibodies. The IgG1-ctrl and IgG4-ctrl antibodies were included as positive controls for FcγR binding of IgG1 and IgG4 molecules with wild-type Fc regions. Shown is the binding response±SD of three separate experiments. Abbreviations: ctrl=control; FcγR=Fc gamma receptor; IgG=immunoglobulin G; PD-1=programmed cell death protein 1; RU=resonance units.

FIG. 35 shows FcγRIa binding of IgG1-PD1 and several other anti-PD-1 antibodies. The binding of IgG1-PD1, nivolumab, pembrolizumab, dostarlimab, and cemiplimab to CHO-S cells transiently expressing human FcγRIa was analyzed by flow cytometry. IgG1-ctrl and IgG1-ctrl-FERR were included as a positive and negative control, respectively. Abbreviations: ctrl=control; FcγR=Fc gamma receptor; FERR=L234F/L235E/G236R-K409R; huIgG=human immunoglobulin G; PD-1=programmed cell death protein 1; PE=R-phycoerythrin.

FIG. 36 shows total human IgG in mouse plasma samples. Mice were injected intravenously with 1 or 10 mg/kg IgG1-PD1 at t=0 and serial plasma samples were taken at 10 min, 4 h, 1 d, 2 d, 8 d, 14 d, and 21 d after injection. Total huIgG in plasma samples was determined by ECLIA for each mouse. Data are represented as mean huIgG concentration±SD of three individual mice. Dashed lines indicate the plasma concentrations of wild-type (wt) huIgG predicted by a two-compartment model based on IgG clearance in humans (Bleeker et al., 2001, Blood. 98(10):3136-42). Dotted lines indicate the LLOQ and ULOQ. Abbreviations: huIgG=human IgG; IgG=immunoglobulin G; LLOQ=lower limit of quantitation; PD-1=programmed cell death protein 1; SD=standard deviation; ULOQ=upper limit of quantitation.

FIG. 37 shows antitumor activity of IgG1-PD1 in human PD-1 knock-in mice. The MC38 colon cancer syngeneic tumor model was established by SC implantation in hPD-1 KI mice. Mice were administered 0.5, 2, or 10 mg/kg IgG1-PD1 or pembrolizumab or 10 mg/kg IgG1-ctrl-FERR 2QW×3 (9 mice per group). (A) Average tumor volume±SEM in each group, until the last time point the group was complete. (B) Tumor volumes of the different groups on the last day all groups were complete (Day 11). Data shown are the tumor volumes in individual mice in each treatment group, as well as mean tumor volume±SEM per treatment group. Mann-Whitney analysis was used to compare tumor volumes of the treatment groups to the IgG1-ctrl-FERR-treated group, with *p<0.05, **p<0.01, and ***p<0.001. C. Progression-free survival, defined as the percentage of mice with tumor volume smaller than 500 mm³, is shown as a Kaplan-Meier curve. Analysis excluded one mouse from the 2 mg/kg IgG1-PD1 group that was found dead due to undetermined cause on day 16, before the tumor volume had exceeded 500 mm³. Abbreviations: 2QW×3=twice per week for three weeks; ctrl=control; FERR=L234F/L235E/G236R/K409R mutations; IgG=immunoglobulin G; KI=knock-in; PD-1=programmed cell death protein 1; SC=subcutaneous; SEM=standard error of the mean.

FIG. 38 shows peripheral T-cell count dynamics in human PD-1 knock-in mice treated with IgG1-PD1. The MC38 colon cancer syngeneic tumor model was established by SC implantation in hPD-1 KI mice. Mice were administered 0.5 or 10 mg/kg IgG1-PD1, 10 mg/kg pembrolizumab, or 10 mg/kg IgG1-ctrl-FERR on Day 0, 3, and 7 (12 mice per group). Peripheral blood samples were collected after euthanasia from four mice per group on Day 2, 4, and 8 and analyzed by flow cytometry. Shown is the mean±SD of the number of CD3+ (A), CD4+ (B), and CD8+ (C) T cells per μL blood within the viable CD45+ leukocyte subpopulation. Mann-Whitney analysis was used to compare the treatment groups to the IgG1-ctrl-FERR-treated group, and the 10 mg/kg IgG1-PD1 group with the 10 mg/kg pembrolizumab group, with *p<0.05. Abbreviations: ctrl=control; FERR=L234F/L235E/G236R/K409R mutations; IgG=immunoglobulin G; KI=knock-in; PD-1=programmed cell death protein 1; SC=subcutaneous; SD=standard deviation.

FIG. 39 shows PD markers on splenic T-cell subsets in human PD-1 knock-in mice treated with IgG1-PD1. The MC38 colon cancer syngeneic tumor model was established by SC implantation in hPD-1 KI mice. Mice were administered 0.5 or 10 mg/kg IgG1-PD1, 10 mg/kg pembrolizumab, or 10 mg/kg IgG1-ctrl-FERR on Day 0, 3, and 7 (12 mice per group). Spleens were harvested on Day 2, 4, and 8 (n=4 mice per group and timepoint) and analyzed by flow cytometry. Shown is the mean±SD of the percentage of effector memory (CD44+CD62L⁻), central memory (CD44+CD62L+), and naïve (CD44−CD62L+) CD8+ T cells (A), and the percentage of MHC class II+ cells within the total CD8+ T-cell population (B) in the spleen on Day 8. Mann-Whitney analysis was used to compare the treatment groups to the IgG1-ctrl-FERR-treated group, and the 10 mg/kg IgG1-PD1 group with the 10 mg/kg pembrolizumab group, with *p<0.05. Abbreviations: ctrl=control; FERR=L234F/L235E/G236R/K409R mutations; IgG=immunoglobulin G; KI=knock-in; PD-1=programmed cell death protein 1; SC=subcutaneous; SD=standard deviation.

FIG. 40 shows Changes in intratumoral cells in human PD-1 knock-in mice treated with IgG1-PD1. The MC38 colon cancer syngeneic tumor model was established by SC implantation in hPD-1 KI mice. Mice were administered 0.5 or 10 mg/kg IgG1-PD1, 10 mg/kg pembrolizumab, or 10 mg/kg IgG1-ctrl-FERR on Day 0, 3, and 7 (12 mice per group). Xenograft tumors were excised on Day 8 (n=4 mice per group) and analyzed by IHC. Shown is the mean±SD of the percentage of CD3⁺ T cells (A), CD4⁺ T cells (B), CD8⁺ T cells (C), and GZMB+ cells (D) of all nucleated cells on Day 8. Mann-Whitney analysis was used to compare the treatment groups to the IgG1-ctrl-FERR-treated group, and the 10 mg/kg IgG1-PD1 group with the 10 mg/kg pembrolizumab group, with *p<0.05. Abbreviations: ctrl=control; FERR=L234F/L235E/G236R/K409R mutations; GZMB=granzyme B; IgG=immunoglobulin G; KI=knock-in; PD-1=programmed cell death protein 1; SC=subcutaneous; SD=standard deviation.

FIGS. 41A-41B show binding of IgG1-PD1 and other anti-PD-1 antibodies to human monocyte-derived FcγR+M2c-like macrophages. (FIG. 41A) Expression of FcγRIa, FcγRII, FcγRIIIa, and PD-1, relative to the relevant isotype controls and unstained M2c-like macrophages, visualized in overlay histograms of normalized data, of one representative donor out of three donors tested. (FIG. 41B) The binding of IgG1-PD1, pembrolizumab, nivolumab, and control antibodies to human monocyte-derived FcγR+M2c-like macrophages after 24 h of incubation was analyzed by flow cytometry. Binding is shown relative to that of the background control (binding with secondary antibody only, indicated by the dotted black line). Dots represent three individual donors measured in two independent experiments, and bar graphs and error bars represent the mean±SD of the three donors, respectively. Abbreviations: ctrl=control; FERR=L234F/L235E/G236R/K409R mutations; PD-1=programmed cell death protein 1; SD=standard deviation.

FIG. 42 shows FcγR signaling induced by membrane-bound IgG1-PD1 and other anti-PD-1 antibodies. FcγR signaling induced by membrane-bound IgG1-PD1 and several other anti-PD-1 antibodies was tested using cell-based bioluminescent FcγRI (A), FcγRIIa-R131 (B), FcγRIIa-H131 (C), and FcγRIIb (D) reporter assays. IgG1-CD52-E430G with the hexamerization-enhancing E430G mutation was included as a positive control. Data shown are mean relative light units±SD of duplicate wells in one representative experiment out of three experiments. Abbreviations: Ab=antibody; FERR=L234F/L235E/G236R/K409R mutations; PD-1=programmed cell death protein 1; RLU=relative light units; SD=standard deviation.

FIGS. 43A-43B show proliferation of polyclonally activated CD4+ and CD8+ T cells induced by IgG1-CD27-A-P329R-E345R in combination with DuoBody-PD-L1×4-1BB. CTV-labeled human healthy donor PBMC were incubated with CD3 antibody and IgG1-CD27-A-P329R-E345R and/or DuoBody-PD-L1×4-1BB for four days. CTV dilution in T cells was analyzed by flow cytometry and used to calculate the expansion index. Data shown are from (FIG. 43A) CD4+ and (FIG. 43B) CD8+ T cells in samples stimulated with 0.1 μg/mL CD3 antibody. Values present expansion indices of single replicates from one representative donor out of six donors tested in four experiments performed. Abbreviations: CD=cluster of differentiation; CTV=cell trace violet; PBMC=peripheral blood mononuclear cells; PD-L1=programmed cell death 1 ligand 1; TCR=T-cell receptor.

FIG. 44 shows the effect of IgG1-CD27-A-P329R-E345R or IgG1-CD27-131A in combination with DuoBody-PD-L1×4-1BB, IgG1-PD1, pembrolizumab, nivolumab or atezolizumab on T-cell proliferation in vitro. Human CD8+ T cells were electroporated with RNA encoding a CLDN6-specific TCR and RNA encoding PD-1, and labeled with CFSE. The T cells were then co-cultured with iDCs electroporated with CLDN6, in the presence of (A) IgG1-CD27-A-P329R-E345R (1 or 10 μg/mL) and/or DuoBody-PD-L1×4-1BB (0.2 μg/mL), or in the presence of (B) IgG1-CD27-A-P329R-E345R (0.1, 1 or 10 μg/mL), IgG1-CD27-131A (10 μg/mL), IgG1-PD1 (0.8 μg/mL), pembrolizumab (0.8 μg/mL), nivolumab (1.6 μg/mL), atezolizumab (0.4 μg/mL) or a combination of IgG1-CD27-A-P329R-E345R or IgG1-CD27-131A and one of the anti-PD-(L)1-antibodies, for 4 d. CFSE dilution in T cells was analyzed by flow cytometry and used to calculate the expansion index. Data from one representative donor out of three to seven donors tested in three independent experiments are shown (IgG1-CD27-A-P329R-E345R, IgG1-PD1, pembrolizumab: n=7; nivolumab, atezolizumab: n=6; IgG1-CD27-131A: n=3). Error bars indicate SD of duplicate wells. Dotted line represents expansion index of CD8+ T cells co-cultured with iDCs without antibody treatment. CFSE, Carboxyfluorescein succinimidyl ester; CLDN6, claudin-6; iDC, immature dendritic cell; PD-1, programmed cell death protein 1; SD, standard deviation; TCR, T-cell receptor.

FIG. 45 shows the effect of IgG1-CD27-A-P329R-E345R or IgG1-CD27-131A in combination with DuoBody-PD-L1×4-1BB, IgG1-PD1, pembrolizumab, nivolumab or atezolizumab on cytokine secretion in vitro. Human CD8+ T cells expressing a CLDN6-specific TCR and PD-1 were co-cultured with CLDN6-expressing iDCs as in FIG. 37, in the presence of (A) IgG1-CD27-A-P329R-E345R (1 or 10 μg/mL) and/or DuoBody-PD-L1×4-1BB (0.2 μg/mL), or in the presence of (B) IgG1-CD27-A-P329R-E345R (0.1, 1 or 10 μg/mL), IgG1-CD27-131A (10 μg/mL), IgG1-PD1 (0.8 μg/mL), pembrolizumab (0.8 μg/mL), nivolumab (1.6 μg/mL), atezolizumab (0.4 μg/mL) or a combination of IgG1-CD27-A-P329R-E345R or IgG1-CD27-131A and one of the anti-PD-(L)1-antibodies. After 2 (A) or 4 (B) d, cytokine concentrations of (A) IFNγ, GM-CSF, TNFα, and IL-2 or (B) IFNγ were determined in the supernatants by multiplexed ECLIA. Data from one representative donor out of four (A) or three to seven (B, IgG1-CD27-A-P329R-E345R, IgG1-PD1, pembrolizumab: n=7; nivolumab, atezolizumab: n=6; IgG1-CD27-131A: n=3) donors tested are shown. Error bars indicate SD of triplicate wells. Dotted lines represent cytokine concentrations in CD8+ T cell/iDC co-cultures without antibody treatment. CFSE, Carboxyfluorescein succinimidyl ester; CLDN6, claudin-6; ECLIA, electrochemiluminescence immunoassay; iDC, immature dendritic cell; PD-1, programmed cell death protein 1; SD, standard deviation; TCR, T-cell receptor.

FIG. 46 shows the effect of combined IgG1-CD27-A-P329R-E345R and DuoBody-PD-L1×4-1BB treatment on CD8+ T-cell cytotoxicity in vitro. CD8+ T-cell mediated cytotoxic activity towards MDA-MB-231_hCLDN6 cells was evaluated by real-time cell analysis. CLDN6-TCR-expressing CD8+ T cells were co-cultured with hCLDN6-MDA-MB-231 cells for 5 d in the presence of IgG1-CD27-A-P329R-E345R (1 or 10 μg/mL), DuoBody-PD-L1×4-1BB (0.2 μg/mL), a combination of both, or the negative control antibody IgG1-b12-P329R-E345R (10 μg/mL). Cell index values were derived from impedance measurements conducted at 2-hour intervals. (A) Cell index curves of one donor, out of six donors evaluated. Error bars indicate SD of duplicate wells. (B) AUC analysis was performed using cell index data over the entire duration of the 5-d co-culture. AUC of each treatment condition was normalized to IgG1-b12-P329R-E345R-treated cultures from the same donor. Pooled data from two individual experiments are shown. Error bars indicate SD (n=6, average of duplicate wells for each donor). *, P<0.05; Friedman test with Dunn's multiple comparisons test.

FIG. 47 shows the effect of combined IgG1-CD27-A-P329R-E345R and DuoBody-PD-L1×4-1BB treatment on GzmB and CD107a expression in vitro. CLDN6-TCR-expressing CD8+ T cells were co-cultured with MDA-MB-231_hCLDN6 cells for 2 d in the presence of IgG1-CD27-A-P329R-E345R (1 or 10 μg/mL), DuoBody-PD-L1×4-1BB (0.2 μg/mL), a combination of both, or the negative control antibody IgG1-b12-P329R-E345R (10 μg/mL). Intracellular expression of GzmB and CD107a was analyzed by flow cytometry. The percentages of CD8+ T cells expressing (A) GzmB, (B) CD107a, or (C) both GzmB and CD107a (normalized to IgG1-b12-P329R-E345R, as indicated by dotted lines) in CD8+ T cells are shown. Dotted line and grey shading indicate the mean percentage and the range (i.e. maximum and minimum) of GzmB+CD107a+ cells in co-cultures treated with IgG1-b12-P329R-E345R. Pooled data from two individual experiments (n=6 donors) are shown. Error bars indicate SD. *, P<0.05; Friedman test with Dunn's multiple comparisons test.

FIG. 48 shows cell numbers of NSCLC TIL subsets. Tumor fragments derived from a human NSCLC specimen were cultured with low-dose IL-2 only, or in the presence of 1 μg/mL IgG1-CD27-A-P329R-E345R, 0.2 μg/mL DuoBody-PD-L1×4-1BB, or a combination of both. Absolute cell counts of the indicated cell subsets were determined by flow cytometry after 14 d. Symbols represent replicate wells, lines represent average of replicates from one experiment, out of three experiments performed. NK, natural killer; PD-L1, programmed cell death 1 ligand 1; SD, standard deviation.

FIG. 49 shows cytokine concentrations as measured in CD8 mixed-lymphocyte reactions (MLR) assays, treated with IgG1-PD1 (1 μg/mL) or pembrolizumab (1 μg/mL) in the absence or presence of 10 μg/mL IgG1-CD27-A-P329R-E345R. Concentration levels (pg/ml) of (A) IFNγ and (B) GM-CSF are shown. Pooled data from two individual experiments (n=2 donors) are shown. Error bars indicate SD.

FIGS. 50A-50B show synergy analyses for cytokine concentrations as measured in CD8 MLR assays, treated with IgG1-PD1 (1 μg/mL) or pembrolizumab (1 μg/mL) in the absence or presence of 10 μg/mL IgG1-CD27-A-P329R-E345R. The concentrations of IFNγ in each treatment condition were normalized by subtracting the control values (no treatment control wells) and expressed as percentage of the maximal value in the assay (IFNγ induction). The IFNγ induction values represent an average of two replicates. The interaction between the two antibodies combined was analyzed using the SynergyFinder package (v3.2.2)1 in R (v4.1.0). The synergistic effect was defined as the excess of observed effect over expected effect as calculated by two reference models (synergy scoring models): Highest Single Agent (HSA) and Bliss. HSA and Bliss synergy analyses plots are shown for 2 different donor pairs (A and B).

FIG. 51 shows the effect of combined IgG1-CD27-A-P329R-E345R and PD-1/PD-L1 inhibitor treatment on CD8+ T-cell cytotoxicity in vitro. CD8+ T-cell mediated cytotoxic activity towards MDA-MB-231_hCLDN6 cells was evaluated by real-time cell analysis. PD-1- and CLDN6-TCR-expressing CD8+ T cells were co-cultured with MDA-MB-231_hCLDN6 cells for 5-6 d in the presence of IgG1-CD27-A-P329R-E345R (10 μg/mL), IgG1-PD1 (0.8 μg/mL), pembrolizumab (0.8 μg/mL), nivolumab (1.6 μg/mL), atezolizumab (0.4 μg/mL), or the non-binding control antibody IgG1-b12-P329R-E345R (10 μg/mL), either as single agent or as a combination of IgG1-CD27-A-P329R-E345R and a PD-1/PD-L1 inhibitor. Cell index values were derived from impedance measurements conducted at 2-3-hour intervals. (A) Cell index curves of one donor, out of 11-14 donors evaluated (B) AUC analysis was performed using cell index data over the entire duration of the 5-6 days co-culture. AUC of each treatment condition was normalized to IgG1-b12-P329R-E345R-treated cultures from the same donor (dotted line). Pooled data from 11-14 donors tested in six individual experiments are shown. Error bars indicate SD (symbols indicate average of duplicate wells for each donor). *, P<0.05; **, P<0.01; ***, P<0.001; Friedman test with Dunn's multiple comparisons test.

FIG. 52 shows the effect of combined IgG1-CD27-A-P329R-E345R and PD-1/PD-L1 inhibitor treatment on GzmB and CD107a expression by CD8+ T cells in vitro. PD-1- and CLDN6-TCR-expressing CD8+ T cells were co-cultured with MDA-MB-231_hCLDN6 cells for 2 d in the presence of IgG1-CD27-A-P329R-E345R (10 μg/mL), IgG1-PD1 (0.8 μg/mL), pembrolizumab (0.8 μg/mL), nivolumab (1.6 μg/mL), atezolizumab (0.4 μg/mL), or the non-binding control antibody IgG1-b12-P329R-E345R (10 μg/mL), either as single agent or as a combination of IgG1-CD27-A-P329R-E345R and a PD-1/PD-L1 inhibitor. Intracellular expression of GzmB and CD107a was analyzed by flow cytometry. The percentages of CD8+ T cells expressing both GzmB and CD107a are shown as pooled data from 11-14 donors tested in six individual experiments. Dotted line indicates the mean of GzmB+CD107a+ cells in co-cultures treated with IgG1-b12-P329R-E345R. Error bars indicate SD (symbols indicate average of duplicate wells for each donor). *, P<0.05; **, P<0.01; ***, P<0.001; ****, P<0.0001; Friedman test with Dunn's multiple comparisons test.

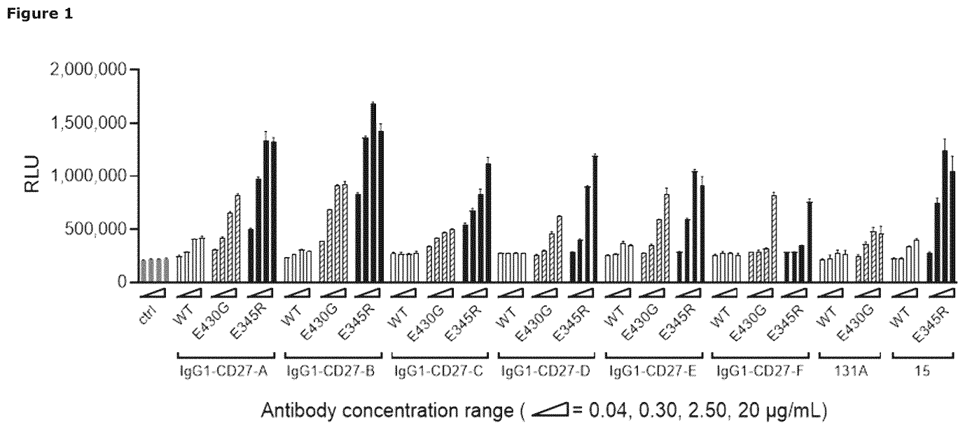

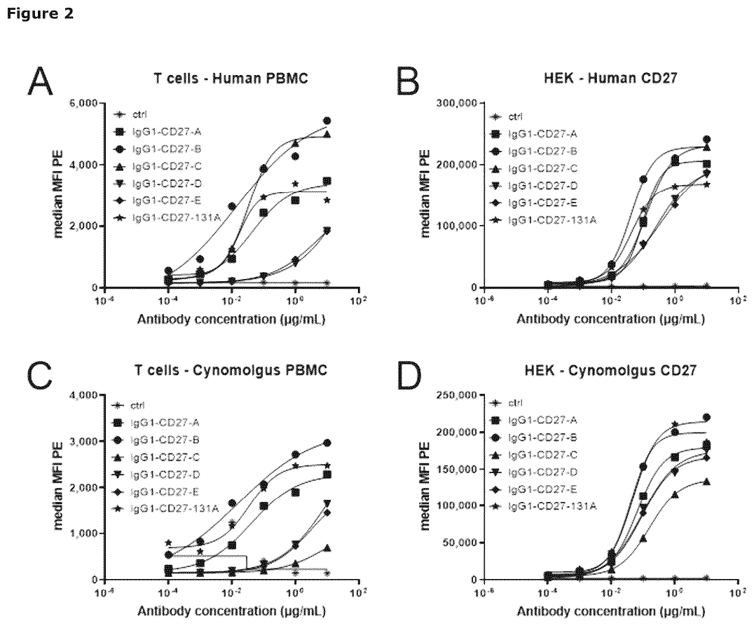

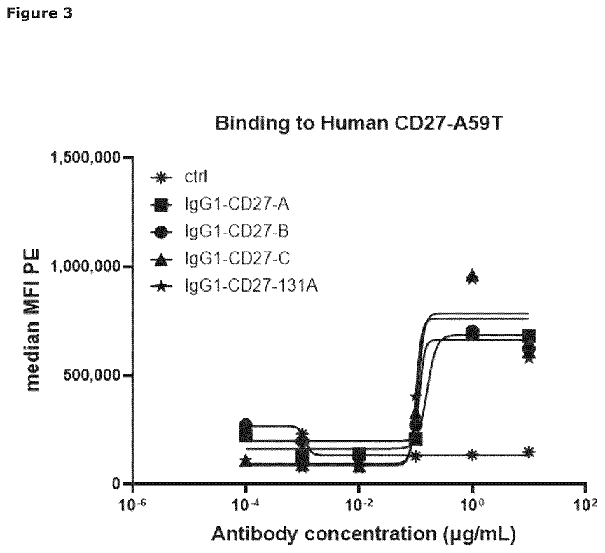

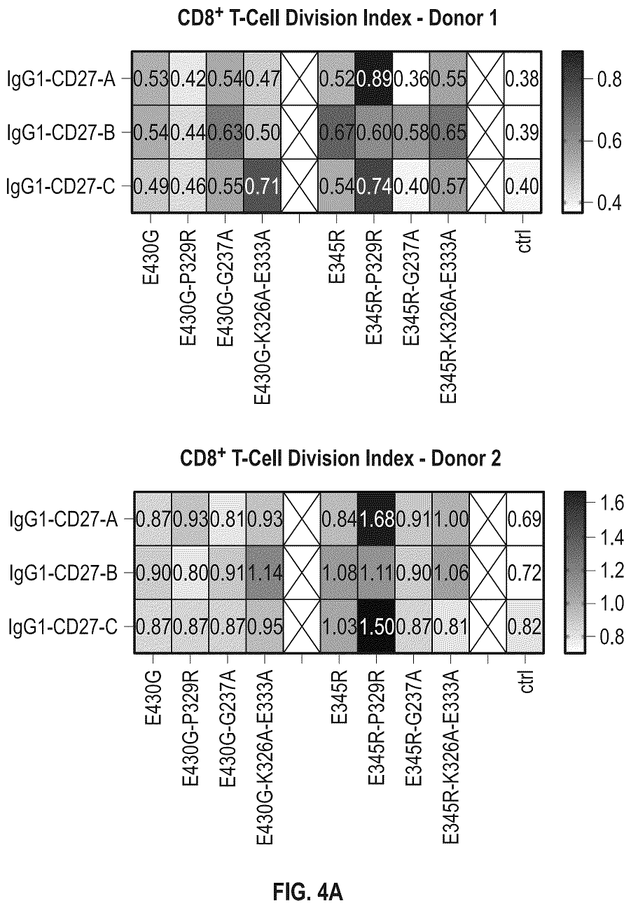

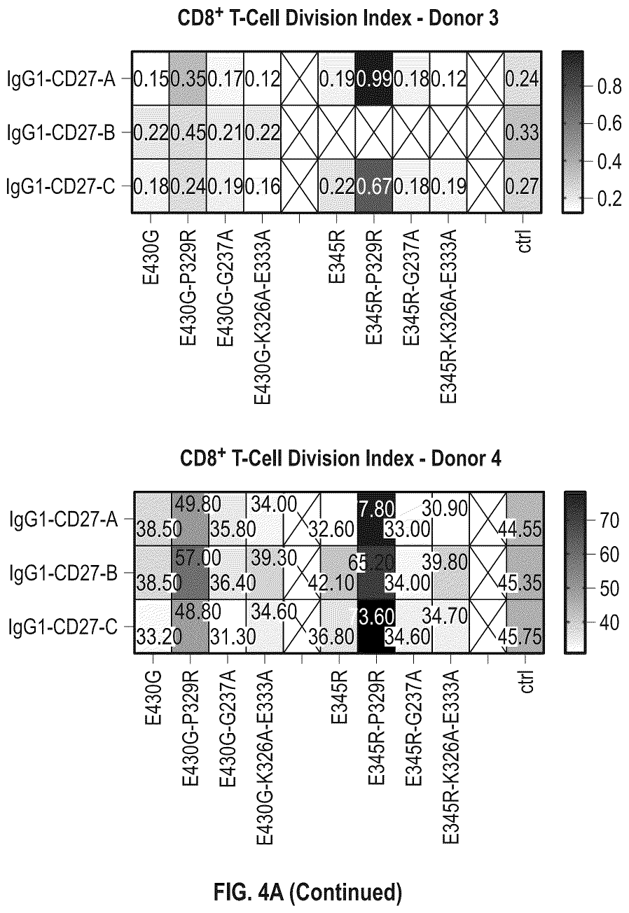

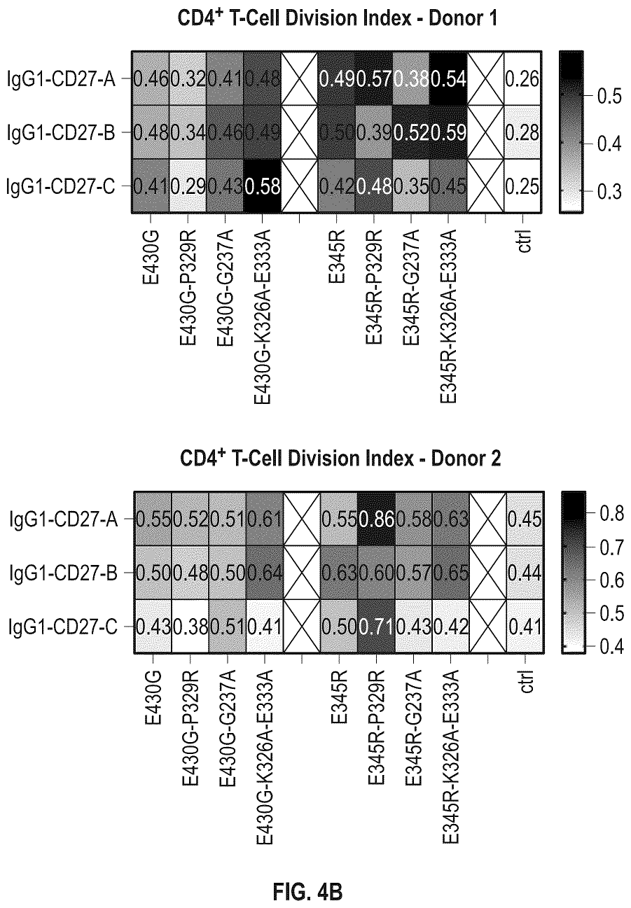

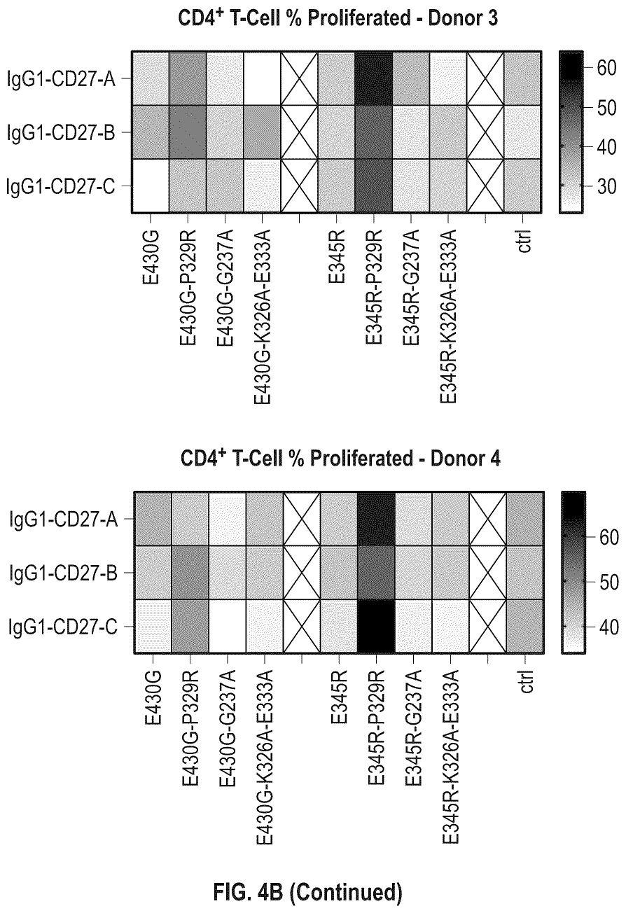

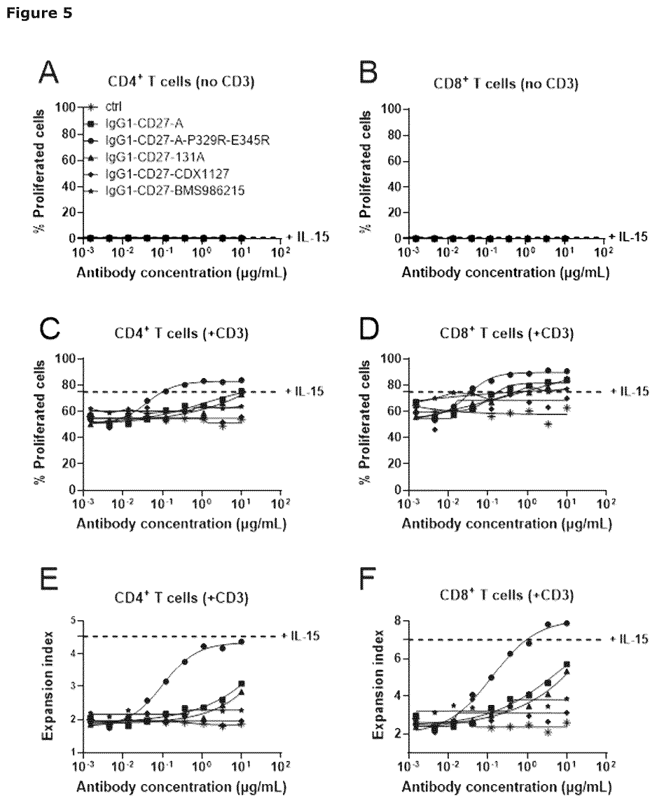

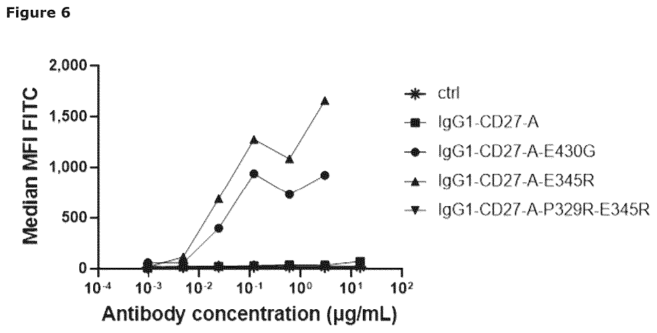

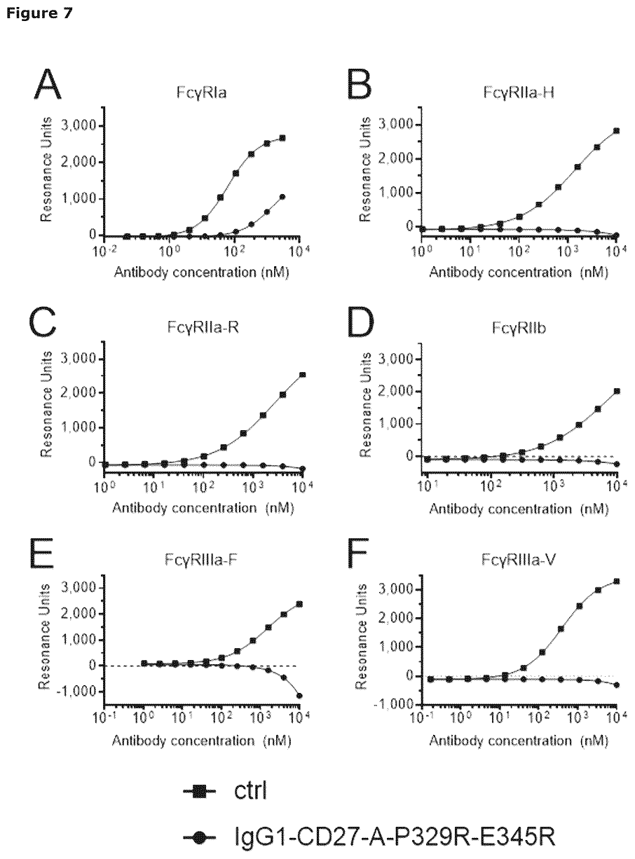

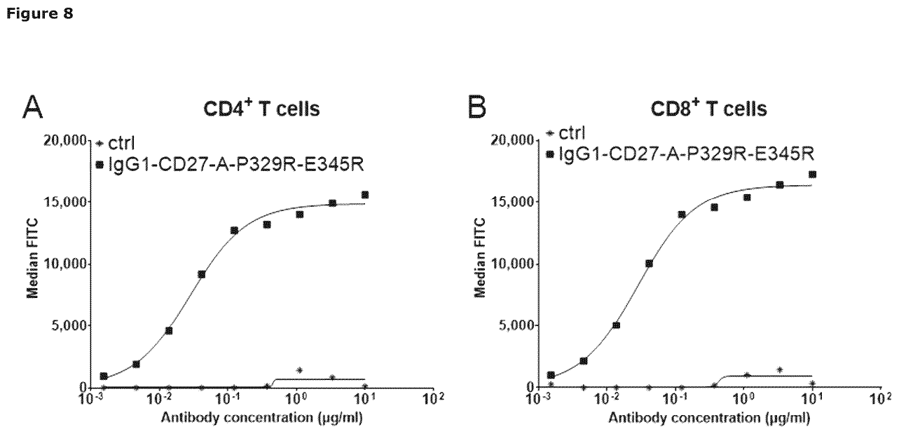

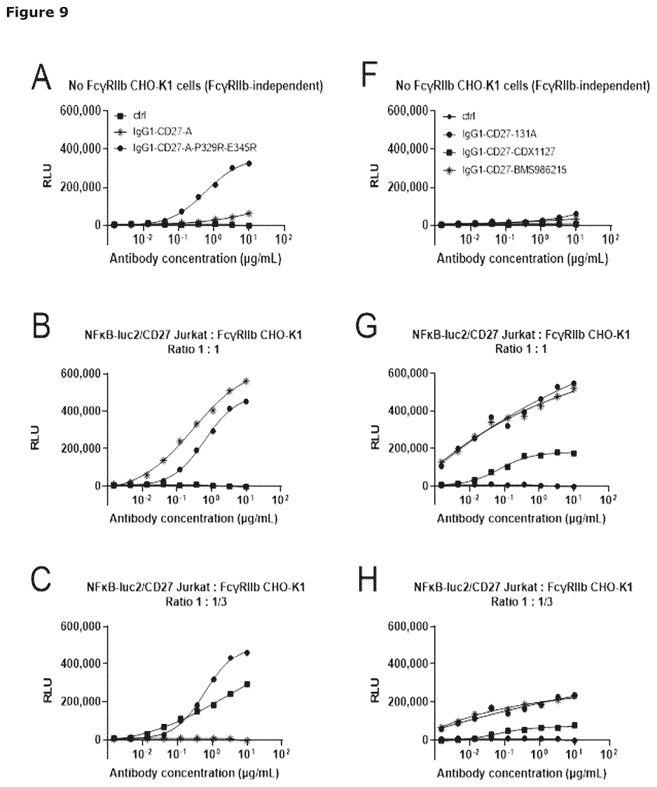

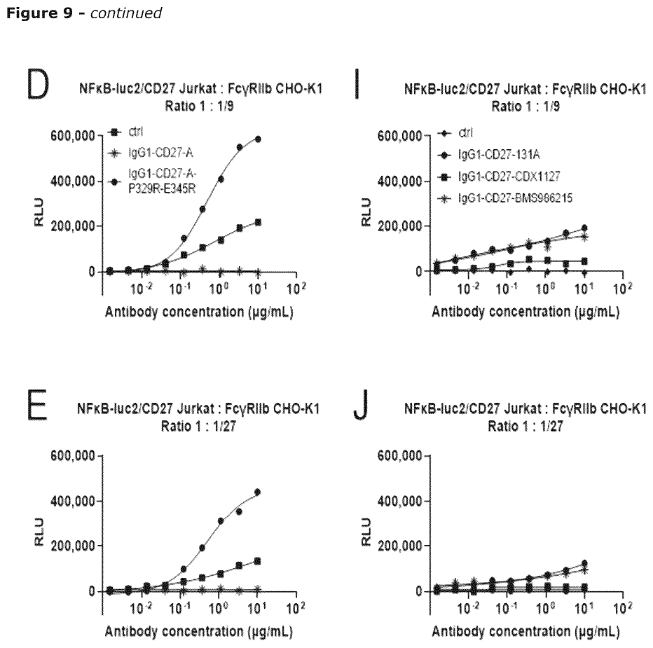

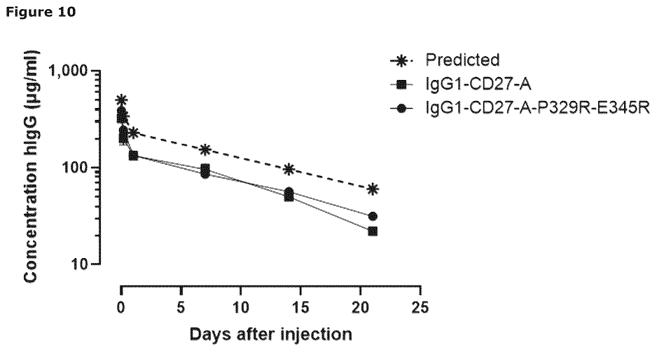

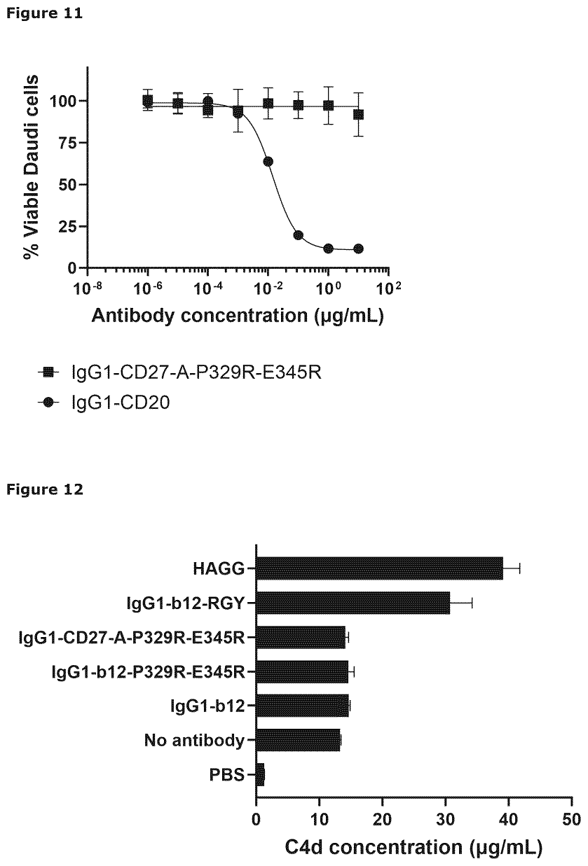

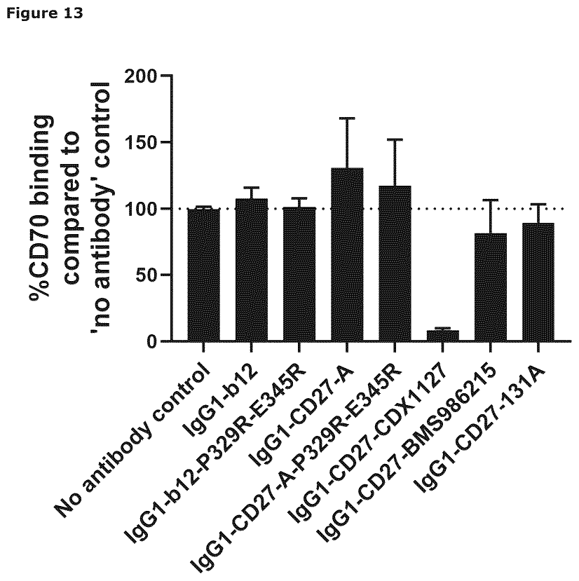

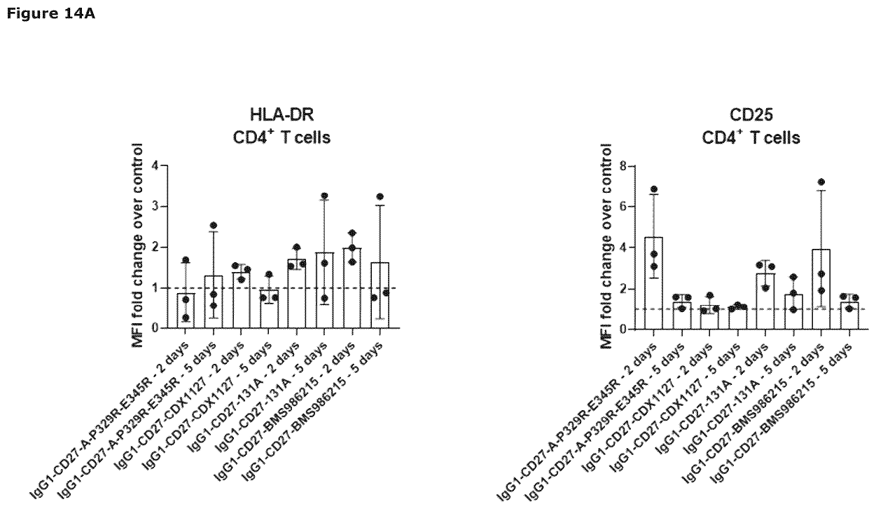

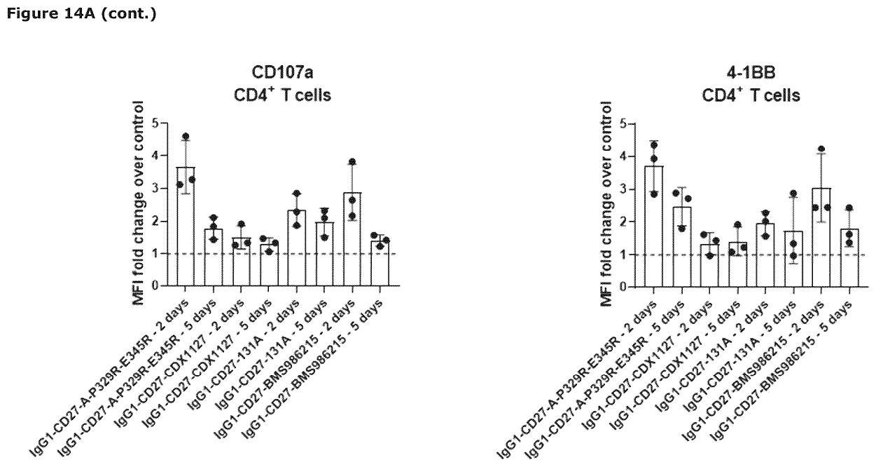

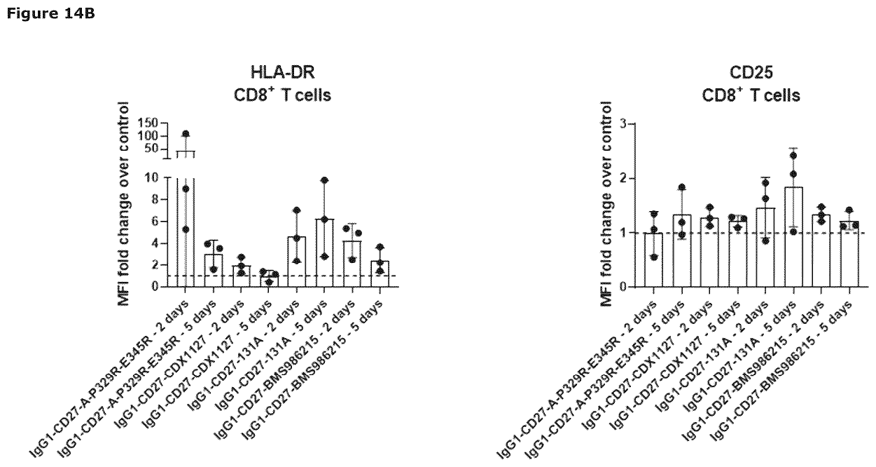

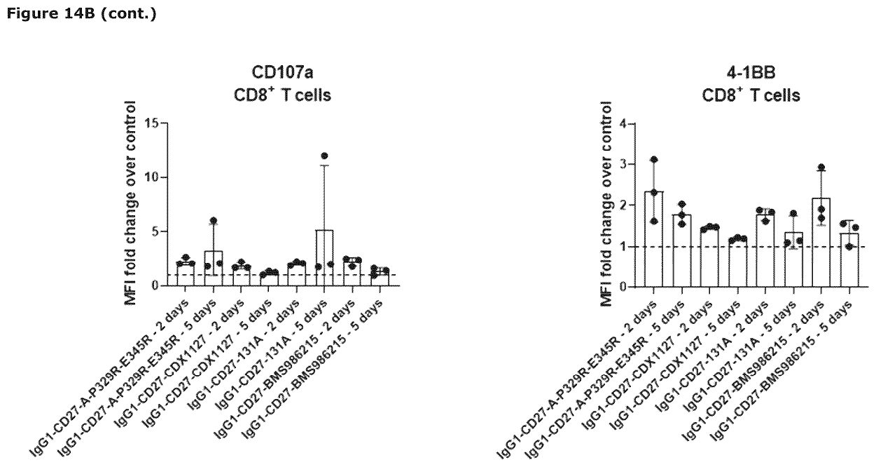

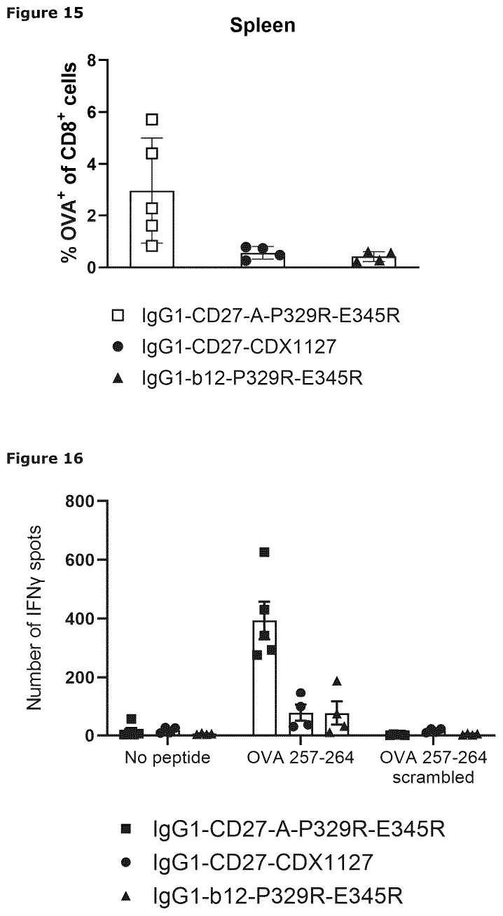

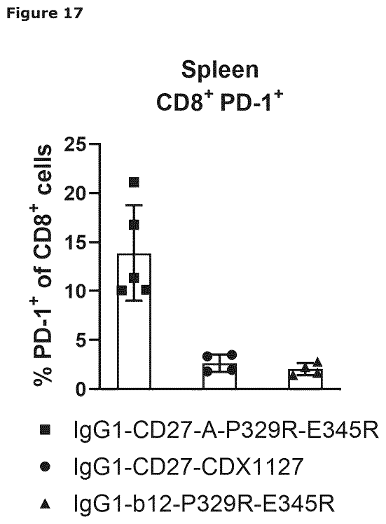

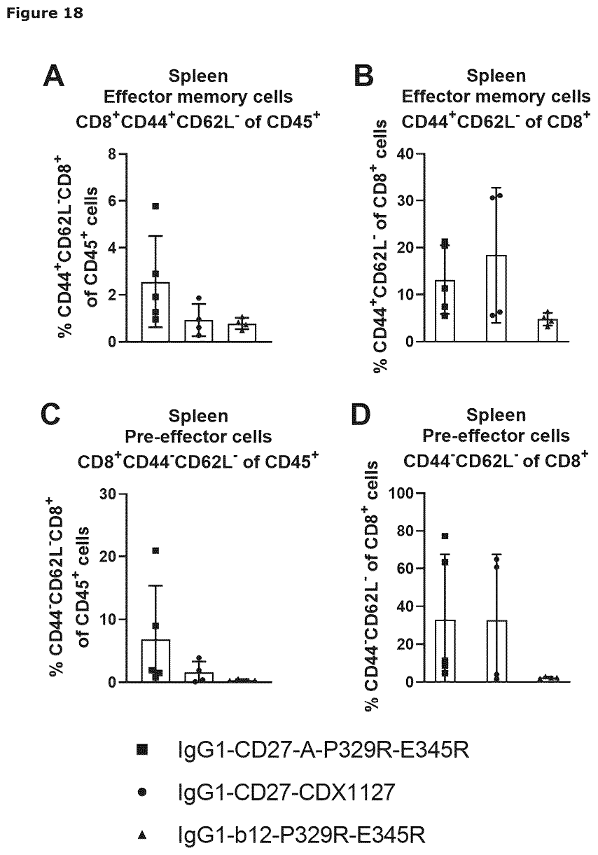

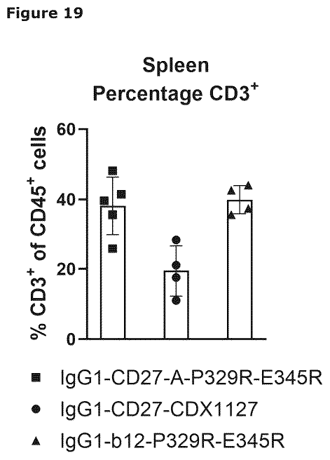

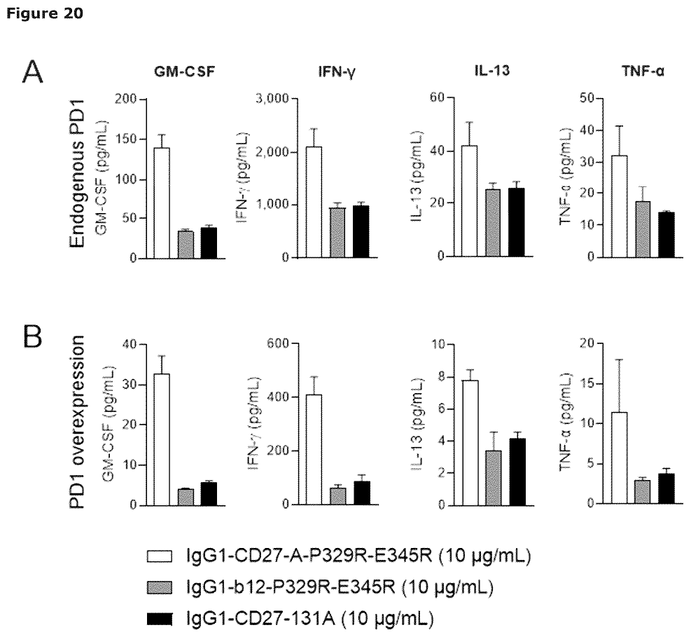

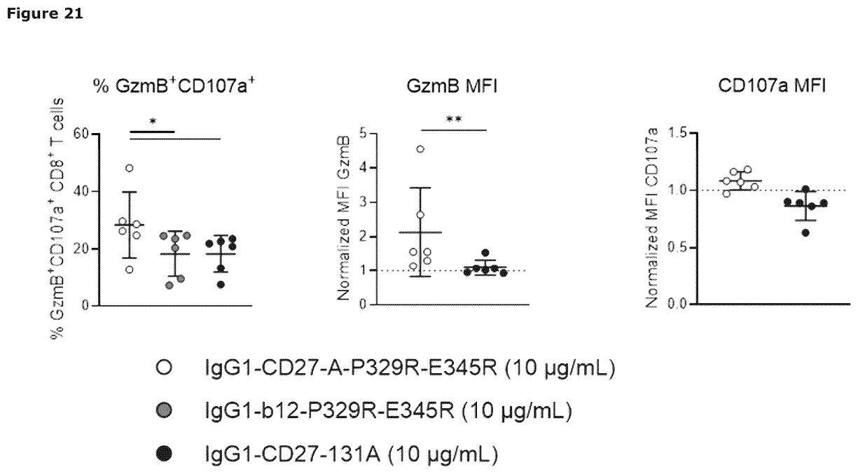

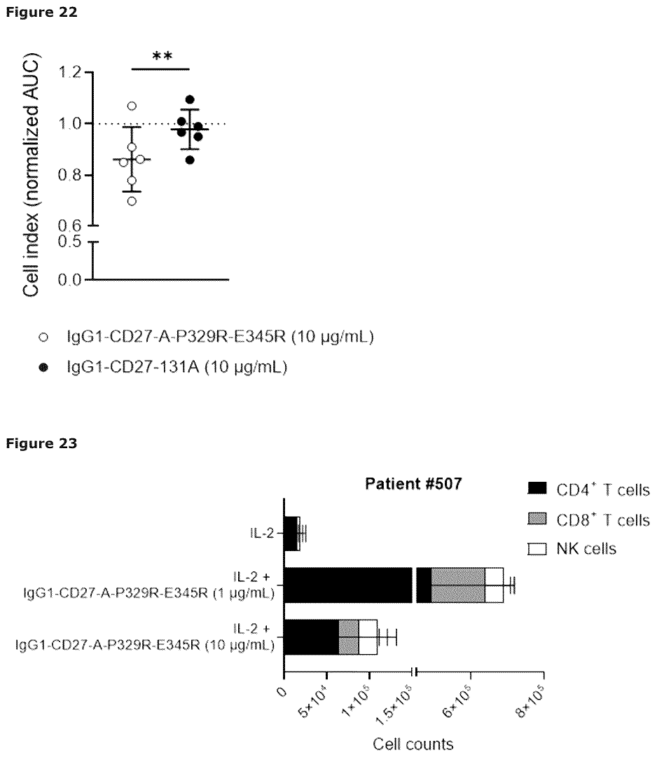

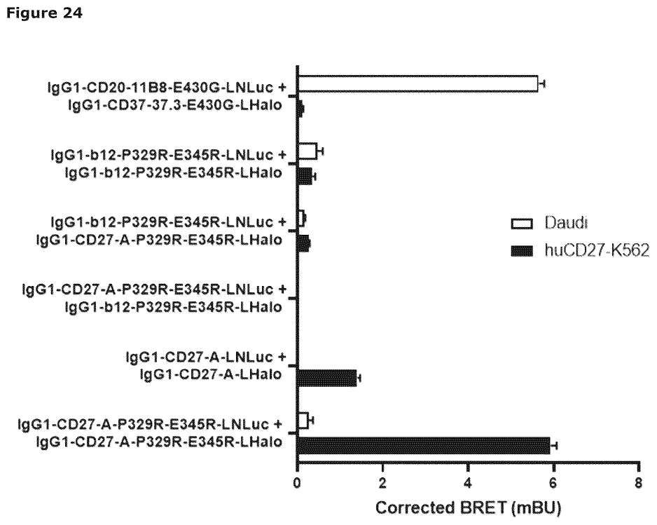

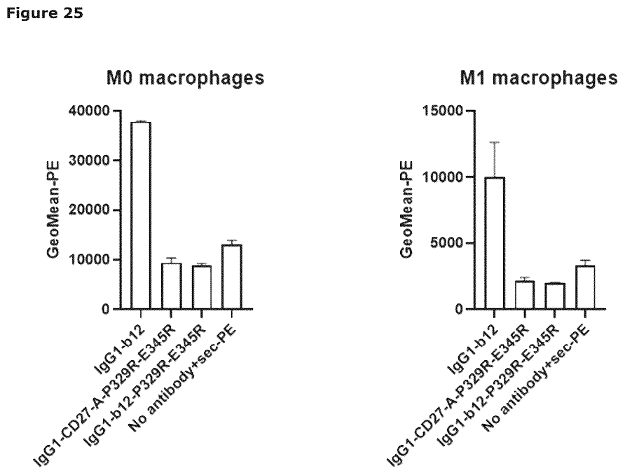

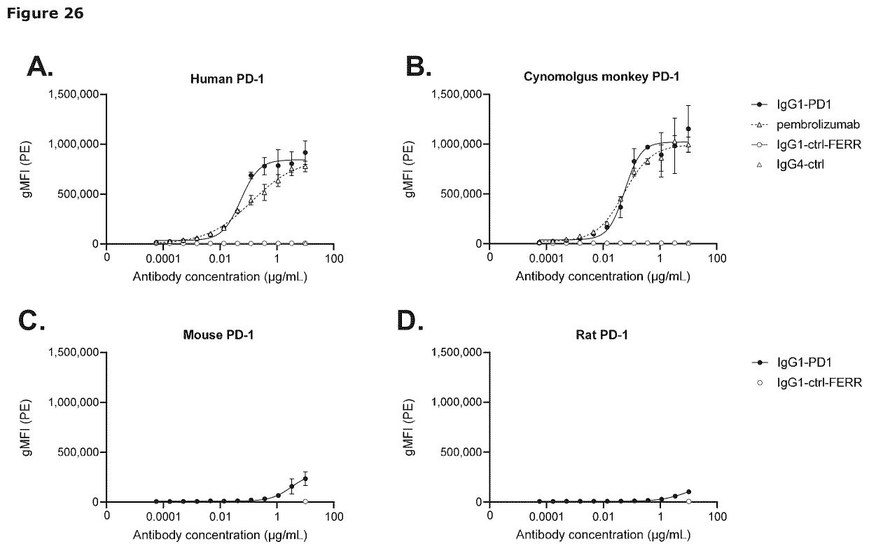

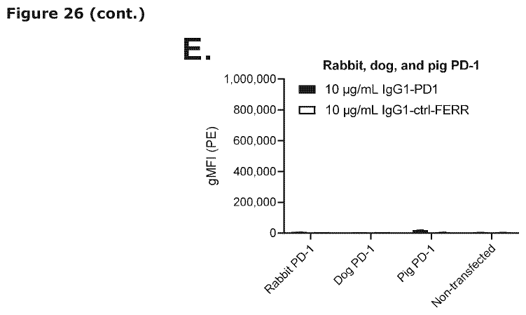

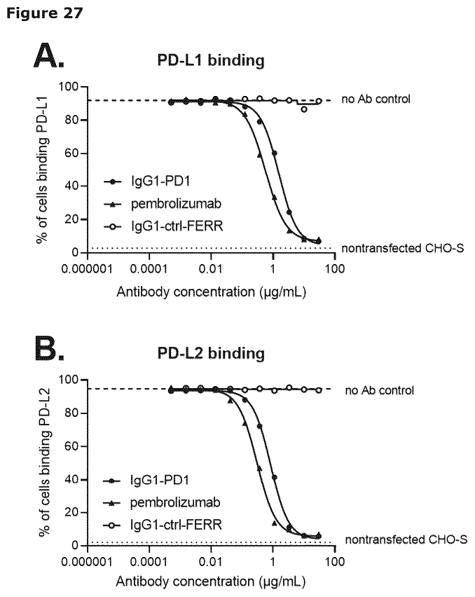

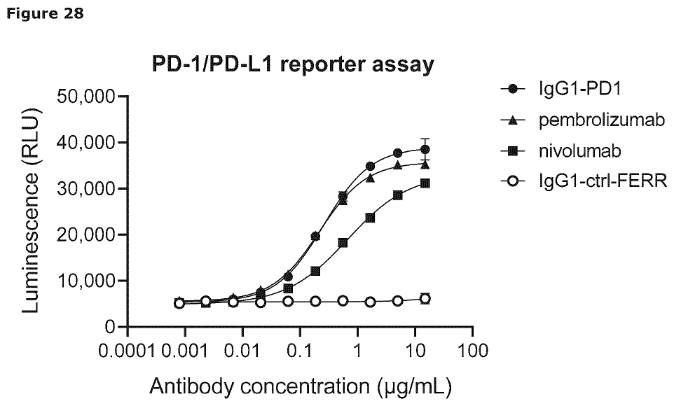

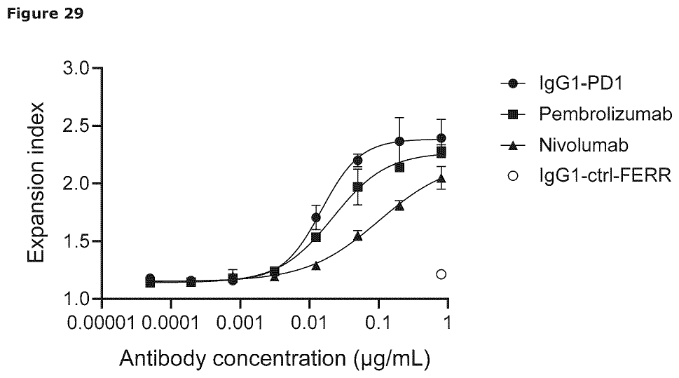

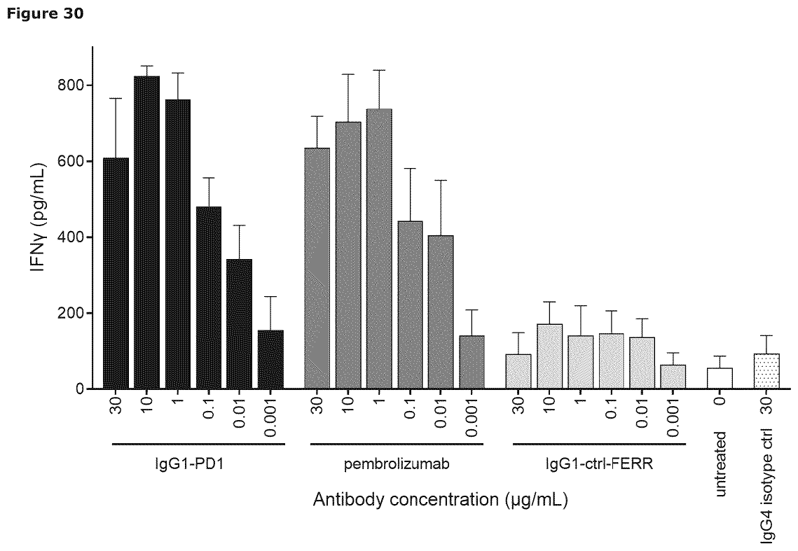

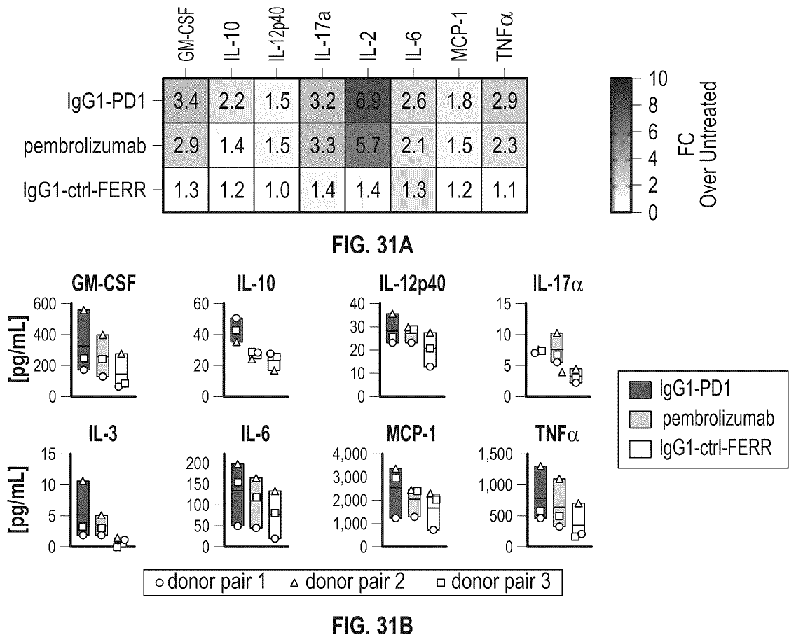

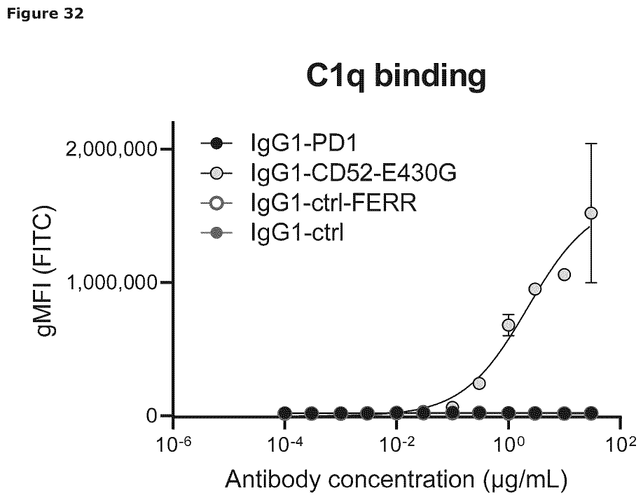

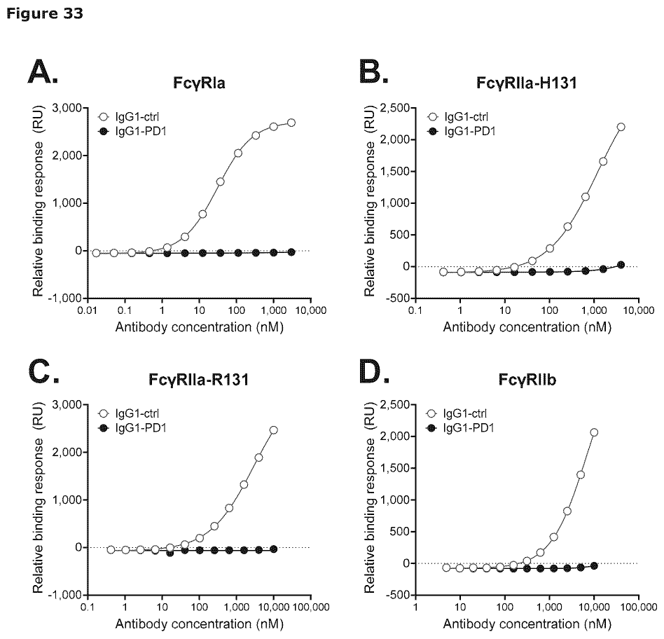

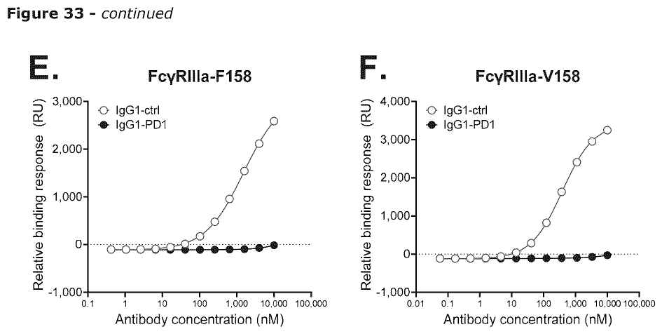

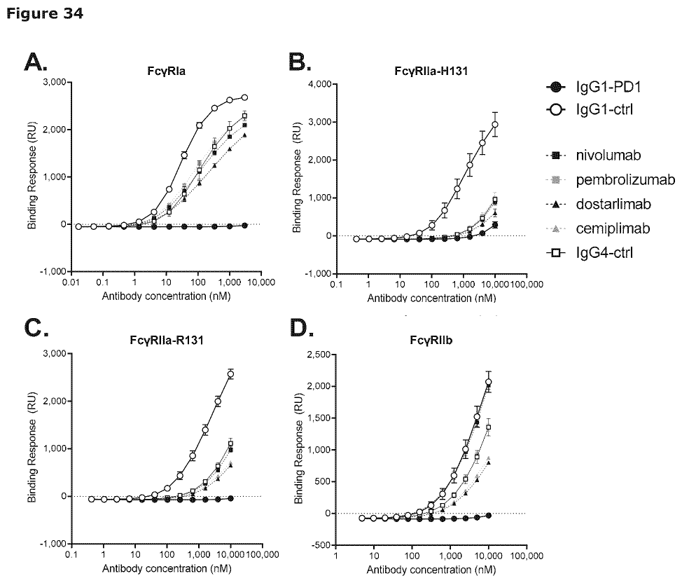

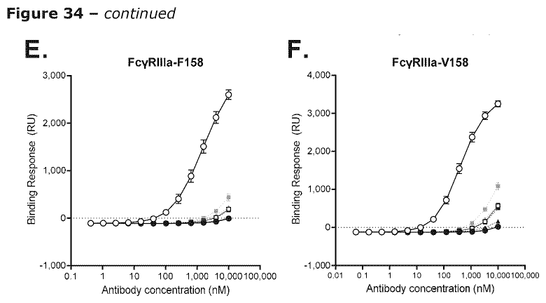

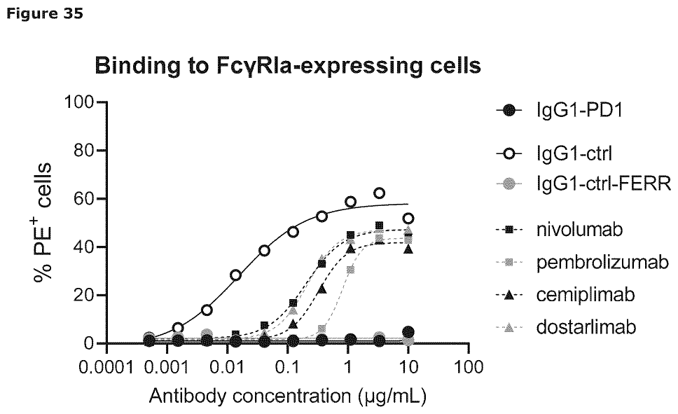

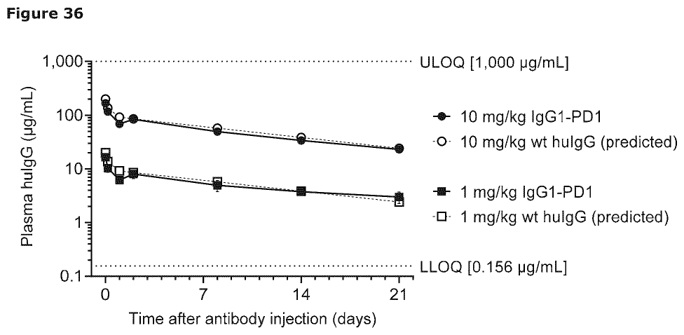

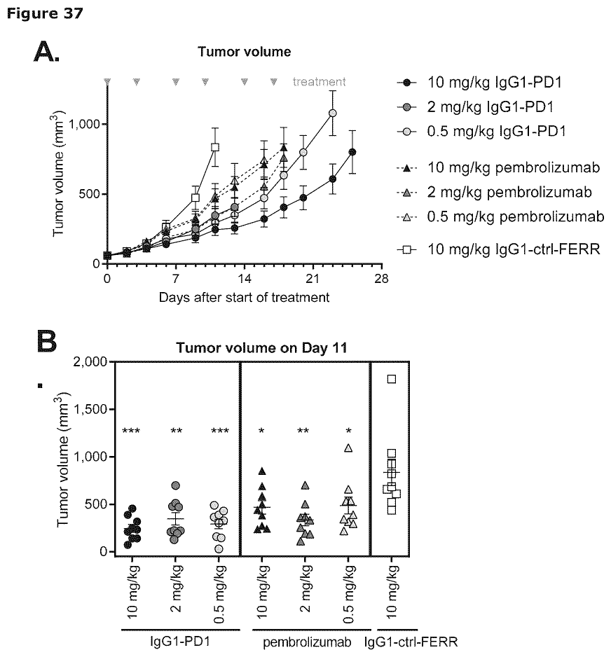

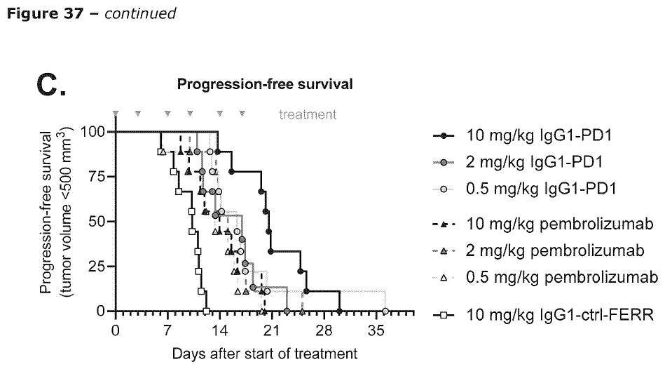

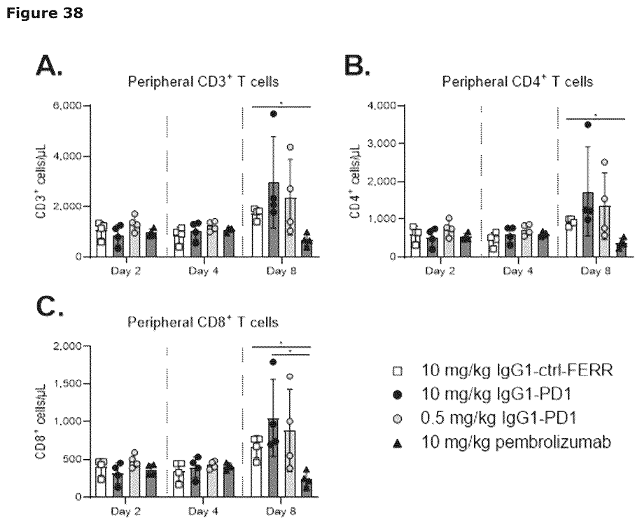

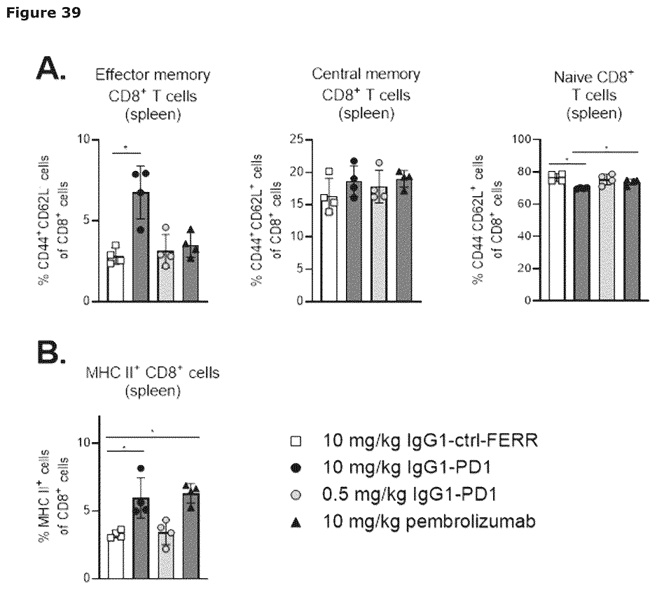

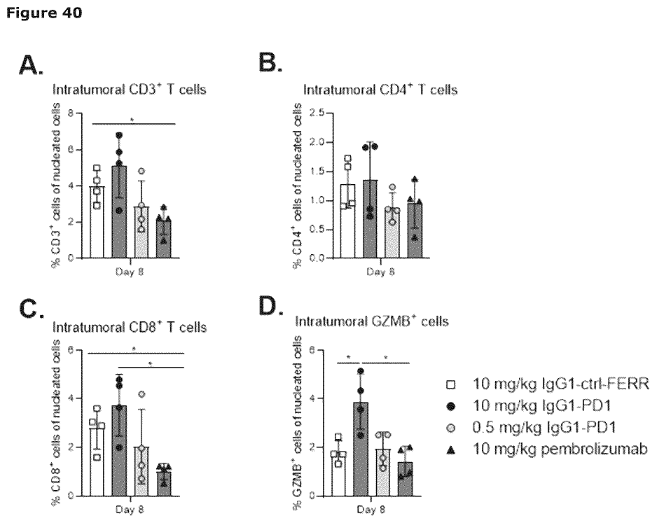

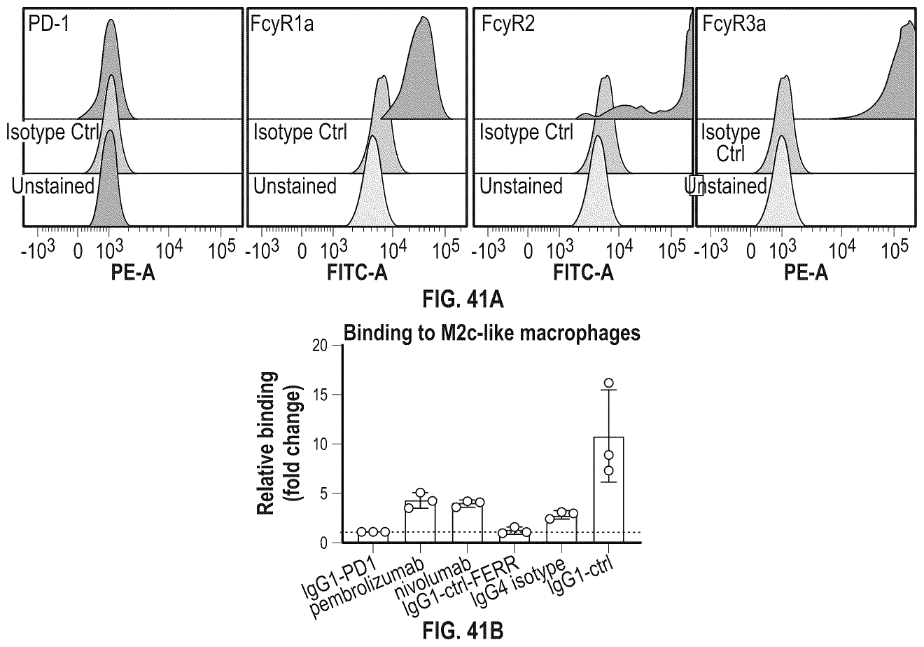

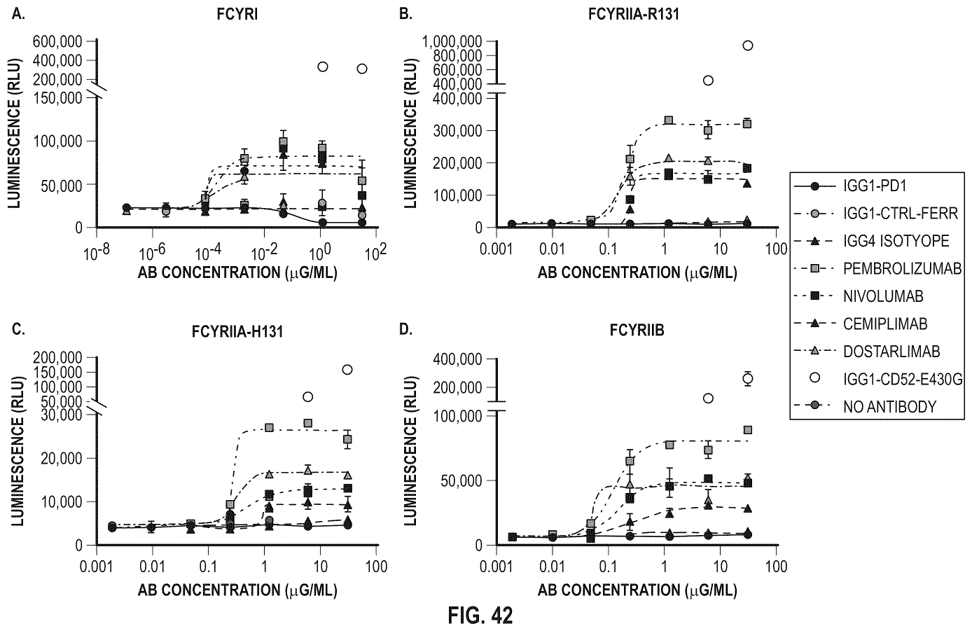

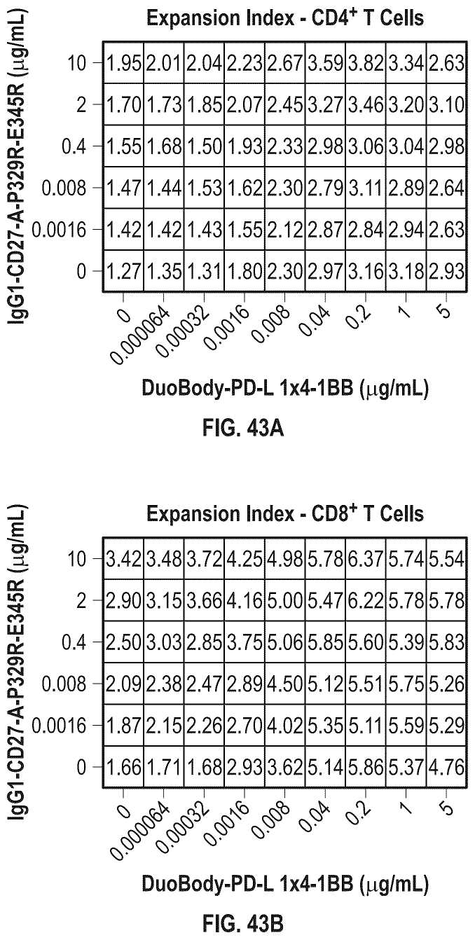

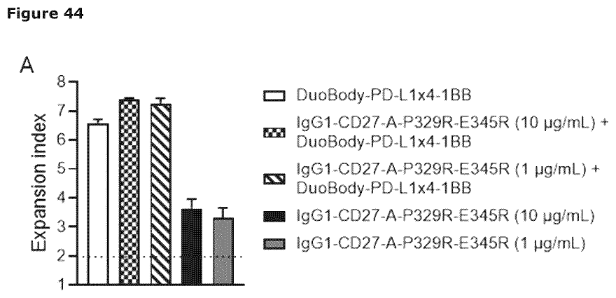

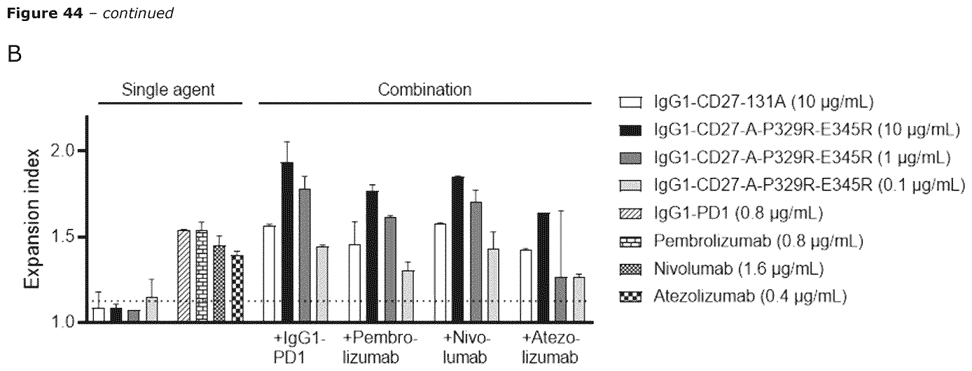

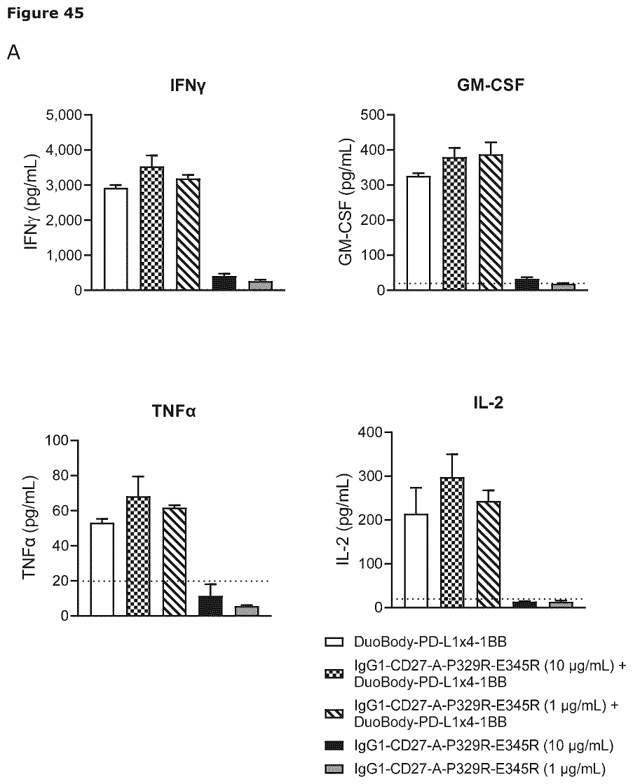

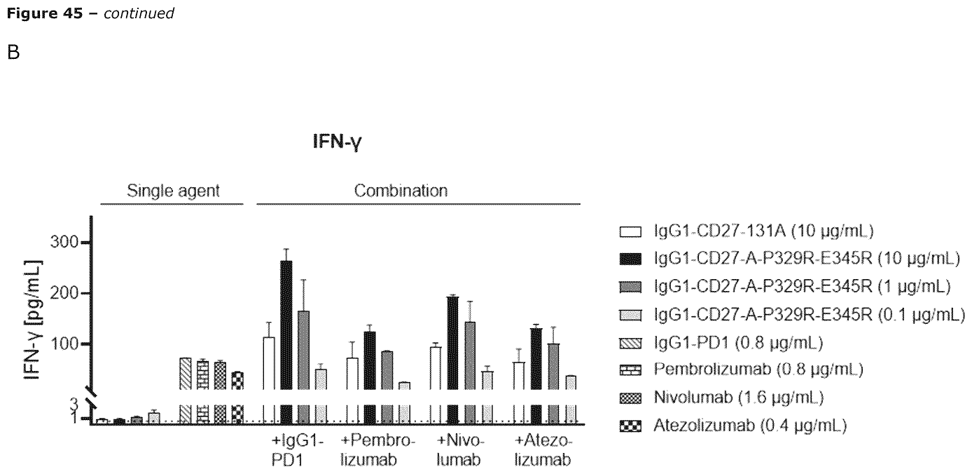

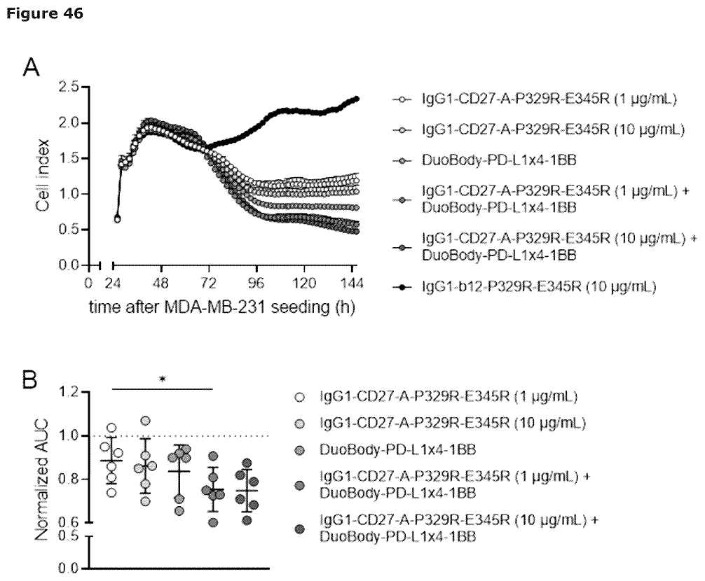

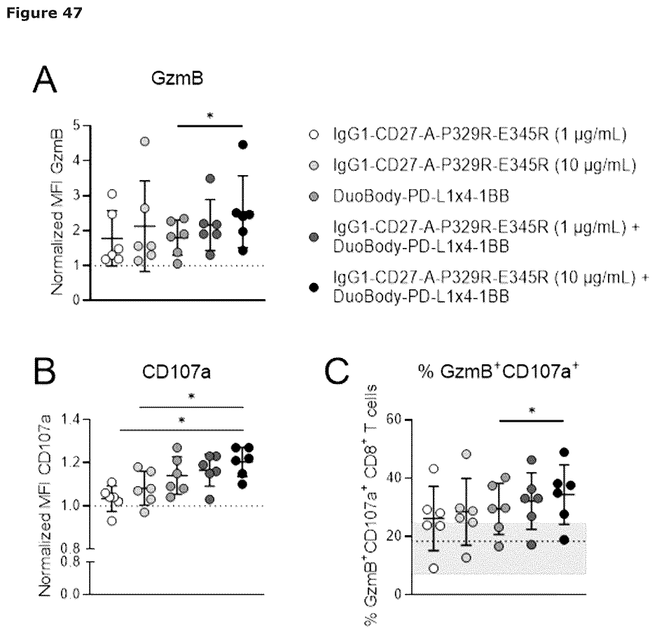

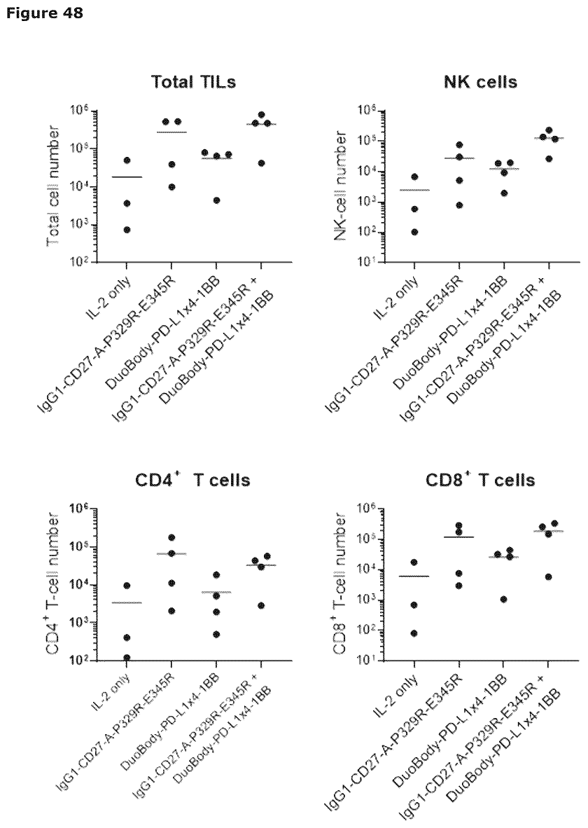

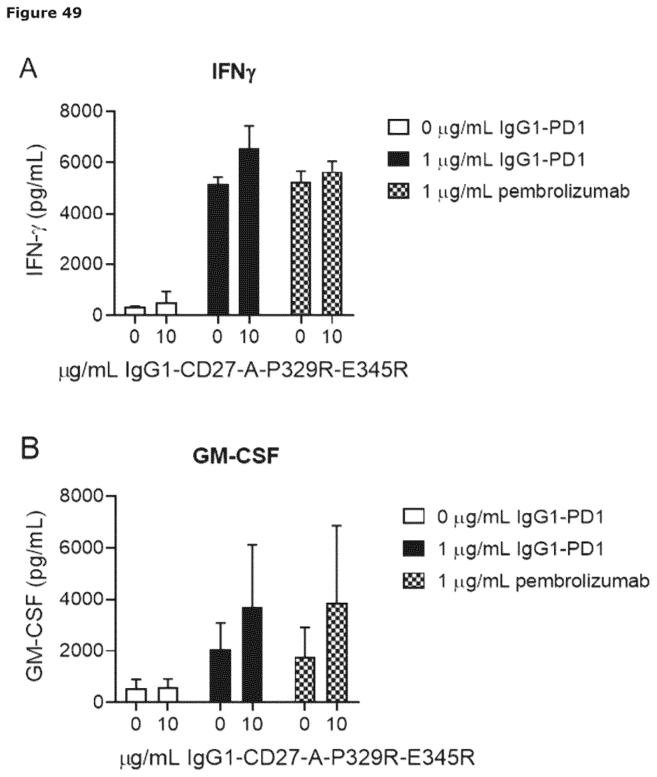

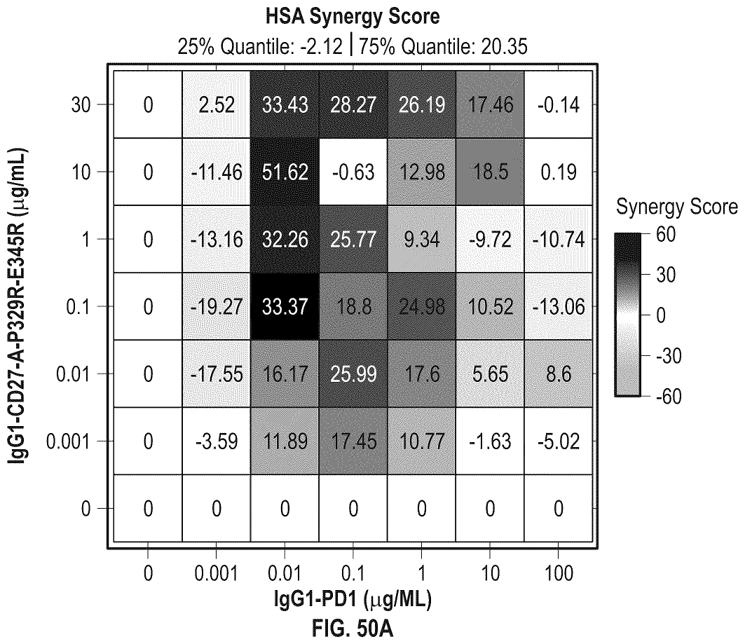

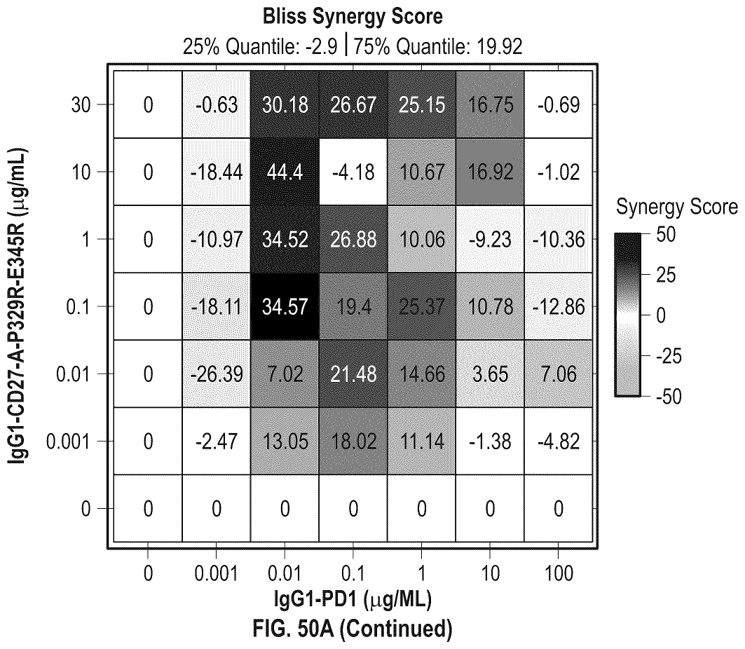

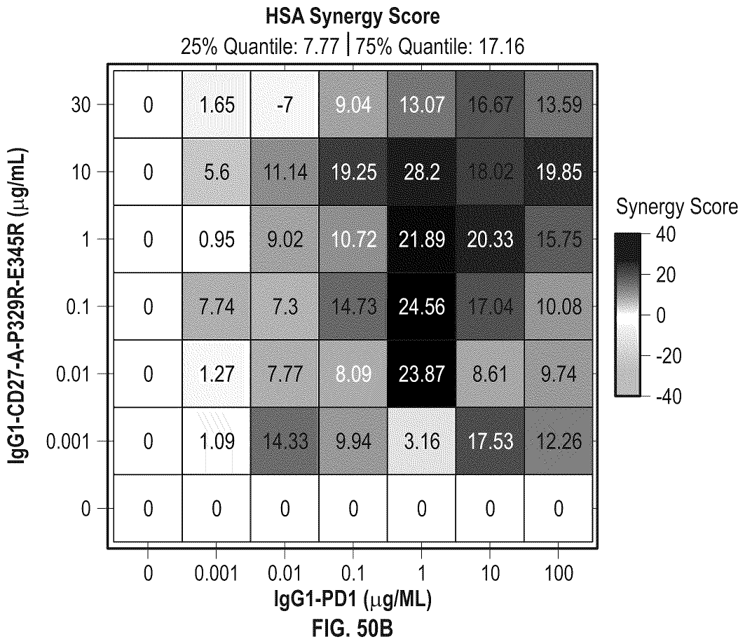

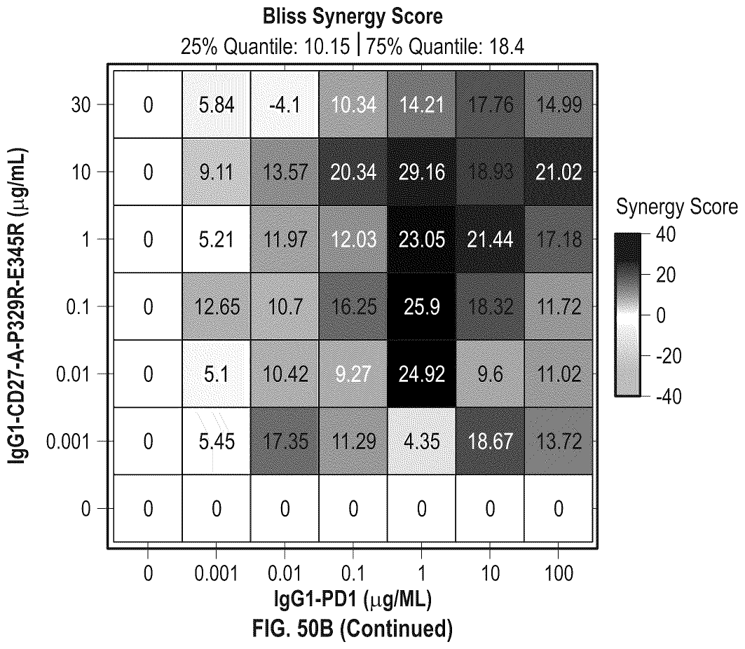

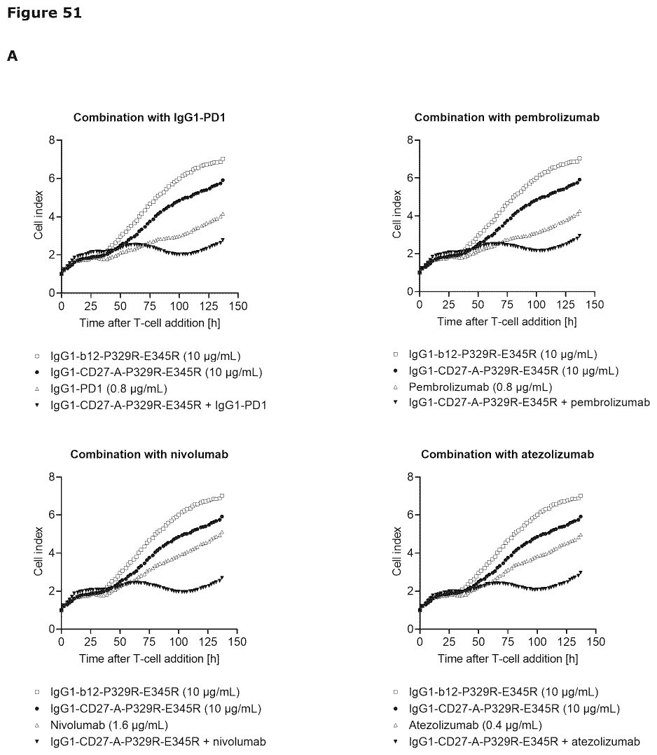

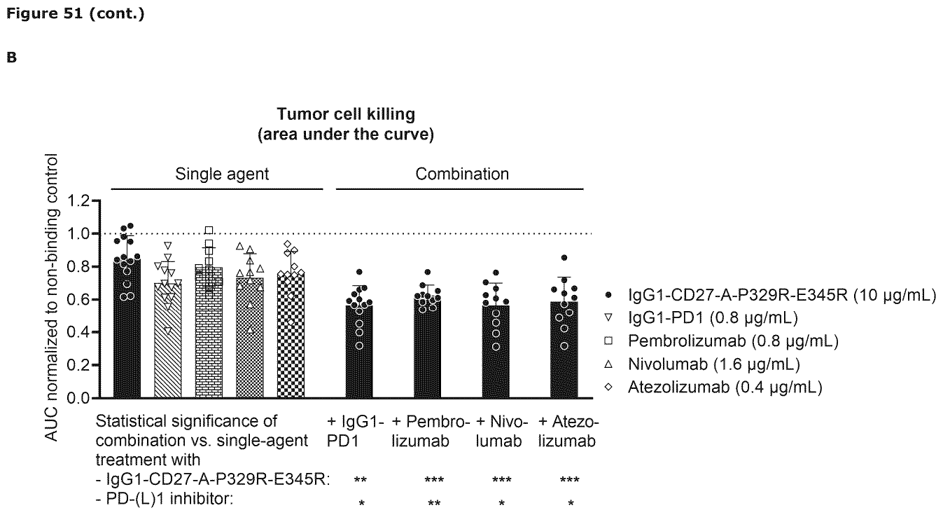

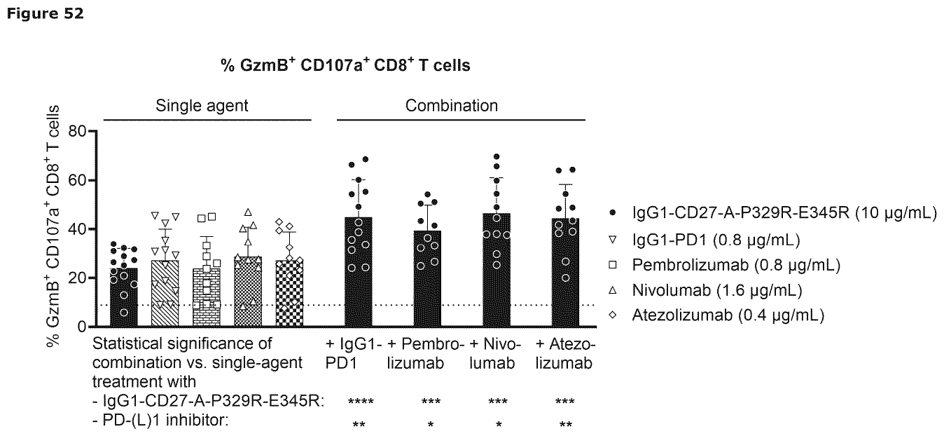

1. A method for reducing progression or preventing progression of a tumor or treating cancer in a subject, said method comprising administering to said subject i) a binding agent comprising at least one binding region binding to CD27; and ii) a PD1/PD-L1 inhibitor.

2. The method of claim 1, wherein said binding agent comprises a heavy chain variable (VH) region CDR1, CDR2, and CDR3 comprising the sequences as set forth in SEQ ID NOs: 5, 6, and 7, respectively, and a light chain variable (VL) region CDR1, CDR2, and CDR3 comprising the sequences as set forth in SEQ ID NO: 9, 10 and 11, respectively.

3. The method of claim 1 or 2, wherein said binding agent comprises two binding regions capable of binding to human CD27 wherein said antibody comprises the heavy chain variable (VH) region CDR1, CDR2, and CDR3 comprising the sequences as set forth in SEQ ID NOs: 5, 6, and 7, respectively, and the light chain variable (VL) region CDR1, CDR2, and CDR3 comprising the sequences as set forth in SEQ ID NO: 9, 10, and 11, respectively.

4. The method of any one of the preceding claims, wherein said binding agent comprises a VH region comprising a sequence as set forth in SEQ ID NO: 4.

5. The method of any one of the preceding claims, wherein said binding agent comprises a VL region comprising a sequence as set forth in SEQ ID NO: 8.

6. The method of any one of the preceding claims, wherein said binding agent comprises the VH and VL regions comprising the sequences as set forth in SEQ ID NO: 4 and SEQ ID NO: 8, respectively.

7. The method of any one of the preceding claims, wherein said binding agent is an antibody, preferably a human or a humanized antibody.

8. The method of any one of the preceding claims, wherein the antibody is a full-length antibody further comprising a light chain constant region (CL) and a heavy chain constant region (CH).

9. The method of claim 8, wherein the light chain constant region is human kappa.

10. The method of claim 8, wherein the light chain constant region is human lambda.

11. The method of any one of the preceding claims, wherein said binding agent further comprises a heavy chain constant region, which is of a human IgG isotype, optionally of a modified human IgG.

12. The method of claim 11, wherein the human IgG or modified human IgG is selected from IgG1, IgG2, IgG3 or IgG4, such as human IgG1.

13. The method of claim 11 or 12, wherein the IgG is a modified human IgG comprising one or more amino acid substitutions.

14. The method of any one of claims 11 to 13, wherein the modified human IgG is a modified human IgG1 comprising one or more amino acid substitutions, such as two or more amino acid substitutions.

15. The method of any one of claims 11 to 14, wherein the modified human IgG heavy chain constant region comprises at most 10 amino acid substitutions, such as at most 9, such as at most 8, such as at most 7, such as at most 6, such as at most 5, such as at most 4, such as at most 3, such as at most 2 amino acid substitutions.

16. The method of any one of claims 11 to 15, wherein said substitution in the heavy chain constant region induces increased CD27 agonism compared to an identical antibody except for comprising a wild type IgG1 antibody heavy chain constant region.

17. The method of any one of claims 11 to 16, wherein the amino acid residue at the position corresponding to position E345 or E430 in a human IgG1 heavy chain according to Eu numbering is selected from the group comprising: A, C, D, F, G, H, I, K, L, M, N, Q, R, S, T, V, W and Y.

18. The method of any one of claims 11 to 17, wherein the amino acid residue at the position corresponding to position E345 in a human IgG1 heavy chain according to Eu numbering is R.

19. The method of any one of claims 11 to 18, wherein the amino acid residue at the position corresponding to position E430 in a human IgG1 heavy chain according to Eu numbering is G.

20. The method of any one of claims 11 to 19, wherein the amino acid residue at the position corresponding to position P329 in a human IgG1 heavy chain according to Eu numbering is R.

21. The method of any one of claims 11 to 20, wherein the amino acid residue at the positions corresponding to position E345 and P329 in a human IgG1 heavy chain according to Eu numbering are both R.

22. The method of any one of claims 11 to 21, wherein the binding agent has a pharmacokinetic profile as the parent antibody comprising a wild type IgG1 heavy chain constant region.

23. The method of any one of the preceding claims, wherein the binding agent comprises the heavy chain constant region comprising a sequence selected from the group comprising: SEQ ID No 12, 13, 14, 15, 18, 19, 20, 21, 22, 23, 27, 28, 29, 30, 31, 32, 33, 34 and 36.

24. The method of any one of the preceding claims, wherein the binding agent comprises the heavy chain constant region comprising the sequence as set forth in SEQ ID No 15.

25. The method of any one of the preceding claims, wherein said binding agent comprises a heavy chain constant region, which is modified so that the binding agent induces one or more Fc-mediated effector functions to a lesser extent relative to a parent antibody.

26. The method of claim 25, wherein the one or more Fc-mediated effector functions is decreased by at least 20%, such as by at least 30% or by at least 40%, or by at least 50% or by at least 60% or by at least 70%, or by at least 80% or by at least 90%.

27. The method of claim 25 or 26, wherein the binding agent does not induce one or more Fc-mediated effector functions.

28. The method of any one of claims 25 to 27, wherein the one or more Fc-mediated effector functions is selected from the following group: complement-dependent cytotoxicity (CDC), complement-dependent cell-mediated cytotoxicity (CDCC), complement activation, antibody-dependent cell-mediated cytotoxicity (ADCC), antibody-dependent cell-mediated phagocytosis (ADCP), C1q binding and FcγR binding.

29. The method of any one of claims 25 to 28, wherein the binding agent does not induce C1q binding when measured by the method of Example 8.

30. The method of any one of the preceding claims, wherein the binding agent is a monovalent antibody.

31. The method of any one of the preceding claims, wherein the binding agent is a bivalent antibody.

32. The method of any one of the preceding claims, wherein the binding agent is a monospecific antibody.

33. The method of any one of the preceding claims, wherein the binding agent is a bispecific antibody comprising a first antigen binding region capable of binding human CD27 according to any one of the preceding claims and comprising a second antigen binding region capable of binding to a different epitope on human CD27 or capable of binding a different target.

34. The method of any one of the preceding claims, wherein CD27 is human CD27, in particular said human CD27 comprises the sequence as set forth in SEQ ID NO: 1 or the human CD27 variant as set forth in SEQ ID NO: 2.

35. The method of any one of the preceding claims, wherein said binding agent comprises:
e. The VH region comprising the amino acid sequence set forth in SEQ ID No: 4;
f. The VL region comprising the amino acid sequence set forth in SEQ ID No: 8;
g. The CH region comprising the amino acid sequence set forth in SEQ ID No: 15; and
h. The CL region comprising the amino acid sequence set forth in SEQ ID No: 17.

36. The method of any one of the preceding claims, wherein said binding agent comprises a heavy chain comprising the amino acid sequence set forth in SEQ ID NO: 35 and a light chain comprising the amino acid sequence set forth in SEQ ID NO: 25.

37. The method of any one of the preceding claims, wherein PD-L1 is human PD-L1, in particular human PD-L1 comprising the sequence set forth in SEQ ID NO: 98.

38. The method of any one of the preceding claims, wherein PD1 is human PD1, preferably the PD1 has or comprises the amino acid sequence as set forth in SEQ ID NO: 58 or SEQ ID NO: 59, or the amino acid sequence of PD1 has at least 70%, at least 75%, at least 80%, at least 85%, at least 90%, at least 95%, at least 97%, at least 99%, or 100% identity to the amino acid sequence as set forth in SEQ ID NO: 58 or SEQ ID NO: 59, or is an immunogenic fragment thereof.

39. The method of any one of the preceding claims, wherein the PD1/PD-L1 inhibitor is an antibody binding to PD1 or PD-L1, preferably an antibody which is an antagonist of PD1/PD-L1 interaction and/or is a PD1 or PD-L1 blocking antibody.

40. The method of any one of the preceding claims, wherein the PD1/PD-L1 inhibitor is an antibody of an isotype selected from the group consisting of IgG1, IgG2, IgG3, and IgG4, such as of the IgG1 isotype.

41. The method of any one of the preceding claims, wherein the PD1/PD-L1 inhibitor is a full-length antibody or antibody fragment, such as a full-length IgG1 antibody.

42. The method of any one of the preceding claims, wherein the PD1/PD-L1 inhibitor is a monospecific antibody.

43. The method of any one of the preceding claims, wherein said PD1/PD-L1 inhibitor is an antibody binding to PD1 comprising a heavy chain variable region (VH) comprising the CDR1, CDR2 and CDR3 sequences set forth in SEQ ID NO: 99, 100 and 101, respectively, and a light chain variable region (VL) comprising the CDR1, CDR2, and CDR3 sequences set forth in SEQ ID NO: 102, LAS and SEQ ID NO: 103, respectively.

44. The method of any one of the preceding claims, wherein said PD1/PD-L1 inhibitor is an antibody binding to PD1 comprising a VH region comprising the amino acid sequence of SEQ ID NO: 104 and a VL region comprising the amino acid sequence of SEQ ID NO: 105.

45. The method of any one of the preceding claims, wherein said PD1/PD-L1 inhibitor is an antibody binding to PD1 comprising a heavy chain comprising the amino acid sequence of SEQ ID NO: 106 and a light chain comprising the amino acid sequence of SEQ ID NO: 107.

46. The method of any one of the preceding claims, wherein
a) said binding agent is an antibody comprises a heavy chain comprising the amino acid sequence set forth in SEQ ID NO: 35 and a light chain comprising the amino acid sequence set forth in SEQ ID NO: 25;
b) said PD1/PD-L1 inhibitor is Pembrolizumab or a biosimilar thereof.

47. The method of any one of claims 1-42, wherein
a) said binding agent is an antibody comprises a heavy chain comprising the amino acid sequence set forth in SEQ ID NO: 35 and a light chain comprising the amino acid sequence set forth in SEQ ID NO: 25;
b) said PD1/PD-L1 inhibitor is Nivolumab or a biosimilar thereof.

48. The method of any one of claims 1-42, wherein
a) said binding agent is an antibody comprises a heavy chain comprising the amino acid sequence set forth in SEQ ID NO: 35 and a light chain comprising the amino acid sequence set forth in SEQ ID NO: 25;
b) said PD1/PD-L1 inhibitor is Atezolizumab or a biosimilar thereof.

49. The method of any one of claims 1-42, wherein said PD1/PD-L1 inhibitor is an antibody binding to PD1, or an antigen binding fragment thereof, wherein said antibody binding to PD1 comprises a VH region CDR1, CDR2, and CDR3 comprising the sequences as set forth in SEQ ID NOs: 49, 46, and 45, respectively, and a VL region CDR1, CDR2, and CDR3 comprising the sequences as set forth in SEQ ID NO: 52, QAS and SEQ ID NO: 50, respectively.

50. The method of claim 49, wherein the antibody binding to PD1 comprises a heavy chain variable region (VH) comprising a sequence having at least 70%, at least 75%, at least 80%, at least 85%, at least 90%, at least 95%, at least 97%, at least 99%, or 100% identity to the amino acid sequence of the VH sequence as set forth in SEQ ID NO: 56.

51. The method of claim 50, wherein the antibody binding to PD1 comprises a heavy chain variable region (VH), wherein the VH comprises the sequence as set forth in SEQ ID NO: 56.

52. The method of any one of claims 49-51, wherein the antibody binding to PD1 comprises a light chain variable region (VL) comprising a sequence having at least 70%, at least 75%, at least 80%, at least 85%, at least 90%, at least 95%, at least 97%, at least 99%, or 100% identity to the amino acid sequence of the VL sequence as set forth in SEQ ID NO: 57.

53. The method of claim 52, wherein the antibody binding to PD1 comprises a light chain variable region (VL), wherein the VL comprises the sequence as set forth in SEQ ID NO: 57.

54. The method of any one of claims 49-53, wherein the antibody binding to PD1 comprises a heavy chain variable region (VH) and a light chain variable region (VL), wherein the VH comprises or has the sequence as set forth in SEQ ID NO: 56 and the VL comprises or has the sequence as set forth in SEQ ID NO: 57.

55. The method of any one of claims 49-54, wherein the antibody binding to PD1 comprises a heavy chain constant region, wherein the heavy chain constant region comprises an aromatic or non-polar amino acid at the position corresponding to position 234 in a human IgG1 heavy chain according to EU numbering and an amino acid other than glycine at the position corresponding to position 236 in a human IgG1 heavy chain according to EU numbering.

56. The method of claim 55, wherein the amino acid at the position corresponding to position 236 is a basic amino acid.

57. The method of claim 56, wherein the basic amino acid is selected from the group consisting of lysine, arginine and histidine.

58. The method of claim 56 or 57, wherein the basic amino acid is arginine (G236R).

59. The method of any one of claims 55-58, wherein the amino acid at the position corresponding to position 234 is an aromatic amino acid.

60. The method of claim 59, wherein the aromatic amino acid is selected from the group consisting of phenylalanine, tryptophan and tyrosine.

61. The method of any one of claims 55-58, wherein the amino acid at the position corresponding to position 234 is a non-polar amino acid.

62. The method of claim 61, wherein the non-polar amino acid is selected from the group consisting of alanine, valine, leucine, isoleucine, proline, phenylalanine, methionine and tryptophan.

63. The method of claim 61 or 62, wherein the non-polar amino acid is selected from the group consisting of isoleucine, proline, phenylalanine, methionine and tryptophan.

64. The method of any one of claims 55-63, wherein the amino acid at the corresponding to position 234 is phenylalanine (L234F).

65. The method of any one of claims 55-64, wherein the amino acid at the position corresponding to position 235 in a human IgG1 heavy chain according to EU numbering in said heavy chain constant region of the antibody binding to PD1 is an acidic amino acid.

66. The method of claim 65, wherein the acidic amino acid is aspartate or glutamate.

67. The method of any one of claims 55-66, wherein the amino acid at the position corresponding to position 235 in a human IgG1 heavy chain according to EU numbering in said heavy chain constant region of the antibody binding to PD1 is glutamate (L235E).

68. The method of any one of claims 55-67, wherein the amino acids at the position corresponding to positions 234, 235 and 236 in said heavy chain constant region of the antibody binding to PD1 are a non-polar or an aromatic amino acid at position 234, an acidic amino acid at position 235 and a basic amino acid at position 236.

69. The method of any one of claims 55-68, wherein the amino acid corresponding to position 234 is phenylalanine, the amino acid corresponding to position 235 is glutamate, and the amino acid corresponding to position 236 is arginine in said heavy chain constant region of the antibody binding to PD1 (L234F/L235E/G236R).

70. The method of any one of claims 49-69, wherein the heavy chain constant region of the antibody binding to PD1 comprises a sequence having at least 70%, at least 75%, at least
80%, at least 85%, at least 90%, at least 95%, at least 97%, at least 99%, or 100% identity to the amino acid sequence of the HC sequence as set forth in SEQ ID NO: 38.

71. The method of any one of claims 49-70, wherein the heavy chain constant region of the antibody binding to PD1 comprises the sequence as set forth in SEQ ID NO: 38.

72. The method of any one of claims 49-71, wherein the isotype of the heavy chain constant region of the antibody binding to PD1 is IgG1.

73. The method of any one of claims 49-72, wherein the antibody binding to PD1 comprises a heavy chain having the sequence as set forth in SEQ ID NO: 139, and a light chain having the sequence as set forth in SEQ ID NO: 140.

74. The method of any one of claims 49-73, wherein the antibody binding to PD1 is a monoclonal, chimeric or humanized antibody or a fragment of such an antibody.

75. The method of any one of claims 49-74, wherein the antibody binding to PD1 has a reduced or depleted Fc-mediated effector function.

76. The method of any one of claims 49-75, wherein binding of complement protein C1q to the constant region of the antibody binding to PD1 is reduced compared to a wild-type antibody, preferably by at least 70%, at least 80%, at least 90%, at least 95%, at least 97% or 100%.

77. The method of any one of claims 49-76, wherein binding to one or more of the IgG Fc-gamma receptors to the antibody binding to PD1 is reduced compared to a wild-type antibody, preferably by at least 70%, at least 80%, at least 90%, at least 95%, at least 97% or 100%.

78. The method of claim 77, wherein the one or more IgG Fc-gamma receptors are selected from at least one of Fc-gamma RI, Fc-gamma RII and Fc-gamma RIII.

79. The method of claim 77 or 78, wherein the IgG Fc-gamma receptor is Fc-gamma RI.

80. The method of any one of claims 49-79, wherein the antibody binding to PD1 is not capable of inducing Fc-gamma RI-mediated effector functions or wherein the induced Fc-gamma RI-mediated effector functions are reduced compared to a wild-type antibody, preferably by at least 70%, at least 80%, at least 90%, at least 95%, at least 97% or 100%.

81. The method of any one of claims 49-80, wherein the antibody binding to PD1 is not capable of inducing at least one of complement dependent cytotoxicity (CDC) mediated lysis, antibody dependent cellular cytotoxicity (ADCC) mediated lysis, apoptosis, homotypic adhesion and/or phagocytosis or wherein at least one of complement dependent cytotoxicity (CDC) mediated lysis, antibody dependent cellular cytotoxicity (ADCC) mediated lysis, apoptosis, homotypic adhesion and/or phagocytosis is induced in a reduced extent, preferably reduced by at least 70%, at least 80%, at least 90%, at least 95%, at least 97% or 100%.

82. The method of any one of claims 49-81, wherein binding of neonatal Fc receptor (FcRn) to the antibody binding to PD1 is unaffected, as compared to a wild-type antibody.

83. The method of any one of claims 49-82, the antibody binding to PD1 binds to a native epitope of PD1 present on the surface of living cells.

84. The method of any one of claims 49-83, wherein the antibody binding to PD1 is a multispecific antibody comprising a first antigen binding region binding to PD1 and at least one further antigen binding region binding to another antigen.

85. The method of claim 84, wherein the antibody binding to PD1 is a bispecific antibody comprising a first antigen binding region binding to PD1 and a second antigen binding region binding to another antigen.

86. The method of claim 84 or 85, wherein the first antigen binding region binding to PD1 comprises the heavy chain variable region (VH) and/or the light chain variable region (VL) as set forth in any one of claims 50 to 54.

87. The method of any one of claims 49-86, wherein
a) the binding agent comprises a VH region comprising the amino acid sequence set forth in SEQ ID No: 4, a VL region comprising the amino acid sequence set forth in SEQ ID No: 8;
b) the antibody binding to PD1 comprises a VH region and a VL region, wherein the VH comprises or has the sequence as set forth in SEQ ID NO: 56 and the VL comprises or has the sequence as set forth in SEQ ID NO: 57.

88. The method of any one of claims 49-87, wherein
a) said binding agent is an antibody comprising a VH region comprising the amino acid sequence set forth in SEQ ID No: 4, a VL region comprising the amino acid sequence set forth in SEQ ID No: 8, a CH region comprising the amino acid sequence set forth in SEQ ID No: 15, and a CL region comprising the amino acid sequence set forth in SEQ ID No: 17;
b) said antibody binding to PD1 comprising a VH region comprising the amino acid sequence set forth in SEQ ID No: 56, a VL region comprising the amino acid sequence set forth in SEQ ID No: 57, a CH region comprising the amino acid sequence set forth in SEQ ID No: 38, and a CL region comprising the amino acid sequence set forth in SEQ ID No: 42.

89. The method of any one of claims 1-41, wherein the PD1/PD-L1 inhibitor is a multispecific antibody, such as a bispecific antibody.

90. The method of claim 89, wherein the PD1/PD-L1 inhibitor is a PD-L1 inhibitor comprising a first binding region binding to CD137 and a second binding region binding to PD-L1.

91. The method of claim 90, wherein CD137 is human CD137, in particular human CD137 comprising the sequence set forth in SEQ ID NO: 97.

92. The method of claim 90 or 91, wherein
a) the first binding region of the PD-L1 inhibitor comprises a heavy chain variable region (VH) comprising the CDR1, CDR2, and CDR3 sequences of SEQ ID NO: 79, and a light chain variable region (VL) comprising the CDR1, CDR2, and CDR3 sequences of SEQ ID NO: 83; and
b) the second binding region of the PD-L1 inhibitor comprises a heavy chain variable region (VH) comprising the CDR1, CDR2, and CDR3 sequences of SEQ ID NO: 86, and a light chain variable region (VL) comprising the CDR1, CDR2, and CDR3 sequences of SEQ ID NO:
90.

93. The method of any one of claims 90-92, wherein a) the first binding region of the PD-L1 inhibitor comprises a heavy chain variable region (VH) comprising the CDR1, CDR2, and CDR3 sequences set forth in: SEQ ID NO: 80, 81, and 82, respectively, and a light chain variable region (VL) comprising the CDR1, CDR2, and CDR3 sequences set forth in: SEQ ID NO: 84, GAS, and SEQ ID NO: 85, respectively; and b) the second binding region of the PD-L1 inhibitor comprises a heavy chain variable region (VH) comprising the CDR1, CDR2, and CDR3 sequences set forth in: SEQ ID NO: 87, 88, and 89, respectively, and a light chain variable region (VL) comprising the CDR1, CDR2, and CDR3 sequences set forth in: SEQ ID NO: 91, DDN, and SEQ ID NO: 92, respectively.

94. The method of any one of claims 90-93, wherein
a) the first binding region of the PD-L1 inhibitor comprises a heavy chain variable region (VH) comprising the amino acid sequence set forth in SEQ ID NO: 79 and a light chain variable region (VL) region comprising the amino acid sequence set forth in SEQ ID NO: 83; and
b) the second binding region of the PD-L1 inhibitor comprises a heavy chain variable region (VH) comprising the amino acid sequence set forth in SEQ ID NO: 86 and a light chain variable region (VL) region comprising the amino acid sequence set forth in SEQ ID NO: 90.

95. The method of any one of claims 90-94, wherein the PD-L1 inhibitor is an antibody comprising a first binding arm and a second binding arm, wherein the first binding arm comprises
i) a polypeptide comprising said first heavy chain variable region (VH) and a first heavy chain constant region (CH), and
ii) a polypeptide comprising said first light chain variable region (VL) and a first light chain constant region (CL);
and the second binding arm comprises
iii) a polypeptide comprising said second heavy chain variable region (VH) and a second heavy chain constant region (CH), and
iv) a polypeptide comprising said second light chain variable region (VL) and a second light chain constant region (CL).

96. The method of any one of claims 90-95, wherein said PD-L1 inhibitor comprises
i) a first heavy chain and light chain comprising said antigen binding region capable of binding to CD137, the first heavy chain comprising a first heavy chain constant region and the first light chain comprising a first light chain constant region; and
ii) a second heavy chain and light chain comprising said antigen binding region capable of binding PD-L1, the second heavy chain comprising a second heavy chain constant region and the second light chain comprising a second light chain constant region.

97. The method of claim 95 or 96, wherein (i) the amino acid in the position corresponding to F405 in a human IgG1 heavy chain according to EU numbering is L in said first heavy chain constant region (CH), and the amino acid in the position corresponding to K409 in a human IgG1 heavy chain according to EU numbering is R in said second heavy chain constant region (CH), or (ii) the amino acid in the position corresponding to K409 in a human IgG1 heavy chain according to EU numbering is R in said first heavy chain, and the amino acid in the position corresponding to F405 in a human IgG1 heavy chain according to EU numbering is L in said second heavy chain.

98. The method of any one of claim 95-97, wherein the positions corresponding to positions L234 and L235 in a human IgG1 heavy chain according to EU numbering are F and E, respectively, in said first and second heavy chains.

99. The method of any one of claim 95-98, wherein the positions corresponding to positions L234, L235, and D265 in a human IgG1 heavy chain according to EU numbering are F, E, and A, respectively, in said first and second heavy chain constant regions (HCs).

100. The method of any one of claims 95-99, wherein the positions corresponding to positions L234 and L235 in a human IgG1 heavy chain according to EU numbering of both the first and second heavy chain constant regions are F and E, respectively, and wherein (i) the position corresponding to F405 in a human IgG1 heavy chain according to EU numbering of the first heavy chain constant region is L, and the position corresponding to K409 in a human IgG1 heavy chain according to EU numbering of the second heavy chain is R, or (ii) the position corresponding to K409 in a human IgG1 heavy chain according to EU numbering of the first heavy chain constant region is R, and the position corresponding to F405 in a human IgG1 heavy chain according to EU numbering of the second heavy chain is L.

101. The method of any one of claims 95-100, wherein the positions corresponding to positions L234, L235, and D265 in a human IgG1 heavy chain according to EU numbering of both the first and second heavy chain constant regions are F, E, and A, respectively, and wherein (i) the position corresponding to F405 in a human IgG1 heavy chain according to EU numbering of the first heavy chain constant region is L, and the position corresponding to K409 in a human IgG1 heavy chain according to EU numbering of the second heavy chain constant region is R, or (ii) the position corresponding to K409 in a human IgG1 heavy chain according to EU numbering of the first heavy chain is R, and the position corresponding to F405 in a human IgG1 heavy chain according to EU numbering of the second heavy chain is L.

102. The method of any one of claims 95-101, wherein the constant region of said first and/or second heavy chain, such as the second heavy chain, comprises or consists essentially of or consists of an amino acid sequence selected from the group consisting of
a) the sequence set forth in SEQ ID NO: 94 or 96[IgG1-Fc_FEAL];
b) a subsequence of the sequence in a), such as a subsequence, wherein 1, 2, 3, 4, 5, 6, 7, 8, 9 or 10 consecutive amino acids has/have been deleted, starting from the N-terminus or C-terminus of the sequence defined in a); and
c) a sequence having at most 6 substitutions, such as at most 5 substitutions, at most 4 substitutions, at most 3, at most 2 substitutions or at most 1 substitution, compared to the amino acid sequence defined in a) or b).

103. The method of any one of claims 95-102, wherein the constant region of said first and/or second heavy chain, such as the first heavy chain, comprises or consists essentially of or consists of an amino acid sequence selected from the group consisting of
a) the sequence set forth in SEQ ID NO: 93 or 95 [IgG1-Fc_FEAR];
b) a subsequence of the sequence in a), such as a subsequence, wherein 1, 2, 3, 4, 5, 6, 7, 8, 9 or 10 consecutive amino acids has/have been deleted, starting from the N-terminus or C-terminus of the sequence defined in a); and
c) a sequence having at most 6 substitutions, such as at most 5 substitutions, at most
4, at most 3, at most 2 substitutions or at most 1 substitution, compared to the amino acid sequence defined in a) or b).

104. The method of any one of any one of claims 95-103, wherein said PD-L1 inhibitor comprises a kappa (κ) light chain constant region.

105. The method of any one of any one of claims 95-104, wherein said PD-L1 inhibitor comprises a lambda (λ) light chain constant region.

106. The method of any one of any one of claims 95-105, wherein said first light chain constant region is a kappa (κ) light chain constant region or a lambda (λ) light chain constant region.

107. The method of any one of any one of claims 95-106, wherein said second light chain constant region is a lambda (λ) light chain constant region or a kappa (κ) light chain constant region.

108. The method of any one of any one of claims 95-107, wherein said first light chain constant region is a kappa (κ) light chain constant region and said second light chain constant region is a lambda (λ) light chain constant region or said first light chain constant region is a lambda (λ) light chain constant region and said second light chain constant region is a kappa (κ) light chain constant region.

109. The method of any one of claims 104-108, wherein the kappa (κ) light chain comprises an amino acid sequence selected from the group consisting of
a) the sequence set forth in SEQ ID NO: 16,
b) a subsequence of the sequence in a), such as a subsequence, wherein 1, 2, 3, 4, 5, 6, 7, 8, 9 or 10 consecutive amino acids has/have been deleted, starting from the N-terminus or C-terminus of the sequence defined in a); and
c) a sequence having at most 10 substitutions, such as at most 9 substitutions, at most
8, at most 7, at most 6, at most 5, at most 4 substitutions, at most 3, at most 2 substitutions or at most 1 substitution, compared to the amino acid sequence defined in a) or b).

110. The method of any one of claims 105-109, wherein the lambda (λ) light chain comprises an amino acid sequence selected from the group consisting of
a) the sequence set forth in SEQ ID NO: 17,
b) a subsequence of the sequence in a), such as a subsequence, wherein 1, 2, 3, 4, 5, 6, 7, 8, 9 or 10 consecutive amino acids has/have been deleted, starting from the N-terminus or C-terminus of the sequence defined in a); and
c) a sequence having at most 10 substitutions, such as at most 9 substitutions, at most
8, at most 7, at most 6, at most 5, at most 4 substitutions, at most 3, at most 2 substitutions or at most 1 substitution, compared to the amino acid sequence defined in a) or b).

111. The method of any one of claims 90-110, wherein the PD-L1 inhibitor is an antibody of the IgG1m(f) allotype.

112. The method of an one of claims 90-111, wherein the PD-L1 inhibitor is a bispecific antibody binding to CD137 and PD-L1, the bispecific antibody having i) a first heavy chain comprising the amino acid sequence set forth in SEQ ID NO: 75 and a first light chain comprising the amino acid sequence set forth in SEQ ID NO: 76, and ii) a second heavy chain comprising the amino acid sequence set forth in SEQ ID NO: 77 and a second light chain comprising the amino acid sequence set forth in SEQ ID NO: 78.

113. The method according to any one of claims 90-112, wherein the PD-L1 inhibitor is acasunlimab or a biosimilar thereof.

114. The method of any one of claims 90-113, wherein
a) the binding agent comprises a heavy chain variable (VH) region CDR1, CDR2, and CDR3 comprising the sequences as set forth in SEQ ID NOs: 5, 6, and 7, respectively, and a light chain variable (VL) region CDR1, CDR2, and CDR3 comprising the sequences as set forth in SEQ ID NO: 9, 10, and 11, respectively;
b) the first binding region of the PD-L1 inhibitor comprises a heavy chain variable region (VH) comprising the CDR1, CDR2, and CDR3 sequences set forth in: SEQ ID NO: 80, 81, and
82, respectively, and a light chain variable region (VL) comprising the CDR1, CDR2, and CDR3 sequences set forth in: SEQ ID NO: 84, GAS, and SEQ ID NO: 85, respectively; and
c) the second binding region of the PD-L1 inhibitor comprises a heavy chain variable region (VH) comprising the CDR1, CDR2, and CDR3 sequences set forth in: SEQ ID NO: 87, 88, and 89, respectively, and a light chain variable region (VL) comprising the CDR1, CDR2, and CDR3 sequences set forth in: SEQ ID NO: 91, DDN, and SEQ ID NO: 92, respectively.

115. The method of any one of claims 90-114, wherein
a) the binding agent comprises a VH region comprising the amino acid sequence set forth in SEQ ID No: 4, a VL region comprising the amino acid sequence set forth in SEQ ID No: 8;
b) the first binding region of the PD-L1 inhibitor comprises a heavy chain variable region (VH) comprising the amino acid sequence set forth in SEQ ID NO: 79 and a light chain variable region (VL) region comprising the amino acid sequence set forth in SEQ ID NO: 83; and
c) the second binding region of the PD-L1 inhibitor comprises a heavy chain variable region (VH) comprising the amino acid sequence set forth in SEQ ID NO: 86 and a light chain variable region (VL) region comprising the amino acid sequence set forth in SEQ ID NO: 90.

116. The method of any one of claims 90-115, wherein
a) said binding agent is an antibody comprising a VH region comprising the amino acid sequence set forth in SEQ ID No: 4, a VL region comprising the amino acid sequence set forth in SEQ ID No: 8, a CH region comprising the amino acid sequence set forth in SEQ ID No: 15, and a CL region comprising the amino acid sequence set forth in SEQ ID No: 17;
b) said PD-L1 inhibitor is an antibody comprising a first binding arm and a second binding arm, the first binding arm comprising the first binding region and a second binding arm comprising the second binding region;
c) the first binding arm of the PD-L1 inhibitor comprises a VH region comprising the amino acid sequence set forth in SEQ ID No: 79, a VL region comprising the amino acid sequence set forth in SEQ ID No: 83; a CH region comprising the amino acid sequence set forth in SEQ ID No: 95, and a CL region comprising the amino acid sequence set forth in SEQ ID No: 16; and
d) the second binding arm of the PD-L1 inhibitor comprises a VH region comprising the amino acid sequence set forth in SEQ ID No: 86, a VL region comprising the amino acid sequence set forth in SEQ ID No: 90, a CH region comprising the amino acid sequence set forth in SEQ ID No: 96, and a CL region comprising the amino acid sequence set forth in SEQ ID No: 17.

117. The method of any one of claims 90-116, wherein
c) said binding agent comprises a heavy chain comprising the amino acid sequence set forth in SEQ ID NO: 35 and a light chain comprising the amino acid sequence set forth in SEQ ID NO: 25;
d) said PD-L1 inhibitor is a bispecific antibody binding to CD137 and PD-L1, the bispecific antibody having i) a first heavy chain comprising the amino acid sequence set forth in SEQ ID NO: 75 and a first light chain comprising the amino acid sequence set forth in SEQ ID NO:
76, and ii) a second heavy chain comprising the amino acid sequence set forth in SEQ ID NO: 77 and a second light chain comprising the amino acid sequence set forth in SEQ ID NO:
78.

118. The method of any one of claims 90-117, wherein
c) said binding agent comprises a heavy chain comprising the amino acid sequence set forth in SEQ ID NO: 35 and a light chain comprising the amino acid sequence set forth in SEQ ID NO: 25;
d) said PD-L1 inhibitor is acasunlimab or a biosimilar thereof.

119. The method of any one of claims 1-38, wherein the PD1/PD-L1 inhibitor is a PD1 inhibitor selected from Pembrolizumab, Nivolumab, Cemiplimab, Dostarlimab, JTX-4014, Spartalizumab, Camrelizumab, Sintilimab, Tislelizumab, Toripalimab, INCMGA00012 (MGA012), AMP-224, AMP-514, or a respective biosimilar thereof.

120. The method of any one of claims 1-38, wherein the PD1 inhibitor is selected from Pembrolizumab, Nivolumab, Cemiplimab, Dostarlimab, JTX-4014, Spartalizumab, Camrelizumab, Sintilimab, Tislelizumab, Toripalimab, INCMGA00012 (MGA012), AMP-514, or a respective biosimilar thereof.

121. The method of any one of claims 1-38, wherein the PD1/PD-L1 inhibitor is a PD-L1 inhibitor selected from Atezolizumab, Avelumab, Durvalumab, KN035, CK-301, Acasunlimab, AUNP12, CA-170, BMS-986189, or a respective biosimilar thereof.

122. The method of any one of claims 1-38, wherein the PD-L1 inhibitor is selected from Atezolizumab, Avelumab, Durvalumab, KN035, CK-301, Acasunlimab, or a respective biosimilar thereof.

123. The method of any one of the preceding claims, wherein the subject is a human subject.

124. The method of any one of the preceding claims, wherein the tumor or cancer is a solid tumor.

125. The method according to any one of the preceding claims, wherein said tumor is a PD-L1 positive tumor.

126. The method of any one of the preceding claims, wherein the tumor or cancer is head and neck squamous cell carcinoma (HNSCC), such as HNSCC of the oral cavity, pharynx or larynx.

127. The method of claim 126, wherein the HNSCC is recurrent, unresectable or metastatic.

128. The method of any one of claims 1-125, wherein the tumor or cancer is non-small cell lung cancer (NSCLC), such as a squamous or non-squamous NSCLC.

129. The method of claim 128, wherein the NSCLC is recurrent, unresectable or metastatic.

130. The method of claim 128 or 129, wherein the NSCLC does not have an epidermal growth factor (EGFR)-sensitizing mutation and/or anaplastic lymphoma (ALK) translocation and/or ROS1 rearrangement.

131. The method of any one of claims 128-130, wherein the NSCLC is NTRK1/2/3 (neurotrophic receptor tyrosine kinase 1/2/3) fusion positive, and/or has a mutation in KRAS (KRAS proto-oncogene, GTPase), BRAF (B-Raf proto-oncogene, serine/threonine kinase), or MET (MET proto-oncogene, receptor tyrosine kinase) gene, and/or has RET (ret proto-oncogene) gene rearrangements, and the subject has received prior treatment with a respective targeted therapy.

132. The method of any one of the preceding claims, wherein the subject has received prior treatment with a PD1 inhibitor or a PD-L1 inhibitor, such as anti-PD1 antibody or an anti-PD-L1 antibody, preferably at least two doses of the PD1 inhibitor or the PD-L1 inhibitor.

133. The method of any one of the preceding claims, wherein the subject has received prior treatment with a platinum-based therapy or an alternative chemotherapy if platinum ineligible, eg a gemcitabine-containing regimen.

134. The method of any one of the preceding claims, wherein the tumor or cancer has relapsed and/or progressed after treatment, such as systemic treatment with a checkpoint inhibitor.

135. The method of any one of the preceding claims, wherein the subject has received at least one prior line of systemic therapy, such as systemic therapy comprising a PD1 inhibitor or a PD-L1 inhibitor, such as an anti-PD1 antibody or an anti-PD-L1 antibody.

136. The method of any one of the preceding claims, wherein the cancer or tumor has relapsed and/or is refractory, or the subject has progressed after treatment with a PD1 inhibitor or a PD-L1 inhibitor, such as an anti PD1 antibody or an anti-PD-L1 antibody, the PD1 inhibitor or PD-L1 inhibitor being administered as monotherapy or as part of a combination therapy.

137. The method of any one of the preceding claims, wherein last prior treatment was with a PD1 inhibitor or PD-L1 inhibitor, such as an anti PD1 antibody or an anti-PD-L1 antibody, the PD1 inhibitor or PD-L1 inhibitor being administered as monotherapy or as part of a combination therapy.

138. The method of any one of the preceding claims, wherein the time from progression on last treatment with a PD1 inhibitor or PD-L1 inhibitor, such as an anti PD1 antibody or an anti-PD-L1 antibody is 6 months or less.

139. The method of any one of the preceding claims, wherein the time from last dosing of a PD1 inhibitor or PD-L1 inhibitor, such as an anti PD1 antibody or an anti-PD-L1 antibody as part of last prior treatment is 6 months or less.

140. The method of any one of the preceding claims, wherein the cancer or tumor has relapsed and/or is refractory, or the subject has progressed during or after
i) platinum doublet chemotherapy following treatment with an anti-PD1 antibody or an anti-PD-L1 antibody, or
ii) treatment with an anti-PD1 antibody or an anti-PD-L1 antibody following platinum doublet chemotherapy.

141. A kit comprising
i) a binding agent comprising at least one binding region binding to CD27 and ii) a PD1/PD-L1 inhibitor.

142. The kit according to claim 141, wherein the binding agent is as defined in any one of claims 1-140 and/or the PD1/PD-L1 inhibitor is as defined in any one of claims 1-140.

143. The kit according to claim 141 or 142, wherein the binding agent, the PD1/PD-L1 inhibitor, and, if present, one or more additional therapeutic agents are for systemic administration, in particular for injection or infusion, such as intravenous injection or infusion.

144. The kit according to any one of claims 141-143 for use in a method for reducing progression or preventing progression of a tumor or treating cancer in a subject.

145. The kit for use according to claim 144, wherein the tumor or cancer is as defined in any one of claims 1-140, and/or the subject is as defined in any one of claims 1-140, and/or the method is/are as defined in any one of claims 1-140.

146. A pharmaceutical composition comprising
i) a binding agent comprising at least one binding region binding to CD27;
ii) a PD1/PD-L1 inhibitor; and
iii) optionally a pharmaceutical acceptable carrier.

147. The pharmaceutical composition according to claim 146, wherein the binding agent is as defined in any one of claims 1-140 and/or the PD1/PD-L1 inhibitor is as defined in any one of claims 1-140.

148. The pharmaceutical composition according to claim 146 or 147 for use in a method for reducing progression or preventing progression of a tumor or treating cancer in a subject.

149. The pharmaceutical composition for use according to claim 148, wherein the tumor or cancer is as defined in any one of claims 1-140, and/or the subject is as defined in any one of claims 1-140, and/or the method is/are as defined in any one of claims 1-140.

150. A binding agent for use in a method for reducing progression or preventing progression of a tumor or treating cancer in a subject, said method comprising administering to said subject i) the binding agent comprising at least one binding region binding to CD27; and ii) a PD1/PD-L1 inhibitor

151. The binding agent for use according to claim 150, wherein the method is as defined in any one of claims 1-140, and/or the binding agent is as defined in any one of claims 1-140, and/or the PD1/PD-L1 inhibitor is as defined in any one of claims 1-140.

152. A PD1/PD-L1 inhibitor for use in a method for reducing progression or preventing progression of a tumor or treating cancer in a subject, said method comprising administering to said subject i) a binding agent comprising at least one binding region binding to CD27; and ii) the PD1/PD-L1 inhibitor.

153. The PD1/PD-L1 inhibitor for use according to claim 152, wherein the method is as defined in any one of claims 1-140, and/or the binding agent is as defined in any one of claims 1-140, and/or the PD1/PD-L1 inhibitor is as defined in any one of claims 1-140.


SEQUENCE LISTING (140 sequences)

SEQ ID NO: 1 - protein, 260 aa, Homo sapiens
MARPHPWWLCVLGTLVGLSATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRILVIFSGMFLVFTLAGALFLHQRRKYRSNKGESPVEPAEPCHYSCPREEEGSTIPIQEDYRKPEPACSP

SEQ ID NO: 2 - protein, 260 aa, synthetic construct
MARPHPWWLCVLGTLVGLSATPAPKSCPERHYWAQGKLCCQMCEPGTFLVKDCDQHRKTAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRILVIFSGMFLVFTLAGALFLHQRRKYRSNKGESPVEPAEPCHYSCPREEEGSTIPIQEDYRKPEPACSP

SEQ ID NO: 3 - protein, 260 aa, synthetic construct
MARPHPWWLCFLGTLVGLSATPAPKSCPERHYWAQGKLCCQMCEPGTFLVKDCDQHRKAAQCHPCIPGVSFSPDHHTRPHCESCRHCNSGLLIRNCTITANAVCACRNGWQCRDKECTECDPPPNPSLTTWPSQALGPHPQPTHLPYVNEMLEARTAGHMQTLADFRHLPARTLSTHWPPQRSLCSSDFIRILVIFSGMFLVFTLAGTLFLHQQRKYRSNKGESPMEPAEPCPYSCPREEEGSTIPIQEDYRKPEPASSP

SEQ ID NO: 4 - protein, 116 aa, synthetic construct
QVQLMQSGSELKKPGASVKVSCRASGYTFTTYAMNWVRQAPGQGPEWMGWINTNTGNPTYAQGFTGRFVFSLDTTVTTTYLQISSLKAEDTAVYFCAREAGSFDYWGQGTLVTVSS

SEQ ID NO: 5 - protein, 8 aa, synthetic construct
GYTFTTYA

SEQ ID NO: 6 - protein, 8 aa, synthetic construct
INTNTGNP

SEQ ID NO: 7 - protein, 9 aa, synthetic construct
AREAGSFDY

SEQ ID NO: 8 - protein, 110 aa, synthetic construct
QSALTQPASVSGSPGQSITISCTGTSSDVYYYNYVSWYQQHPGRAPKLVIYDVSNRPSGVSNRFSGSKSGNTASLTISGLRAEDEADYYCSSYTVNRVWVFGGGTKLTVL

SEQ ID NO: 9 - protein, 9 aa, synthetic construct
SSDVYYYNY

SEQ ID NO: 10 - skipped

SEQ ID NO: 11 - protein, 10 aa, synthetic construct
SSYTVNRVWV

SEQ ID NO: 12 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALRAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALRAPIEKTISKAKGQPRRPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 16 - protein, 107 aa, synthetic construct [sequence omitted]

SEQ ID NO: 17 - protein, 106 aa, synthetic construct
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 18 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 19 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKPVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKPVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEGLHNHYTQKSLSLSPGK

SEQ ID NO: 21 - protein, 326 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 22 - protein, 377 aa, synthetic construct
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 23 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 24 - protein, 446 aa, synthetic construct
QVQLMQSGSELKKPGASVKVSCRASGYTFTTYAMNWVRQAPGQGPEWMGWINTNTGNPTYAQGFTGRFVFSLDTTVTTTYLQISSLKAEDTAVYFCAREAGSFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALRAPIEKTISKAKGQPRRPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 216 aa, synthetic construct
QSALTQPASVSGSPGQSITISCTGTSSDVYYYNYVSWYQQHPGRAPKLVIYDVSNRPSGVSNRFSGSKSGNTASLTISGLRAEDEADYYCSSYTVNRVWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 26 - protein, 250 aa, Mus musculus
MAWPPPYWLCMLGTLVGLSATLAPNSCPDKHYWTGGGLCCRMCEPGTFFVKDCEQDRTAAQCDPCIPGTSFSPDYHTRPHCESCRHCNSGFLIRNCTVTANAECSCSKNWQCRDQECTECDPPLNPALTRQPSETPSPQPPPTHLPHGTEKPSWPLHRQLPNSTVYSQRSSHRPLCSSDCIRIFVTFSSMFLIFVLGAILFFHQRRNHGPNEDRQAVPEEPCPYSCPREEEGSAIPIQEDYRKPEPAFYP

SEQ ID NO: 27 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALRAPIEKTISKAKGQPRRPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 329 aa, synthetic construct; identical to SEQ ID NO: 18

SEQ ID NO: 29 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 30 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALRAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 31 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKPVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 32 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKPVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEGLHNHYTQKSLSLSPG

SEQ ID NO: 33 - protein, 325 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 34 - protein, 376 aa, synthetic construct
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPG

SEQ ID NO: 35 - protein, 445 aa, synthetic construct
QVQLMQSGSELKKPGASVKVSCRASGYTFTTYAMNWVRQAPGQGPEWMGWINTNTGNPTYAQGFTGRFVFSLDTTVTTTYLQISSLKAEDTAVYFCAREAGSFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALRAPIEKTISKAKGQPRRPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 36 - protein, 329 aa, synthetic construct; identical to SEQ ID NO: 15

SEQ ID NO: 37 - protein, 329 aa, synthetic construct; identical to SEQ ID NO: 18

SEQ ID NO: 38 - protein, 329 aa, synthetic construct
  VARIANT 117: note = X is A, V, L, I, P, F, M or W, preferably I, P, F, M or W, more preferably F
  VARIANT 118: note = X is L, D or E, preferably D or E, more preferably E
  VARIANT 119: note = X is not G, preferably K, R or H, more preferably R
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEXXXGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 39 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFERGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPG

SEQ ID NO: 40 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKSLSLSPG

SEQ ID NO: 41 - protein, 327 aa, synthetic construct; identical to SEQ ID NO: 23

SEQ ID NO: 42 - protein, 107 aa, synthetic construct [sequence omitted]

SEQ ID NO: 43 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 44 - protein, 10 aa, synthetic construct
AFYDDYDYNV

SEQ ID NO: 45 - protein, 12 aa, synthetic construct
ARAFYDDYDYNV

SEQ ID NO: 46 - protein, 7 aa, synthetic construct
ISGGTIG

SEQ ID NO: 47 - protein, 16 aa, synthetic construct
IISGGTIGHYASWAKG

SEQ ID NO: 48 - protein, 5 aa, synthetic construct
SYNMG

SEQ ID NO: 49 - protein, 8 aa, synthetic construct
GFSLYSYN

SEQ ID NO: 50 - protein, 11 aa, synthetic construct
AGGYSSSSDTT

SEQ ID NO: 51 - protein, 7 aa, synthetic construct
QASKLET

SEQ ID NO: 52 - protein, 8 aa, synthetic construct
QSVYGNNQ

SEQ ID NO: 53 - protein, 13 aa, synthetic construct
QSSQSVYGNNQLS

SEQ ID NO: 54 - protein, 116 aa, synthetic construct
QSVEESGGRLVTPGTPLTLTCTVSGFSLYSYNMGWVRQAPGKGLEYIGIISGGTIGHYASWAKGRFTISKTSSTTVDLKMTSLTTEDTATYFCARAFYDDYDYNVWGPGTLVTVSS

SEQ ID NO: 55 - protein, 111 aa, synthetic construct
AAVLTQTPSPVSAAVGGTVTISCQSSQSVYGNNQLSWYQQKPGQPPKLLIYQASKLETGVPSRFKGSGSGTQFTLTISDLESDDAATYYCAGGYSSSSDTTFGGGTEVVVK

SEQ ID NO: 56 - protein, 118 aa, synthetic construct
QVQLVESGGGLVQPGTSLRLSCSVSGFSLYSYNMGWVRQAPGKGLEYIGIISGGTIGHYASWAKGRFTISRDTSKTTLYLQMNSLTTEDTATYFCARAFYDDYDYNVWGPGTLVTVSS

SEQ ID NO: 57 - protein, 111 aa, synthetic construct
AIQLTQSPSSLSASVGGTVTITCQSSQSVYGNNQLSWYQQKPGQPPKLLIYQASKLETGVPSRFRGSGSGTQFTLTISSLQSEDFATYYCAGGYSSSSDTTFGGGTEVVVK

SEQ ID NO: 58 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 59 - protein, 147 aa, Homo sapiens
FLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLV

SEQ ID NO: 60 - DNA, 2115 bp, Homo sapiens
  source 1..2115: mol_type = other DNA
agtttcccttccgctcacctccgcctgagcagtggagaaggcggcactctggtggggctgctccaggcatgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccagttccaaaccctggtggttggtgtcgtgggcggcctgctgggcagcctggtgctgctagtctgggtcctggccgtcatctgctcccgggccgcacgagggacaataggagccaggcgcaccggccagcccctgaaggaggacccctcagccgtgcctgtgttctctgtggactatggggagctggatttccagtggcgagagaagaccccggagccccccgtgccctgtgtccctgagcagacggagtatgccaccattgtctttcctagcggaatgggcacctcatcccccgcccgcaggggctcagctgacggccctcggagtgcccagccactgaggcctgaggatggacactgctcttggcccctctgaccggcttccttggccaccagtgttctgcagaccctccaccatgagcccgggtcagcgcatttcctcaggagaagcaggcagggtgcaggccattgcaggccgtccaggggctgagctgcctgggggcgaccggggctccagcctgcacctgcaccaggcacagccccaccacaggactcatgtctcaatgcccacagtgagcccaggcagcaggtgtcaccgtcccctacagggagggccagatgcagtcactgcttcaggtcctgccagcacagagctgcctgcgtccagctccctgaatctctgctgctgctgctgctgctgctgctgctgcctgcggcccggggctgaaggcgccgtggccctgcctgacgccccggagcctcctgcctgaacttgggggctggttggagatggccttggagcagccaaggtgcccctggcagtggcatcccgaaacgccctggacgcagggcccaagactgggcacaggagtgggaggtacatggggctggggactccccaggagttatctgctccctgcaggcctagagaagtttcagggaaggtcagaagagctcctggctgtggtgggcagggcaggaaacccctccacctttacacatgcccaggcagcacctcaggccctttgtggggcagggaagctgaggcagtaagcgggcaggcagagctggaggcctttcaggcccagccagcactctggcctcctgccgccgcattccaccccagcccctcacaccactcgggagagggacatcctacggtcccaaggtcaggagggcagggctggggttgactcaggcccctcccagctgtggccacctgggtgttgggagggcagaagtgcaggcacctagggccccccatgtgcccaccctgggagctctccttggaacccattcctgaaattatttaaaggggttggccgggctcccaccagggcctgggtgggaaggtacaggcgttcccccggggcctagtacccccgccgtggcctatccactcctcacatccacacactgcacccccactcctggggcagggccaccagcatccaggcggccagcaggcacctgagtggctgggacaagggatcccccttccctgtggttctattatattataattataattaaatatgagagcatgctaaggaaaa

SEQ ID NO: 61 - protein, 121 aa, synthetic construct
QVQLQESGPGLVRPSQTLSLTCTVSGFTFTDFYMNWVRQPPGRGLEWIGFIRDKAKGYTTEYNPSVKGRVTMLVDTSKNQFSLRLSSVTAADTAVYYCAREGHTAAPFDYWGQGSLVTVSS

SEQ ID NO: 62 - protein, 8 aa, synthetic construct
GFTFTDFY

SEQ ID NO: 63 - protein, 10 aa, synthetic construct
IRDKAKGYTT

SEQ ID NO: 64 - protein, 12 aa, synthetic construct
AREGHTAAPFDY

SEQ ID NO: 65 - protein, 107 aa, synthetic construct
DIQMTQSPSSLSASVGDRVTITCKASQNIDKYLNWYQQKPGKAPKLLIYNTNNLQTGVPSRFSGSGSGTDFTFTISSLQPEDIATYYCLQHISRPRTFGQGTKVEIK

SEQ ID NO: 66 - protein, 6 aa, synthetic construct
QNIDKY

SEQ ID NO: 67 - protein, 9 aa, synthetic construct
LQHISRPRT

SEQ ID NO: 68 - protein, 127 aa, synthetic construct
QVQLVQSGAEVKKPGASVKVSCQASGYRFSNFVIHWVRQAPGQRFEWMGWINPYNGNKEFSAKFQDRVTFTADTSANTAYMELRSLRSADTAVYYCARVGPYSWDDSPQDNYYMDVWGKGTTVIVSS

SEQ ID NO: 69 - protein, 8 aa, synthetic construct
GYRFSNFV

SEQ ID NO: 70 - protein, 8 aa, synthetic construct
INPYNGNK

SEQ ID NO: 71 - protein, 20 aa, synthetic construct
ARVGPYSWDDSPQDNYYMDV

SEQ ID NO: 72 - protein, 108 aa, synthetic construct
EIVLTQSPGTLSLSPGERATFSCRSSHSIRSRRVAWYQHKPGQAPRLVIHGVSNRASGISDRFSGSGSGTDFTLTITRVEPEDFALYYCQVYGASSYTFGQGTKLERK

SEQ ID NO: 73 - protein, 7 aa, synthetic construct
HSIRSRR

SEQ ID NO: 74 - protein, 9 aa, synthetic construct
QVYGASSYT

SEQ ID NO: 75 - protein, 446 aa, synthetic construct
EVQLVESGGGLVQPGRSLRLSCTASGFSLNDYWMSWVRQAPGKGLEWVGYIDVGGSLYYAASVKGRFTISRDDSKSIAYLQMNSLKTEDTAVYYCARGGLTYGFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPG

SEQ ID NO: 76 - protein, 217 aa, synthetic construct
DIVMTQSPSSLSASVGDRVTITCQASEDISSYLAWYQQKPGKAPKRLIYGASDLASGVPSRFSASGSGTDYTFTISSLQPEDIATYYCHYYATISGLGVAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 77 - protein, 450 aa, synthetic construct
EVQLLEPGGGLVQPGGSLRLSCEASGSTFSTYAMSWVRQAPGKGLEWVSGFSGSGGFTFYADSVRGRFTISRDSSKNTLFLQMSSLRAEDTAVYYCAIPARGYNYGSFQHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPG

SEQ ID NO: 78 - protein, 214 aa, synthetic construct
SYVLTQPPSVSVAPGQTARITCGGNNIGSKSVHWYQQKPGQAPVLVVYDDNDRPSGLPERFSGSNSGNTATLTISRVEAGDEADYYCQVWDSSSDHVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 79 - protein, 117 aa, synthetic construct
EVQLVESGGGLVQPGRSLRLSCTASGFSLNDYWMSWVRQAPGKGLEWVGYIDVGGSLYYAASVKGRFTISRDDSKSIAYLQMNSLKTEDTAVYYCARGGLTYGFDLWGQGTLVTVSS

SEQ ID NO: 80 - protein, 8 aa, synthetic construct
GFSLNDYW

SEQ ID NO: 81 - protein, 7 aa, synthetic construct
IDVGGSL

SEQ ID NO: 82 - protein, 11 aa, synthetic construct
ARGGLTYGFDL

SEQ ID NO: 83 - protein, 110 aa, synthetic construct
DIVMTQSPSSLSASVGDRVTITCQASEDISSYLAWYQQKPGKAPKRLIYGASDLASGVPSRFSASGSGTDYTFTISSLQPEDIATYYCHYYATISGLGVAFGGGTKVEIK

SEQ ID NO: 84 - protein, 6 aa, synthetic construct
EDISSY

SEQ ID NO: 85 - protein, 12 aa, synthetic construct
HYYATISGLGVA

SEQ ID NO: 86 - protein, 121 aa, synthetic construct
EVQLLEPGGGLVQPGGSLRLSCEASGSTFSTYAMSWVRQAPGKGLEWVSGFSGSGGFTFYADSVRGRFTISRDSSKNTLFLQMSSLRAEDTAVYYCAIPARGYNYGSFQHWGQGTLVTVSS

SEQ ID NO: 87 - protein, 8 aa, synthetic construct
GSTFSTYA

SEQ ID NO: 88 - protein, 8 aa, synthetic construct
FSGSGGFT

SEQ ID NO: 89 - protein, 14 aa, synthetic construct
AIPARGYNYGSFQH

SEQ ID NO: 90 - protein, 108 aa, synthetic construct
SYVLTQPPSVSVAPGQTARITCGGNNIGSKSVHWYQQKPGQAPVLVVYDDNDRPSGLPERFSGSNSGNTATLTISRVEAGDEADYYCQVWDSSSDHVVFGGGTKLTVL

SEQ ID NO: 91 - protein, 6 aa, synthetic construct
NIGSKS

SEQ ID NO: 92 - protein, 11 aa, synthetic construct
QVWDSSSDHVV

SEQ ID NO: 93 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 94 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 95 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPG

SEQ ID NO: 96 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPG

SEQ ID NO: 97 - protein, 232 aa, Homo sapiens
LQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 98 - protein, 272 aa, Homo sapiens
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 99 - protein, 8 aa, synthetic construct
GYTFTNYY

SEQ ID NO: 100 - protein, 8 aa, synthetic construct
INPSNGGT

SEQ ID NO: 101 - protein, 13 aa, synthetic construct
ARRDYRFDMGFDY

SEQ ID NO: 102 - protein, 10 aa, synthetic construct
KGVSTSGYSY

SEQ ID NO: 103 - protein, 9 aa, synthetic construct
QHSRDLPLT

SEQ ID NO: 104 - protein, 120 aa, synthetic construct [sequence omitted]

SEQ ID NO: 105 - protein, 111 aa, synthetic construct [sequence omitted]

SEQ ID NO: 106 - protein, 446 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGLTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 107 - protein, 218 aa, synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 108 - protein, 440 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 109 - protein, 214 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 110 - protein, 448 aa, synthetic construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 111 - protein, 214 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 112 - protein, 29 aa, synthetic construct
QSVEESGGRLVTPGTPLTLTCTVSGFSLY

SEQ ID NO: 113 - protein, 14 aa, synthetic construct
WVRQAPGKGLEYIG

SEQ ID NO: 114 - protein, 31 aa, synthetic construct
RFTISKTSSTTVDLKMTSLTTEDTATYFCAR

SEQ ID NO: 115 - protein, 11 aa, synthetic construct
WGPGTLVTVSS

SEQ ID NO: 116 - protein, 23 aa, synthetic construct
AAVLTQTPSPVSAAVGGTVTISC

SEQ ID NO: 117 - protein, 15 aa, synthetic construct
WYQQKPGQPPKLLIY

SEQ ID NO: 118 - protein, 32 aa, synthetic construct
GVPSRFKGSGSGTQFTLTISDLESDDAATYYC

SEQ ID NO: 119 - protein, 10 aa, synthetic construct
FGGGTEVVVK

SEQ ID NO: 120 - protein, 30 aa, synthetic construct
QVQLVESGGGLVQPGTSLRLSCSVSGFSLY

SEQ ID NO: 121 - protein, 14 aa, synthetic construct
WVRQAPGKGLEYIG

SEQ ID NO: 122 - protein, 32 aa, synthetic construct
RFTISRDTSKTTLYLQMNSLTTEDTATYFCAR

SEQ ID NO: 123 - protein, 11 aa, synthetic construct
WGPGTLVTVSS

SEQ ID NO: 124 - protein, 23 aa, synthetic construct
AIQLTQSPSSLSASVGGTVTITC

SEQ ID NO: 125 - protein, 15 aa, synthetic construct
WYQQKPGQPPKLLIY

SEQ ID NO: 126 - protein, 32 aa, synthetic construct
GVPSRFRGSGSGTQFTLTISSLQSEDFATYYC

SEQ ID NO: 127 - protein, 10 aa, synthetic construct
FGGGTEVVVK

SEQ ID NO: 128 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFERGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 129 - protein, 289 aa, Homo sapiens
MRIFAVFIFMTYWHLLNAFTVTVPKDLYVVEYGSNMTIECFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 130 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 131 - protein, 392 aa, synthetic construct
QSALTQPASVSGSPGQSITISCTGTSSDVYYYNYVSWYQQHPGRAPKLVIYDVSNRPSGVSNRFSGSKSGNTASLTISGLRAEDEADYYCSSYTVNRVWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSSLGSSGVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 132 - protein, 529 aa, synthetic construct
QSALTQPASVSGSPGQSITISCTGTSSDVYYYNYVSWYQQHPGRAPKLVIYDVSNRPSGVSNRFSGSKSGNTASLTISGLRAEDEADYYCSSYTVNRVWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSSLEPTTEDLYFQSDNDGSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 133 - protein, 391 aa, synthetic construct
EIVLTQSPGTLSLSPGERATFSCRSSHSIRSRRVAWYQHKPGQAPRLVIHGVSNRASGISDRFSGSGSGTDFTLTITRVEPEDFALYYCQVYGASSYTFGQGTKLERKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECSLGSSGVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 134 - protein, 528 aa, synthetic construct
EIVLTQSPGTLSLSPGERATFSCRSSHSIRSRRVAWYQHKPGQAPRLVIHGVSNRASGISDRFSGSGSGTDFTLTITRVEPEDFALYYCQVYGASSYTFGQGTKLERKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECSLEPTTEDLYFQSDNDGSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 135 - protein, 392 aa, synthetic construct; identical to SEQ ID NO: 131

SEQ ID NO: 136 - protein, 529 aa, synthetic construct; identical to SEQ ID NO: 132

SEQ ID NO: 137 - protein, 390 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSDWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECSLGSSGVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 138 - protein, 527 aa, synthetic construct
DIQMTQSPASLSVSVGETVTITCRASENIRSNLAWYQQKQGKSPQLLVNVATNLADGVPSRFSGSGSGTQYSLKINSLQSEDFGTYYCQHYWGTTWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECSLEPTTEDLYFQSDNDGSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 139 - protein, 447 aa, synthetic construct
QVQLVESGGGLVQPGTSLRLSCSVSGFSLYSYNMGWVRQAPGKGLEYIGIISGGTIGHYASWAKGRFTISRDTSKTTLYLQMNSLTTEDTATYFCARAFYDDYDYNVWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFERGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 140 - protein, 218 aa, synthetic construct
AIQLTQSPSSLSASVGGTVTITCQSSQSVYGNNQLSWYQQKPGQPPKLLIYQASKLETGVPSRFRGSGSGTQFTLTISSLQSEDFATYYCAGGYSSSSDTTFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC